(19) United States
(12) Patent Application Publication
(10) Pub. No.: US 2013/0225524 A1
(43) Pub. Date: Aug. 29, 2013

(54) Chemical Compounds

(71) Applicants: Deping Chai, Collegeville, PA (US); Mariela Colon, Collegeville, PA (US); Christopher Dodson, Collegeville, PA (US); Kevin J. Duffy, Collegeville, PA (US); Antony Nicholas Shaw, Collegeville, PA (US)

(72) Inventors: Deping Chai, Collegeville, PA (US); Mariela Colon, Collegeville, PA (US); Christopher Dodson, Collegeville, PA (US); Kevin J. Duffy, Collegeville, PA (US); Antony Nicholas Shaw, Collegeville, PA (US)

(21) Appl. No.: 13/883,023

(22) PCT Filed: Nov. 3, 2011
(86) PCT No.: PCT/US11/59067

§ 371 Date: May 2, 2013

Related U.S. Application Data
(60) Provisional application No. 61/410,434, filed on Nov. 5, 2010.

(51) Int. Cl. A61K 31/506; A61K 31/4709; A61K 31/4706; A61K 31/496; A61K 31/513; A61K 31/69; A61K 31/517; C07D 401/10; C07D 413/10; C07D 215/54; C07D 401/14; C07D 405/14; C07D 471/04; C07F 5/02; C07D 413/14; A61K 45/06

(52) U.S. Cl. CPC A61K 31/506; A61K 45/06; A61K 31/4709; A61K 31/4706; A61K 31/496; A61K 31/513; A61K 31/69; A61K 31/517; C07D 401/10; C07D 413/10; C07D 215/54; C07D 401/14; C07D 405/14; C07D 471/04; C07F 5/025; C07D 413/14
USPC 514/64; 544/310; 546/161; 544/363; 544/333; 546/113; 546/13; 544/284; 544/123; 514/274; 514/313; 514/253.06; 514/256; 514/300; 514/266.21

(57) ABSTRACT

The invention is directed to substituted quinoline derivatives. Specifically, the invention is directed to compounds according to Formula I:

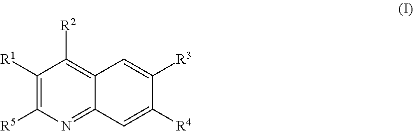

wherein R¹, R², R³, R⁴, and R⁵ are defined herein.

The compounds of the invention are inhibitors of lactate dehydrogenase A and can be useful in the treatment of cancer and diseases associated with tumor cell metabolism, such as cancer, and more specifically cancers of the breast, colon, prostate and lung. Accordingly, the invention is further directed to pharmaceutical compositions comprising a compound of the invention. The invention is still further directed to methods of inhibiting lactate dehydrogenase A activity and treatment of disorders associated therewith using a compound of the invention or a pharmaceutical composition comprising a compound of the invention.

### FIELD OF THE INVENTION

The present invention relates to substituted quinoline derivatives that are inhibitors of the activity of lactate dehydrogenase A (LDH-A). The present invention also relates to pharmaceutical compositions comprising such compounds and methods of using such compounds in the treatment of cancer.

### BACKGROUND OF THE INVENTION

The Warburg effect, also known as aerobic glycolysis, is defined as a high rate of glucose utilization and lactate production despite the presence of sufficient oxygen to oxidize glucose carbon in the mitochondria. Recognition of this unusual metabolic phenomenon stems from experiments performed by the German physiologist Otto Warburg, starting in the 1920s. Today, the glycolytic activity of tumors is exploited clinically by ¹⁸F-deoxyglucose positron emission tomography (FDG-PET), which detects tumors precisely by virtue of their enhanced ability to take up and metabolize glucose compared to normal tissue. The Warburg effect remains the most frequently cited evidence that tumors display dysfunctional metabolism.

The adenosine triphosphate (ATP) required for normal cell growth and survival comes from two main sources: glycolysis and mitochondrial oxidative phosphorylation. Glycolysis occurs by the Embden-Meyerhof pathway that requires NAD+ and involves a series of cytoplasmic enzymes that convert glucose to pyruvate. In oxygenated cells, pyruvate enters the mitochondria and is converted to acetyl-CoA by pyruvate dehydrogenase complex (PDC). This, along with acetyl-CoA produced from fatty acid oxidation, enters the Krebs cycle producing the electron donors NADH and FADH₂. NADH donates electrons to complex I of the electron transport chain (ETC), whilst FADH₂ donates electrons to complex III. Oxygen acts as the final electron acceptor. This process of oxidative phosphorylation produces 36 ATP molecules per glucose molecule. However, in conditions of limited oxygen, pyruvate is converted into lactic acid by lactate dehydrogenase (LDH) in a process termed anaerobic glycolysis. In contrast to oxidative phosphorylation, glycolysis is relatively inefficient, producing only two molecules of ATP per molecule of glucose, but with the regeneration of NAD⁺ to enable glycolysis to continue. The reduction of pyruvate to lactate is facilitated by the increased activity of two key enzymes: pyruvate dehydrogenase kinase 1 (PDK1), which blocks PDH activity, and lactate dehydrogenase A (LDHA), which converts cytosolic pyruvate to lactate. The decrease in the rate of pyruvate entering the TCA cycle and the concurrent increase in lactate production is vital for the growth and survival of tumors. Inhibition of LDHA results in the stimulation of mitochondrial respiration as a compensatory mechanism. Although mitochondrial respiration increases as a result of LDHA inhibition, the proliferative and tumorigenic potential of cancer cells is diminished.

In cancer patients, serum total lactate dehydrogenase (LDH5, a tetramer of LDHA sub-units; the major LDH isoenzyme involved in glycolysis) levels are often increased, and the gene for LDHA, is up-regulated. These features have been linked to poor prognosis in several studies.

Therefore, because of its importance in tumor cell metabolism, LDHA is an attractive oncology target. Inhibition of this enzyme is expected to reduce the ability of the cell to effectively metabolize glucose and reduce tumor cell proliferation and tumor growth.

### REFERENCES

Warburg et al., The metabolism of tumors in the body, J. Gen. Physiol. 8 (1927), pp. 519-530.
Warburg O. On the origin of cancer cells. Science. 1956 Feb. 24; 123 (3191):309-314.
Warburg O. On respiratory impairment in cancer cells. Science. 1956 Aug. 10; 124 (3215):269-270.
Ferreira, L. M. R., Cancer metabolism: The Warburg effect today, Exp. Mol. Pathol. (2010), doI:10.1016/j.yexmp.2010.08.006
Thomas N Seyfried and Laura M Shelton. Cancer as a metabolic disease. Nutr Metab (Lond). 2010 Jan. 27; 7:7.
Fantin, V. R., St-Pierre, J. & Leder, P. Attenuation of LDH-A expression uncovers a link between glycolysis, mitochondrial physiology, and tumor maintenance. Cancer Cell 9, 425-434 (2006).
Sonveaux, P. et al. Targeting lactate-fueled respiration selectively kills hypoxic tumor cells in mice. J. Clin. Invest. 118, 3930-3942 (2008).
Han Xie, Vladimir A. Valera, Maria J. Merino, Angela M. Amato, Sabina Signoretti, William M. Linehan, Vikas P. Sukhatme, and Pankaj Seth. LDH-A inhibition, a therapeutic strategy for treatment of hereditary leiomyomatosis and renal cell cancer. Mol. Cancer. Ther. 2009, 8(3), 626.
Le, A.; Cooper, C. R.; Gouw, A. M.; Dinavahi, R.; Maitra, A.; Deck, L. M.; Royer, R. E.; Vander Jagt, D. L.; Semenza, G. L.; Dang, C. V. Inhibition of lactate dehydrogenase A induces oxidative stress and inhibits tumor progression. Proc. Natl. Acad. Sci USA, 2010, 107(5), 2037-2042.
Granchi C, Bertini S, Macchia M, Minutolo F. Inhibitors of lactate dehydrogenase isoforms and their therapeutic potentials. Curr Med. Chem. 2010; 17(7):672-97.
Koukourakis M I, Kontomanolis E, Giatromanolaki A, Spyridis E, Liberis V. Serum and tissue LDH levels in patients with breast/gynaecological cancer and benign diseases. Gynecol Obstet. Invest. 2009; 67(3):162-8.
Koukourakis M I, Giatromanolaki A, Winter S, Leek R, Spyridis E, Harris A L. Lactate dehydrogenase 5 expression in squamous cell head and neck cancer relates to prognosis following radical or postoperative radiotherapy. Oncology. 2009; 77(5):285-92
Koukourakis, M. I.; Giatromanolaki, A.; Spyridis, E.; Bougioukas, G.; Didilis, V.; Gatter, K. C.; Harris, A. L. Lactate dehydrogenase-5 (LDH-5) overexpression in non-small-cell lung cancer tissues is linked to tumour hypoxia, angiogenic factor production and poor prognosis. Br J. Cancer. 2003 Sep. 1; 89(5):877-85

It is an object of the instant invention to provide novel compounds that are inhibitors of lactate dehydrogenase A (LDH-A).

It is also an object of the present invention to provide pharmaceutical compositions that comprise a pharmaceutical carrier and compounds useful in the methods of the invention.

It is also an object of the present invention to provide a method for treating cancer that comprises administering such inhibitors of lactate dehydrogenase A (LDH-A) activity.

### SUMMARY OF THE INVENTION

The invention is directed to substituted quinoline derivatives. Specifically, the invention is directed to compounds according to Formula I:

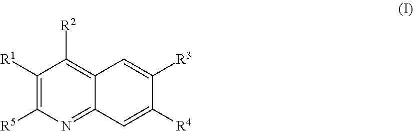

wherein R¹, R², R³, R⁴ and R⁵ are defined below.

This invention relates to a method of treating cancer, which comprises administering to a subject in need thereof an effective amount of a LDH-A inhibiting compound of Formula (I).

This invention relates to a method of treating solid tumors which comprises administering to a subject in need thereof an effective amount of a LDH-A inhibiting compound of Formula (I).

The present invention also relates to the discovery that the compounds of Formula (I) are active as inhibitors of LDH-A.

In a further aspect of the invention there is provided novel processes and novel intermediates useful in preparing the presently invented LDH-A inhibiting compounds.

Included in the present invention are pharmaceutical compositions that comprise a pharmaceutical carrier and compounds useful in the methods of the invention.

Also included in the present invention are methods of co-administering the presently invented LDH-A inhibiting compounds with further active ingredients.

### DETAILED DESCRIPTION OF THE INVENTION

This invention relates to novel compounds of Formula (I):

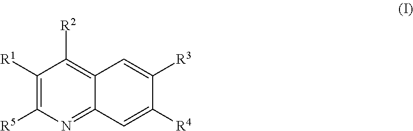

wherein:
R¹ is selected from: —COOH, —CONH₂, —SO₂NH₂, —CONHCH₂R¹⁰, —NHCONH₂, —CONCH₂thiazole and tetrazole,
   where R¹⁰ is selected from:
   —COOH,
   phenyl,
   phenyl substituted with from one to three substituents independently selected from: OH, —COOH, —NH₂, —OCH₃, —SO₂NH₂, —NHCOCH₃ and —CF₃,
   cyclopentyl, and
   cyclopentyl substituted with from one to three substituents independently selected from: OH, —COOH, —NH₂, —OCH₃, —SO₂NH₂, —NHCOCH₃ and —CF₃;
R² is selected from: —NHphenyl and —NHpyridinyl,
   where
   the pyridinyl is substituted with from one to three substituents independently selected from: —COOH, C₁-C₆alkyl —F, —Cl, —Br, and —I,
   the phenyl is substituted with from one to three substituents independently selected from:
      C₁-C₆alkyl,
      —F,
      —Cl,
      —Br,
      —I,
      OH,
      —COOH,
      phenyl,
      phenyl substituted with from one to three substituents independently selected from: C₁-C₆alkyl, —OCH₃, —CF₃, —F, —Cl, —Br and —I,
      heteroaryl,
      heteroaryl substituted with from one to three substituents independently selected from: C₁-C₆alkyl, —OCH₃, —CF₃, —F, —Cl, —Br and —I,
      cycloalkyl,
      cycloalkyl substituted with from one to three substituents independently selected from: C₁-C₆alkyl, —OCH₃, —CF₃, —F, —Cl, —Br and —I,
      heterocycloalkyl,
      heterocylcoalkyl substituted with from one to three substituents
      independently selected from: C₁-C₆alkyl, —OCH₃, —CF₃, —F, —Cl, —Br and —I,
      —B(OH)₂,
      —NHS(O)₂CF₃,
      —NHS(O)₂phenyl,
      —NHS(O)₂CH₂phenyl,
      —S(O)₂OH,
      —CH₂COOH,
      —CO₂CH₃,
      —CO₂CH₂CH₃,
      —NH₂,
      —OCH₃,
      —CO₂-ethyl,
      —NO₂,
      —NH₂,
      —NHCONH₂,
      —NHCOCF₃,
      —C≡N,
      —CH₃,
      —CONH₂, and
      —CF₃;
R³ is selected from:
   hydrogen,
   —OCH₃,
   —F,
   —Cl,
   —Br,
   —I,
   pyrimidinyl,
   pyrimidinyl substituted with from one to three substituents independently selected from: —OCH₃ and OH, and CH₃;
R⁴ is selected from: —F, —Cl, —Br, —I, —CH═CH₂, aryl, substituted aryl, heteroaryl,
   substituted heteroaryl, heterocycloalkyl, and substituted heterocycloalkyl; and
R⁵ is selected from: H, C₁-C₄alkyl, and —CF₃;
   or a salt thereof;
   provided the compound is not 3-{[3-(aminocarbonyl)-7-(3,5-dimethyl-4-isoxazolyl)-6-(methyloxy)-4-quinolinyl]amino}benzoic acid.

Included among the presently invented compounds of Formula (I) are those in which:
R¹ is selected from: —COOH, —CONH₂, —SO₂NH₂, —CONHCH₂R¹⁰, —NHCONH₂, —CONCH₂thiazole and tetrazole,
   where R¹⁰ is selected from:
   —COOH,
   phenyl,
   phenyl substituted with from one to three substituents independently selected from: OH, —COOH, —NH₂, —OCH₃, —SO₂NH₂, —NHCOCH₃ and —CF₃,
   cyclopentyl, and
   cyclopentyl substituted with from one to three substituents independently selected from: OH, —COOH, —NH₂, —OCH₃, —SO₂NH₂, —NHCOCH₃ and —CF₃;
R² is selected from: —NHphenyl and —NHpyridinyl,
   where
   the pyridinyl is substituted with from one to three substituents independently selected from: —COOH, C₁-C₆alkyl —F, —Cl, —Br, and —I,
   the phenyl is substituted with from one to three substituents independently selected from:
      C₁-C₆alkyl,
      —F,
      —Cl,
      —Br,
      —I,
      OH,
      —COOH,
      phenyl,
      phenyl substituted with from one to three substituents independently selected from: C₁-C₆alkyl, —OCH₃, —CF₃, —F, —Cl, —Br and —I,
      cyclopentyl,
      cyclopentyl substituted with from one to three substituents independently selected from: C₁-C₆alkyl, —OCH₃, —CF₃, —F, —Cl, —Br and —I,
      cyclohexyl,
      cyclohexyl substituted with from one to three substituents independently selected from: C₁-C₆alkyl, —OCH₃, —CF₃, —F, —Cl, —Br and —I,
      cyclohexen,
      cyclohexen substituted with from one to three substituents
      independently selected from: C₁-C₆alkyl, —OCH₃, —CF₃, —F, —Cl, —Br and —I,
      cyclopenten,
      cyclopenten substituted with from one to three substituents independently selected from: C₁-C₆alkyl, —OCH₃, —CF₃, —F, —Cl, —Br and —I,
      furanyl,
      furanyl substituted with from one to three substituents independently selected from: C₁-C₆alkyl, —OCH₃, —CF₃, F, —Cl, —Br and —I,
      pyran,
      pyran substituted with from one to three substituents independently selected from: C₁-C₆alkyl, —OCH₃, —CF₃, —F, —Cl, —Br and —I,
      dihydropyran,
      dihydropyran substituted with from one to three substituents independently selected from: C₁-C₆alkyl, —OCH₃, —CF₃, —F, —Cl, —Br and —I,
      tetrahydropyran,
      tetrahydropyran substituted with from one to three substituents independently selected from: C₁-C₆alkyl, —OCH₃, —CF₃, —F, —Cl, —Br and —I,
      tetrahydrofuranyl,
      tetrahydrofuranyl substituted with from one to three substituents independently selected from: C₁-C₆alkyl, —OCH₃, —CF₃, —F, —Cl, —Br and —I,
      piperidinyl,
      piperidinyl substituted with from one to three substituents independently selected from: C₁-C₆alkyl, —OCH₃, —CF₃, —F, —Cl, —Br and —I,
      pyridinyl,
      pyridinyl substituted with from one to three substituents independently selected from: —OCH₃, C₁-C₆alkyl and —CF₃,
      -tetrazole,
      morpholinyl,
      piperazinyl,
      piperazinyl substituted with —CH₃,
      —B(OH)₂,
      —NHS(O)₂CF₃,
      —NHS(O)₂phenyl,
      —NHS(O)₂CH₂phenyl,
      S(O)₂OH,
      —CH₂COOH,
      —CO₂CH₃,
      —CO₂CH₂CH₃,
      —NH₂,
      —OCH₃,
      —CO₂ethyl,
      —NO₂,
      —NH₂,
      —NHCONH₂,
      —NHCOCF₃,
      —C≡N,
      —CH₃,
      —CONH₂, and
      —CF₃;
R³ is selected from:
   hydrogen,
   —OCH₃,
   —F,
   —Cl,
   —Br,
   —I,
   pyrimidinyl,
   pyrimidinyl substituted with from one to three substituents independently selected from: —OCH₃ and OH, and CH₃;
R⁴ is selected from: —F, —Cl, —Br, —I, —CH═CH₂, aryl, substituted aryl, heteroaryl,
   substituted heteroaryl, heterocycloalkyl, and substituted heterocycloalkyl, where,
      aryl is selected from: phenyl, naphthalene, tetrahydronaphthalene and biphenyl,
      heteroaryl is selected form: pyridinyl, pyrazol, pyrimidinyl, isoxazolyl, oxazole, quinolinyl, quinazolinyl, indiazole, indole, thienyl, furan, pyrazin, pyridazine, imidazole, pyrrolopyridin, and thiazole, and
      heterocycloalkyl is selected from: piperazine and pyrrolidine; and
R⁵ is selected from: H, C₁-C₄alkyl, and —CF₃;
   or a salt thereof;
   provided the compound is not 3-{[3-(aminocarbonyl)-7-(3,5-dimethyl-4-isoxazolyl)-6-(methyloxy)-4-quinolinyl]amino}benzoic acid.

Included among the presently invented compounds of Formula (I) are those in which:
R¹ is selected from: —COOH, —CONH₂, —SO₂NH₂, —CONHCH₂R¹⁰, —NHCONH₂, —CONCH₂thiazole and tetrazole,
   where R¹⁰ is selected from:
   —COOH,
   phenyl,
   phenyl substituted with from one to three substituents independently selected from: OH, —COOH, —NH₂, —OCH₃, —SO₂NH₂, —NHCOCH₃ and —CF₃,
   cyclopentyl, and
   cyclopentyl substituted with from one to three substituents independently selected from: OH, —COOH, —NH₂, —OCH₃, —SO₂NH₂, —NHCOCH₃ and —CF₃;
R² is selected from: —NHphenyl and —NHpyridinyl,
   where
   the pyridinyl is substituted with from one to three substituents independently selected from: —COOH, C₁-C₆alkyl —F, —Cl, —Br, and —I,
   the phenyl is substituted with from one to three substituents independently selected from:
      C₁-C₆alkyl,
      —F,
      —Cl,
      —Br,
      —I,
      —OH,
      —COOH,
      phenyl,
      phenyl substituted with from one to three substituents independently selected from: C₁-C₆alkyl, —OCH₃, —CF₃, —F, —Cl, —Br and —I,
      cyclopentyl,
      cyclohexyl,
      cyclohexen,
      cyclopenten,
      furanyl,
      pyran,
      dihydropyran,
      tetrahydropyran,
      tetrahydrofuranyl,
      piperidinyl,
      pyridinyl,
      pyridinyl substituted with from one to three substituents independently selected from: —OCH₃, C₁-C₆alkyl and —CF₃,
      -tetrazole,
      morpholinyl,
      piperazinyl,
      piperazinyl substituted with CH₃,
      —B(OH)₂,
      —NHS(O)₂CF₃,
      —NHS(O)₂phenyl,
      —NHS(O)₂CH₂phenyl,
      —S(O)₂OH,
      —CH₂COOH,
      —CO₂CH₃,
      —CO₂CH₂CH₃,
      —NH₂,
      —OCH₃,
      —CO₂ethyl,
      —NO₂,
      —NH₂,
      —NHCONH₂,
      —NHCOCF₃,
      —C≡N,
      —CH₃,
      —CONH₂, and
      —CF₃;
R³ is selected from:
   hydrogen,
   —OCH₃,
   —F,
   —Cl,
   —Br,
   —I,
   pyrimidinyl,
   pyrimidinyl substituted with from one to three substituents independently selected from: —OCH₃ and OH, and CH₃;
R⁴ is selected from: —F, —Cl, —Br, —I, —CH═CH₂, aryl, substituted aryl, heteroaryl,
   substituted heteroaryl, heterocycloalkyl, and substituted heterocycloalkyl,
      where,
      aryl is selected from: phenyl, naphthalene, tetrahydronaphthalene and biphenyl,
      heteroaryl is selected form: pyridinyl, pyrazol, pyrimidinyl, isoxazolyl, oxazole, quinolinyl, quinazolinyl, indiazole, indole, thienyl, furan, pyrazin, pyridazine, imidazole, pyrrolopyridin, and thiazole, heterocycloalkyl is selected from: piperazine and pyrrolidine; and
R⁵ is selected from: H, C₁-C₄alkyl, and —CF₃;
   or a salt thereof;
   provided the compound is not 3-{[3-(aminocarbonyl)-7-(3,5-dimethyl-4-isoxazolyl)-6-(methyloxy)-4-quinolinyl]amino}benzoic acid.

Included in the presently invented compounds of Formula (I) are:
5-({3-(aminocarbonyl)-7-[2,4-bis(methyloxy)-5-pyrimidinyl]-4-quinolinyl}amino)-3-biphenylcarboxylic acid;
5-({3-(aminocarbonyl)-7-[2,6-bis(methyloxy)-3-pyridinyl]-4-quinolinyl}amino)-1,3-benzenedicarboxylic acid;
3-({3-(aminosulfonyl)-7-[2,4-bis(methyloxy)-5-pyrimidinyl]-4-quinolinyl}amino)-5-cyclopentylbenzoic acid;
5-({3-(aminocarbonyl)-7-[2,4-bis(methyloxy)-5-pyrimidinyl]-4-quinolinyl}amino)-1,3-benzenedicarboxylic acid;
5-{[3-(aminocarbonyl)-7-(3,5-dimethyl-1H-pyrazol-4-yl)-4-quinolinyl]amino}-1,3-benzenedicarboxylic acid;
3-({3-(aminocarbonyl)-7-[2,4-bis(methyloxy)-5-pyrimidinyl]-4-quinolinyl}amino)benzoic acid;
5-{[3-(aminocarbonyl)-7-(3,5-dimethyl-4-isoxazolyl)-4-quinolinyl]amino}-1,3-benzenedicarboxylic acid;
3-({3-(aminocarbonyl)-7-[2,6-bis(methyloxy)-3-pyridinyl]-4-quinolinyl}amino)benzoic acid;
3-{[3-(aminocarbonyl)-7-(3,5-dimethyl-1H-pyrazol-4-yl)-6-(methyloxy)-4-quinolinyl]amino}benzoic acid;
3-{[3-(aminocarbonyl)-7-(3,5-dimethyl-1H-pyrazol-4-yl)-4-quinolinyl]amino}benzoic acid;
5-{[3-({[(3-carboxyphenyl)methyl]amino}carbonyl)-7-(3,5-dimethyl-1H-pyrazol-4-yl)-4-quinolinyl]amino}-1,3-benzenedicarboxylic acid;
3-{[3-(aminocarbonyl)-6-(methyloxy)-7-(4-pyridinyl)-4-quinolinyl]amino}benzoic acid;
3-[({[4-[(3-carboxyphenyl)amino]-7-(3,5-dimethyl-4-isoxazolyl)-3-quinolinyl]carbonyl}amino)methyl]benzoic acid;
3-{[3-(aminocarbonyl)-7-(1,3,5-trimethyl-1H-pyrazol-4-yl)-4-quinolinyl]amino}benzoic acid;
3-({7-(3,5-dimethyl-4-isoxazolyl)-3-[({[3-(trifluoromethyl)phenyl]methyl}amino)carbonyl]-4-quinolinyl}amino)benzoic acid;
3-{[3-(aminocarbonyl)-7-(3-methyl-1H-pyrazol-4-yl)-4-quinolinyl]amino}benzoic acid;
3-{[3-(aminocarbonyl)-7-(4-hydroxyphenyl)-4-quinolinyl]amino}benzoic acid;
3-{[3-(aminocarbonyl)-7-(3,5-dimethyl-4-isoxazolyl)-4-quinolinyl]amino}-5-(methyloxy)benzoic acid;
3-{[3-[({[3,5-bis(methyloxy)phenyl]methyl}amino)carbonyl]-7-(3,5-dimethyl-4-isoxazolyl)-4-quinolinyl]amino}benzoic acid;
3-{[3-(aminocarbonyl)-7-(3,5-dimethyl-1H-pyrazol-4-yl)-4-quinolinyl]amino}-5-[(methyloxy)carbonyl]benzoic acid;
3-{[3-(aminocarbonyl)-7-(3,5-dimethyl-4-isoxazolyl)-4-quinolinyl]amino}benzoic acid;
3-{[3-(aminocarbonyl)-7-(4-pyridinyl)-4-quinolinyl]amino}benzoic acid;
3-({3-(aminocarbonyl)-7-[4-(methyloxy)phenyl]-4-quinolinyl}amino)benzoic acid;
3-({3-(aminocarbonyl)-7-[2-(methyloxy)phenyl]-4-quinolinyl}amino)benzoic acid;
3-{[3-(aminocarbonyl)-7-(2-methyl-3-pyridinyl)-4-quinolinyl]amino}benzoic acid;
3-({3-(aminocarbonyl)-7-[2-chloro-6-(methyloxy)-3-pyridinyl]-4-quinolinyl}amino)benzoic acid;
3-{[3-(aminocarbonyl)-7-(2,5-difluorophenyl)-4-quinolinyl]amino}benzoic acid;
3-{[3-(aminocarbonyl)-7-(3,5-dimethyl-4-isoxazolyl)-4-quinolinyl]amino}-5-nitrobenzoic acid;
3-{[3-(aminocarbonyl)-7-(4-methyl-3-pyridinyl)-4-quinolinyl]amino}benzoic acid;
3-({3-(aminocarbonyl)-7-[2-(methyloxy)-3-pyridinyl]-4-quinolinyl}amino)benzoic acid;
3-{[3-(aminocarbonyl)-7-phenyl-4-quinolinyl]amino}benzoic acid;
3-{[3-(aminocarbonyl)-7-(1,3-oxazol-2-yl)-4-quinolinyl]amino}benzoic acid;
3-{[3-(aminocarbonyl)-7-(2-pyridinyl)-4-quinolinyl]amino}benzoic acid;
3-amino-5-{[3-(aminocarbonyl)-7-(3,5-dimethyl-4-isoxazolyl)-4-quinolinyl]amino}benzoic acid;
3-({3-(aminocarbonyl)-7-[3-(methyloxy)phenyl]-4-quinolinyl}amino)benzoic acid;
5-{[3-(aminocarbonyl)-7-(3,5-dimethyl-4-isoxazolyl)-4-quinolinyl]amino}-2-hydroxybenzoic acid;
5-{[3-(aminocarbonyl)-7-(3,5-dimethyl-4-isoxazolyl)-4-quinolinyl]amino}-2-methylbenzoic acid;
3-({3-(aminocarbonyl)-7-[2-(methyloxy)-4-pyridinyl]-4-quinolinyl}amino)benzoic acid;
3-({3-(aminocarbonyl)-7-[2,4-bis(methyloxy)phenyl]-4-quinolinyl}amino)benzoic acid;
3-{[3-(aminocarbonyl)-7-(1-methyl-1H-pyrazol-4-yl)-4-quinolinyl]amino}benzoic acid;
3-({7-(3,5-dimethyl-4-isoxazolyl)-3-[({[4-(methyloxy)phenyl]methyl}amino)carbonyl]-4-quinolinyl}amino)benzoic acid;
3-{[3-(aminocarbonyl)-7-(3,5-dimethyl-4-isoxazolyl)-4-quinolinyl]amino}-4-(methyloxy)benzoic acid;
3-{[3-(aminocarbonyl)-6-(methyloxy)-7-phenyl-4-quinolinyl]amino}benzoic acid;
3-{[3-(aminocarbonyl)-7-(2-methyl-4-pyridinyl)-4-quinolinyl]amino}benzoic acid;
3-{[3-(aminocarbonyl)-7-(3-hydroxyphenyl)-4-quinolinyl]amino}benzoic acid;
3-({3-(aminocarbonyl)-7-[2-(1-piperazinyl)-4-pyridinyl]-4-quinolinyl}amino)benzoic acid;
3-{[3-(aminocarbonyl)-7-(1-methyl-1H-indazol-7-yl)-4-quinolinyl]amino}benzoic acid;
3-{[3-(aminocarbonyl)-7-(5-pyrimidinyl)-4-quinolinyl]amino}benzoic acid;
3-({3-(aminocarbonyl)-7-[6-(aminocarbonyl)-3-pyridinyl]-4-quinolinyl}amino)benzoic acid;
3-({3-(aminocarbonyl)-7-[2,4-bis(methyloxy)-5-pyrimidinyl]-4-quinolinyl}amino)-5-[2,3-bis(methyloxy)-4-pyridinyl]benzoic acid;
3-({3-(aminocarbonyl)-7-[2,4-bis(methyloxy)-5-pyrimidinyl]-4-quinolinyl}amino)-5-(2-furanyl)benzoic acid;
3-({3-(aminosulfonyl)-7-[2,4-bis(methyloxy)-5-pyrimidinyl]-4-quinolinyl}amino)benzoic acid;
3-({3-(aminocarbonyl)-7-[2,4-bis(methyloxy)-5-pyrimidinyl]-4-quinolinyl}amino)-5-cyclopentylbenzoic acid;
3-({3-(aminocarbonyl)-7-[2,4-bis(methyloxy)-5-pyrimidinyl]-4-quinolinyl}amino)-5-cyclohexylbenzoic acid;
3-({3-(aminocarbonyl)-7-[2,4-bis(methyloxy)-5-pyrimidinyl]-4-quinolinyl}amino)-5-[2-methyl-6-(methyloxy)-4-pyridinyl]benzoic acid;
3-({3-(aminocarbonyl)-7-[2,4-bis(methyloxy)-5-pyrimidinyl]-4-quinolinyl}amino)-5-(2-methylpropyl)benzoic acid;
5-({3-(aminocarbonyl)-7-[2,4-bis(methyloxy)-5-pyrimidinyl]-4-quinolinyl}amino)-3′,4′-bis(methyloxy)-3-biphenylcarboxylic acid;
3-({3-(aminocarbonyl)-7-[2,4-bis(methyloxy)-5-pyrimidinyl]-4-quinolinyl}amino)-5-(1,2-dimethyl-1-propen-1-yl)benzoic acid;
3-({3-(aminocarbonyl)-7-[2,4-bis(methyloxy)-5-pyrimidinyl]-4-quinolinyl}amino)-5-(1-cyclohexen-1-yl)benzoic acid;
3-({3-(aminocarbonyl)-7-[2,4-bis(methyloxy)-5-pyrimidinyl]-4-quinolinyl}amino)-5-(1,2-dimethylpropyl)benzoic acid;
3-({3-(aminocarbonyl)-7-[2,4-bis(methyloxy)-5-pyrimidinyl]-4-quinolinyl}amino)-5-(3-furanyl)benzoic acid;
3-({3-(aminocarbonyl)-7-[2,4-bis(methyloxy)-5-pyrimidinyl]-4-quinolinyl}amino)-5-(1-cyclopenten-1-yl)benzoic acid;
3-({3-(aminocarbonyl)-7-[2,4-bis(methyloxy)-5-pyrimidinyl]-4-quinolinyl}amino)-5-(2-methyl-1-propen-1-yl)benzoic acid;
3-({3-(aminocarbonyl)-7-[2,4-bis(methyloxy)-5-pyrimidinyl]-4-quinolinyl}amino)-5-ethylbenzoic acid;
3-({3-(aminocarbonyl)-7-[2,4-bis(methyloxy)-5-pyrimidinyl]-4-quinolinyl}amino)-5-(1-methylethyl)benzoic acid;
3-({3-(aminocarbonyl)-7-[2,4-bis(methyloxy)-5-pyrimidinyl]-4-quinolinyl}amino)-5-(3,6-dihydro-2H-pyran-4-yl)benzoic acid;
3-({3-(aminocarbonyl)-7-[2,4-bis(methyloxy)-5-pyrimidinyl]-4-quinolinyl}amino)-5-[2-(methyloxy)-6-(trifluoromethyl)-4-pyridinyl]benzoic acid;
3-({3-(aminocarbonyl)-7-[2,4-bis(methyloxy)-5-pyrimidinyl]-4-quinolinyl}amino)-5-(tetrahydro-2H-pyran-4-yl)benzoic acid;
3-({3-(aminocarbonyl)-7-[2,4-bis(methyloxy)-5-pyrimidinyl]-4-quinolinyl}amino)-5-(4-pyridinyl)benzoic acid;
5-({3-(aminocarbonyl)-7-[2,4-bis(methyloxy)-5-pyrimidinyl]-4-quinolinyl}amino)-4′-(1,1-dimethylethyl)-3-biphenylcarboxylic acid;
5-({3-(aminocarbonyl)-7-[2,4-bis(methyloxy)-5-pyrimidinyl]-4-quinolinyl}amino)-4′-(trifluoromethyl)-3-biphenylcarboxylic acid;
3-({3-(aminocarbonyl)-7-[2,4-bis(methyloxy)-5-pyrimidinyl]-4-quinolinyl}amino)-5-(1-methylethenyl)benzoic acid;
3-({3-(aminocarbonyl)-7-[2,4,6-tris(methyloxy)-5-pyrimidinyl]-4-quinolinyl}amino)benzoic acid;
7-[2,4-bis(methyloxy)-5-pyrimidinyl]-4-[(3-(2-furanyl)-5-{[(trifluoromethyl)sulfonyl]amino}phenyl)amino]-3-quinolinecarboxamide;
methyl 3-({3-(aminocarbonyl)-7-[2,4-bis(methyloxy)-5-pyrimidinyl]-4-quinolinyl}amino)-5-{[(trifluoromethyl)sulfonyl]amino}benzoate;
7-[2,4-bis(methyloxy)-5-pyrimidinyl]-4-[(5-{[(trifluoromethyl)sulfonyl]amino}-3-biphenylyl)amino]-3-quinolinecarboxamide;
3-({3-(aminocarbonyl)-7-[2,4-bis(ethyloxy)-5-pyrimidinyl]-4-quinolinyl}amino)benzoic acid;
7-[2,4-bis(methyloxy)-5-pyrimidinyl]-4-[(3-{[(trifluoromethyl)sulfonyl]amino}phenyl)amino]-3-quinolinecarboxamide;
7-[2,4-bis(methyloxy)-5-pyrimidinyl]-4-[(3-(3-furanyl)-5-{[(trifluoromethyl)sulfonyl]amino}phenyl)amino]-3-quinolinecarboxamide;
5-({3-(aminocarbonyl)-7-[2,4-bis(methyloxy)-5-pyrimidinyl]-4-quinolinyl}amino)-4′-chloro-3-biphenylcarboxylic acid;
5-({3-(aminocarbonyl)-7-[2,4-bis(methyloxy)-5-pyrimidinyl]-4-quinolinyl}amino)-2-(3-furanyl)benzoic acid;
3-{[3′-(aminocarbonyl)-2-(methyloxy)-3,7′-biquinolin-4′-yl]amino}benzoic acid;
5-({3-(aminocarbonyl)-7-[2,4-bis(methyloxy)-5-pyrimidinyl]-4-quinolinyl}amino)-2-(2-furanyl)benzoic acid;
3-[(3-(aminocarbonyl)-7-{2,4-bis[(phenylmethyl)oxy]-5-pyrimidinyl}-4-quinolinyl)amino]benzoic acid;
4-({3-(aminocarbonyl)-7-[2,4-bis(methyloxy)-5-pyrimidinyl]-4-quinolinyl}amino)-2-biphenylcarboxylic acid;
5-{[3-(aminocarbonyl)-7-(3,5-dimethyl-4-isoxazolyl)-4-quinolinyl]amino}-3-biphenylcarboxylic acid;
3-{[3-(aminocarbonyl)-7-(3,5-dimethyl-4-isoxazolyl)-4-quinolinyl]amino}-5-(2-furanyl)benzoic acid;
3-{[3-(aminocarbonyl)-7-(2,4-dioxo-1,2,3,4-tetrahydro-5-pyrimidinyl)-4-quinolinyl]amino}benzoic acid;
3-{[3-(aminocarbonyl)-7-(2,3-dihydroxy-4-pyridinyl)-4-quinolinyl]amino}benzoic acid;
4-{3-(aminocarbonyl)-4-[(3-carboxyphenyl)amino]-7-quinolinyl}-2-pyridinecarboxylic acid;
ethyl 5-({3-(aminocarbonyl)-7-[2,4-bis(methyloxy)-5-pyrimidinyl]-4-quinolinyl}amino)-2′-fluoro-6′-(methyloxy)-3-biphenylcarboxylate;
3-({3-(aminocarbonyl)-7-[4-(methyloxy)-3-pyridinyl]-4-quinolinyl}amino)benzoic acid;
3-{[3-(aminocarbonyl)-7-(1H-pyrrolo[2,3-b]pyridin-5-yl)-4-quinolinyl]amino}benzoic acid;
3-{[3-(aminocarbonyl)-7-(3,5-dimethyl-4-isoxazolyl)-4-quinolinyl]amino}-5-[(methyloxy)carbonyl]benzoic acid;
ethyl 3-({3-(aminocarbonyl)-7-[2,4-bis(methyloxy)-5-pyrimidinyl]-4-quinolinyl}amino)benzoate;
3-{[3′-(aminocarbonyl)-3,7′-biquinolin-4′-yl]amino}benzoic acid;
3-({3-(aminocarbonyl)-7-[5-(hydroxymethyl)-1,3-dimethyl-1H-pyrazol-4-yl]-4-quinolinyl}amino)benzoic acid;
3-({3-(aminocarbonyl)-7-[5-(ethyloxy)-3-methyl-1H-pyrazol-4-yl]-4-quinolinyl}amino)benzoic acid;
3-[(3-(aminocarbonyl)-7-{2,4-bis[(1-methylethyl)oxy]-5-pyrimidinyl}-4-quinolinyl)amino]benzoic acid;
3-{[3-(aminocarbonyl)-7-(1H-indol-3-yl)-4-quinolinyl]amino}benzoic acid;
3-{[3-(aminocarbonyl)-7-(3,5-dimethyl-4-isoxazolyl)-4-quinolinyl]amino}-5-(dihydroxyboranyl)benzoic acid;
3-({3-(aminocarbonyl)-7-[2,3-bis(methyloxy)-4-pyridinyl]-4-quinolinyl}amino)benzoic acid;
4-{[3-(aminocarbonyl)-7-(3,5-dimethyl-4-isoxazolyl)-4-quinolinyl]amino}-1,2-benzenedicarboxylic acid;
3-{[3-(aminocarbonyl)-7-(3,5-dimethyl-4-isoxazolyl)-4-quinolinyl]amino}-5-chlorobenzoic acid;
3-{[3-(aminocarbonyl)-7-(3,5-dimethyl-4-isoxazolyl)-4-quinolinyl]amino}-5-(3-furanyl)benzoic acid;
3-{[3-(aminocarbonyl)-7-(3,5-dimethyl-4-isoxazolyl)-4-quinolinyl]amino}-5-iodobenzoic acid;
3-({3-(aminocarbonyl)-7-[2-methyl-6-(methyloxy)-4-pyridinyl]-4-quinolinyl}amino)benzoic acid;
3-({3-(aminocarbonyl)-7-[5-(methyloxy)-3-pyridinyl]-4-quinolinyl}amino)benzoic acid;
3-{[3-(aminocarbonyl)-7-(6-cyano-3-pyridinyl)-4-quinolinyl]amino}benzoic acid;
3-{[3-(aminocarbonyl)-7-(3,5-dimethyl-4-isoxazolyl)-4-quinolinyl]amino}-5-(trifluoromethyl)benzoic acid;
3-{[3-(aminocarbonyl)-7-(3,5-dimethyl-4-isoxazolyl)-4-quinolinyl]amino}-5-hydroxybenzoic acid;
3-{[3-(aminocarbonyl)-7-(2-chloro-6-methyl-4-pyridinyl)-4-quinolinyl]amino}benzoic acid;
3-{[3-(aminocarbonyl)-7-(4-oxo-1,4-dihydro-6-quinazolinyl)-4-quinolinyl]amino}benzoic acid;
3-{[3-(aminocarbonyl)-7-(2-oxo-1,2-dihydro-5-pyrimidinyl)-4-quinolinyl]amino}benzoic acid;
3-{[3-(aminocarbonyl)-7-(3,5-dimethyl-4-isoxazolyl)-4-quinolinyl]amino}-5-bromobenzoic acid;
3-{[3-[({[4-(acetylamino)phenyl]methyl}amino)carbonyl]-7-(3,5-dimethyl-4-isoxazolyl)-4-quinolinyl]amino}benzoic acid;
ethyl 3-({3-[(aminocarbonyl)amino]-7-[2,4-bis(methyloxy)-5-pyrimidinyl]-4-quinolinyl}amino)benzoate;
4-({3-[(aminocarbonyl)amino]phenyl}amino)-7-[2,4-bis(methyloxy)-5-pyrimidinyl]-3-quinolinecarboxamide;
methyl 3-({3-(aminocarbonyl)-7-[2,4-bis(methyloxy)-5-pyrimidinyl]-4-quinolinyl}amino)-5-cyanobenzoate;
methyl 5-({3-(aminocarbonyl)-7-[2,4-bis(methyloxy)-5-pyrimidinyl]-4-quinolinyl}amino)-2-(2-furanyl)benzoate;
methyl 4-({3-(aminocarbonyl)-7-[2,4-bis(methyloxy)-5-pyrimidinyl]-4-quinolinyl}amino)-2-biphenylcarboxylate;
3-({3-(aminocarbonyl)-7-[2,6-bis(methyloxy)-4-pyrimidinyl]-4-quinolinyl}amino)benzoic acid;
3-({3-(aminocarbonyl)-7-[2-(methyloxy)-5-pyrimidinyl]-4-quinolinyl}amino)benzoic acid;
3-{[3-(aminocarbonyl)-7-(2-chloro-1-methyl-1H-imidazol-5-yl)-4-quinolinyl]amino}benzoic acid;
3-{[3-(aminocarbonyl)-7-(2-amino-5-pyrimidinyl)-4-quinolinyl]amino}benzoic acid;
7-[2,4-bis(methyloxy)-5-pyrimidinyl]-4-({3-[(trifluoroacetyl)amino]phenyl}amino)-3-quinolinecarboxamide;
3-({3-(aminocarbonyl)-7-[2-(aminocarbonyl)-4-pyridinyl]-4-quinolinyl}amino)benzoic acid;
3-[(7-(3,5-dimethyl-4-isoxazolyl)-3-{[(2-hydroxycyclopentyl)amino]carbonyl}-4-quinolinyl)amino]benzoic acid;
7-[2,4-bis(methyloxy)-5-pyrimidinyl]-4-[(3-bromo-5-nitrophenyl)amino]-3-quinolinecarboxamide;
ethyl 3-({3-(aminocarbonyl)-7-[2-methyl-6-(methyloxy)-4-pyridinyl]-4-quinolinyl}amino)benzoate;
7-(3,5-dimethyl-4-isoxazolyl)-4-[(3-{[(trifluoromethyl)sulfonyl]amino}phenyl)amino]-3-quinolinecarboxamide;
3-{[3-(aminocarbonyl)-7-(4-amino-2-chloro-5-pyrimidinyl)-4-quinolinyl]amino}benzoic acid;
3-({3-(aminocarbonyl)-7-[2-(methyloxy)-6-(trifluoromethyl)-4-pyridinyl]-4-quinolinyl}amino)benzoic acid;
3-({3-(aminocarbonyl)-6-[2,4-bis(methyloxy)-5-pyrimidinyl]-4-quinolinyl}amino)benzoic acid;
5-{[3-(aminocarbonyl)-7-(3,5-dimethyl-4-isoxazolyl)-4-quinolinyl]amino}-2-chlorobenzoic acid;
3-{[3-(aminocarbonyl)-7-(2-cyano-5-pyrimidinyl)-4-quinolinyl]amino}benzoic acid;
3-{[3-[({[4-(aminosulfonyl)phenyl]methyl}amino)carbonyl]-7-(3,5-dimethyl-4-isoxazolyl)-4-quinolinyl]amino}benzoic acid;
3-{[3-(aminocarbonyl)-7-(3,5-dimethyl-4-isoxazolyl)-4-quinolinyl]amino}benzenesulfonic acid;
3-({3-(aminocarbonyl)-7-[4-amino-2-(methyloxy)-5-pyrimidinyl]-4-quinolinyl}amino)benzoic acid;
methyl 5-({3-(aminocarbonyl)-7-[2,4-bis(methyloxy)-5-pyrimidinyl]-4-quinolinyl}amino)-3-biphenylcarboxylate;
3-{[3-(aminocarbonyl)-7-(1,2-dimethyl-1H-imidazol-5-yl)-4-quinolinyl]amino}benzoic acid;
5-{[3-(aminocarbonyl)-7-(3,5-dimethyl-4-isoxazolyl)-4-quinolinyl]amino}-3-pyridinecarboxylic acid;
3-({3-(aminocarbonyl)-7-[4-(dimethylamino)-2-(methyloxy)-5-pyrimidinyl]-4-quinolinyl}amino)benzoic acid;
ethyl 3-[(3-(aminocarbonyl)-7-{2,4-bis[(phenylmethyl)oxy]-5-pyrimidinyl}-4-quinolinyl)amino]benzoate;
3-({2-amino-3-(aminocarbonyl)-7-[2,4-bis(methyloxy)-5-pyrimidinyl]-4-quinolinyl}amino)benzoic acid;
7-[2,4-bis(methyloxy)-5-pyrimidinyl]-4-[(3-(methyloxy)-5-{[(trifluoromethyl)sulfonyl]amino}phenyl)amino]-3-quinolinecarboxamide;
3-({3-(aminocarbonyl)-7-[2,4-bis(methyloxy)-5-pyrimidinyl]-4-quinolinyl}amino)-5-{[(trifluoromethyl)sulfonyl]amino}benzoic acid;
3-({3-(aminocarbonyl)-7-[2,4-bis(methyloxy)-5-pyrimidinyl]-4-quinolinyl}amino)-5-(4-morpholinyl)benzoic acid;
3-({3-(aminocarbonyl)-7-[2,4-bis(methyloxy)-5-pyrimidinyl]-4-quinolinyl}amino)-5-(4-methyl-1-piperazinyl)benzoic acid;
7-[2,4-bis(methyloxy)-5-pyrimidinyl]-4-[(3-(tetrahydro-3-furanyl)-5-{[(trifluoromethyl)sulfonyl]amino}phenyl)amino]-3-quinolinecarboxamide;
4-[(3-amino-5-cyclopentylphenyl)amino]-7-[2,4-bis(methyloxy)-5-pyrimidinyl]-3-quinolinecarboxamide;
3-({3-(aminocarbonyl)-7-[2,4-bis(ethyloxy)-5-pyrimidinyl]-4-quinolinyl}amino)-5-(1-cyclohexen-1-yl)benzoic acid;
3-{[3-(aminosulfonyl)-7-(3,5-dimethyl-1H-pyrazol-4-yl)-4-quinolinyl]amino}benzoic acid;
3-({3-(aminocarbonyl)-7-[2,4-bis(methyloxy)-5-pyrimidinyl]-4-quinolinyl}amino)-5-(1-piperidinyl)benzoic acid;
7-[2,4-bis(methyloxy)-5-pyrimidinyl]-4-({3-[(phenylsulfonyl)amino]phenyl}amino)-3-quinolinecarboxamide;
7-[2,4-bis(methyloxy)-5-pyrimidinyl]-4-[(3-{[(phenylmethyl)sulfonyl]amino}phenyl)amino]-3-quinolinecarboxamide; and
3-({3-(aminocarbonyl)-7-[2,4-bis(methyloxy)-5-pyrimidinyl]-2-methyl-4-quinolinyl}amino)benzoic acid;
   or a salt thereof including a pharmaceutically acceptable salt thereof.

The skilled artisan will appreciate that salts, including pharmaceutically acceptable salts, of the compounds according to Formula I may be prepared. Indeed, in certain embodiments of the invention, salts including pharmaceutically-acceptable salts of the compounds according to Formula I may be preferred over the respective free base. Accordingly, the invention is further directed to salts, including pharmaceutically-acceptable salts, of the compounds according to Formula I.

The salts of the compounds of the invention are readily prepared by those of skill in the art.

The pharmaceutically acceptable salts of the compounds of the invention are readily prepared by those of skill in the art.

The compounds according to Formula I may contain one or more asymmetric centers (also referred to as a chiral center) and may, therefore, exist as individual enantiomers, diastereomers, or other stereoisomeric forms, or as mixtures thereof. Chiral centers, such as chiral carbon atoms, may also be present in a substituent such as an alkyl group. Where the stereochemistry of a chiral center present in Formula I, or in any chemical structure illustrated herein, if not specified the structure is intended to encompass all individual stereoisomers and all mixtures thereof. Thus, compounds according to Formula I containing one or more chiral centers may be used as racemic mixtures, enantiomerically enriched mixtures, or as enantiomerically pure individual stereoisomers.

The compounds according to Formula I may also contain double bonds or other centers of geometric asymmetry. Where the stereochemistry of a center of geometric asymmetry present in Formula I, or in any chemical structure illustrated herein, is not specified, the structure is intended to encompass the trans (E) geometric isomer, the cis (Z) geometric isomer, and all mixtures thereof. Likewise, all tautomeric forms are also included in Formula I whether such tautomers exist in equilibrium or predominately in one form.

The compounds of Formula I or salts, including pharmaceutically acceptable salts, thereof may exist in solid or liquid form. In the solid state, the compounds of the invention may exist in crystalline or noncrystalline form, or as a mixture thereof. For compounds of the invention that are in crystalline form, the skilled artisan will appreciate that pharmaceutically acceptable solvates may be formed wherein solvent molecules are incorporated into the crystalline lattice during crystallization. Solvates wherein water is the solvent that is incorporated into the crystalline lattice are typically referred to as “hydrates.” Hydrates include stoichiometric hydrates as well as compositions containing vaiable amounts of water. The invention includes all such solvates.

The skilled artisan will further appreciate that certain compounds of Formula I or salts, including pharmaceutically acceptable salts thereof that exist in crystalline form, including the various solvates thereof, may exhibit polymorphism (i.e. the capacity to occur in different crystalline structures). These different crystalline forms are typically known as “polymorphs.” The invention includes all such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions. The invention includes all such polymorphs.

### DEFINITIONS

“Alkyl” refers to a hydrocarbon chain having the specified number of member atoms. For example, C₁-C₆ alkyl refers to an alkyl group having from 1 to 6 member atoms. Alkyl groups may be saturated, unsaturated, straight or branched. Representative branched alkyl groups have one, two, or three branches. Unless otherwise defined, alkyl includes methyl, ethyl, ethylene, propyl (n-propyl and isopropyl), butene, and butyl (n-butyl, isobutyl, and t-butyl).
“Alkoxy” refers to an —O-alkyl group wherein “alkyl” is as defined herein. For example, C₁-C₄alkoxy refers to an alkoxy group having from 1 to 4 member atoms. Representative branched alkoxy groups have one, two, or three branches. Examples of such groups include methoxy, ethoxy, propoxy, and butoxy.
“Aryl” refers to an aromatic hydrocarbon ring. Aryl groups are monocyclic ring systems or bicyclic ring systems. Examples of such monocyclic aryl rings include phenyl naphthalene, tetrahydronaphthalene and biphenyl. Examples of such bicyclic aryl rings include napthyl and rings wherein phenyl is fused to a cycloalkyl or cycloalkenyl ring having 5, 6, or 7 member atoms.
“Cycloalkyl” refers to a saturated or unsaturated non aromatic hydrocarbon ring having the specified number of member atoms. Cycloalkyl groups are monocyclic ring systems. For example, C₃-C₇ cycloalkyl refers to a cycloalkyl group having from 3 to 7 member atoms. Examples of cycloalkyl as used herein includes cyclopropyl, cyclobutyl, cyclopentyl, cyclohexyl, cyclohexen, cyclobutenyl, cyclopenten, cyclopentenyl and cyclohexenyl.
“Heterocycloalkyl” refers to a saturated or unsaturated ring containing from 1 to 4 heteroatoms as member atoms in the ring. However, heterocycloalkyl rings are not aromatic. Heterocycloalkyl groups containing more than one heteroatom may contain different heteroatoms. Heterocycloalkyl groups are monocyclic ring systems or a monocyclic ring fused with and aryl ring or to a heteroaryl ring having from 4 to 11 member atoms. In certain embodiments, heterocycloalkyl is saturated. In other embodiments, heterocycloalkyl is unsaturated but not aromatic. Heterocycloalkyl includes pyrrolidinyl, tetrahydrofuranyl, dihydrofuranyl, pyranyl, tetrahydropyranyl, dihydropyranyl, tetrahydrothienyl, pyrazolidinyl, oxazolidinyl, thiazolidinyl, piperidinyl, homopiperidinyl, piperazinyl, morpholinyl, thiamorpholinyl, 1,3-dioxolanyl, 1,3-dioxanyl, 1,4-dioxanyl, 1,3-oxathiolanyl, 1,3-oxathianyl, 1,3-dithianyl, 1,3oxazolidin-2-one, hexahydro-1H-azepin, 4,5,6,7,tetrahydro-1H-benzimidazol, piperidinyl, 1,2,3,6-tetrahydro-pyridinyl and azetidinyl, suitably tetrahydrofuranyl or piperidinyl.
“Halo” or “Halogen” refers to the halogen radicals fluoro, chloro, bromo, and iodo.
“Heteroaryl” refers to an aromatic ring containing from 1 to 4 heteroatoms as member atoms in the ring. Heteroaryl groups containing more than one heteroatom may contain different heteroatoms. Heteroaryl groups are monocyclic or bicyclic ring systems. Monocyclic heteroaryl rings have 5 or 6 member atoms. Bicyclic heteroaryl rings have from 6 to 11 member atoms. Unless otherwise defined, heteroaryl includes: pyrrolyl, pyrazolyl, imidazolyl, oxazolyl, isoxazolyl, thiazolyl, isothiazolyl, furanyl, furazanyl, thienyl, triazolyl, pyridinyl, pyrimidinyl, pyridazinyl, pyrazinyl, triazinyl, tetrazinyl, quinolinyl, quinazolinyl, indiazole, indole, thienyl, pyrrolopyridin, pyran, dihydropyran, tetrahydropyran, tetrahydrofuranyl, piperidinyl, tetrazole, morpholinyl, and piperazinyl,
“Heteroatom” refers to a nitrogen, sulphur or oxygen atom.
“Pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

Unless otherwise defined, the term “Substituted” as used herein, is meant that the subject chemical moiety has from one to four substituents, suitably from one to three substituents, suitably one or two substituents, independently selected from the group consisting of: piperazinyl, OH, —NH₂, —NO₂, —F, —Cl, —Br, —I, —OC₁-C₆alkyl, —CO₂ethyl, —C₁-C₆alkyl, —CONH₂, —OCH₂phenyl, oxo, —COOH, hydroxyC₁-C₆alkyl, —C≡N, —CF₃, —N(CH₃)₂, —NHCH₃.

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

Ac (acetyl);
Ac₂O (acetic anhydride);
ACN (acetonitrile);
AlBN (azobis(isobutyronitrile));
BINAP (2,2′-bis(diphenylphosphino)-1,1′-binaphthyl);
BMS (borane-dimethyl sulphide complex);
Bn (benzyl);
Boc (tert-Butoxycarbonyl);
Boc₂O (di-tert-butyl dicarbonate);
BOP (Benzotriazole-1-yl-oxy-tris-(dimethylamino)-phosphonium hexafluorophosphate);
CAN (cerric ammonium nitrate);
Cbz (benzyloxycarbonyl);
CSI (chlorosulfonyl isocyanate);
DABCO (1,4-Diazabicyclo[2.2.2]octane);
DAST ((Diethylamino)sulfur trifluoride);
DBU (1,8-Diazabicyclo[5.4.0]undec-7-ene);

### DCC (Dicyclohexyl Carbodiimide);

DCE (1,2-dichloroethane);
DCM (dichloromethane);
DDQ (2,3-Dichloro-5,6-dicyano-1,4-benzoquinone);
ATP (adenosine triphosphate);
Bis-pinacolatodiboron (4,4,4′,4′,5,5,5′,5′-Octamethyl-2,2′-bi-1,3,2-dioxaborolane);
BSA (bovine serum albumin);
C18 (refers to 18-carbon alkyl groups on silicon in HPLC stationary phase)
CH₃CN (acetonitrile);
Cy (cyclohexyl);
DCM (dichloromethane);
DIPEA (Hünig's base, N-ethyl-N-(1-methylethyl)-2-propanamine);
DMAP (4-dimethylaminopyridine);
DME (1,2-dimethoxyethane);

### DMF (N,N-dimethylformamide);

DMSO (dimethylsulfoxide);
DPPA (diphenyl phosphoryl azide);
EDC (N-(3-dimethylaminopropyl)-N′ ethylcarbodiimide);
EDTA (ethylenediaminetetraacetic acid);
EtOAc (ethyl acetate);
EtOH (ethanol);
Et₂O (diethyl ether);
HEPES (4-(2-hydroxyethyl)-1-piperazine ethane sulfonic acid);
HATU (O-(7-Azabenzotriazol-1-yl)-N,N,N′,N′-tetramethyluronium hexafluorophosphate);
HOAt (1-hydroxy-7-azabenzotriazole);
HOBt (1-hydroxybenzotriazole);
HOAc (acetic acid);
HPLC (high pressure liquid chromatography);
HMDS (hexamethyldisilazide);

### Hunig's Base (N,N-Diisopropylethylamine);

IPA (isopropyl alcohol);
Indoline (2,3-dihydro-1H-indole);
KHMDS (potassium hexamethyldisilazide);
LAH (lithium aluminum hydride);
LDA (lithium diisopropylamide);
LHMDS (lithium hexamethyldisilazide)
MeOH (methanol);
MTBE (methyl tert-butyl ether);
mCPBA (m-chloroperbezoic acid);
NaHMDS (sodium hexamethyldisilazide);

### NBS (N-bromosuccinimide);

PE (petroleum ether);
Pd₂(dba)₃ (Tris(dibenzylideneacetone)dipalladium(0);
Pd(dppf)Cl₂ ([1,1′-Bis(diphenylphosphino)ferrocene]dichloropalladium(II));
PyBOP (benzotriazol-1-yl-oxytripyrrolidinophosphonium hexafluorophosphate);
PyBrOP (bromotripyrrolidinophosphonium hexafluorophosphate);
RPHPLC (reverse phase high pressure liquid chromatography);
SFC (supercritical fluid chromatography);
SGC (silica gel chromatography);
TEA (triethylamine);
TEMPO (2,2,6,6-Tetramethylpiperidine 1-oxyl, free radical);
TFA (trifluoroacetic acid); and
THF (tetrahydrofuran).

All references to ether are to diethyl ether and brine refers to a saturated aqueous solution of NaCl.

### Compound Preparation

The novel compounds of Formula (I) are generally prepared as shown in Schemes 1 to 8 below, or by analogous methods, provided the ‘R’ substituents in Formula (I) do not include any such substituents that render inoperative the processes of any of Schemes 1 to 8. Further, the skilled artisan will appreciate that if a substituent described herein is not compatible with the synthetic methods described herein, the substituent may be protected with a suitable protecting group that is stable to the reaction conditions. All of the starting materials are commercially available or are readily made from commercially available starting materials by those of skill in the art.

### Schemes

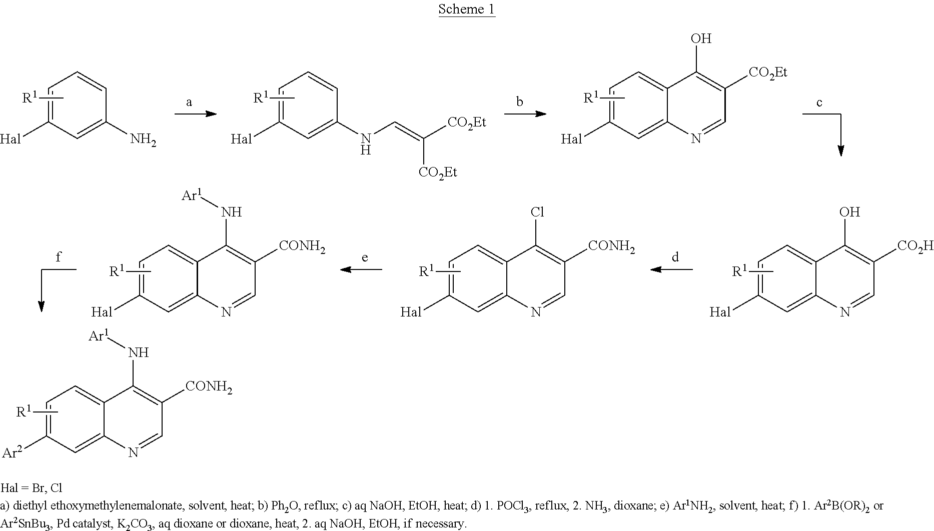

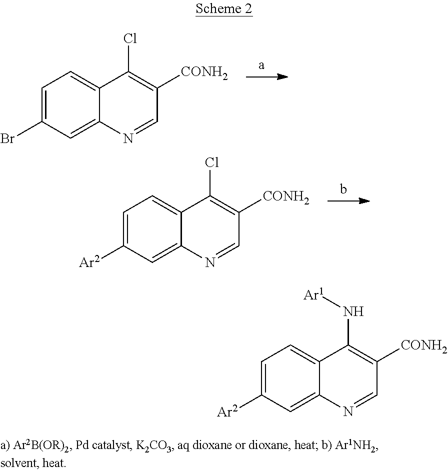

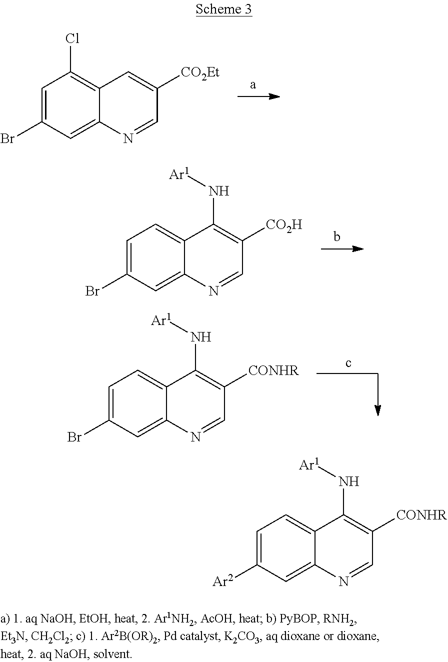

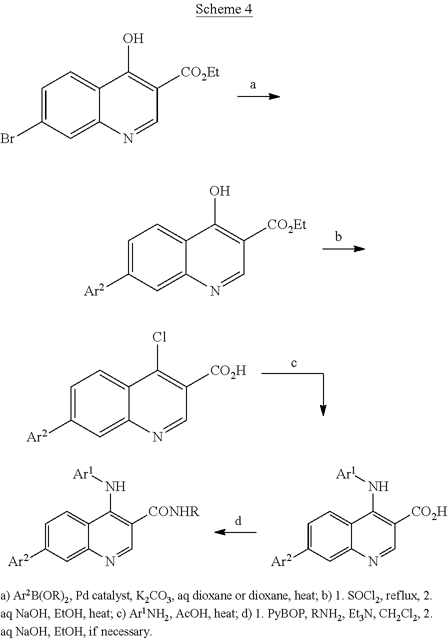

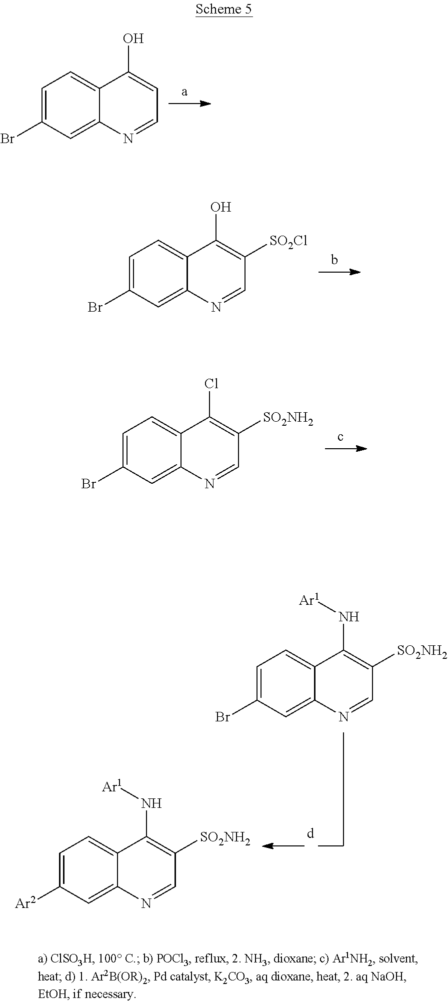

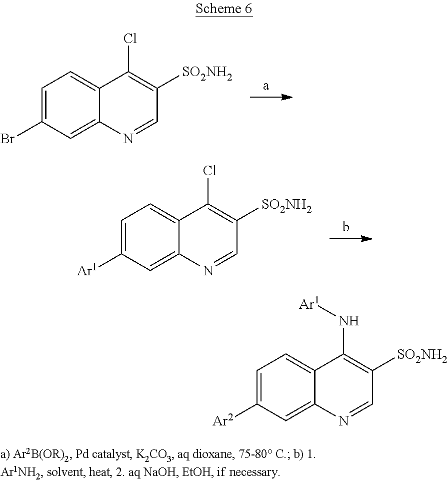

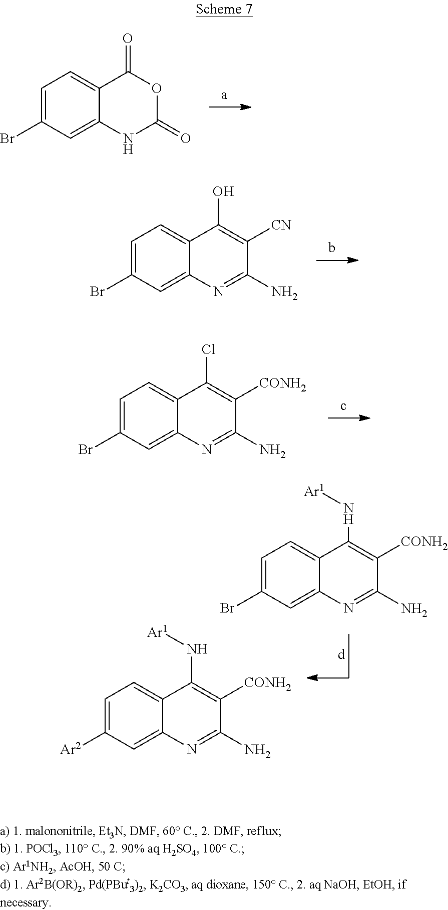

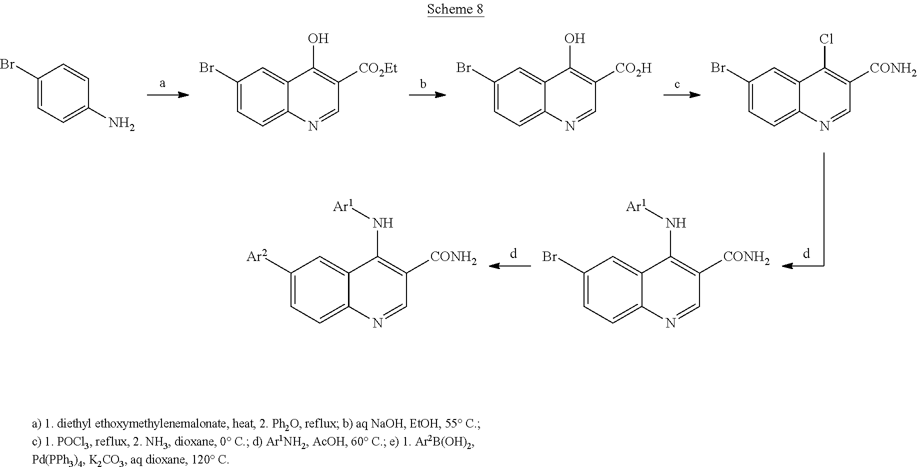

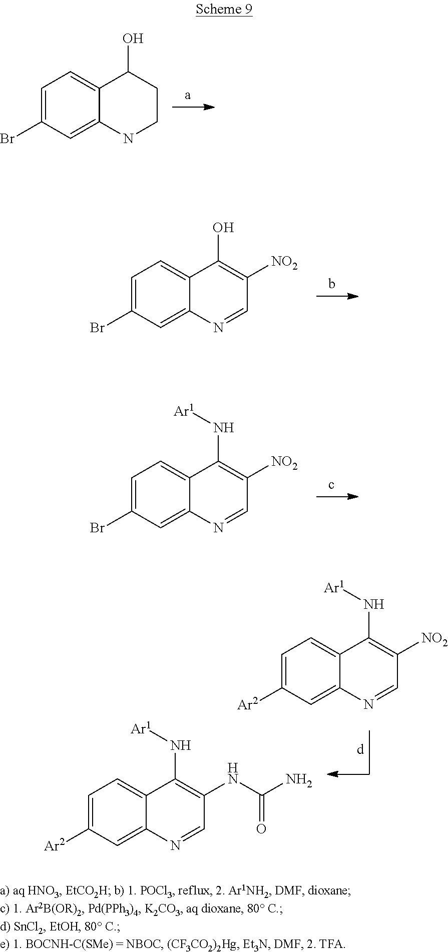

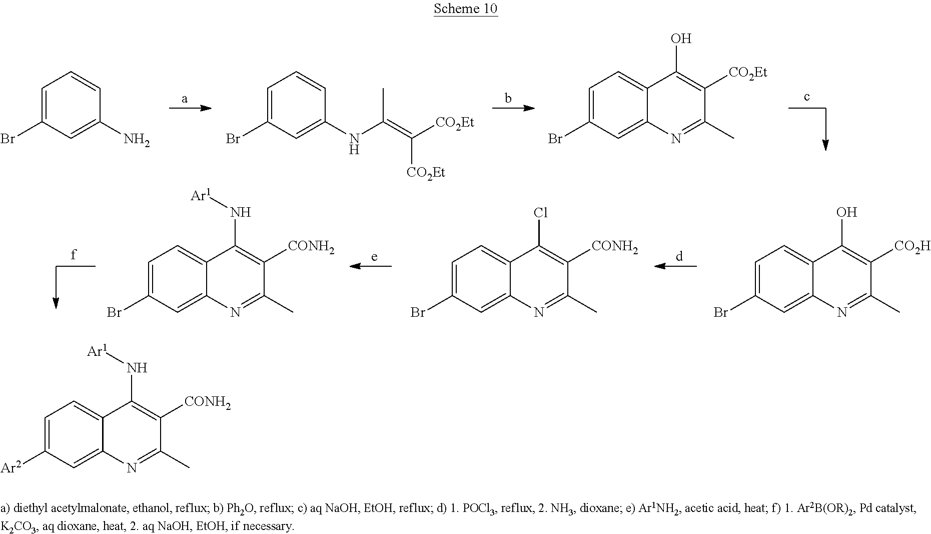

### DEFINITIONS

HPLC=high performance liquid chromatography
LCMS=liquid chromatography/mass spectrometry
MgSO₄=anhydrous magnesium sulfate
PyBOP=benzotriazol-1-yloxy-trispyrrolidinophosphonium hexafluorophosphate
Pd₂(dba)₃=tris(dibenzylideneacetone)dipalladium (0)
BINAP=2,2′-bis(diphenylphosphino)-1,1′-binaphthalene

### Methods of Use

The compounds according to Formula I and pharmaceutically acceptable salts thereof are inhibitors of lactate dehydrogenase A. These compounds are potentially useful in the treatment of conditions wherein the underlying pathology is attributable to (but not limited to) tumor cell metabolism, for example, cancer and more specifically cancers of the breast, colon, and lung, pancreas and skin. Accordingly, another aspect the invention is directed to methods of treating such conditions.

Suitably, the present invention relates to a method for treating or lessening the severity of breast cancer, including inflammatory breast cancer, ductal carcinoma, and lobular carcinoma.

Suitably the present invention relates to a method for treating or lessening the severity of colon cancer.

Suitably the present invention relates to a method for treating or lessening the severity of pancreatic cancer.

Suitably the present invention relates to a method for treating or lessening the severity of skin cancer, including melanoma.

Suitably the present invention relates to a method for treating or lessening the severity of lung cancer including small cell lung cancer, non-small cell lung cancer, squamous cell carcinoma, adenocarcinoma, and large cell carcinoma.

Suitably, the present invention relates to a method for treating or lessening the severity of a cancer selected from: brain (gliomas), glioblastomas, astrocytomas, glioblastoma multiforme, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid,

Lymphoblastic T cell leukemia, Chronic myelogenous leukemia, Chronic lymphocytic leukemia, Hairy-cell leukemia, acute lymphoblastic leukemia, acute myelogenous leukemia, Chronic neutrophilic leukemia, Acute lymphoblastic T cell leukemia, Plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia,
malignant lymphoma, hodgkins lymphoma, non-hodgkins lymphoma, lymphoblastic T cell lymphoma, Burkitt's lymphoma, follicular lymphoma,
neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

Suitably, the present invention relates to a method for treating or lessening the severity of a cancer selected from: brain (gliomas), glioblastomas, astrocytomas, glioblastoma multiforme, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

The methods of treatment of the invention comprise administering an effective amount of a compound according to Formula I or a pharmaceutically acceptable salt, thereof to a patient in need thereof.

The invention also provides a compound according to Formula I or a pharmaceutically-acceptable salt thereof for use in medical therapy, and particularly in cancer therapy. Thus, in further aspect, the invention is directed to the use of a compound according to Formula I or a pharmaceutically acceptable salt thereof in the preparation of a medicament for the treatment of a disorder attributable to (but not limited to) tumor cell metabolism, such as cancer.

By the term “treating” and derivatives thereof as used herein, is meant prophylactic and therapeutic therapy. Prophylactic therapy is appropriate, for example, when a subject is considered at high risk for developing cancer, or when a subject has been exposed to a carcinogen.

As used herein, the term “effective amount” and derivatives thereof means that amount of a drug or pharmaceutical agent that will elicit the biological or medical response of a tissue, system, animal or human that is being sought, for instance, by a researcher or clinician. Furthermore, the term “therapeutically effective amount” and derivatives thereof means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, “patient” or “subject” refers to a human or other animal. Suitably the patient or subject is a human.

The compounds of Formula I or pharmaceutically acceptable salts thereof may be administered by any suitable route of administration, including systemic administration. Systemic administration includes oral administration, and parenteral administration. Parenteral administration refers to routes of administration other than enteral, transdermal, or by inhalation, and is typically by injection or infusion. Parenteral administration includes intravenous, intramuscular, and subcutaneous injection or infusion.

The compounds of Formula I or pharmaceutically acceptable salts thereof may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Additionally, the compounds of Formula I or pharmaceutically-acceptable salts thereof may be administered as prodrugs. As used herein, a “prodrug” of a compound of the invention is a functional derivative of the compound which, upon administration to a patient, eventually liberates the compound of the invention in vivo. Administration of a compound of the invention as a prodrug may enable the skilled artisan to do one or more of the following: (a) modify the onset of the compound in vivo; (b) modify the duration of action of the compound in vivo; (C) modify the transportation or distribution of the compound in vivo; (d) modify the solubility of the compound in vivo; and (e) overcome a side effect or other difficulty encountered with the compound. Where a —COOH or —OH group is present, pharmaceutically acceptable esters can be employed, for example methyl, ethyl, and the like for —COOH, and acetate maleate and the like for —OH, and those esters known in the art for modifying solubility or hydrolysis characteristics.

The compounds of Formula I and pharmaceutically acceptable salts thereof may be co-administered with at least one other active ingredient known to be useful in the treatment of cancer.

By the term “co-administration” as used herein is meant either simultaneous administration or any manner of separate sequential administration of a lactate dehydrogenase A inhibiting compound, as described herein, and a further active ingredient or ingredients, known to be useful in the treatment of cancer, including chemotherapy and radiation treatment. The term further active ingredient or ingredients, as used herein, includes any compound or therapeutic agent known to or that demonstrates advantageous properties when administered to a patient in need of treatment for cancer. Preferably, if the administration is not simultaneous, the compounds are administered in a close time proximity to each other. Furthermore, it does not matter if the compounds are administered in the same dosage form, e.g. one compound may be administered by injection and another compound may be administered orally.

Typically, any anti-neoplastic agent that has activity versus a susceptible tumor being treated may be co-administered in the treatment of cancer in the present invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

Examples of a further active ingredient or ingredients (anti-neoplastic agent) for use in combination or co-administered with the presently invented lactate dehydrogenase A inhibiting compounds are chemotherapeutic agents.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al. J. Am. Chem., Soc., 93:2325. 1971), who characterized its structure by chemical and X-ray crystallographic methods. One mechanism for its activity relates to paclitaxel's capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Attaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Intern, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3S)—N-carboxy-3-phenylisoserine,N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine [R—(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine [1,1-cyclobutane-dicarboxylate(2-)-O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also known as Actinomycin D, is commercially available in injectable form as COSMEGEN®. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S,10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leucopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leucopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mercaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis as well as incorporation into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leucopenia, thrombocytopenia, and anemia, is the most common dose limiting side effect of thioguanine administration. However, gastrointestinal side effects also occur and can be dose limiting. Other purine analogs include pentostatin, erythrohydroxynonyladenine, fludarabine phosphate, and cladribine.

Gemcitabine, 2′-deoxy-2′,2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leucopenia, thrombocytopenia, and anemia, is the most common dose limiting side effect of gemcitabine administration.

Methotrexate, N-[4[[(2,4-diamino-6-pteridinyl)methyl]methylamino]benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary, and bladder. Myelosuppression (leucopenia, thrombocytopenia, and anemia) and mucositis are expected side effect of methotrexate administration.

Camptothecins, including, camptothecin and camptothecin derivatives, are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®.

Irinotecan is a derivative of camptothecin which binds, along with its active metabolite SN-38, to the topoisomerase I-DNA complex. It is believed that cytotoxicity occurs as a result of irreparable double strand breaks caused by interaction of the topoisomerase I:DNA:irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I-DNA complex and prevents religation of single strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Also of interest, is the camptothecin derivative of Formula A following, including the racemic mixture (R,S) form as well as the R and S enantiomers:

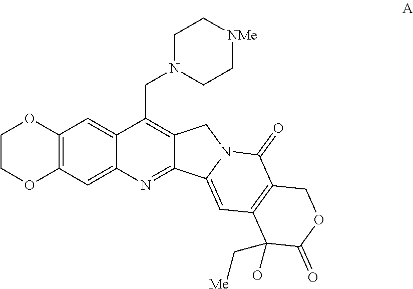 [SMILES: CCC1(O)C(=O)OCc2c1cc1n(c2=O)Cc2c-1nc1cc3c(cc1c2CN1CCN(C)CC1)OCCO3]

known by the chemical name “7-(4-methylpiperazino-methylene)-10,11-ethylenedioxy-20(R,S)-camptothecin (racemic mixture) or “7-(4-methylpiperazino-methylene)-10,11-ethylenedioxy-20(R)-camptothecin (R enantiomer) or “7-(4-methylpiperazino-methylene)-10,11-ethylenedioxy-20(S)-camptothecin (S enantiomer). Such compound as well as related compounds are described, including methods of making, in U.S. Pat. Nos. 6,063,923; 5,342,947; 5,559,235; 5,491,237 and pending U.S. patent application Ser. No. 08/977,217 filed Nov. 24, 1997.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such as those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment of prostatic carcinoma, for instance, LHRH agonists and antagagonists such as goserelin acetate and luprolide.

Signal transduction pathway inhibitors are those inhibitors, which block or inhibit a chemical process which evokes an intracellular change. As used herein this change is cell proliferation or differentiation. Signal tranduction inhibitors useful in the present invention include inhibitors of receptor tyrosine kinases, non-receptor tyrosine kinases, SH2/SH3 domain blockers, serine/threonine kinases, phosphotidylinositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth factor receptors as targets”, New Molecular Targets for Cancer Chemotherapy, ed. Workman, Paul and Kerr, David, CRC press 1994, London.

Tyrosine kinases, which are not growth factor receptor kinases are termed non-receptor tyrosine kinases. Non-receptor tyrosine kinases for use in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S, and Corey, S. J., (1999) Journal of Hematotherapy and Stem Cell Research 8 (5): 465 80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Suitably, the pharmaceutically active compounds of the invention are used in combination with inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta); IkB kinase family (IKKa, IKKb), PKB family kinases, akt kinase family members, PDK1 and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60. 1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; Pearce, L. R et al. Nature Reviews Molecular Cell Biology (2010) 11, 9-22. and Martinez-Iacaci, L., et al, Int. J. Cancer (2000), 88(1), 44-52.

Suitably, the pharmaceutically active compounds of the invention are used in combination with a MEK inhibitor. Suitably, N-{3-[3-cyclopropyl-5-(2-fluoro-4-iodo-phenylamino)-6,8-dimethyl-2,4,7-trioxo-3,4,6,7-tetrahydro-2H-pyrido[4,3-d]pyrimidin-1-yl]phenyl}acetamide, or a pharmaceutically acceptable salt or solvate, suitably the dimethyl sulfoxide solvate, thereof, which is disclosed and claimed in International Application No. PCT/JP2005/011082, having an International filing date of Jun. 10, 2005; International Publication Number WO 2005/121142 and an International Publication date of Dec. 22, 2005, the entire disclosure of which is hereby incorporated by reference. N-{3-[3-cyclopropyl-5-(2-fluoro-4-iodo-phenylamino)-6,8-dimethyl-2,4,7-trioxo-3,4,6,7-tetrahydro-2H-pyrido[4,3-d]pyrimidin-1-yl]phenyl}acetamide, can be prepared as described in United States Patent Publication No. US 2006/0014768, Published Jan. 19, 2006, the entire disclosure of which is hereby incorporated by reference.

Suitably, the pharmaceutically active compounds of the invention are used in combination with a B-Raf inhibitor. Suitably, N-{3-[5-(2-Amino-4-pyrimidinyl)-2-(1,1-dimethylethyl)-1,3-thiazol-4-yl]-2-fluorophenyl}-2,6-difluorobenzenesulfonamide, or a pharmaceutically acceptable salt thereof, which is disclosed and claimed, in International Application No. PCT/US2009/042682, having an International filing date of May 4, 2009, the entire disclosure of which is hereby incorporated by reference. N-{3-[5-(2-Amino-4-pyrimidinyl)-2-(1,1-dimethylethyl)-1,3-thiazol-4-yl]-2-fluorophenyl}-2,6-difluorobenzenesulfonamide can be prepared as described in International Application No. PCT/US2009/042682.

Suitably, the pharmaceutically active compounds of the invention are used in combination with an Akt inhibitor. Suitably, N-{(1S)-2-amino-1-[(3,4-difluorophenyl)methyl]ethyl}-5-chloro-4-(4-chloro-1-methyl-1H-pyrazol-5-yl)-2-furancarboxamide or a pharmaceutically acceptable salt thereof, which is disclosed and claimed in International Application No. PCT/US2008/053269, having an International filing date of Feb. 7, 2008; International Publication Number WO 2008/098104 and an International Publication date of Aug. 14, 2008, the entire disclosure of which is hereby incorporated by reference. N-{(1S)-2-amino-1-[(3,4-difluorophenyl)methyl]ethyl}-5-chloro-4-(4-chloro-1-methyl-1H-pyrazol-5-yl)-2-furancarboxamide is the compound of example 224 and can be prepared as described in International Application No. PCT/US2008/053269.

Suitably, the pharmaceutically active compounds of the invention are used in combination with an Akt inhibitor. Suitably, N-{(1S)-2-amino-1-[(3-fluorophenyl)methyl]ethyl}-5-chloro-4-(4-chloro-1-methyl-1H-pyrazol-5-yl)-2-thiophenecarboxamide or a pharmaceutically acceptable salt thereof, which is disclosed and claimed in International Application No. PCT/US2008/053269, having an International filing date of Feb. 7, 2008; International Publication Number WO 2008/098104 and an International Publication date of Aug. 14, 2008, the entire disclosure of which is hereby incorporated by reference. N-{(1S)-2-amino-1-[(3-fluorophenyl)methyl]ethyl}-5-chloro-4-(4-chloro-1-methyl-1H-pyrazol-5-yl)-2-thiophenecarboxamide is the compound of example and can be prepared as described in International Application No. PCT/US2008/053269. Suitably, N-{(1S)-2-amino-1-[(3-fluorophenyl)methyl]ethyl}-5-chloro-4-(4-chloro-1-methyl-1H-pyrazol-5-yl)-2-thiophenecarboxamide is in the form of a hydrochloride salt. The salt form can be prepared by one of skill in the art from the description in International Application No. PCT/US2010/022323, having an International filing date of Jan. 28, 2010.

Combinations with drugs against other metabolic targets, e.g. ATP citrate lyase (ACLY), hexokinase (HK), pyruvate kinase M2 (PKM2), pyruvate dehydrogenase kinase 1 (PDK1), fatty acid synthase (FASN), PI3Kβ and isocitrate dehydrogenase 1 (IDH1), should also be effective.

Inhibitors of Phosphotidylinositol-3 Kinase family members including blockers of PI3-kinase, ATM, DNA-PK, and Ku may also be useful in the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also of interest in the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type or mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99 102; and BioChim. Biophys. Acta, (19899) 1423(3):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al, Monoclonal Antibody Therapy for Solid Tumors, Cancer Treat. Rev., (2000), 26(4), 269-286); Herceptin® erbB2 antibody (see Tyrosine Kinase Signalling in Breast cancer:erbB Family Receptor Tyrosine Kniases, Breast cancer Res., 2000, 2(3), 176-183); and 2CB VEGFR2 specific antibody (see Brekken, R. A. et al, Selective Inhibition of VEGFR2 Activity by a monoclonal Anti-VEGF antibody blocks tumor growth in mice, Cancer Res. (2000) 60, 5117-5124).

Non-receptor kinase angiogenesis inhibitors may also be useful in the present invention. Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Accordingly, non-receptor tyrosine kinase inhibitors may be used in combination with the compounds of the present invention. For example, anti-VEGF antibodies, which do not recognize VEGFR (the receptor tyrosine kinase), but bind to the ligand; small molecule inhibitors of integrin (alpha beta₃) that will inhibit angiogenesis; endostatin and angiostatin (non-RTK) may also prove useful in combination with the disclosed compounds. (See Bruns C J et al (2000), Cancer Res., 60: 2926-2935; Schreiber A B, Winkler M E, and Derynck R. (1986), Science, 232: 1250-1253; Yen L et al. (2000), Oncogene 19: 3460-3469).

Agents used in immunotherapeutic regimens may also be useful in combination with the compounds of Formula (I). There are a number of immunologic strategies to generate an immune response. These strategies are generally in the realm of tumor vaccinations. The efficacy of immunologic approaches may be greatly enhanced through combined inhibition of signaling pathways using a small molecule inhibitor. Discussion of the immunologic/tumor vaccine approach against erbB2/EGFR are found in Reilly R T et al. (2000), Cancer Res. 60: 3569-3576; and Chen Y, Hu D, Eling D J, Robbins J, and Kipps T J. (1998), Cancer Res. 58: 1965-1971.

Agents used in proapoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family (i.e., mcl-1). Therefore, strategies designed to downregulate the expression of bcl-2 in tumors have demonstrated clinical benefit and are now in Phase II/III trials, namely Genta's G3139 bcl-2 antisense oligonucleotide. Such proapoptotic strategies using the antisense oligonucleotide strategy for bcl-2 are discussed in Water J S et al. (2000), J. Clin. Oncol. 18: 1812-1823; and Kitada S et al. (1994), Antisense Res. Dev. 4: 71-79.

Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230.

Further, p21WAF1/CIP1 has been described as a potent and universal inhibitor of cyclin-dependent kinases (Cdks) (Ball et al., Progress in Cell Cycle Res., 3: 125 (1997)). Compounds that are known to induce expression of p21WAF1/CIP1 have been implicated in the suppression of cell proliferation and as having tumor suppressing activity (Richon et al., Proc. Nat. Acad. Sci. U.S.A. 97(18): 10014-10019 (2000)), and are included as cell cycle signaling inhibitors. Histone deacetylase (HDAC) inhibitors are implicated in the transcriptional activation of p21WAF1/CIP1 (Vigushin et al., Anticancer Drugs, 13(1): 1-13 (January 2002)), and are suitable cell cycle signaling inhibitors for use herein.

Examples of such HDAC inhibitors include:
1. Vorinostat, including pharmaceutically acceptable salts thereof. Marks et al., Nature Biotechnology 25, 84 to 90 (2007); Stenger, Community Oncology 4, 384-386 (2007).
Vorinostat has the following chemical structure and name:

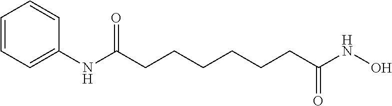 [SMILES: O=C(CCCCCCC(=O)Nc1ccccc1)NO]

-N-hydroxy-N′-phenyl-octanediamide.
2. Romidepsin, including pharmaceutically acceptable salts thereof. Vinodhkumar et al., Biomedicine & Pharmacotherapy 62 (2008) 85-93. Romidepsin, has the following chemical structure and name:

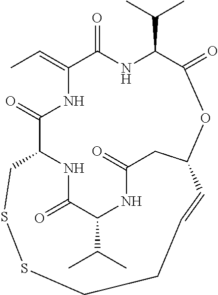 [SMILES: C/C=C1\NC(=O)[C@H]2CSSCC/C=C/[C@H](CC(=O)N[C@H](C(C)C)C(=O)N2)OC(=O)[C@H](C(C)C)NC1=O]

-(1S,4S,7Z,10S,16E,21R)-7-ethylidene-4,21-di(propan-2-yl)-2-oxa-12,13-dithia-5,8,20,23-tetrazabicyclo[8.7.6]tricos-16-ene-3,6,9,19,22-pentone.
3. Panobinostat, including pharmaceutically acceptable salts thereof. Drugs of the Future 32(4): 315-322 (2007).
Panobinostat, has the following chemical structure and name:

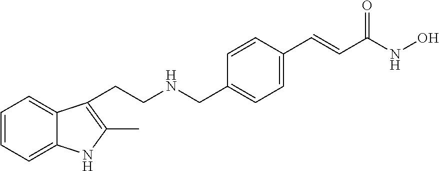 [SMILES: Cc1[nH]c2ccccc2c1CCNCc1ccc(/C=C/C(=O)NO)cc1]

-(2E)-N-hydroxy-3-[4-({[2-(2-methyl-1H-indol-3-yl)ethyl]amino}methyl)phenyl]acrylamide.
4. Valproic acid, including pharmaceutically acceptable salts thereof. Gottlicher, et al., EMBO J. 20(24): 6969-6978 (2001).
Valproic acid, has the following chemical structure and name:

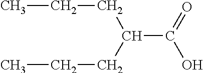

2-propylpentanoic acid.
5. Mocetinostat (MGCD0103), including pharmaceutically acceptable salts thereof. Balasubramanian et al., Cancer Letters 280: 211-221 (2009). Mocetinostat, has the following chemical structure and name:

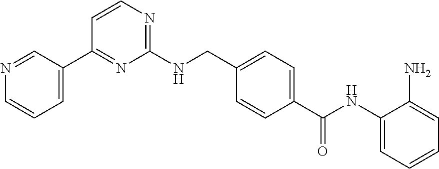 [SMILES: Nc1ccccc1NC(=O)c1ccc(CNc2nccc(-c3cccnc3)n2)cc1]

N-(2-Aminophenyl)-4-[[(4-pyridin-3-ylpyrimidin-2-yl)amino]methyl]benzamide.

Further examples of such HDAC inhibitors are included in Bertrand European Journal of Medicinal Chemistry 45, (2010) 2095-2116, particularly the compounds of table 3 therein as indicated below.

Hydroxamic acids

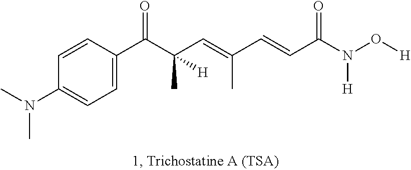 [SMILES: CC(/C=C/C(=O)NO)=C\[C@@H](C)C(=O)c1ccc(N(C)C)cc1]

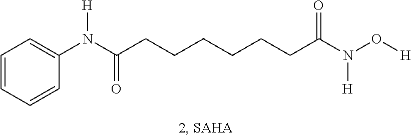 [SMILES: O=C(CCCCCCC(=O)Nc1ccccc1)NO]

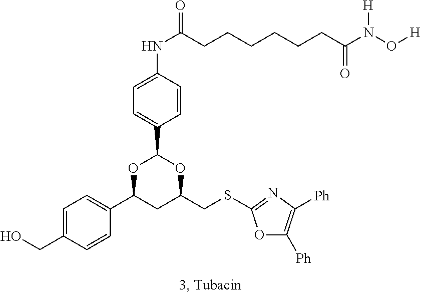 [SMILES: O=C(CCCCCCC(=O)Nc1ccc([C@H]2O[C@@H](CSc3nc(-c4ccccc4)c(-c4ccccc4)o3)C[C@@H](c3ccc(CO)cc3)O2)cc1)NO]

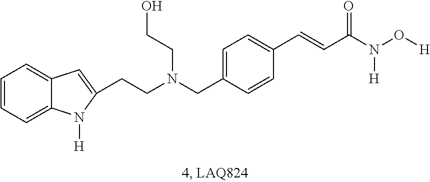 [SMILES: O=C(/C=C/c1ccc(CN(CCO)CCc2cc3ccccc3[nH]2)cc1)NO]

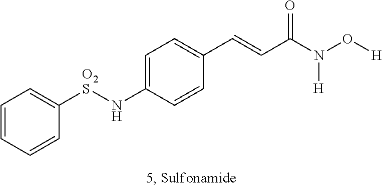 [SMILES: O=C(/C=C/c1ccc(NS(=O)(=O)c2ccccc2)cc1)NO]

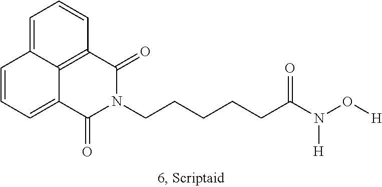 [SMILES: O=C(CCCCCN1C(=O)c2cccc3cccc(c23)C1=O)NO]

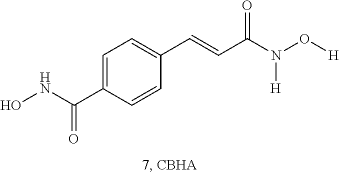 [SMILES: O=C(/C=C/c1ccc(C(=O)NO)cc1)NO]

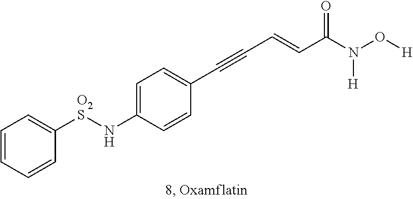 [SMILES: O=C(/C=C/C#Cc1ccc(NS(=O)(=O)c2ccccc2)cc1)NO]

Cyclic tetrapeptides

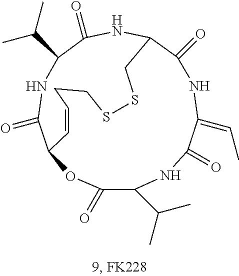 [SMILES: C/C=C1/NC(=O)C2CSSCC/C=C\[C@@H](OC(=O)C(C(C)C)NC1=O)C(=O)N[C@@H](C(C)C)C(=O)N2]

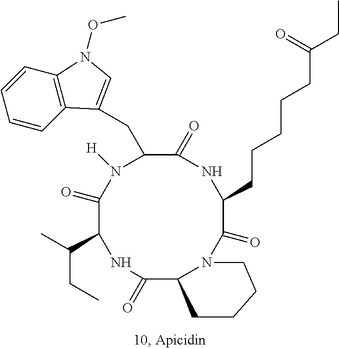 [SMILES: CCC(=O)CCCCC[C@@H]1NC(=O)C(Cc2cn(OC)c3ccccc23)NC(=O)[C@H](C(C)CC)NC(=O)[C@@H]2CCCCN2C1=O]

Short chain carboxylic acids

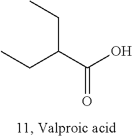

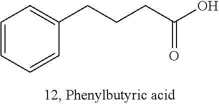 [SMILES: O=C(O)CCCc1ccccc1]

Benzamides

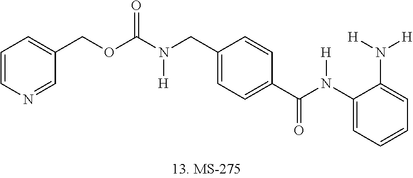 [SMILES: Nc1ccccc1NC(=O)c1ccc(CNC(=O)OCc2cccnc2)cc1]

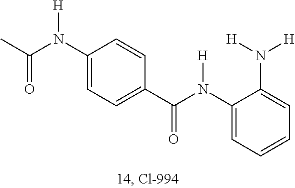 [SMILES: CC(=O)Nc1ccc(C(=O)Nc2ccccc2N)cc1]

Keto derivatives

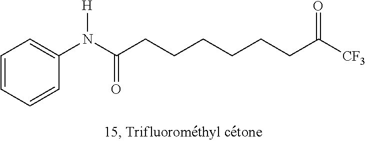 [SMILES: O=C(CCCCCCC(=O)C(F)(F)F)Nc1ccccc1]

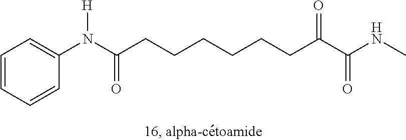 [SMILES: CNC(=O)C(=O)CCCCCCC(=O)Nc1ccccc1]

Proteasome inhibitors are drugs that block the action of proteasomes, cellular complexes that break down proteins, like the p53 protein. Several proteasome inhibitors are marketed or are being studied in the treatment of cancer. Suitable proteasome inhibitors for use herein include:

1. Bortezomib (Velcade®), including pharmaceutically acceptable salts thereof. Adams J, Kauffman M (2004), Cancer Invest 22 (2): 304-11.

Bortezomib has the following chemical structure and name.

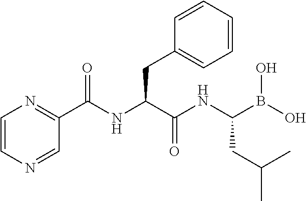 [SMILES: CC(C)C[C@H](NC(=O)[C@H](Cc1ccccc1)NC(=O)c1cnccn1)B(O)O]

### [(1R)-3-methyl-1-({(2S)-3-phenyl-2-[(pyrazin-2-ylcarbonyl)amino]propanoyl}amino)butyl]boronic acid

2. Disulfuram, including pharmaceutically acceptable salts thereof. Bouma et al. (1998). J. Antimicrob. Chemother. 42 (6): 817-20. Disulfuram has the following chemical structure and name.

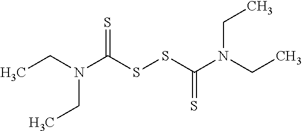 [SMILES: CCN(CC)C(=S)SSC(=S)N(CC)CC]

### 1,1′,1″,1′″[disulfanediylbis(carbonothioylnitrilo)]tetraethane

3. Epigallocatechin gallate (EGCG), including pharmaceutically acceptable salts thereof. Williamson et al., (December 2006), The Journal of Allergy and Clinical Immunology 118 (6): 1369-74.

Epigallocatechin gallate has the following chemical structure and name.

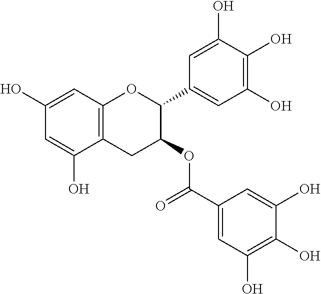 [SMILES: O=C(O[C@H]1Cc2c(O)cc(O)cc2O[C@@H]1c1cc(O)c(O)c(O)c1)c1cc(O)c(O)c(O)c1]

### [(2R,3R)-5,7-dihydroxy-2-(3,4,5-trihydroxyphenyl)chroman-3-yl]3,4,5-trihydroxybenzoate

4. Salinosporamide A, including pharmaceutically acceptable salts thereof. Feling et at., (2003), Angew. Chem. Int. Ed. Engl. 42 (3): 355-7.

Salinosporamide A has the following chemical structure and name.

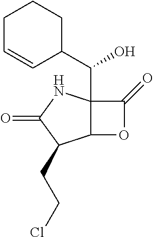 [SMILES: O=C1NC2([C@@H](O)C3C=CCCC3)C(=O)OC2[C@H]1CCCl]

### (4R,5S)-4-(2-chloroethyl)-1-((1S)-cyclohex-2-enyl(hydroxy)methyl)-5-methyl-6-oxa-2-azabicyclo3.2.0heptane-3,7-dione

Inhibitors of cancer metabolism—Many tumor cells show a markedly different metabolism from that of normal tissues. For example, the rate of glycolysis, the metabolic process that converts glucose to pyruvate, is increased, and the pyruvate generated is reduced to lactate, rather than being further oxidized in the mitochondria via the tricarboxylic acid (TCA) cycle. This effect is often seen even under aerobic conditions and is known as the Warburg Effect.

Lactate dehydrogenase A (LDH-A), an isoform of lactate dehydrogenase expressed in muscle cells, plays a pivotal role in tumor cell metabolism by performing the reduction of pyruvate to lactate, which can then be exported out of the cell. The enzyme has been shown to be upregulated in many tumor types. The alteration of glucose metabolism described in the Warburg effect is critical for growth and proliferation of cancer cells and knocking down LDH-A using RNA-i has been shown to lead to a reduction in cell proliferation and tumor growth in xenograft models.
D. A. Tennant et. al., Nature Reviews, 2010, 267.
P. Leder, et. al., Cancer Cell, 2006, 9, 425.

Inhibitors of cancer metabolism, including other inhibitors of LDH-A, are suitable for use in combination with the compounds of this invention.

In one embodiment, the cancer treatment method of the claimed invention includes the co-administration a compound of Formula (I) and/or a pharmaceutically acceptable salt thereof and at least one anti-neoplastic agent, such as one selected from the group consisting of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, and cell cycle signaling inhibitors.

### Compositions

The pharmaceutically active compounds within the scope of this invention are useful as lactate dehydrogenase A inhibitors in mammals, particularly humans, in need thereof.

The present invention therefore provides a method of treating cancer and other conditions requiring lactate dehydrogenase A inhibition, which comprises administering an effective amount of a compound of Formula (I) or a pharmaceutically acceptable salt thereof. The compounds of Formula (I) also provide for a method of treating the above indicated disease states because of their demonstrated ability to act as lactate dehydrogenase A inhibitors. The drug may be administered to a patient in need thereof by any conventional route of administration, including, but not limited to, intravenous, intramuscular, oral, subcutaneous, intradermal, and parenteral.

The pharmaceutically active compounds of the present invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier or diluent may include any prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will be in the form of a syrup, elixir, emulsion, soft gelatin capsule, sterile injectable liquid such as an ampoule, or an aqueous or nonaqueous liquid suspension.

The pharmaceutical preparations are made following conventional techniques of a pharmaceutical chemist involving mixing, granulating, and compressing, when necessary, for tablet forms, or mixing, filling and dissolving the ingredients, as appropriate, to give the desired oral or parenteral products.

Doses of the presently invented pharmaceutically active compounds in a pharmaceutical dosage unit as described above will be an efficacious, nontoxic quantity preferably selected from the range of 0.001-100 mg/kg of active compound, preferably 0.001-50 mg/kg. When treating a human patient in need of a lactate dehydrogenase A inhibitor, the selected dose is administered preferably from 1-6 times daily, orally or parenterally. Preferred forms of parenteral administration include topically, rectally, transdermally, by injection and continuously by infusion. Oral dosage units for human administration preferably contain from 0.05 to 3500 mg of active compound. Oral administration, which uses lower dosages, is preferred. Parenteral administration, at high dosages, however, also can be used when safe and convenient for the patient.

Optimal dosages to be administered may be readily determined by those skilled in the art, and will vary with the particular lactate dehydrogenase A inhibitor in use, the strength of the preparation, the mode of administration, and the advancement of the disease condition. Additional factors depending on the particular patient being treated will result in a need to adjust dosages, including patient age, weight, diet, and time of administration.

The method of this invention of inducing lactate dehydrogenase A inhibitory activity in mammals, including humans, comprises administering to a subject in need of such activity an effective lactate dehydrogenase A inhibiting amount of a pharmaceutically active compound of the present invention.

The invention also provides for the use of a compound of Formula (I) or a pharmaceutically acceptable salt thereof in the manufacture of a medicament for use as a lactate dehydrogenase A inhibitor.

The invention also provides for the use of a compound of Formula (I) or a pharmaceutically acceptable salt thereof in the manufacture of a medicament for use in therapy.

The invention also provides for the use of a compound of Formula (I) or a pharmaceutically acceptable salt thereof in the manufacture of a medicament for use in treating cancer.

The invention also provides for a pharmaceutical composition for use as a lactate dehydrogenase A inhibitor which comprises a compound of Formula (I) or a pharmaceutically acceptable salt thereof and a pharmaceutically acceptable carrier.

The invention also provides for a pharmaceutical composition for use in the treatment of cancer which comprises a compound of Formula (I) or a pharmaceutically acceptable salt thereof and a pharmaceutically acceptable carrier.

In addition, the pharmaceutically active compounds of the present invention can be co-administered with further active ingredients, such as other compounds known to treat cancer, or compounds known to have utility when used in combination with a lactate dehydrogenase A inhibitor.

Without further elaboration, it is believed that one skilled in the art can, using the preceding description, utilize the present invention to its fullest extent. The following Examples are, therefore, to be construed as merely illustrative and not a limitation of the scope of the present invention in any way.

### EXAMPLES

The following examples illustrate the invention. These examples are not intended to limit the scope of the present invention, but rather to provide guidance to the skilled artisan to prepare and use the compounds, compositions, and methods of the present invention.

While particular embodiments of the present invention are described, the skilled artisan will appreciate that various changes and modifications can be made without departing from the spirit and scope of the invention.

When a chemical intermediate is used in multiple examples, one method of preparation of the intermediate is described. It is understood that, regardless of the description, analogous methods of preparing the chemical intermediate may have been used where the intermediate is utilized in other examples.

### Experimental

### Example 1

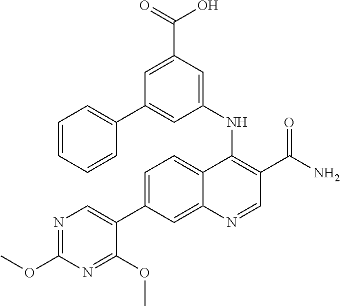 [SMILES: COc1ncc(-c2ccc3c(Nc4cc(C(=O)O)cc(-c5ccccc5)c4)c(C(N)=O)cnc3c2)c(OC)n1]

### 5-({3-(aminocarbonyl)-7-[2,4-bis(methyloxy)-5-pyrimidinyl]-4-quinolinyl}amino)-3-biphenylcarboxylic acid

### a) 7-bromo-4-chloro-3-quinolinecarboxamide

A mixture of 7-bromo-4-hydroxy-3-quinolinecarboxylic acid (3 g, 11.19 mmol) in phosphorus oxychloride (10 mL, 107 mmol) was heated under reflux overnight, then cooled and azeotroped twice with toluene (25 mL). The brown oil was dissolved in tetrahydrofuran (25 mL) then added dropwise to a cold (ice) solution of 30% aqueous ammonia (50 mL, 2311 mmol). After stirring for 2 h, the solid was filtered and washed with water then a little diethyl ether to afford the title compound (2.93 g, 10.26 mmol, 92%) as an off white solid. MS (ES+) m/e 285 [M+H]⁺.

### b) 7-[2,4-bis(methyloxy)-5-pyrimidinyl]-4-chloro-3-quinolinecarboxamide

To a suspension of 7-bromo-4-chloro-3-quinolinecarboxamide (1000 mg, 3.50 mmol), [2,4-bis(methyloxy)-5-pyrimidinyl]boronic acid (773 mg, 4.20 mmol) and potassium carbonate (968 mg, 7.00 mmol) in 1,4-dioxane (18 mL) and water (6.00 mL) was added tetrakis(triphenylphosphine)palladium(0) (202 mg, 0.175 mmol). The mixture was kept stirring at 75° C. overnight. After cooling, the reaction was diluted with water. The precipitate was collected, washed with water, methanol and acetone, and dried under reduced pressure to afford the title compound (1025 mg, 2.97 mmol, 85% yield) as a brown solid. ¹H NMR (400 MHz, DMSO-d6) δ ppm 8.90 (s, 1H), 8.64 (s, 1H), 8.33 (d, J=8.8 Hz, 1H), 8.31 (d, J=1.5 Hz, 1H), 8.23 (br. s., 1H), 8.03 (dd, J=8.7, 1.6 Hz, 1H), 7.98 (br. s., 1H), 4.01 (s, 3H), 3.99 (s, 3H).

### c) 5-amino-3-biphenylcarboxylic acid

To a suspension of 3-amino-5-bromobenzoic acid (600 mg, 2.78 mmol), phenylboronic acid (677 mg, 5.55 mmol) and potassium carbonate (768 mg, 5.55 mmol) in 1,4-dioxane (12 mL) and water (4.00 mL) was added tetrakis(triphenylphosphine)palladium(0) (160 mg, 0.139 mmol). The mixture was heated to 100° C. overnight. After cooling, the mixture was diluted with water, acidified with 6N HCl and extracted with ethyl acetate. The organic layer was dried over MgSO₄, filtered and concentrated under reduced pressure. The precipitate was collected and washed with methylene chloride and ether to afford 5-amino-3-biphenylcarboxylic acid (310 mg, 1.454 mmol, 52.3% yield) as an off white solid. ¹H NMR (400 MHz, DMSO-d6) δ ppm 12.72 (br. s., 1H), 7.58 (d, J=1.5 Hz, 1H), 7.56 (s, 1H), 7.45 (t, J=7.6 Hz, 2H), 7.31-7.39 (m, 2H), 7.17-7.20 (m, 1H), 7.04 (t, J=2.0 Hz, 1H), 5.47 (br. s., 2H).

### d) 5-({3-(aminocarbonyl)-7-[2,4-bis(methyloxy)-5-pyrimidinyl]-4-quinolinyl}amino)-3-biphenylcarboxylic acid

To a suspension of 7-[2,4-bis(methyloxy)-5-pyrimidinyl]-4-chloro-3-quinolinecarboxamide (60 mg, 0.174 mmol) in acetic acid (10 mL) was added 5-amino-3-biphenylcarboxylic acid (37.1 mg, 0.174 mmol). The reaction mixture was stirred at room temperature overnight and was purified through preparative HPLC (YMC 75×30 mm column, 0.1% TFA in water and 0.1% TFA in acetonitrile) to afford 5-({3-(aminocarbonyl)-7-[2,4-bis(methyloxy)-5-pyrimidinyl]-4-quinolinyl}amino)-3-biphenylcarboxylic acid (35 mg, 0.055 mmol, 31.6% yield) as a yellow solid. ¹H NMR (400 MHz, DMSO-d6) δ ppm 13.30 (br. s., 1H), 11.55 (br. s., 1H), 8.98 (s, 1H), 8.66 (s, 1H), 8.35 (d, J=9.1 Hz, 1H), 8.26 (d, J=1.5 Hz, 2H), 8.06 (s, 1H), 7.92 (dd, J=9.0, 1.6 Hz, 1H), 7.82 (s, 1H), 7.78 (s, 1H), 7.68-7.76 (m, 3H), 7.45-7.52 (m, 2H), 7.38-7.44 (m, 1H), 4.01 (s, 3H), 3.99 (s, 3H).

### Example 2

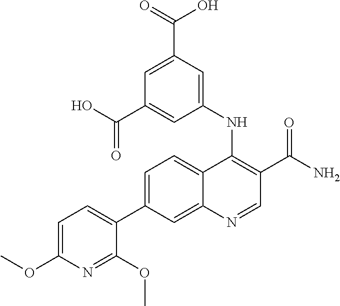 [SMILES: COc1ccc(-c2ccc3c(Nc4cc(C(=O)O)cc(C(=O)O)c4)c(C(N)=O)cnc3c2)c(OC)n1]

### 5-({3-(aminocarbonyl)-7-[2,6-bis(methyloxy)-3-pyridinyl]-4-quinolinyl}amino)-1,3-benzenedicarboxylic acid, trifluoroacetate salt

### a) dimethyl 5-({3-(aminocarbonyl)-7-[2,6-bis(methyloxy)-3-pyridinyl]-4-quinolinyl}amino)-1,3-benzenedicarboxylate

A solution of dimethyl 5-{[3-(aminocarbonyl)-7-bromo-4-quinolinyl]amino}-1,3-benzenedicarboxylate (example 4a, 280 mg, 0.611 mmol), [2,6-dimethoxy)-3-pyridinyl]boronic acid (335 mg, 1.833 mmol), potassium carbonate (253 mg, 1.833 mmol), and tetrakis(triphenylphosphine)palladium(0) (31.8 mg, 0.027 mmol) in 1,4-dioxane (3 ml) and water (1.000 ml) was heated to 120° C. for 20 min. in a Biotage Initiator® microwave synthesizer. Upon cooling, the reaction mixture was filtered to obtain a green solid, which contained a mixture of di-acid compound and di-ester (118 mg). This mixture was directly taken to the next step for hydrolysis of both the esters. The filtrate was diluted with water and extracted with ethyl acetate twice. The combined organic portions were dried (MgSO₄) and concentrated. Following purification via flash column chromatography (0.1% triethylamine, 10% methanol in dichloromethane), the desired fractions were concentrated and the residue triturated in ethyl ether to obtain the title compound (0.150 g, 48%) as a yellow solid. ¹H NMR (400 MHz, DMSO-d₆) ppm 10.00 (s, 1H), 8.95 (s, 1H), 8.12-8.23 (m, 2H), 8.03-8.07 (m, 1H), 7.99 (d, J=8.3 Hz, 1H), 7.94 (d, J=8.8 Hz, 1H), 7.68-7.76 (m, 3H), 7.50-7.62 (m, 1H), 6.55 (d, J=8.1 Hz, 1H), 3.96 (s, 3H), 3.94 (s, 3H), 3.84 (s, 6H).

### b) 5-({3-(aminocarbonyl)-7-[2,6-bis(methyloxy)-3-pyridinyl]-4-quinolinyl}amino)-1,3-benzenedicarboxylic acid

To a suspension of dimethyl 5-({3-(aminocarbonyl)-7-[2,6-bis(methyloxy)-3-pyridinyl]-4-quinolinyl}amino)-1,3-benzenedicarboxylate (150 mg, 0.290 mmol) in ethanol (2.0 mL) was added 1N aqueous sodium hydroxide (3 ml, 2.308 mmol). After stirring 2 h at ambient temperature, the reaction mixture was concentrated to an aqueous residue, which was then neutralized with 6N aqueous hydrochloric acid. The resulting solution was purified using Reverse Phase HPLC (ODS column 0.1 TFA in acetonitrile and water) to obtain the title compound (0.030 g, 17%) as a yellow solid. ¹H NMR (400 MHz, DMSO-d₆) ppm 8.94 (s, 1H), 8.12-8.29 (m, 4H), 7.98 (d, J=8.3 Hz, 1H), 7.78-7.93 (m, 3H), 7.66 (br. s., 1H), 6.59 (d, J=8.1 Hz, 1H), 3.98 (s, 3H), 3.95 (s, 3H).

### Example 3

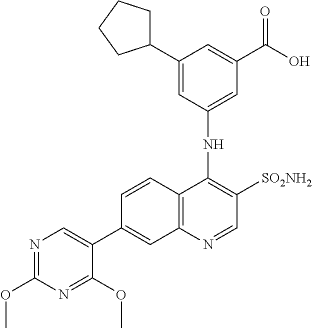 [SMILES: COc1ncc(-c2ccc3c(Nc4cc(C(=O)O)cc(C5CCCC5)c4)c(S(N)(=O)=O)cnc3c2)c(OC)n1]

### 3-({3-(aminosulfonyl)-7-[2,4-bis(methyloxy)-5-pyrimidinyl]-4-quinolinyl}amino)-5-cyclopentylbenzoic acid, trifluoroacetate salt

### a) 7-[2,4-bis(methyloxy)-5-pyrimidinyl]-4-chloro-3-quinolinesulfonamide

A mixture of 7-bromo-4-chloro-3-quinolinesulfonamide (example 52b, 0.500 g, 1.56 mmol), [2,4-bis(methyloxy)-5-pyrimidinyl]boronic acid (0.343 g, 1.87 mmol), potassium carbonate (0.516 g, 3.73 mmol), tetrakis(triphenylphosphine)palladium(0) (0.090 g, 0.078 mmol), 1,4-dioxane (8 mL) and water (2 mL) was stirred at 80° C. under nitrogen for 2.5 h, then cooled. Water (20 mL) was added and the mixture stirred 10 min. The solid was filtered off, washed with water and dried to give the title compound (0.429 g, 73%) as a cream solid. ¹H NMR (400 MHz, DMSO-d₆) ppm 4.00 (s, 3H) 4.03 (s, 3H) 8.04 (s, 2H) 8.12 (dd, J=8.84, 1.77 Hz, 1H) 8.38 (d, J=1.52 Hz, 1H) 8.47 (d, J=9.09 Hz, 1H) 8.68 (s, 1H) 9.30 (s, 1H).

### b) 3-({3-(aminosulfonyl)-7-[2,4-bis(methyloxy)-5-pyrimidinyl]-4-quinolinyl}amino)-5-cyclopentylbenzoic acid, trifluoroacetate salt

3-amino-5-cyclopentylbenzoic acid (example 53b, 26.9 mg, 0.131 mmol) was added to a stirred solution of 7-[2,4-bis(methyloxy)-5-pyrimidinyl]-4-chloro-3-quinolinesulfonamide (example, 50 mg, 0.131 mmol) in acetic acid (2 mL) at rt. The reaction was stirred for 24 h, then the precipitate was filtered, washed with acetic acid, and dried in vacuo. Purification by reverse-phase HPLC (10-50% acetonitrile/water w/0.1% TFA) afforded the title compound (27 mg, 0.041 mmol, 31.0% yield) as a yellow solid. ¹H NMR (DMSO-d₆) d: 9.14 (s, 1H), 8.59 (s, 1H), 8.25 (s, 1H), 7.94 (br. s., 2H), 7.52-7.72 (m, 3H), 7.34 (s, 1H), 7.18 (s, 1H), 3.98 (s, 3H), 3.97 (s, 3H), 2.91-3.08 (m, 1H), 1.99 (d, J=7.6 Hz, 2H), 1.55-1.77 (m, 4H), 1.35-1.55 (m, 2H). MS (ES+) m/e 550 [M+H]⁺.

### Example 4

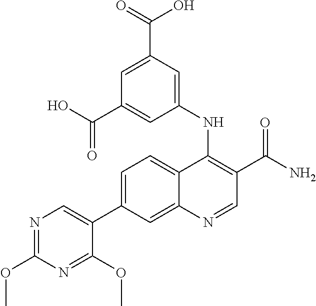 [SMILES: COc1ncc(-c2ccc3c(Nc4cc(C(=O)O)cc(C(=O)O)c4)c(C(N)=O)cnc3c2)c(OC)n1]

### 5-({3-(aminocarbonyl)-7-[2,4-bis(methyloxy)-5-pyrimidinyl]-4-quiuinolinyl}amino)-1,3-benzenedicarboxylic acid

### a) dimethyl 5-{[3-(aminocarbonyl)-7-bromo-4-quinolinyl]amino}-1,3-benzenedicarboxylate

A mixture of 7-bromo-4-chloro-3-quinolinecarboxamide (example 1a, 3.0 g, 10.51 mmol) and dimethyl-5-aminoisophthalate (2.198 g, 10.51 mmol) in ethanol (10 mL) and acetic acid (1 mL) was heated to 80° C. overnight in an oil bath. Upon cooling, a solid precipitated out and was filtered, then washed with ethyl ether and dried to obtain the title compound (4.00 g, 83%) as a pale yellow solid. MS (ES+) m/e 458/460 [M+H]⁺.

### b) 5-({3-(aminocarbonyl)-7-[2,4-bis(methyloxy)-5-pyrimidinyl]-4-quinolinyl}amino)-1,3-benzenedicarboxylic acid

A solution of dimethyl 5-{[3-(aminocarbonyl)-7-bromo-4-quinolinyl]amino}-1,3-benzenedicarboxylate (200 mg, 0.436 mmol), 2,4-dimethoxypyrimidine-5-boronic acid (241 mg, 1.309 mmol), potassium carbonate (181 mg, 1.309 mmol), and tetrakis(triphenylphosphine)palladium(0) (22.69 mg, 0.020 mmol) in 1,4-dioxane (2.0 ml) and water (0.667 ml) was heated to 100° C. for min. in a Biotage Initiator® microwave synthesizer. Upon cooling, the reaction mixture was diluted with water, extracted with ethyl acetate dried using magnesium sulfate and concentrated in vacuo. The residue was purified via flash column chromatography (0-100% ethyl acetate in hexanes, then 10% methanol in ethyl acetate). Sodium hydroxide (1N aq solution) (2 ml, 2.000 mmol) was added to a slurry of the obtained solid (75% by LC-MS) as a slurry in ethanol (5 ml). After stirring for 2 h at ambient temperature, the reaction was concentrated to an aq residue, which was neutralized with 6N aqueous hydrochloric acid. The resulting precipitate was filtered, washed with water, and purified using Reverse Phase HPLC (0.1% TFA in acetonitrile and water) to obtain the title compound (0.009 g, 3%) as an off white solid. ¹H NMR (400 MHz, DMSO-d₆) ppm 13.38 (br. s., 2H), 11.09 (br. s., 1H), 8.94 (s, 1H), 8.66 (s, 1H), 8.23-8.35 (m, 3H), 8.20 (br. s., 1H), 7.85-7.98 (m, 3H), 7.66 (br. s., 1H), 4.02 (s, 3H), 3.99 (s, 3H).

### Example 5

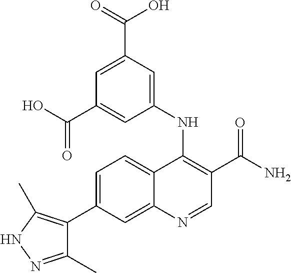 [SMILES: Cc1n[nH]c(C)c1-c1ccc2c(Nc3cc(C(=O)O)cc(C(=O)O)c3)c(C(N)=O)cnc2c1]

### 5-{[3-(aminocarbonyl)-7-(3,5-dimethyl-1H-pyrazol-4-yl)-4-quinolinyl]amino}-1,3-benzenedicarboxylic acid

### a) dimethyl 5-{[3-(aminocarbonyl)-7-(3,5-dimethyl-1H-pyrazol-4-yl)-4-quinolinyl]amino}-1,3-benzenedicarboxylate

A solution of dimethyl 5-{[3-(aminocarbonyl)-7-bromo-4-quinolinyl]amino}-1,3-benzenedicarboxylate (example 4a, 1.2 g, 2.62 mmol), 3,5-dimethyl-1H-pyrazol-4-boronic acid (1.745 g, 7.86 mmol), potassium carbonate (1.086 g, 7.86 mmol), and tetrakis(triphenylphosphine)palladium(0) (0.136 g, 0.118 mmol) in 1,4-dioxane (2.0 ml) and water (0.667 ml) was heated to 100° C. for 20 min. in a Biotage Initiator® microwave synthesizer. Upon cooling, the reaction mixture was diluted with water and extracted with ethyl acetate twice. The combined organic portions were dried (MgSO₄) and concentrated. Following purification via flash column chromatography (10% methanol in dichloromethane), the desired fractions were concentrated and the residue triturated in ethyl ether to obtain the title compound (0.370 g, 30%) as a yellow solid. MS (ES+) m/e 474 [M+H]⁺.

### b) 5-{[3-(aminocarbonyl)-7-(3,5-dimethyl-1H-pyrazol-4-yl)-4-quinolinyl]amino}-1,3-benzenedicarboxylic acid

To a suspension of dimethyl 5-{[3-(aminocarbonyl)-7-(3,5-dimethyl-1H-pyrazol-4-yl)-4-quinolinyl]amino}-1,3-benzenedicarboxylate (370 mg, 0.781 mmol) in methanol (3 mL) was added 1N aqueous sodium hydroxide (3 ml, 3.00 mmol). After stirring overnight at ambient temperature, the reaction mixture was concentrated to an aqueous residue, which was then neutralized with 6N aqueous hydrochloric acid. The resulting precipitate was filtered, washed with water, and dried in vacuo to obtain the title compound (200 mg, 57%) as a yellow solid. ¹H NMR (400 MHz, DMSO-d₆) ppm 11.22 (br. s., 1H), 9.16 (s, 1H), 8.44 (br. s., 1H), 8.31-8.41 (m, 2H), 8.16 (s, 1H), 8.07 (s, 2H), 7.84 (br. s., 1H), 7.79 (d, J=8.8 Hz, 1H), 2.50 (s, 6H).

### Example 6

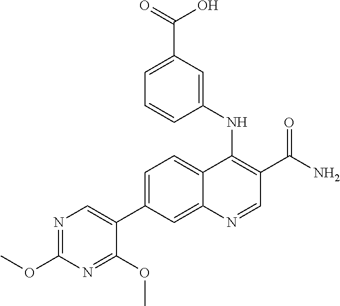 [SMILES: COc1ncc(-c2ccc3c(Nc4cccc(C(=O)O)c4)c(C(N)=O)cnc3c2)c(OC)n1]

### 3-({3-(aminocarbonyl)-7-[2,4-bis(methyloxy)-5-pyrimidinyl]-4-quinoliny}amino)benzoic acid

### a) ethyl 3-{[3-(aminocarbonyl)-7-bromo-4-quinolinyl]amino}benzoate

A mixture of 7-bromo-4-chloro-3-quinolinecarboxamide (example 1a, 2.08 g, 7.28 mmol) and ethyl 3-aminobenzoate (1.203 g, 7.28 mmol) in 1,4-dioxane (10 mL) was heated to 110° C. for 2 h in an oil bath. Upon cooling, a solid precipitated out and it was filtered, then washed with ethyl ether and dried to obtain the title compound (2.53 g, 84%) as a pale yellow solid. ¹H NMR (400 MHz, DMSO-d₆) ppm 11.58 (br. s., 1H), 8.99 (s, 1H), 8.26 (d, J=1.8 Hz, 2H), 8.15 (d, J=9.1 Hz, 1H), 7.83 (s, 1H), 7.81 (s, 2H), 7.72 (s, 1H), 7.50-7.55 (m, 1H), 7.44-7.50 (m, 1H), 4.32 (q, J=7.1 Hz, 2H), 1.31 (t, J=7.1 Hz, 3H).

### b) ethyl 3-({3-(aminocarbonyl)-7-[2,4-bis(methyloxy)-5-pyrimidinyl]-4-quinolinyl}amino)benzoate

A solution of ethyl 3-{[3-(aminocarbonyl)-7-bromo-4-quinolinyl]amino}benzoate (200 mg, 0.483 mmol), 2,4-dimethoxy-pyrimidine-5-boronic acid (266 mg, 1.448 mmol), potassium carbonate (200 mg, 1.448 mmol), and tetrakis(triphenylphosphine)palladium(0) (40 mg, 0.035 mmol) in 1,4-dioxane (2.0 ml) and water (0.667 ml) was heated to 100° C. for 20 min. in a Biotage Initiator® microwave synthesizer. Upon cooling, the reaction mixture was diluted with water, extracted with ethyl acetate dried using magnesium sulfate and concentrated in vacuo. The residue was purified via flash column chromatography (10% methanol in dichloromethane). The fractions containing the product were concentrated and triturated with ethyl ether to obtain the title compound ethyl 3-({3-(aminocarbonyl)-7-[2,4-bis(methyloxy)-5-pyrimidinyl]-4-quinolinyl}amino)benzoate (150 mg, 0.317 mmol, 65.6% yield) as a yellow solid. ¹H NMR (400 MHz, DMSO-d₆) ppm 10.33 (s, 1H), 8.99 (s, 1H), 8.42 (s, 1H), 8.24 (br. s., 1H), 8.11 (d, J=1.8 Hz, 1H), 7.81 (d, J=8.8 Hz, 1H), 7.68 (br. s., 1H), 7.52-7.61 (m, 3H), 7.38 (t, 1H), 7.19 (dd, J=7.8, 1.8 Hz, 1H), 4.27 (q, J=7.1 Hz, 2H), 3.90 (s, 3H), 3.45 (s, 3H), 1.28 (t, J=7.1 Hz, 3H).

### c) 3-({3-(aminocarbonyl)-7-[2,4-bis(methyloxy)-5-pyrimidinyl]-4-quinolinyl}amino)benzoic acid

To a suspension of ethyl 3-({3-(aminocarbonyl)-7-[2,4-bis(methyloxy)-5-pyrimidinyl]-4-quinolinyl}amino)benzoate (70 mg, 0.148 mmol) in ethanol (1.0 mL) was added 1N aqueous sodium hydroxide (1 ml, 1.273 mmol). After stirring overnight at ambient temperature, the reaction was concentrated to an aq residue and quenched with 6N aqueous hydrochloric acid.

The resulting precipitate was filtered, washed with water, and dried in vacuo to obtained the title compound (0.033 g, 50%) as a yellow solid. ¹H NMR (400 MHz, DMSO-d₆) ppm 10.63 (s, 1H), 9.04 (s, 1H), 8.54 (s, 1H), 8.38 (br. s., 1H), 8.13 (s, 1H), 7.69-7.82 (m, 2H), 7.59 (d, J=7.6 Hz, 1H), 7.53 (dd, J=9.0, 1.1 Hz, 1H), 7.50 (s, 1H), 7.28 (t, J=7.8 Hz, 1H), 7.08 (d, J=7.6 Hz, 1H), 3.96 (s, 3H), 3.95 (s, 3H).

### Example 7

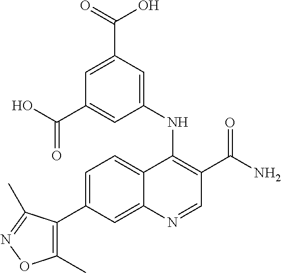 [SMILES: Cc1noc(C)c1-c1ccc2c(Nc3cc(C(=O)O)cc(C(=O)O)c3)c(C(N)=O)cnc2c1]

### 5-{[3-(aminocarbonyl)-7-(3,5-dimethyl-4-isoxazolyl)-4-quinolinyl]amino}-1,3-benzenedicarboxylic acid

### a) 5-{[3-(aminocarbonyl)-7-(3,5-dimethyl-4-isoxazolyl)-4-quinolinyl]amino}-1,3-benzenedicarboxylic acid

To a suspension of 4-chloro-7-(3,5-dimethyl-4-isoxazolyl)-3-quinolinecarboxamide (example 86a, 120 mg, 0.398 mmol) in acetic acid (10 mL) was added 5-amino-1,3-benzenedicarboxylic acid (144 mg, 0.795 mmol). The reaction mixture was stirred at 100° C. for 2 hours. After cooling, the reaction was quenched with water. The precipitate was collected, washed with water, acetone and dried to afford 5-{[3-(aminocarbonyl)-7-(3,5-dimethyl-4-isoxazolyl)-4-quinolinyl]amino}-1,3-benzenedicarboxylic acid (114 mg, 0.255 mmol, 64.2% yield) as a yellow solid. ¹H NMR (400 MHz, DMSO-d6) δ ppm 13.38 (br. s., 1H), 11.76 (br. s., 1H), 9.01 (s, 1H), 8.52 (d, J=8.8 Hz, 1H), 8.34 (s, 1H), 8.28 (s, 1H),8.18 (d, J=1.5 Hz, 1H), 8.05 (d, J=1.3 Hz, 1H), 7.81 (dd, J=8.8, 1.8 Hz, 1H), 7.69 (s, 1H), 2.55 (s, 3H), 2.36 (s, 3H).

### Example 8

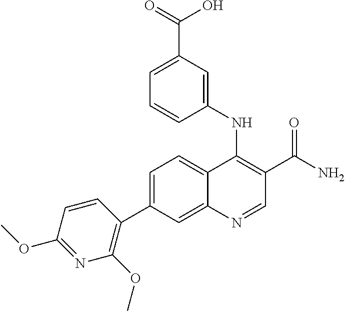 [SMILES: COc1ccc(-c2ccc3c(Nc4cccc(C(=O)O)c4)c(C(N)=O)cnc3c2)c(OC)n1]

### 3-({3-(aminocarbonyl)-7-[2,6-bis(methyloxy)-3-pyridinyl]-4-quinolinyl}amino)benzoic acid

### a) ethyl 3-({3-(aminocarbonyl)-7-[2,6-bis(methyloxy)-3-pyridinyl]-4-quinolinyl}amino)benzoate

A solution of ethyl 3-{[3-(aminocarbonyl)-7-bromo-4-quinolinyl]amino}benzoate (example 6a, 115 mg, 0.278 mmol), [2,6-bis(methyloxy)-3-pyridinyl]boronic acid (152 mg, 0.833 mmol), potassium carbonate (115 mg, 0.833 mmol), and tetrakis(triphenylphosphine)palladium(0) (16.04 mg, 0.014 mmol) in 1,4-dioxane (2.0 ml) and water (0.667 ml) was heated to 120° C. for 20 min. in a Biotage Initiator® microwave synthesizer. Upon cooling, the reaction mixture was diluted with water (2 ml) and then extracted using ethyl acetate twice. The organic portion was dried over magnesium sulfate, filtered and concentrated. Following purification using flash chromatography (10-100% ethyl acetate in hexanes) the desired fractions were concentrated under reduced pressure. The obtained solid was triturated in ethyl ether to obtain the title compound (0.060 g, 46%) as an off white solid. ¹H NMR (400 MHz, DMSO-d₆) ppm 10.35 (s, 1H), 8.99 (s, 1H), 8.24 (br. s., 1H), 8.13 (br. s., 1H), 7.90 (d, J=8.1 Hz, 1H), 7.80 (d, J=8.6 Hz, 1H), 7.49-7.70 (m, 4H), 7.38 (t, J=8.8 Hz, 1H), 7.10-7.25 (m, 1H), 6.53 (d, J=8.1 Hz, 1H), 4.27 (q, J=7.1 Hz, 2H), 3.95 (s, 3H), 3.93 (s, 3H), 1.27 (t, J=7.1 Hz, 3H).

### b) 3-({3-(aminocarbonyl)-7-[2,6-bis(methyloxy)-3-pyridinyl]-4-quinolinyl}amino)benzoic acid

To a suspension of ethyl 3-({3-(aminocarbonyl)-7-[2,6-bis(methyloxy)-3-pyridinyl]-4-quinolinyl}amino)benzoate (42 mg, 0.089 mmol) in ethanol (1.0 mL) was added 1N aqueous sodium hydroxide (1 ml, 1.000 mmol). After stirring 3 h at ambient temperature, the reaction mixture was concentrated to an aqueous residue, which was then neutralized with 6N aqueous hydrochloric acid. The resulting precipitate was filtered, washed with water, and dried in vacuo to obtain the title compound as an orange solid. ¹H NMR (400 MHz, DMSO-d₆) ppm 11.75 (br. s., 1H), 9.05 (s, 1H), 8.40 (br. s., 1H), 8.33 (d, J=1.3 Hz, 1H), 8.08 (d, J=9.1 Hz, 1H), 7.72-7.86 (m, 4H), 7.40-7.58 (m, 2H), 6.59 (s, 1H), 6.57 (s, 1H), 3.97 (s, 3H), 3.94 (s, 3H).

### Example 9

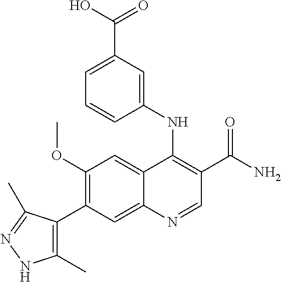 [SMILES: COc1cc2c(Nc3cccc(C(=O)O)c3)c(C(N)=O)cnc2cc1-c1c(C)n[nH]c1C]

### 3-{[3-(aminocarbonyl)-7-(3,5-dimethyl-1H-pyrazol-4-yl)-6-(methyloxy)-4-quinolinyl]amino}benzoic acid

### a) diethyl ({[3-chloro-4-(methyloxy)phenyl]amino}methylidene)propanedioate

A mixture of was 3-chloro-4-methoxyaniline (9.46 g, 60.0 mmol), diethyl ethoxymethylenemalonate (13.0 g, 60.0 mmol) and ethanol (60 mL) was stirred under reflux for 3 h, then cooled. The mixture was diluted with 50% aqueous ethanol (100 mL). The solid was filtered off, washed with 50% aqueous ethanol and dried to give the title compound (17.2 g, 87%) as a cream solid. ¹H NMR (400 MHz, DMSO-d₆) ppm 1.25 (t, J=7.07 Hz, 6H) 3.85 (s, 3H) 4.16 (br. s., 4H) 7.16 (d, J=8.84 Hz, 1H) 7.35 (dd, J=8.84, 2.78 Hz, 1H) 7.59 (d, J=2.78 Hz, 1H) 8.29 (s, 1H) 10.63 (br. s., 1H).

### b) ethyl 7-chloro-4-hydroxy-6-(methyloxy)-3-quinolinecarboxylate

Diphenyl ether (50 mL) was brought to reflux and diethyl ({[3-chloro-4-(methyloxy)phenyl]amino}methylidene)propanedioate (6.00 g, 18.3 mmol) added portionwise. After the addition, the solution was refluxed for 15 min, then cooled and diluted with ether (50 mL). The precipitate was filtered off, washed with ether and dried to give the title compound (4.51 g, 87%) as a cream solid. ¹H NMR (400 MHz, DMSO-d₆) ppm 1.28 (t, 3H) 3.95 (s, 3H) 4.22 (q, J=7.24 Hz, 2H) 7.69 (s, 1H) 7.75 (s, 1H) 8.55 (s, 1H).

### c) 7-chloro-4-hydroxy-6-(methyloxy)-3-quinolinecarboxylic acid

A mixture of ethyl 7-chloro-4-hydroxy-6-(methyloxy)-3-quinolinecarboxylate (4.50 g, 16.0 mmol), 1M aqueous sodium hydroxide (80 mL, 80 mmol) and ethanol was stirred at 80° C. for 1 h, then cooled. The mixture was concentrated to half its volume under reduced pressure and acidified to pH 1 with 1 M aqueous hydrochloric acid. The precipitate was filtered off, washed with water and dried to give the title compound (4.13 g, 100%) as a cream solid. ¹H NMR (400 MHz, DMSO-d₆) ppm 4.02 (s, 3H) 7.77 (s, 1H) 7.94 (s, 1H) 8.90 (s, 1H) 13.39 (br. s., 1H) 15.39 (s, 1H).

### d) 4,7-dichloro-6-(methyloxy)-3-quinolinecarboxamide

A mixture of 7-chloro-4-hydroxy-6-(methyloxy)-3-quinolinecarboxylic acid (2.14 g, 8.44 mmol) and thionyl chloride (30 mL, 411 mmol) was heated under reflux for 2 h. The starting material was sparingly soluble. Phosphorus oxychloride (6.00 mL, 64.4 mmol) was added and the mixture refluxed for 18 h, giving a pale yellow solution. The volatiles were removed under reduced pressure and the residue azeotroped with toluene once. The crude acid chloride was slurried in dioxane (50 mL) and cooled to 0° C. under nitrogen. 0.5 M ammonia in dioxane (169 mL, 84 mmol) was added and the mixture stirred for 2 h while warming to room temperature. The solvent was removed under reduced pressure and the residue slurried in water (150 mL). The solid was filtered off, washed with water and dried to give the title compound (1.93 g, 84%) as a cream solid. ¹H NMR (400 MHz, DMSO-d₆) ppm 4.09 (s, 3H) 7.64 (s, 1H) 7.97 (br. s., 1H) 8.20 (br. s., 1H) 8.26 (s, 1H) 8.76 (s, 1H).

### e) methyl 3-{[3-(aminocarbonyl)-7-chloro-6-(methyloxy)-4-quinolinyl]amino}benzoate

A mixture of 4,7-dichloro-6-(methyloxy)-3-quinolinecarboxamide (1.43 g, 5.27 mmol), methyl 3-aminobenzoate (1.04 g, 6.88 mmol) and acetic acid (20 mL) was heated at 50° C. for 1 h. On cooling to room temperature, a precipitate separated and was filtered off, washed with acetic acid and dried to give the title compound (1.93 g, 95%) as a yellow solid. ¹H NMR (400 MHz, DMSO-d₆) ppm 3.86 (s, 3H) 3.87 (s, 3H) 7.47-7.63 (m, 3H) 7.79-7.85 (m, 2H) 7.91 (s, 1H) 8.14 (s, 1H) 8.20 (s, 1H) 8.87 (s, 1H) 11.31 (br. s., 1H).

### f) methyl 3-{[3-(aminocarbonyl)-7-(3,5-dimethyl-1H-pyrazol-4-yl)-6-(methyloxy)-4-quinolinyl]amino}benzoate

A mixture of methyl 3-{[3-(aminocarbonyl)-7-chloro-6-(methyloxy)-4-quinolinyl]amino}benzoate (0.154 g, 0.400 mmol), 3,5-dimethylpyrazole-4-boronic acid pinacol ester (0.178 g, 0.800 mmol), bis(tri-t-butylphosphine)palladium(0) (0.020 g, 0.040 mmol) and potassium carbonate (0.166 g, 3.00 mmol) in 1,4-dioxane (2 mL) and water (0.6 mL) was stirred at 120° C. in a microwave synthesiser for 0.5 h, then cooled and diluted with water (20 mL). The precipitate was filtered off, washed with water and dried, then chromatographed (silica gel, 2-10% methanol/dichloromethane) to give the title compound (0.063 g, 35%) as a yellow powder. ¹H NMR (400 MHz, DMSO-d₆) ppm 2.09 (br. s., 6H) 3.54 (s, 3H) 3.81 (s, 3H) 7.21 (s, 1H) 7.22-7.26 (m, 1H) 7.41 (t, J=7.83 Hz, 1H) 7.51-7.63 (m, 3H) 7.69 (s, 1H) 8.13 (br. s., 1H) 8.84 (s, 1H) 10.09 (s, 1H) 12.34 (br. s., 1H).

### g) 3-{[3-(aminocarbonyl)-7-(3,5-dimethyl-1H-pyrazol-4-yl)-6-(methyloxy)-4-quinolinyl]amino}benzoic acid

1M aqueous sodium hydroxide (1.20 mL, 1.20 mmol) was added to a solution of methyl 3-{[3-(aminocarbonyl)-7-(3,5-dimethyl-1H-pyrazol-4-yl)-6-(methyloxy)-4-quinolinyl]amino}benzoate (0.052 g, 0.117 mmol) in ethanol (3 mL) and the mixture stirred at room temperature for 18 h, then diluted with water (20 mL). The pH was adjusted to 2 with 1M aqueous hydrochloric acid.

The solvent was removed under reduced pressure and the residue triturated with water. The solid was filtered off, washed with a small amount of water and dried to give the title compound (0.034 g, 68%) as a pale yellow solid. ¹H NMR (400 MHz, DMSO-d₆) ppm 2.13 (s, 6H) 3.72 (s, 3H) 7.51-7.61 (m, 2H) 7.66 (br. s., 1H) 7.74 (s, 1H) 7.80-7.93 (m, 3H) 8.19 (br. s., 1H) 8.88 (s, 1H) 11.54 (br. s., 1H) 14.87 (br. s., 1H).

### Example 10

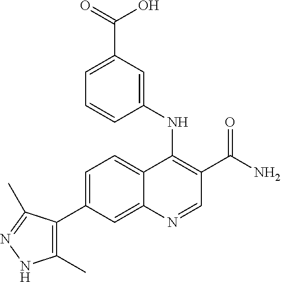 [SMILES: Cc1n[nH]c(C)c1-c1ccc2c(Nc3cccc(C(=O)O)c3)c(C(N)=O)cnc2c1]

### 3-{[3-(aminocarbonyl)-7-(3,5-dimethyl-1H-pyrazol-4-yl)-4-quinolinyl]amino}benzoic acid, dihydrochloride salt

A solution of ethyl 3-{[3-(aminocarbonyl)-7-bromo-4-quinolinyl]amino}benzoate (example 6a, 137 mg, 0.331 mmol), (3,5-dimethyl-1H-pyrazol-4-yl)boronic acid pinacol ester (220 mg, 0.992 mmol), potassium carbonate (137 mg, 0.992 mmol), and tetrakis(triphenylphosphine)palladium(0) (17.20 mg, 0.015 mmol) in 1,4-dioxane (3 ml) and water (1.000 ml) was heated to 120° C. for 20 min. in a Biotage Initiator® microwave synthesizer. Upon cooling, the reaction mixture was concentrated in vacuo, diluted with ethanol (3 ml) and then treated with sodium hydroxide (1M aq solution) (2 ml, 2.000 mmol). Following stirring overnight at room temperature, the ethanol was removed in vacuo, the residue diluted with water and extracted using ethyl acetate. The aqueous portion was then adjusted to pH˜4 using 1N hydrochloric acid. The resulting solid was filtered and dried in vacuo to obtain the title compound (0.111 g, 71%) as a yellow solid. ¹H NMR (400 MHz, DMSO-d₆) ppm 12.75 (br. s., 1H), 10.51 (br. s., 1H), 9.00 (s, 1H), 8.26 (br. s., 1H), 7.77-7.89 (m, 2H), 7.68 (br. s., 1H), 7.59 (d, J=7.6 Hz, 1H), 7.52 (br. s., 1H), 7.35-7.44 (m, 2H), 7.24 (dd, J=8.1, 1.5 Hz, 1H), 2.28 (s, 6H).

### Example 11

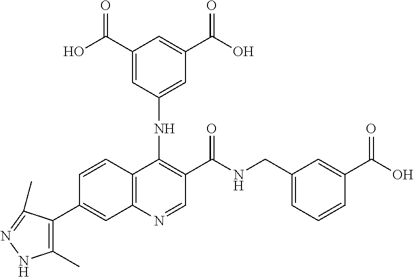 [SMILES: Cc1n[nH]c(C)c1-c1ccc2c(Nc3cc(C(=O)O)cc(C(=O)O)c3)c(C(=O)NCc3cccc(C(=O)O)c3)cnc2c1]

### 5-{[3-({[(3-carboxyphenyl)methyl]amino}carbonyl)-7-(3,5-dimethyl-1H-pyrazol-4-yl)-4-quinolinyl]amino}-1,3-benzenedicarboxylic acid

### a) 4-({3,5-bis[(methyloxy)carbonyl]phenyl}amino)-7-bromo-3-quinolinecarboxylic acid

A 50 mL flask was charged with ethyl 7-bromo-4-chloro-3-quinolinecarboxylate (A. C. Barrios Sosa et. al., Bioorganic & Medicinal Chemistry Letters, 2004, 14(9), 2155-2158; 500 mg, 1.590 mmol) and sodium hydroxide (0.262 mL, 1.574 mmol) in ethanol (12 mL). Stirring for 1 hour at 50° C. a white precipitate formed. The reaction was then concentrated under vacuum, and dimethyl 5-amino-1,3-benzenedicarboxylate (166 mg, 0.795 mmol) and acetic acid (10 mL) were added. The reaction was heated to 60° C. for 2 h, and the observed precipitate filtered to give 4-({3,5-bis[(methyloxy)carbonyl]phenyl}amino)-7-bromo-3-quinolinecarboxylic acid (278 mg, 0.605 mmol, 38.1% yield) as a pale yellow solid. ¹H NMR (DMSO-d₆): 9.09 (s, 1H), 8.22 (d, J=2.0 Hz, 1H), 8.10-8.16 (m, 1H), 7.83-7.91 (m, 1H), 7.81 (d, J=1.3 Hz, 2H), 7.63 (dd, J=9.0, 1.9 Hz, 1H), 3.86 (s, 6H).

### b) dimethyl 5-[(7-bromo-3-{[({3-[(methyloxy)carbonyl]phenyl}methyl)amino]carbonyl}-4-quinolinyl)amino]-1,3-benzenedicarboxylate

Benzotriazol-1-yloxy-trispyrrolidinophosphonium hexafluorophosphate (469 mg, 0.901 mmol) was added to a stirred solution of 4-({3,5-bis[(methyloxy)carbonyl]phenyl}amino)-7-bromo-3-quinolinecarboxylic acid (276 mg, 0.601 mmol), methyl 3-aminomethyl benzoate (133 mg, 0.661 mmol), and triethylamine (0.335 mL, 2.404 mmol) in dichloromethane (3 mL) at room temperature, and the mixture stirred for overnight. A white precipitate formed, which was filtered and washed with dichloromethane to give dimethyl 5-[(7-bromo-3-{[({3-[(methyloxy)carbonyl]phenyl}methyl)amino]carbonyl}-4-quinolinyl)amino]-1,3-benzenedicarboxylate (258 mg, 0.425 mmol, 70.8% yield). ¹H NMR (400 MHz, DMSO-d₆) ppm 3.84 (s, 9H) 4.11 (d, J=5.56 Hz, 2H) 7.30-7.43 (m, 2H) 7.70 (s, 2H) 7.72-7.82 (m, 2H) 7.91 (s, 1H) 8.22 (s, 1H) 8.27 (d, J=8.84 Hz, 1H) 8.85 (s, 1H) 9.23 (br. s., 1H) 9.77 (s, 1H).

### c) 5-{[3-({[(3-carboxyphenyl)methyl]amino}carbonyl-7-(3,5-dimethyl-1H-pyrazol-4-yl)-4-quinolinyl]amino}-1,3-benzenedicarboxylic acid

Dimethyl 5-[(7-bromo-3-{[({3-[(methyloxy)carbonyl]phenyl}methyl)amino]carbonyl}-4-quinolinyl)amino]-1,3-benzenedicarboxylate (258 mg, 0.425 mmol), 3,5-dimethyl-4-(4,4,5,5-tetramethyl-1,3,2-dioxaborolan-2-yl)-1H-pyrazole (189 mg, 0.851 mmol), tetrakis(triphenylphosphine)palladium(0) (24.58 mg, 0.021 mmol), and potassium carbonate (118 mg, 0.851 mmol) in 1,4-dioxane (2 mL)/water (0.5 mL) were added to a sealed microwave tube and heated at 120° C. for 30 minutes. Reaction was concentrated under reduced pressure, dissolved in methanol (3 mL), and 6N sodium hydroxide (0.213 mL, 1.276 mmol) was added. The mixture was stirred at 60° C. for 2 hours. Purification by HPLC (20-70% acetonitrile/water w/0.1% TFA) afforded 5-{[3-({[(3-carboxyphenyl)methyl]amino}carbonyl)-7-(3,5-dimethyl-1H-pyrazol-4-yl)-4-quinolinyl]amino}-1,3-benzenedicarboxylic acid (35 mg, 0.060 mmol, 14.19% yield) as a white solid. ¹H NMR (400 MHz, METHANOL-d4) ppm 2.68 (s, 6H) 4.15 (s, 2H) 6.37 (s, 1H) 7.41-7.54 (m, 2H) 7.81 (dd, J=8.84, 1.52 Hz, 1H) 7.87-8.01 (m, 4H) 8.17 (d, J=1.26 Hz, 2H) 8.49 (d, J=8.59 Hz, 1H) 8.58 (s, 1H) 8.81 (s, 1H).

### Example 12

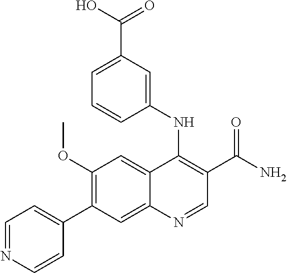 [SMILES: COc1cc2c(Nc3cccc(C(=O)O)c3)c(C(N)=O)cnc2cc1-c1ccncc1]

### 3-{[3-(aminocarbonyl)-6-(methyloxy)-7-(4-pyridinyl)-4-quinolinyl]amino}benzoic acid

### a) methyl 3-{[3-(aminocarbonyl)-6-(methyloxy)-7-(4-pyridinyl)-4-quinolinyl]amino}benzoate

A mixture of methyl 3-{[3-(aminocarbonyl)-7-chloro-6-(methyloxy)-4-quinolinyl]amino}benzoate (example 9e, 0.154 g, 0.400 mmol), 4-pyridineboronic acid (0.098 g, 0.800 mmol), bis(tri-t-butylphosphine)palladium(0) (0.020 g, 0.040 mmol) and potassium carbonate (0.166 g, 3.00 mmol) in 1,4-dioxane (2 mL) and water (0.6 mL) was stirred at 120° C. in a microwave synthesiser for 0.5 h, then cooled to room temperature and chromatographed (silica gel, 3-10% methanol/dichloromethane) to give the title compound (0.098 g, 57%) as a yellow solid. ¹H NMR (400 MHz, DMSO-d₆) ppm 3.59 (s, 3H) 3.83 (s, 3H) 7.18-7.25 (m, 1H) 7.32 (s, 1H) 7.42 (t, J=7.96 Hz, 1H) 7.56-7.63 (m, 3H) 7.63-7.66 (m, 2H) 7.98 (s, 1H) 8.17 (br. s., 1H) 8.63-8.69 (m, 2H) 8.87 (s, 1H) 10.10 (s, 1H).

### b) 3-{[3-(aminocarbonyl)-6-(methyloxy)-7-(4-pyridinyl)-4-quinolinyl]amino}benzoic acid

1M aqueous sodium hydroxide (2.00 mL, 2.00 mmol) was added to a solution of methyl 3-{[3-(aminocarbonyl)-6-(methyloxy)-7-(4-pyridinyl)-4-quinolinyl]amino}benzoate (0.080 g, 0.187 mmol) in ethanol (5 mL) and the mixture stirred at room temperature for 18 h, then diluted with water (30 mL). The pH was adjusted to 2 with 1M aqueous hydrochloric acid and the solvent removed under reduced pressure. The residue was triturated with water (3 mL) and the solid filtered off and dried to give the title compound (0.050 g, 65%) as a pale yellow solid. ¹H NMR (400 MHz, DMSO-d₆) ppm 3.81 (s, 3H) 7.53-7.61 (m, 2H) 7.66 (s, 1H) 7.83-7.88 (m, 1H) 7.89 (s, 1H) 7.96-8.03 (m, 3H) 8.20 (s, 1H) 8.25 (br. s., 1H) 8.88-8.94 (m, 3H) 11.67 (br. s., 1H) 13.18 (br. s., 1H).

### Example 13

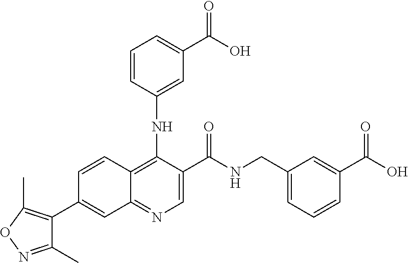 [SMILES: Cc1noc(C)c1-c1ccc2c(Nc3cccc(C(=O)O)c3)c(C(=O)NCc3cccc(C(=O)O)c3)cnc2c1]

### 3-[({[4-[(3-carboxyphenyl)amino]-7-(3,5-dimethyl-4-isoxazolyl)-3-quinolinyl]carbonyl}amino)methyl]benzoic acid

### a) ethyl 7-(3,5-dimethyl-4-isoxazolyl)-4-hydroxy-3-quinolinecarboxylate

ethyl 7-bromo-4-hydroxy-3-quinolinecarboxylate (2.43 g, 8.21 mmol), (3,5-dimethyl-4-isoxazolyl)boronic acid (1.735 g, 12.31 mmol), tetrakis(triphenylphosphine)palladium(0) (0.474 g, 0.410 mmol), and potassium carbonate (1.701 g, 12.31 mmol) in 1,4-dioxane (15 mL)/water (5.00 mL) were added to a round bottom flask and heated to reflux. After 1 hour the initial solids dissolved to give a clear, dark solution. LCMS analysis confirmed complete product formation. The flask was removed from the heat, and a cream colored precipitate was observed to crash out upon cooling. 15 mL of water were then added and a thick precipitate formed. The precipitate was filtered and washed with water, ether and ethanol to give ethyl 7-(3,5-dimethyl-4-isoxazolyl)-4-hydroxy-3-quinolinecarboxylate (1.72 g, 5.51 mmol, 67.1% yield) as a cream solid. ¹H NMR (DMSO-d₆): 8.56-8.66 (m, 1H), 8.22 (d, J=8.3 Hz, 1H), 7.63 (s, 1H), 7.36-7.51 (m, 1H), 4.12-4.34 (m, 2H), 2.48 (s, 3H), 2.30 (s, 3H), 1.17-1.42 (m, 3H).

### b) 4-chloro-7-(3,5-dimethyl-4-isoxazolyl)-3-quinolinecarboxylic acid

A 50 mL flask was charged with ethyl 7-(3,5-dimethyl-4-isoxazolyl)-4-hydroxy-3-quinolinecarboxylate (1.72 g, 5.51 mmol) and neat thionyl chloride (10 ml, 137 mmol). The reaction was stirred at reflux (80° C.) for 4 h. The solution was then concentrated under reduced pressure, and coevaporated with toluene. A slurry was made using diethyl ether under sonication, and the precipitate filtered. The powder was dissolved in ethanol (12 mL), and sodium hydroxide (1.836 ml, 11.01 mmol) was added. After stirring for 1 hour at 50° C., an additional 5 mL of ethanol was then added, followed by the addition of 50 mL diethyl ether. The mixture was cooled at 4° C. overnight, and the white precipitate filtered to give crude 4-chloro-7-(3,5-dimethyl-4-isoxazolyl)-3-quinolinecarboxylic acid (1.198 g, 3.96 mmol, 71.9% yield). The material was taken directly to the next step without any further purification or analysis.

### c) 7-(3,5-dimethyl-4-isoxazolyl)-4-({3-[(ethyloxy)carbonyl]phenyl}amino)-3-quinolinecarboxylic acid

A 50 mL flask was charged with 4-chloro-7-(3,5-dimethyl-4-isoxazolyl)-3-quinolinecarboxylic acid (1.198 g, 3.96 mmol) and ethyl 3-aminobenzoate (0.850 g, 5.14 mmol) in acetic acid (20 mL). The reaction was stirred at 60° C. for 1 hour. The solvent was concentrated and ethanol added to make a slurry. The precipitate was filtered then dried to give 7-(3,5-dimethyl-4-isoxazolyl)-4-({3-[(ethyloxy)carbonyl]phenyl}amino)-3-quinolinecarboxylic acid (1.34 g, 3.11 mmol, 78% yield) as tan crystals. MS (ES+) m/e 432 [M+H]⁺.

### d) methyl 3-[({[7-(3,5-dimethyl-4-isoxazolyl)-4-({3-[(ethyloxy)carbonyl]phenyl}amino)-3-quinolinyl]carbonyl}amino)methyl]benzoate

Benzotriazol-1-yloxy-trispyrrolidinophosphonium hexafluorophosphate (181 mg, 0.348 mmol) was added to a stirred solution of 7-(3,5-dimethyl-4-isoxazolyl)-4-({3-[(ethyloxy)carbonyl]phenyl}amino)-3-quinolinecarboxylic acid (100 mg, 0.232 mmol), 3-aminomethylbenzoic acid methyl ester hydrochloride (48.9 mg, 0.243 mmol), and triethylamine (0.097 mL, 0.695 mmol) in dichloromethane (2 mL) at room temperature, and the mixture stirred for 2 h. The solution was filtered and purified by HPLC (10-80% Acetonitrile/water with 0.1% TFA) to give methyl 3-[({[7-(3,5-dimethyl-4-isoxazolyl)-4-({3-[(ethyloxy)carbonyl]phenyl}amino)-3-quinolinyl]carbonyl}amino)methyl]benzoate (164 mg, 0.283 mmol, 122% yield) as yellow solid. ¹H NMR (METHANOL-d₄): 8.78-8.90 (m, 1H), 8.45 (d, J=8.8 Hz, 1H), 7.96 (d, J=1.5 Hz, 3H), 7.93 (d, J=1.3 Hz, 2H), 7.76 (dd, J=9.0, 1.6 Hz, 1H), 7.42-7.61 (m, 4H), 4.33-4.48 (m, 2H), 4.12-4.24 (m, 2H), 3.91-3.99 (m, 3H), 2.56 (s, 3H), 2.39 (s, 3H), 1.33-1.47 (m, 3H).

### e) 3-[({[4-[(3-carboxyphenyl)amino]-7-(3,5-dimethyl-4-isoxazolyl)-3-quinolinyl]carbonyl}amino)methyl]benzoic acid

6M sodium hydroxide (0.142 mL, 0.850 mmol) was added to a stirred solution of methyl 3-[({[7-(3,5-dimethyl-4-isoxazolyl)-4-({3-[(ethyloxy)carbonyl]phenyl}amino)-3-quinolinyl]carbonyl}amino)methyl]benzoate (164 mg, 0.283 mmol) in ethanol (1 mL) and the mixture stirred at 60° C. for 1 hour. HPLC purification (5-70% acetonitrile/water with 0.1% TFA 10 min gradient) afforded 3-[({[4-[(3-carboxyphenyl)amino]-7-(3,5-dimethyl-4-isoxazolyl)-3-quinolinyl]carbonyl}amino)methyl]benzoic acid (60 mg, 0.112 mmol, 39.5% yield) as a white solid. ¹H NMR (400 MHz, METHANOL-d4) ppm 2.38 (s, 3H) 2.55 (s, 3H) 4.21 (s, 2H) 7.46 (d, J=7.58 Hz, 1H) 7.51 (s, 1H) 7.55-7.62 (m, 2H) 7.72 (dd, J=8.84, 1.26 Hz, 1H) 7.91-8.04 (m, 5H) 8.41 (d, J=9.09 Hz, 1H) 8.81-8.90 (m, 1H).

### Example 14

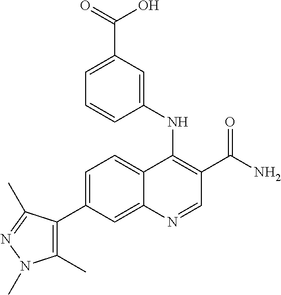 [SMILES: Cc1nn(C)c(C)c1-c1ccc2c(Nc3cccc(C(=O)O)c3)c(C(N)=O)cnc2c1]

### 3-{[3-(aminocarbonyl)-7-(1,3,5-trimethyl-1H-pyrazol-4-yl)-4-quinolinyl]amino}benzoic acid

### a) 4,7-dichloro-3-quinolinecarboxamide

A mixture of 7-chloro-4-hydroxy-3-quinolinecarboxylic acid (3.00 g, 13.4 mmol) and phosphorus oxychloride (20.0 mL, 215 mmol) was stirred at a gentle reflux for 2 h, then cooled. The excess phosphorus oxychloride was removed under reduced pressure and the solid residue azeotroped twice with toluene, then slurried in dioxane (60 mL). Ammonia was bubbled through the mixture while cooling in ice until the gas evolved showed pH 8. The mixture was stirred at room temperature for 0.25 h, then diluted with ether (60 mL) and filtered. The solid was washed with ether, water and dried to give the title compound (1.87 g, 58%) as a light brown solid. ¹H NMR (400 MHz, DMSO-d₆) ppm 7.88 (dd, J=8.97, 2.15 Hz, 1H) 8.02 (br. s., 1H) 8.20-8.26 (m, 2H) 8.33 (d, J=9.09 Hz, 1H) 8.93 (s, 1H).

### b) ethyl 3-{[3-(aminocarbonyl)-7-chloro-4-quinolinyl]amino}benzoate

A mixture of crude 4,7-dichloro-3-quinolinecarboxamide (1.70 g, 7.05 mmol) and ethyl 3-aminobenzoate (1.165 g, 7.05 mmol) in acetic acid (15 mL) was heated thermally to 50° C. for 0.5 h. Upon cooling, the reaction mixture was diluted in water as a solid precipitated out. This solid was filtered, then washed with more water and dried in vacuo to obtain the title compound (2.75 g, 89%) as a cream solid. ¹H NMR (400 MHz, DMSO-d₆) ppm 11.69 (br. s., 1H), 9.01 (s, 1H), 8.19-8.31 (m, 2H), 8.14 (d, J=1.5 Hz, 1H), 7.79-7.89 (m, 2H), 7.65-7.75 (m, 2H), 7.42-7.58 (m, 2H), 4.32 (q, J=7.1 Hz, 2H), 1.31 (t, J=7.1 Hz, 3H).

### c) ethyl 3-{[3-(aminocarbonyl)-7-(1,3,5-trimethyl-1H-pyrazol-4-yl)-4-quinolinyl]amino}benzoate

A solution of ethyl 3-{[3-(aminocarbonyl)-7-chloro-4-quinolinyl]amino}benzoate (155 mg, 0.419 mmol), 1,3,5-trimethyl-4-(4,4,5,5-tetramethyl-1,3,2-dioxaborolan-2-yl)-1H-pyrazole (297 mg, 1.257 mmol), potassium carbonate (174 mg, 1.257 mmol), and tetrakis(triphenylphosphine)palladium(0) (21.80 mg, 0.019 mmol) in 1,4-dioxane (2.0 ml) and water (0.667 ml) was heated to 120° C. for 20 min. in a Biotage Initiator® microwave synthesizer. Upon cooling, the reaction mixture was diluted with water, extracted with ethyl acetate twice, dried (MgSO₄) and concentrated in vacuo. The residue was purified via flash column chromatography (10% methanol in dichloromethane) to obtain the title compound as an amber oil, which was slurried into ethyl ether to obtain the title compound (0.086 g, 46%) as an off white solid. MS (ES+) m/e 444 [M+H]⁺.

### d) 3-{[3-(aminocarbonyl)-7-(1,3,5-trimethyl-1H-pyrazol-4-yl)-4-quinolinyl]amino}benzoic acid

To a suspension of ethyl 3-{[3-(aminocarbonyl)-7-(1,3,5-trimethyl-1H-pyrazol-4-yl)-4-quinolinyl]amino}benzoate (73 mg, 0.165 mmol) in ethanol (2.0 mL) was added 1N aqueous sodium hydroxide (2 ml, 2.000 mmol). After stirring 3 h at ambient temperature, the reaction was concentrated to an aqueous residue, then neutralized with 1N aqueous hydrochloric acid. The resulting precipitate was filtered, washed with water, and dried in vacuo to obtain 3-{[3-(aminocarbonyl)-7-(1,3,5-trimethyl-1H-pyrazol-4-yl)-4-quinolinyl]amino}benzoic acid (43 mg, 0.104 mmol, 62.9% yield) as a light yellow solid. ¹H NMR (400 MHz, DMSO-d₆) ppm 11.87 (br. s., 1H), 8.98 (s, 1H), 8.26 (br. s., 1H), 8.13 (d, J=9.1 Hz, 1H), 7.83-7.91 (m, 3H), 7.78 (br. s., 1H), 7.60 (dd, J=9.1, 1.6 Hz, 1H), 7.56 (d, J=5.3 Hz, 1H), 3.75 (s, 3H), 2.33 (s, 3H), 2.23 (s, 3H).

### Example 15

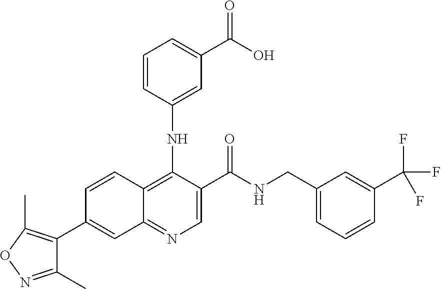 [SMILES: Cc1noc(C)c1-c1ccc2c(Nc3cccc(C(=O)O)c3)c(C(=O)NCc3cccc(C(F)(F)F)c3)cnc2c1]

### 3-({7-(3,5-dimethyl-4-isoxazolyl)-3-[({[3-(trifluoromethyl)phenyl]methyl}amino)carbonyl]-4-quinolinyl}amino)benzoic acid

### a) ethyl 3-({7-(3,5-dimethyl-4-isoxazolyl)-3-[({[3-(trifluoromethyl)phenyl]methyl}amino)carbonyl]-4-quinolinyl}amino)benzoate

Benzotriazol-1-yloxy-trispyrrolidinophosphonium hexafluorophosphate (133 mg, 0.255 mmol) was added to a stirred solution of 7-(3,5-dimethyl-4-isoxazolyl)-4-({3-[(ethyloxy)carbonyl]phenyl}amino)-3-quinolinecarboxylic acid (example 13c, 100 mg, 0.232 mmol), 3-(trifluoromethyl)benzylamine (0.033 mL, 0.232 mmol), and triethylamine (0.097 mL, 0.695 mmol) in dichloromethane (2 mL) at room temperature, and the mixture stirred for 1 h. The solution was filtered and purified by HPLC (10-80% Acetonitrile/water with 0.1% TFA) to give ethyl 3-({7-(3,5-dimethyl-4-isoxazolyl)-3-[({[3-(trifluoromethyl)phenyl]methyl}amino)carbonyl]-4-quinolinyl}amino)benzoate (117 mg, 0.199 mmol, 86% yield) as a pale yellow solid. ¹H NMR (METHANOL-d₄): 8.87 (s, 1H), 8.43 (d, J=8.8 Hz, 1H), 7.92-8.02 (m, 3H), 7.74 (dd, J=9.0, 1.6 Hz, 1H), 7.57-7.64 (m, 3H), 7.50-7.57 (m, 4H), 4.39 (q, 2H), 4.23 (s, 2H), 2.56 (s, 3H), 2.38 (s, 3H), 1.40 (t, J=7.2 Hz, 3H).

### b) 3-({7-(3,5-dimethyl-4-isoxazolyl)-3-[({[3-(trifluoromethyl)phenyl]methyl}amino)carbonyl]-4-quinolinyl}amino)benzoic acid

6M sodium hydroxide (0.066 mL, 0.398 mmol) was added to a stirred solution of ethyl 3-({7-(3,5-dimethyl-4-isoxazolyl)-3-[({[3-(trifluoromethyl)phenyl]methyl}amino)carbonyl]-4-quinolinyl}amino)benzoate (117 mg, 0.199 mmol) in ethanol (1 mL) and the mixture stirred at 60° C. for 1 hour. HPLC purification (5-70% acetonitrile/water with 0.1% TFA @ 10 min gradient) afforded 3-({7-(3,5-dimethyl-4-isoxazolyl)-3-[({[3-(trifluoromethyl)phenyl]methyl}amino)carbonyl]-4-quinolinyl}amino)benzoic acid (49 mg, 0.087 mmol, 44.0% yield) as a white solid. ¹H NMR (METHANOL-d₄) 8.86 (s, 1H), 8.41 (d, J=8.8 Hz, 1H), 7.92-8.07 (m, 3H), 7.73 (dd, J=9.0, 1.6 Hz, 1H), 7.49-7.67 (m, 6H), 4.24 (s, 2H), 3.28-3.35 (m, 2H), 2.55 (s, 3H), 2.38 (s, 3H).

### Example 16

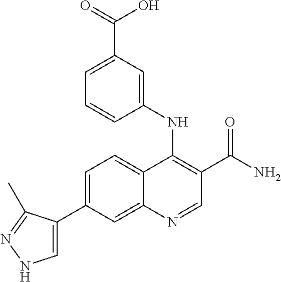 [SMILES: Cc1n[nH]cc1-c1ccc2c(Nc3cccc(C(=O)O)c3)c(C(N)=O)cnc2c1]

### 3-{[3-(aminocarbonyl)-7-(3-methyl-1H-pyrazol-4-yl)-4-quinolinyl]amino}benzoic acid, dihydrochloride salt

A solution of ethyl 3-{[3-(aminocarbonyl)-7-bromo-4-quinolinyl]amino}benzoate (example 6a, 135 mg, 0.326 mmol), (3-methyl-11H-pyrazol-4-yl)boronic acid pinacol ester (203 mg, 0.978 mmol), potassium carbonate (135 mg, 0.978 mmol), and tetrakis(triphenylphosphine)palladium(0) (16.95 mg, 0.015 mmol) in 1,4-dioxane (3 ml) and water (1.000 ml) was heated to 120° C. for 20 min. in a Biotage Initiator® microwave synthesizer. Upon cooling, the reaction mixture was concentrated in vacuo, diluted with ethanol (2 ml) and then treated with sodium hydroxide (1M aq solution) (2 ml, 2.000 mmol). Following stirring overnight at room temperature, the ethanol was removed in vacuo, the residue diluted with water and extracted using ethyl acetate. The aqueous portion was then adjusted to pH˜4 using 1N hydrochloric acid. The resulting solid was filtered and dried in vacuo to obtain the title compound (0.093 g, 62%) as a yellow solid. ¹H NMR (400 MHz, DMSO-d₆) ppm 10.45 (s, 1H), 9.00 (s, 1H), 8.27 (br. s., 1H), 8.00 (d, J=8.8 Hz, 1H), 7.99 (s, 1H), 7.74 (d, J=9.1 Hz, 1H), 7.68 (br. s., 1H), 7.54-7.60 (m, 2H), 7.48 (s, 1H), 7.37 (t, J=7.8 Hz, 1H), 7.21 (dd, J=7.8, 1.5 Hz, 1H), 2.47 (s, 3H).

### Example 17

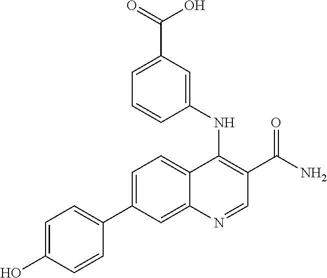 [SMILES: NC(=O)c1cnc2cc(-c3ccc(O)cc3)ccc2c1Nc1cccc(C(=O)O)c1]

### 3-{[3-(aminocarbonyl)-7-(4-hydroxyphenyl)-4-quinolinyl]amino}benzoic acid, bistrifluoroacetate salt

### a) 3-({3-(aminocarbonyl)-7-[4-(methyloxy)phenyl]-4-quinolinyl}amino)benzoic acid, hydrochloride salt

A solution of ethyl 3-{[3-(aminocarbonyl)-7-bromo-4-quinolinyl]amino}benzoate (example 6a, 130 mg, 0.314 mmol), 4-methoxy phenyl boronic acid (71.5 mg, 0.471 mmol), potassium carbonate (130 mg, 0.941 mmol), and tetrakis(triphenylphosphine)palladium(0) (16.32 mg, 0.014 mmol) in 1,4-dioxane (2.0 ml) and water (0.667 mL) was heated to 120° C. for 20 min. in a Biotage Initiator® microwave synthesizer. Upon cooling, the reaction mixture was concentrated in vacuo and diluted with ethanol (3 ml) and then treated with sodium hydroxide (1N aq solution) (2 mL, 2.000 mmol). Following stirring overnight at room temperature, the ethanol was removed in vacuo, the residue diluted with water and extracted using ethyl acetate. The aquoeus layer was then adjusted to pH˜4 using 1N hydrochloric acid. The resulting solid was filtered and dried in vacuo to obtain the title compound (0.100 g, 66%) as a yellow solid. ¹H NMR (400 MHz, DMSO-d₆) ppm 10.47 (br. s., 1H), 9.02 (s, 1H), 8.28 (br. s., 1H), 8.17 (d, J=1.5 Hz, 1H), 7.78-7.82 (m, 1H), 7.82 (d, J=8.6 Hz, 2H), 7.73 (dd, J=9.0, 1.9 Hz, 1H), 7.70 (s, 1H), 7.58 (d, J=7.6 Hz, 1H), 7.49 (s, 1H), 7.36 (t, J=7.8 Hz, 1H), 7.14-7.25 (m, 1H), 7.07 (d, J=8.6 Hz, 2H), 3.82 (s, 3H).

### b) 3-{[3-(aminocarbonyl)-7-(4-hydroxyphenyl)-4-quinolinyl]amino}benzoic acid, bistrifluoroacetate salt

A solution of 3-{[3-(aminocarbonyl)-7-(4-methyloxyphenyl)-4-quinolinyl]amino}benzoic acid (80 mg, 0.194 mmol) in dichloromethane (10 mL) was treated with boron tribromide (1M solution in dichloromethane) (1.6 mL, 1.600 mmol) at room temperature overnight. The reaction mixture was filtered and the solid treated with water, then filtered again. The filtrate was concentrated and purified together with the filtered material (Reverse Phase HPLC 0.1% TFA in acetonitrile and water) to obtain the title compound (0.090 g, 74%) as a bright yellow solid. ¹H NMR (400 MHz, DMSO-d₆) ppm 13.18 (br. s., 1H), 11.80 (br. s., 1H), 10.02 (br. s., 1H), 8.96 (s, 1H), 8.27 (br. s., 1H), 8.15 (d, J=9.1 Hz, 1H), 8.12 (s, 1H), 7.95 (d, J=9.1 Hz, 1H), 7.86 (dd, J=5.6, 3.3 Hz, 1H), 7.84 (s, 1H), 7.78 (br. s., 1H), 7.72 (d, J=8.6 Hz, 2H), 7.55 (d, J=4.3 Hz, 2H), 6.95 (d, J=8.6 Hz, 2H).

### Example 18

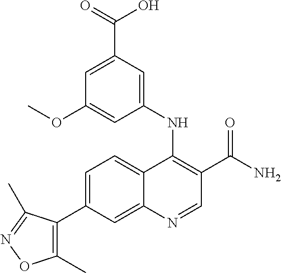 [SMILES: COc1cc(Nc2c(C(N)=O)cnc3cc(-c4c(C)noc4C)ccc23)cc(C(=O)O)c1]

### 3-{[3-(aminocarbonyl)-7-(3,5-dimethyl-4-isoxazolyl)-4-quinolinyl]amino}-5-(methyloxy)benzoic acid, trifluoroacetate salt

### a) 3-{[3-(aminocarbonyl)-7-bromo-4-quinolinyl]amino}-5-(methyloxy)benzoic acid

To a suspension of 7-bromo-4-chloro-3-quinolinecarboxamide (example 1a, 100 mg, 0.350 mmol) in acetic acid (10 mL) was added 3-amino-5-(methyloxy)benzoic acid (64.4 mg, 0.385 mmol). The reaction mixture was stirred at 100° C. for one hour. After cooling, the precipitate was collected, washed with ether and dried in the air to afford 3-{[3-(aminocarbonyl)-7-bromo-4-quinolinyl]amino}-5-(methyloxy)benzoic acid (105 mg, 0.252 mmol, 72.0% yield) as a yellow solid. ¹H NMR (400 MHz, DMSO-d6) ppm 11.87 (br. s., 1H), 9.04 (s, 1H), 8.35 (br. s., 1H), 8.33 (d, J=2.0 Hz, 1H), 8.19 (d, J=9.3 Hz, 1H), 7.83 (dd, J=9.1, 2.0 Hz, 1H), 7.79 (br. s., 1H), 7.41 (s, 1H), 7.34 (dd, J=2.3, 1.3 Hz, 1H), 7.13 (t, J=2.1 Hz, 1H), 3.80 (s, 3H).

### b) 3-{[3-(aminocarbonyl)-7-(3,5-dimethyl-4-isoxazolyl)-4-quinolinyl]amino}-5-(methyloxy)benzoic acid, trifluoroacetate salt

To a suspension of 3-{[3-(aminocarbonyl)-7-bromo-4-quinolinyl]amino}-5-(methyloxy)benzoic acid (100 mg, 0.240 mmol), (3,5-dimethyl-4-isoxazolyl)boronic acid (67.7 mg, 0.481 mmol) and tetrakis(triphenylphosphine)palladium(0) (13.88 mg, 0.012 mmol) in 1,4-dioxane (6 mL) and water (2.000 mL) was added potassium carbonate (66.4 mg, 0.481 mmol). After heating at 120° C. for 0.5 h, then cooling, the reaction mixture was purified through preparative HPLC (YMC 75×30 mm column, 0.1% TFA in water and 0.1% TFA in acetonitrile) to afford 3-{[3-(aminocarbonyl)-7-(3,5-dimethyl-4-isoxazolyl)-4-quinolinyl]amino}-5-(methyloxy)benzoic acid (22 mg, 0.040 mmol, 16.76% yield) as an off white solid. ¹H NMR (400 MHz, DMSO-d6) δ ppm 13.18 (br. s., 1H), 11.53 (br. s., 1H), 9.02 (s, 1H), 8.18-8.30 (m, 2H), 8.00 (d, J=1.5 Hz, 1H), 7.79 (br. s., 1H), 7.71 (dd, J=8.8, 1.3 Hz, 1H), 7.38 (s, 1H), 7.31 (s, 1H), 7.11 (s, 1H), 3.80 (s, 3H), 2.53 (s, 3H), 2.34 (s, 3H).

### Example 19

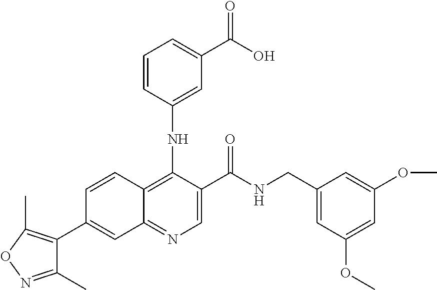 [SMILES: COc1cc(CNC(=O)c2cnc3cc(-c4c(C)noc4C)ccc3c2Nc2cccc(C(=O)O)c2)cc(OC)c1]

### 3-{[3-[({[3,5-bis(methyloxy)phenyl]methyl}amino)carbonyl]-7-(3,5-dimethyl-4-isoxazolyl)-4-quinolinyl]amino}benzoic acid

### a) ethyl 3-{[3-[({[3,5-bis(methyloxy)phenyl]methyl}amino)carbonyl]-7-(3,5-dimethyl-4-isoxazolyl)-4-quinolinyl]amino}benzoate

Benzotriazol-1-yloxy-trispyrrolidinophosphonium hexafluorophosphate (181 mg, 0.348 mmol) was added to a stirred solution of 7-(3,5-dimethyl-4-isoxazolyl)-4-({3-[(ethyloxy)carbonyl]phenyl}amino)-3-quinolinecarboxylic acid (example 13c, 100 mg, 0.232 mmol), 3,5-dimethoxybenzylamine (0.037 mL, 0.243 mmol), and triethylamine (0.097 mL, 0.695 mmol) in dichloromethane (2 mL) at room temperature, and the mixture stirred for 2 h. The solution was filtered and purified by HPLC (10-80% Acetonitrile/water with 0.1% TFA) to give ethyl 3-{[3-[({[3,5-bis(methyloxy)phenyl]methyl}amino)carbonyl]-7-(3,5-dimethyl-4-isoxazolyl)-4-quinolinyl]amino}benzoate (139 mg, 0.239 mmol, 103% yield) as a yellow solid. ¹H NMR (METHANOL-d₄): 8.73-8.86 (m, 1H), 8.47 (d, J=8.8 Hz, 1H), 7.97 (d, J=1.3 Hz, 3H), 7.74 (dd, J=8.8, 1.8 Hz, 1H), 7.47-7.65 (m, 2H), 6.36-6.49 (m, 3H), 4.38 (q, 2H), 4.02 (d, 2H), 3.77 (s, 6H), 2.56 (s, 3H), 2.39 (s, 3H), 1.39 (t, 3H).

### b) 3-{[3-[({[3,5-bis(methyloxy)phenyl]methyl}amino)carbonyl]-7-(3,5-dimethyl-4-isoxazolyl)-4-quinolinyl]amino}benzoic acid

6M sodium hydroxide (0.080 mL, 0.479 mmol) was added to a stirred solution of ethyl 3-{[3-[({[3,5-bis(methyloxy)phenyl]methyl}amino)carbonyl]-7-(3,5-dimethyl-4-isoxazolyl)-4-quinolinyl]amino}benzoate (139 mg, 0.239 mmol) in ethanol (1 mL) and the mixture stirred at 60° C. for 1 hour. HPLC purification (5-70% acetonitrile/water with 0.1% TFA @ 10 min gradient) afforded 3-{[3-[({[3,5-bis(methyloxy)phenyl]methyl}amino)carbonyl]-7-(3,5-dimethyl-4-isoxazolyl)-4-quinolinyl]amino}benzoic acid (65 mg, 0.118 mmol, 49.1% yield) as a white solid. ¹H NMR (400 MHz, METHANOL-d4) ppm 2.39 (s, 3H) 2.56 (s, 3H) 3.68-3.89 (m, 6H) 4.05 (s, 2H) 6.32-6.54 (m, 3H) 7.56 (d, J=2.02 Hz, 2H) 7.67-7.81 (m, 1H) 7.89-8.06 (m, 3H) 8.36-8.52 (m, 1H) 8.81 (s, 1H).

### Example 20

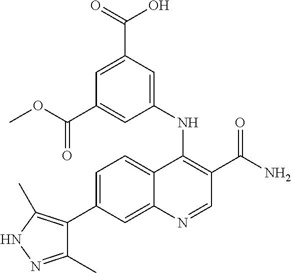 [SMILES: COC(=O)c1cc(Nc2c(C(N)=O)cnc3cc(-c4c(C)n[nH]c4C)ccc23)cc(C(=O)O)c1]

### 3-{[3-(aminocarbonyl)-7-(3,5-dimethyl-1H-pyrazol-4-yl)-4-quinolinyl]amino}-5-[(methyloxy)carbonyl]benzoic acid, trifluoroacetate salt

A solution of dimethyl 5-{[3-(aminocarbonyl)-7-bromo-4-quinolinyl]amino}-1,3-benzenedicarboxylate (example 4a, 97 mg, 0.212 mmol), 3,5-dimethyl-4-(4,4,5,5-tetramethyl-1,3,2-dioxaborolan-2-yl)-1H-pyrazole (141 mg, 0.635 mmol), potassium carbonate (88 mg, 0.635 mmol), and tetrakis(triphenylphosphine)palladium(0) (12.23 mg, 10.58 μmol) in 1,4-dioxane (2.0 ml) and water (0.667 ml) was heated to 120° C. for 20 min. in a Biotage Initiator® microwave synthesizer. Upon cooling, the reaction mixture was filtered and concentrated in vacuo. The residue was purified via Reverse Phase HPLC (0.1% TFA in acetonitrile and water) to obtain the title compound (0.010 g, 8%) as a yellow solid. ¹H NMR (400 MHz, DMSO-d₆) ppm 11.29 (br. s., 1H), 8.94 (s, 1H), 8.26-8.38 (m, 2H), 8.18 (br. s., 1H), 7.96-8.05 (m, 2H), 7.91 (d, J=1.3 Hz, 1H), 7.72 (d, J=7.8 Hz, 1H), 7.65 (br. s., 1H), 3.89 (s, 3H), 2.33 (s, 6H).

### Example 21

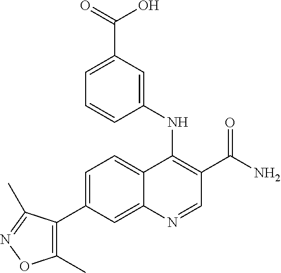 [SMILES: Cc1noc(C)c1-c1ccc2c(Nc3cccc(C(=O)O)c3)c(C(N)=O)cnc2c1]

### 3-{[3-(aminocarbonyl)-7-(3,5-dimethyl-)-4-quinolinyl]amino}benzoic acid, trifluoroacetate salt

### 3-{[3-(aminocarbonyl)-7-(3,5-dimethyl-4-isoxazolyl)-4-quinolinyl]amino}benzoic acid, trifluoroacetate salt

ethyl 3-{[3-(aminocarbonyl)-7-bromo-4-quinolinyl]amino}benzoate (example 6a, 168 mg, 0.406 mmol), (3,5-dimethyl-4-isoxazolyl)boronic acid (159 mg, 1.14 mmol), potassium carbonate (140 mg, 1.014 mmol), and tetrakis(triphenylphosphine)palladium(0) (21.09 mg, 0.018 mmol) were combined as a solution in 1,4-dioxane (3 ml) and water (1.000 mL) and heated to 120° C. for 20 min. in a Biotage Initiator® microwave synthesizer. Upon cooling, the reaction mixture was concentrated. The obtained residue was dissolved into ethanol (5 ml) and treated with NaOH (1N aq solution) (2 mL, 2.000 mmol). After stirring at 80° C. for 1 h, the mixture was concentrated under vacuum and the residue extracted with ethyl acetate. The aqueous layer was then made acidic using 1N hydrochloric acid (pH˜3). The solid was filtered, washed with water and purified using Reverse Phase Gilson (0.1% TFA in water and acetonitrile) to obtain the title compound (0.025 g, 10%) as a yellow solid. ¹H NMR (400 MHz, DMSO-d₆) ppm 13.15 (br. s., 1H), 11.52 (br. s., 1H), 9.01 (s, 1H), 8.25 (br. s., 1H), 8.17 (d, J=9.1 Hz, 1H), 7.98 (d, J=1.5 Hz, 1H), 7.79-7.85 (m, 1H), 7.75-7.81 (m, 2H), 7.68 (d, J=8.6 Hz, 1H), 7.46-7.56 (m, 2H), 2.52 (s, 3H), 2.33 (s, 3H).

### Example 22

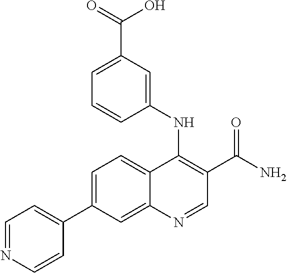 [SMILES: NC(=O)c1cnc2cc(-c3ccncc3)ccc2c1Nc1cccc(C(=O)O)c1]

### 3-{[3-(aminocarbonyl)-7-(4-pyridinyl)-4-quinolinyl]amino}benzoic acid, bistrifluoroacetate salt

A solution of ethyl 3-{[3-(aminocarbonyl)-7-bromo-4-quinolinyl]amino}benzoate (example 6a, 144 mg, 0.348 mmol), 4-pyridinyl boronic acid (64.1 mg, 0.521 mmol), potassium carbonate (144 mg, 1.043 mmol), and tetrakis(triphenylphosphine)palladium(0) (18.08 mg, 0.016 mmol) in 1,4-dioxane (2.0 ml) and water (2 ml) was heated to 100° C. for min. in a Biotage Initiator® microwave synthesizer. Upon cooling, the reaction mixture was diluted with water and extracted with ethyl acetate. The organic portion was concentrated and the residue diluted with ethanol (0.667 ml) and treated with sodium hydroxide (1M aq solution) (0.348 ml, 0.348 mmol). Following stirring overnight at room temperature, the ethanol was concentrated, the residue diluted with water, then adjusted to pH ˜4 using 1N hydrochloric acid. The resulting solid was purified using Reverse Phase HPLC (0.1% TFA in acetonitrile and water) to obtain the title compound (0.044 g, 21%) as a yellow solid. ¹H NMR (400 MHz, DMSO-d₆) ppm 12.96 (br. s., 1H), 10.41 (s, 1H), 9.05 (s, 1H), 8.69 (d, J=6.1 Hz, 2H), 8.41 (d, J=1.8 Hz, 1H), 8.29 (s, 1H), 7.90-7.97 (m, 2H), 7.85-7.91 (m, 2H), 7.72 (br. s., 1H), 7.59 (d, J=7.8 Hz, 1H), 7.52 (s, 1H), 7.39 (t, J=7.8 Hz, 1H), 7.24 (dd, J=8.0, 1.6 Hz, 1H).

### Example 23

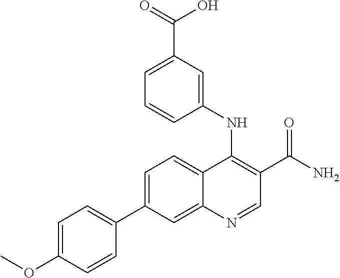 [SMILES: COc1ccc(-c2ccc3c(Nc4cccc(C(=O)O)c4)c(C(N)=O)cnc3c2)cc1]

### 3-({3-(aminocarbonyl)-7-[4-(methyloxy)phenyl]-4-quinolinyl}amino)benzoic acid, hydrochloride salt

See example 17a.

### Example 24

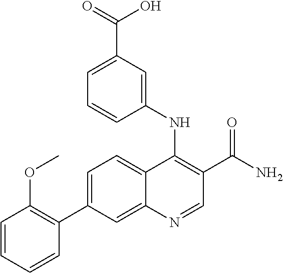 [SMILES: COc1ccccc1-c1ccc2c(Nc3cccc(C(=O)O)c3)c(C(N)=O)cnc2c1]

### 3-({3-(aminocarbonyl)-7-[2-(methyloxy)phenyl]-4-quinolinyl}amino)benzoic acid

### a) ethyl 3-({3-(aminocarbonyl)-7-[2-(methyloxy)phenyl]-4-quinolinyl}amino)benzoate

A solution of ethyl 3-{[3-(aminocarbonyl)-7-chloro-4-quinolinyl]amino}benzoate (example 14b, 250 mg, 0.676 mmol), 2-methoxyphenylboronic acid (308 mg, 2.028 mmol), potassium carbonate (280 mg, 2.028 mmol), and tetrakis(triphenylphosphine)palladium(0) (39.1 mg, 0.034 mmol) in 1,4-dioxane (2.0 ml) and water (0.667 ml) was heated to 100° C. for 20 min. in a Biotage Initiator® microwave synthesizer. Upon cooling, the mixture was diluted with water and extracted with ethyl acetate (3×). The combined organic portions were combined and concentrated under vacuo. The residue was purified using Reverse Phase HPLC (0.1% TFA in acetonitrile and water). The desired fractions were combined and concentrated to a solid, which was dissolved in ethyl acetate and washed with 1N aqueous sodium hydroxide. The organic portion was dried (MgSO₄), dried and concentrated to obtain the title compound (0.105 g, 35%) as a yellow solid. MS (ES+) m/e 442 [M+H]⁺.

### b) 3-({3-(aminocarbonyl)-7-[2-(methyloxy)phenyl]-4-quinolinyl}amino)benzoic acid

To a suspension of ethyl 3-({3-(aminocarbonyl)-7-[2-(methyloxy)phenyl]-4-quinolinyl}amino)benzoate (79 mg, 0.180 mmol) in ethanol (1.0 mL) was added 1N aqueous sodium hydroxide (0.180 ml, 0.180 mmol). After stirring overnight at ambient temperature, the ethanol was evaporated in vacuo. The residue was diluted with water and extracted with ethyl acetate twice. The aqueous portion was made acidic (pH˜4) with 1N aqueous hydrochloric acid and the resulting precipitate was filtered, washed with water, and dried in vacuo to to obtain 3-({3-(aminocarbonyl)-7-[2-(methyloxy)phenyl]-4-quinolinyl}amino)benzoic acid (66 mg, 0.160 mmol, 89% yield) as a yellow solid. ¹H NMR (400 MHz, DMSO-d₆) ppm 10.37 (s, 1H), 9.01 (s, 1H), 8.27 (br. s., 1H), 8.06 (d, J=1.5 Hz, 1H), 7.81 (d, J=8.8 Hz, 1H), 7.69 (br. s., 1H), 7.54-7.60 (m, 2H), 7.52 (s, 1H), 7.34-7.47 (m, 3H), 7.22 (dd, J=8.1, 1.5 Hz, 1H), 7.17 (d, J=7.8 Hz, 1H), 7.08 (td, J=7.4, 0.9 Hz, 1H), 3.79 (s, 3H).

### Example 25

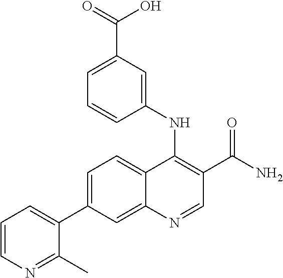 [SMILES: Cc1ncccc1-c1ccc2c(Nc3cccc(C(=O)O)c3)c(C(N)=O)cnc2c1]

### 3-{[3-(aminocarbonyl)-7-(2-methyl-3-pyridinyl)-4-quinolinyl]amino}benzoic acid

### a) ethyl 3-{[3-(aminocarbonyl)-7-(2-methyl-3-pyridinyl)-4-quinolinyl]amino}benzoate

A solution of ethyl 3-{[3-(aminocarbonyl)-7-chloro-4-quinolinyl]amino}benzoate (example 14b, 134 mg, 0.362 mmol), 2-methylpyridine-3-boronic acid (149 mg, 1.087 mmol), potassium carbonate (226 mg, 1.635 mmol), and tetrakis(triphenylphosphine)palladium(0) (20 mg, 0.017 mmol) in 1,4-dioxane (2.0 ml) and water (0.667 ml) was heated to 120° C. for 20 min. in a Biotage Initiator®microwave synthesizer. Upon cooling, the reaction mixture was diluted with water and extracted with ethyl acetate twice. The combined organic portions were dried (MgSO₄) and concentrated. Following purification via flash column chromatography (0-10% methanol in dichloromethane) the desired fractions were concentrated and the residue triturated in ethyl ether to obtain the title compound (0.062 g, 40%) as a light yellow solid. ¹H NMR (400 MHz, DMSO-d₆) ppm 10.35 (s, 1H), 9.02 (s, 1H), 8.52 (dd, J=4.8, 1.5 Hz, 1H), 8.24 (br. s., 1H), 7.96 (d, J=1.8 Hz, 1H), 7.93 (d, J=8.6 Hz, 1H), 7.75 (dd, J=7.7, 1.6 Hz, 1H), 7.68 (br. s., 1H), 7.54-7.62 (m, 2H), 7.51 (dd, J=8.6, 1.8 Hz, 1H), 7.37-7.43 (m, 1H), 7.36 (dd, J=7.7, 4.9 Hz, 1H), 7.25 (dd, J=8.1, 1.5 Hz, 1H), 4.27 (q, J=7.1 Hz, 2H), 2.47 (s, 3H), 1.27 (t, J=7.1 Hz, 3H).

### b) 3-{[3-(aminocarbonyl)-7-(2-methyl-3-pyridinyl)-4-quinolinyl]amino}benzoic acid

To a suspension of ethyl 3-{[3-(aminocarbonyl)-7-(2-methyl-3-pyridinyl)-4-quinolinyl]amino}benzoate (50 mg, 0.117 mmol) in ethanol (3.0 mL) was added 1N aqueous sodium hydroxide (2 ml, 2.000 mmol). After stirring 2 h at ambient temperature, the reaction mixture was concentrated to an aqueous residue, which was then neutralized with 6N aqueous hydrochloric acid. The resulting precipitate was filtered, washed with water, and dried in vacuo to obtain the title compound (0.048 g, 103%) as a bright yellow solid. ¹H NMR (400 MHz, DMSO-d₆) ppm 12.96 (br. s., 1H), 10.44 (s, 1H), 9.04 (s, 1H), 8.52 (dd, J=4.8, 1.5 Hz, 1H), 8.27 (br. s., 1H), 7.96 (s, 1H), 7.92 (d, J=8.6 Hz, 1H), 7.76 (dd, J=7.6, 1.5 Hz, 1H), 7.71 (br. s., 1H), 7.59 (d, J=7.8 Hz, 1H), 7.53 (s, 1H), 7.50 (dd, J=8.7, 1.4 Hz, 1H), 7.37-7.42 (m, 1H), 7.35 (dd, J=7.7, 4.9 Hz, 1H), 7.26 (dd, J=7.6, 1.8 Hz, 1H), 2.46 (s, 3H).

### Example 26

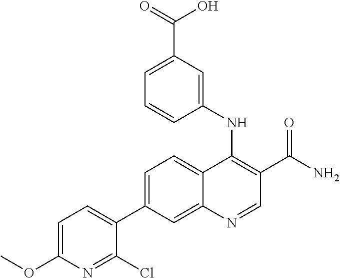 [SMILES: COc1ccc(-c2ccc3c(Nc4cccc(C(=O)O)c4)c(C(N)=O)cnc3c2)c(Cl)n1]

### 3-({3-(aminocarbonyl)-7-[2-chloro-6-(methyloxy)-3-pyridinyl]-4-quinolinyl}amino)benzoic acid

A solution of ethyl 3-{[3-(aminocarbonyl)-7-bromo-4-quinolinyl]amino}benzoate (example 6a, 100 mg, 0.241 mmol), 2-chloro-6-methoxypyridine-3-boronic acid (136 mg, 0.724 mmol), potassium carbonate (100 mg, 0.724 mmol), and tetrakis(triphenylphosphine)palladium(0) (13.95 mg, 0.012 mmol) in 1,4-dioxane (2.0 ml) and water (0.667 ml) was heated to 100° C. for 20 min. in a Biotage Initiator® microwave synthesizer. Upon cooling, the reaction mixture was diluted with water, extracted with ethyl acetate dried using magnesium sulfate and concentrated in vacuo. The residue was purified via flash column chromatography (0-100% ethyl acetate in hexanes, then 10% methanol in ethyl acetate). The obtained solid (75% by LC-MS) was treated with 1N aq sodium hydroxide (1 ml, 1.000 mmol) as a solution in ethanol (2 mL). After stirring overnight at ambient temperature, the reaction was concentrated to an aqueous residue, which was neutralized with 6N aqueous hydrochloric acid. The resulting precipitate was filtered, washed with water, and purified using Reverse Phase HPLC (0.1% TFA in acetonitrile and water) to obtain the title compound (0.016 g, 15%) as a yellow solid. ¹H NMR (400 MHz, DMSO-d₆) ppm 11.57 (br. s., 1H), 9.01 (br. s., 1H), 8.17-8.39 (m, 2H), 7.88-8.16 (m, 2H), 7.64-7.87 (m, 3H), 7.51 (br. s., 2H), 7.04 (d, J=6.8 Hz, 1H), 3.93 (br. s., 3H).

### Example 27

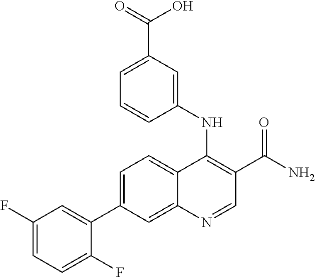 [SMILES: NC(=O)c1cnc2cc(-c3cc(F)ccc3F)ccc2c1Nc1cccc(C(=O)O)c1]

### 3-{[3-(aminocarbonyl)-7-(2,5-difluorophenyl)-4-quinolinyl]amino}benzoic acid

### a) ethyl 3-{[3-(aminocarbonyl)-7-(2,5-difluorophenyl)-4-quinolinyl]amino}benzoate

A solution of ethyl 3-{[3-(aminocarbonyl)-7-chloro-4-quinolinyl]amino}benzoate (example 14b, 175 mg, 0.473 mmol), (2,5-difluorophenyl)boronic acid (200 mg, 1.267 mmol), potassium carbonate (175 mg, 1.266 mmol), and tetrakis(triphenylphosphine)palladium(0) (68 mg, 0.059 mmol) in 1,4-dioxane (2.0 ml) and water (0.667 ml) was heated to 120° C. for 20 min. in a Biotage Initiator®microwave synthesizer. Upon cooling, the mixture was diluted with water and extracted with ethyl acetate (3×). The combined organic portions were combined and concentrated under vacuo. The resulting residue was purified via flash chromatography (10% methanol in dichloromethane) to obtain an amber oil. This oil was slurried into ethyl ether and filtered to obtain a mixture of mainly starting material and product. This crude was used in the next step. MS (ES+) m/e 448 [M+H]⁺.

### b) 3-{[3-(aminocarbonyl)-7-(2,5-difluorophenyl)-4-quinolinyl]amino}benzoic acid

A solution of ethyl 3-{[3-(aminocarbonyl)-7-(2,5-difluorophenyl)-4-quinolinyl]amino}benzoate (98 mg, 0.219 mmol) in ethanol (2 mL) was treated with sodium hydroxide (1N aq solution) (2 mL, 2.000 mmol). After stirring overnight at room temperature, the ethanol was removed in vacuo and the aqueous residue was acidified to pH˜4 using 1N hydrochloric acid. The resulting solid was filtered and purified using Reverse Phase HPLC (0.1% TFA in acetonitrile and water) to obtain the title compound (0.015 g, 16%) as a light yellow solid. ¹H NMR (400 MHz, DMSO-d₆) ppm 11.54 (br. s., 1H), 8.99 (s, 1H), 8.26 (br. s., 1H), 8.24 (d, J=8.8 Hz, 1H), 8.21 (s, 1H), 7.77-7.89 (m, 3H), 7.74 (br. s., 1H), 7.64 (ddd, J=9.1, 6.1, 3.3 Hz, 1H), 7.45-7.56 (m, 3H), 7.35-7.44 (m, 1H).

### Example 28

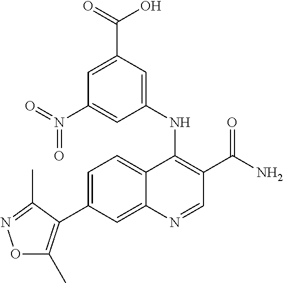 [SMILES: Cc1noc(C)c1-c1ccc2c(Nc3cc(C(=O)O)cc([N+](=O)[O-])c3)c(C(N)=O)cnc2c1]

### 3-{[3-(aminocarbonyl)-7-(3,5-dimethyl-4-isoxazolyl)-4-quinolinyl]amino}-5-nitrobenzoic acid

### a) 3-{[3-(aminocarbonyl)-7-(3,5-dimethyl-4-isoxazolyl)-4-quinolinyl]amino}-5-nitrobenzoic acid

To a suspension of 4-chloro-7-(3,5-dimethyl-4-isoxazolyl)-3-quinolinecarboxamide (example 86a, 100 mg, 0.331 mmol) in acetic acid (10 mL) was added 3-amino-5-nitrobenzoic acid (60.4 mg, 0.331 mmol). The reaction mixture was stirred at 100° C. for 2 hours. After cooling, the precipitate was collected, washed with ether and dried under reduced pressure to afford the title compound (105 mg, 0.235 mmol, 70.8% yield) as a yellow solid. ¹H NMR (400 MHz, DMSO-d6) δ ppm 11.56 (br. s., 1H), 9.01 (s, 1H), 8.65 (d, J=9.1 Hz, 1H), 8.44 (s, 1H), 8.32 (s, 1H), 8.25 (s, 1H), 8.19 (s, 1H), 8.17 (d, J=1.5 Hz, 1H), 7.87 (dd, J=9.0, 1.6 Hz, 1H), 7.66 (s, 1H), 2.56 (s, 3H), 2.37 (s, 3H).

### Example 29

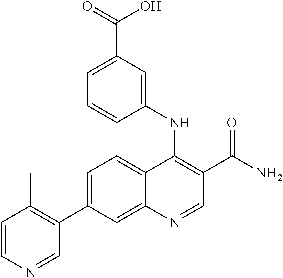 [SMILES: Cc1ccncc1-c1ccc2c(Nc3cccc(C(=O)O)c3)c(C(N)=O)cnc2c1]

### 3-{[3-(aminocarbonyl)-7-(4-methyl-3-pyridinyl)-4-quinolinyl]amino}benzoic acid

### a) ethyl 3-{[3-(aminocarbonyl)-7-(4-methyl-3-pyridinyl)-4-quinolinyl]amino}benzoate

A solution of ethyl 3-{[3-(aminocarbonyl)-7-chloro-4-quinolinyl]amino}benzoate (example 14b, 134 mg, 0.362 mmol), 4-methylpyridine-3-boronic acid (149 mg, 1.087 mmol), potassium carbonate (205 mg, 1.483 mmol), and tetrakis(triphenylphosphine)palladium(0) (28 mg, 0.024 mmol) in 1,4-dioxane (2.0 ml) and water (0.667 ml) was heated to 120° C. for 20 min. in a Biotage Initiator® microwave synthesizer. Upon cooling, the reaction mixture was diluted with water and extracted with ethyl acetate twice. The combined organic portions were dried (MgSO₄) and concentrated. Following purification via flash column chromatography (0-10% methanol in dichloromethane) the desired fractions were concentrated and the residue triturated in ethyl ether to obtain the title compound (0.065 g, 42%) as a light yellow solid. ¹H NMR (400 MHz, DMSO-d₆) ppm 10.35 (s, 1H), 9.02 (s, 1H), 8.50 (s, 1H), 8.49 (d, J=5.1 Hz, 1H), 8.24 (br. s., 1H), 7.97 (d, J=1.8 Hz, 1H), 7.94 (d, J=8.8 Hz, 1H), 7.68 (br. s., 1H), 7.56-7.62 (m, 2H), 7.51 (dd, J=8.7, 1.9 Hz, 1H), 7.35-7.44 (m, 2H), 7.21-7.28 (m, 1H), 4.27 (q, J=7.1 Hz, 2H), 2.31 (s, 3H), 1.27 (t, J=7.1 Hz, 3H).

### b) 3-{[3-(aminocarbonyl)-7-(4-methyl-3-pyridinyl)-4-quinolinyl]amino}benzoic acid

To a suspension of ethyl 3-{[3-(aminocarbonyl)-7-(4-methyl-3-pyridinyl)-4-quinolinyl]amino}benzoate (48 mg, 0.113 mmol) in ethanol (3.0 mL) was added 1N aqueous sodium hydroxide (1.126 ml, 1.126 mmol). After stirring 2 h at ambient temperature, the reaction mixture was concentrated to an aqueous residue, which was then neutralized with 6N aqueous hydrochloric acid. The resulting precipitate was filtered, washed with water, and dried in vacuo to obtain the title compound (0.038 g, 87%) as an orange solid. ¹H NMR (400 MHz, DMSO-d₆) ppm 10.54 (br. s., 1H), 9.03 (s, 1H), 8.51 (br. s., 2H), 8.27 (br. s., 1H), 7.97 (s, 2H), 7.95 (s, 1H), 7.71 (br. s., 1H), 7.61 (d, J=7.6 Hz, 1H), 7.56 (s, 1H), 7.53 (dd, J=8.7, 1.4 Hz, 1H), 7.35-7.45 (m, 2H), 7.28 (dd, J=8.1, 1.4 Hz, 1H), 2.31 (s, 3H).

### Example 30

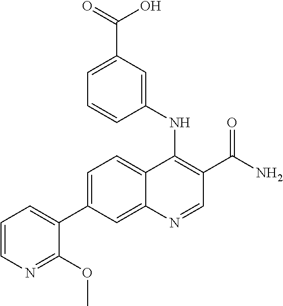 [SMILES: COc1ncccc1-c1ccc2c(Nc3cccc(C(=O)O)c3)c(C(N)=O)cnc2c1]

### 3-({3-(aminocarbonyl)-7-[2-(methyloxy)-3-pyridinyl]-4-quinolinyl}amino)benzoic acid

A solution of ethyl 3-{[3-(aminocarbonyl)-7-chloro-4-quinolinyl]amino}benzoate (example 14b, 134 mg, 0.362 mmol), 2-methoxypyridinyl-3-boronic acid (166 mg, 1.087 mmol), potassium carbonate (150 mg, 1.087 mmol), and tetrakis(triphenylphosphine)palladium(0) (20.94 mg, 0.018 mmol) in 1,4-dioxane (2.0 ml) and water (3 ml) was heated to 120° C. for 20 min. in a Biotage Initiator® microwave synthesizer. Upon cooling, the reaction mixture was concentrated and diluted with ethanol (3 mL) then treated with 1N sodium hydroxide. Following stirring overnight at room temperature, the mixture was concentrated and neutralized using 1N hydrochloric acid. The resulting solid was purified using Reverse Phase HPLC (ODS column 0.1 TFA in acetonitrile and water) to obtain the title compound (0.030 g, 16%) as an off white solid. ¹H NMR (400 MHz, DMSO-d₆) ppm 13.16 (br. s., 1H), 8.99 (s, 1H), 8.30 (dd, J=4.9, 1.8 Hz, 1H), 8.26 (br. s., 1H), 8.24 (d, J=1.5 Hz, 1H), 8.10-8.19 (m, 1H), 7.98 (dd, J=7.5, 1.9 Hz, 1H), 7.73-7.89 (m, 4H), 7.46-7.60 (m, 2H), 7.20 (dd, J=7.5, 4.9 Hz, 1H), 3.94 (s, 3H).

### Example 31

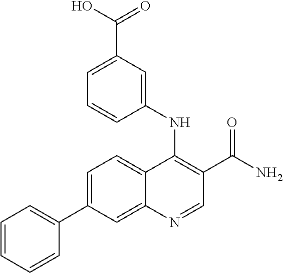 [SMILES: NC(=O)c1cnc2cc(-c3ccccc3)ccc2c1Nc1cccc(C(=O)O)c1]

### 3-{[3-(aminocarbonyl)-7-phenyl-4-quinolinyl]amino}benzoic acid

A mixture of ethyl 3-{[3-(aminocarbonyl)-7-chloro-4-quinolinyl]amino}benzoate (example 14b, 0.082 g, 0.222 mmol), phenylboronic acid (0.054 g, 0.443 mmol), bis(tri-t-butylphosphine)palladium(0) (0.006 g, 0.011 mmol) and potassium carbonate (0.061 g, 0.443 mmol) in 1,4-dioxane (1 mL) and water (0.25 mL) was stirred at 120° C. in a microwave synthesiser for 0.5 h, then cooled. 6M aqueous sodium hydroxide (0.50 mL, 3.00 mmol) was added, followed by ethanol (3 mL). The mixture was stirred for 18 h, then water (5 mL) added and the mixture filtered through a PTFE micropore filter. The pH was adjusted to 4 with 6M aqueous hydrochloric acid. The solid was filtered off, washed with water, dried and purified by reverse-phase preparative HPLC (ODS, 10-90% acetonitrile/water+0.1% trifluoroacetic acid) to give the title compound (0.064 g, 75%) as a pale yellow solid. ¹H NMR (400 MHz, DMSO-d₆) ppm 7.46-7.55 (m, 3H) 7.56-7.61 (m, 2H) 7.75-7.84 (m, 3H) 7.85-7.89 (m, 2H) 7.96-8.02 (m, 1H) 8.20 (d, J=9.09 Hz, 1H) 8.22 (d, J=1.77 Hz, 1H) 8.27 (br. s., 1H) 9.01 (s, 1H).

### Example 32

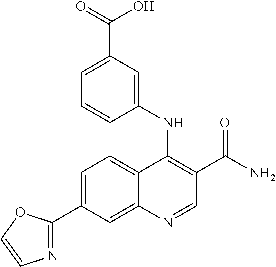 [SMILES: NC(=O)c1cnc2cc(-c3ncco3)ccc2c1Nc1cccc(C(=O)O)c1]

### 3-{[3-(aminocarbonyl)-7-(1,3-oxazol-2-yl)-4-quinolinyl]amino}benzoic acid, bistrifluoroacetate salt

To a solution of ethyl 3-{[3-(aminocarbonyl)-7-bromo-4-quinolinyl]amino}benzoate (example 6a, 130 mg, 0.314 mmol) in 1,4-dioxane (1.5 ml) was added 2-(tributylstannanyl)-1,3-oxazole (300 mg, 0.838 mmol) and tetrakis(triphenylphosphine)palladium(0) (16.32 mg, 0.014 mmol) followed by heating to 150° C. for 20 min. in a Biotage Initiator® microwave synthesizer. Upon cooling, the reaction mixture was concentrated in vacuo, diluted with ethanol (2 ml) and then treated with sodium hydroxide (1M aq solution) (2 ml, 2.000 mmol). Following stirring overnight at room temperature, the ethanol was removed in vacuo, the residue diluted with water and extracted using ethyl acetate. The aqueous portion was then adjusted to pH˜4 using 1N hydrochloric acid. The resulting solid was filtered and purified using Reverse Phase (0.1% TFA in acetonitrile and water) to obtain the title compound (0.025 g, 13%) as a yellow solid. ¹H NMR (400 MHz, DMSO-d₆) ppm 13.11 (br. s., 1H), 11.26 (br. s., 1H), 9.02 (s, 1H), 8.52 (d, J=1.8 Hz, 1H), 8.41 (s, 1H), 8.25-8.32 (m, 1H), 8.19-8.24 (m, 1H), 8.13 (d, J=9.1 Hz, 1H), 7.77 (d, J=8.1 Hz, 1H), 7.74 (br. s., 2H), 7.55 (s, 1H), 7.49 (t, J=7.7 Hz, 1H), 7.41-7.46 (m, 1H).

### Example 33

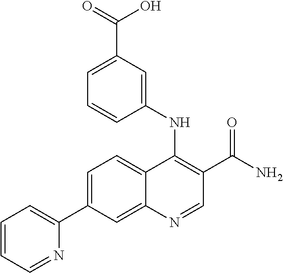 [SMILES: NC(=O)c1cnc2cc(-c3ccccn3)ccc2c1Nc1cccc(C(=O)O)c1]

### 3-{[3-(aminocarbonyl)-7-(2-pyridinyl)-4-quinolinyl]amino}benzoic acid, bistrifluoroacetate salt

A solution of ethyl 3-{[3-(aminocarbonyl)-7-bromo-4-quinolinyl]amino}benzoate (example 6a, 100 mg, 0.241 mmol), 2-(tributylstannanyl)pyridine (178 mg, 0.483 mmol), and tetrakis(triphenylphosphine)palladium(0) (12.55 mg, 10.86 μmol) in 1,4-dioxane (2.0 ml) was heated to 150° C. for 20 min. in a Biotage Initiator® microwave synthesizer. Upon cooling, the reaction mixture was concentrated in vacuo, diluted with ethanol (2 ml) and then treated with sodium hydroxide (1M aq solution) (2 ml, 2.000 mmol). Following stirring overnight at room temperature, the ethanol was removed in vacuo, the residue diluted with water and extracted using ethyl acetate. The aqueous portion was then adjusted to pH˜4 using 1N hydrochloric acid. The solution was purified using Reverse Phase (0.1% TFA in acetonitrile and water) to obtain the title compound (0.028 g, 19%) as a bright yellow solid. ¹H NMR (400 MHz, DMSO-d₆) ppm 13.17 (br. s., 1H), 11.65 (br. s., 1H), 9.00 (s, 1H), 8.79 (ddd, J=4.7, 1.7, 0.9 Hz, 1H), 8.75 (d, J=1.8 Hz, 1H), 8.31-8.40 (m, 1H), 8.26 (s, 1H), 8.27 (d, J=9.1 Hz, 1H), 8.21 (d, J=8.1 Hz, 1H), 8.02 (td, J=7.7, 1.8 Hz, 1H), 7.80-7.89 (m, 2H), 7.77 (br. s., 1H), 7.47-7.58 (m, 3H).

### Example 34

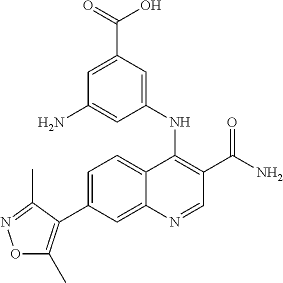 [SMILES: Cc1noc(C)c1-c1ccc2c(Nc3cc(N)cc(C(=O)O)c3)c(C(N)=O)cnc2c1]

### 3-amino-5-{[3-(aminocarbonyl)-7-(3,5-dimethyl-4-isoxazolyl)-4-quinolinyl]amino}benzoic acid, trifluoroacetate salt

### a) 3-{[3-(aminocarbonyl)-7-(3,5-dimethyl-4-isoxazolyl)-4-quinolinyl]amino}-5-nitrobenzoic acid

To a suspension of 4-chloro-7-(3,5-dimethyl-4-isoxazolyl)-3-quinolinecarboxamide (example 86a, 100 mg, 0.331 mmol) in acetic acid (10 mL) was added 3-amino-5-nitrobenzoic acid (60.4 mg, 0.331 mmol). The reaction mixture was stirred at 100° C. for 2 hours. After cooling, the precipitate was collected, washed with ether and dried under reduced pressure to afford 3-{[3-(aminocarbonyl)-7-(3,5-dimethyl-4-isoxazolyl)-4-quinolinyl]amino}-5-nitrobenzoic acid (105 mg, 0.235 mmol, 70.8% yield) as a yellow solid. ¹H NMR (400 MHz, DMSO-d6) δ ppm 11.56 (br. s., 1H), 9.01 (s, 1H), 8.65 (d, J=9.1 Hz, 1H), 8.44 (s, 1H), 8.32 (s, 1H), 8.25 (s, 1H), 8.19 (s, 1H), 8.17 (d, J=1.5 Hz, 1H), 7.87 (dd, J=9.0, 1.6 Hz, 1H), 7.66 (s, 1H), 2.56 (s, 3H), 2.37 (s, 3H).

### b) 3-amino-5-{[3-(aminocarbonyl)-7-(3,5-dimethyl-4-isoxazolyl)-4-quinolinyl]amino}benzoic acid

To a suspension of 3-{[3-(aminocarbonyl)-7-(3,5-dimethyl-4-isoxazolyl)-4-quinolinyl]amino}-5-nitrobenzoic acid (85 mg, 0.190 mmol) in methanol (15 mL) was added palladium on carbon (6.07 mg, 0.057 mmol). The reaction mixture was hydrogenated under a balloon of hydrogen overnight. After removing hydrogen, the mixture was filtered and purified through preparative HPLC (YMC 75×30 mm column, 0.1% TFA in water and 0.1% TFA in acetonitrile), and triturated to afford the title compound (74 mg, 0.139 mmol, 73.3% yield) as a yellow solid. ¹H NMR (400 MHz, DMSO-d6) δ ppm 12.40 (br. s., 1H), 9.06 (s, 1H), 8.41 (br. s., 1H), 8.05 (d, J=8.8 Hz, 1H), 7.99 (d, J=1.3 Hz, 2H), 7.66 (dd, J=8.8, 1.5 Hz, 1H), 7.19 (s, 1H), 7.00 (s, 1H), 6.75 (s, 1H), 2.51 (s, 3H), 2.33 (s, 3H).

### Example 35

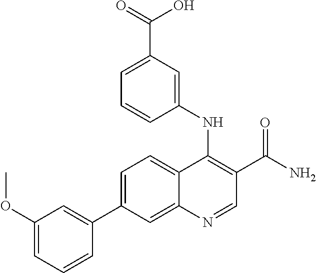 [SMILES: COc1cccc(-c2ccc3c(Nc4cccc(C(=O)O)c4)c(C(N)=O)cnc3c2)c1]

### 3-({3-(aminocarbonyl)-7-[3-(methyloxy)phenyl]-4-quinolinyl}amino)benzoic acid

### a) ethyl 3-({3-(aminocarbonyl)-7-[3-(methyloxy)phenyl]-4-quinolinyl}amino)benzoate

A solution of ethyl 3-{[3-(aminocarbonyl)-7-chloro-4-quinolinyl]amino}benzoate (example 14b, 300 mg, 0.811 mmol), 3-methoxyphenylboronic acid (370 mg, 2.434 mmol), potassium carbonate (336 mg, 2.434 mmol), and tetrakis(triphenylphosphine)palladium(0) (46.9 mg, 0.041 mmol) in 1,4-dioxane 2.0 ml) and water (0.667 ml) was heated to 120° C. for 20 min. in a Biotage Initiator® microwave synthesizer. Upon cooling, the reaction mixture was diluted with water and then extracted using ethyl acetate twice. The organic portion was dried over magnesium sulfate, filtered and concentrated. Following purification using flash chromatography (10-100% ethyl acetate in hexanes), the desired fractions were concentrated under reduced pressure. The obtained solid was triturated in ethyl ether to obtain the title compound (0.142 g, 40%) as an off white solid. ¹H NMR (400 MHz, DMSO-d₆) ppm 10.38 (s, 1H), 9.03 (s, 1H), 8.25 (d, J=1.5 Hz, 2H), 7.86-7.93 (m, 1H), 7.79 (dd, J=8.8, 1.8 Hz, 1H), 7.68 (br. s., 1H), 7.59 (d, J=7.8 Hz, 1H), 7.57 (br. s., 1H), 7.32-7.47 (m, 4H), 7.22 (dd, J=7.8, 1.5 Hz, 1H), 7.01 (dt, J=6.9, 2.5 Hz, 1H), 4.27 (q, J=7.1 Hz, 2H), 3.86 (s, 3H), 1.27 (t, J=7.1 Hz, 3H).

### b) 3-({3-(aminocarbonyl)-7-[3-(methyloxy)phenyl]-4-quinolinyl}amino)benzoic acid

To a suspension of ethyl 3-({3-(aminocarbonyl)-7-[3-(methyloxy)phenyl]-4-quinolinyl}amino)benzoate (135 mg, 0.306 mmol) in ethanol (1.0 mL) was added 1N aqueous sodium hydroxide (1 ml, 1.000 mmol). After stirring overnight at ambient temperature, the reaction was concentrated to an aqueous residue, which was neutralized with 6N aqueous hydrochloric acid. The resulting precipitate was filtered, washed with water, and dried in vacuo to obtain the title compound (0.128 g, 101%) as a pale yellow solid. ¹H NMR (400 MHz, DMSO-d₆) ppm 13.13 (br. s., 1H), 11.63 (br. s., 1H), 8.96-9.09 (m, 1H), 8.33 (br. s., 1H), 8.28 (d, J=1.8 Hz, 1H), 8.15 (d, J=8.8 Hz, 1H), 7.96 (dd, J=9.0, 1.6 Hz, 1H), 7.82 (d, J=6.8 Hz, 1H), 7.74-7.80 (m, 2H), 7.45-7.57 (m, 3H), 7.39-7.43 (m, 1H), 7.37 (t, J=2.0 Hz, 1H), 7.08 (dd, J=8.1, 1.8 Hz, 1H), 3.86 (s, 3H).

### Example 36

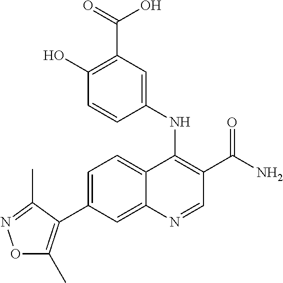 [SMILES: Cc1noc(C)c1-c1ccc2c(Nc3ccc(O)c(C(=O)O)c3)c(C(N)=O)cnc2c1]

### 5-{[3-(aminocarbonyl)-7-(3,5-dimethyl-4-isoxazolyl)-4-quinolinyl]amino}-2-hydroxybenzoic acid, trifluoroacetate salt

### a) 5-{[3-(aminocarbonyl)-7-bromo-4-quinolinyl]amino}-2-hydroxybenzoic acid

To a suspension of 7-bromo-4-chloro-3-quinolinecarboxamide (example 1a, 100 mg, 0.350 mmol) in acetic acid (10 mL) was added 5-amino-2-hydroxybenzoic acid (0.054 g, 0.350 mmol). The reaction mixture was stirred at 100° C. for 2 hours. After cooling, the reaction was quenched with water, the precipitate was collected and dried in air to afford 5-{[3-(aminocarbonyl)-7-bromo-4-quinolinyl]amino}-2-hydroxybenzoic acid (58 mg, 0.144 mmol, 41.2% yield) as a brown solid. ¹H NMR (400 MHz, DMSO-d6) δ ppm 12.08 (br. s., 1H), 8.98 (br. s., 1H), 8.35 (br. s., 1H), 8.30 (s, 1H), 8.11 (br. s., 1H), 7.78 (d, J=8.1 Hz, 2H), 7.73 (d, J=2.5 Hz, 1H), 7.47 (dd, J=8.8, 2.5 Hz, 1H), 7.02 (d, J=8.6 Hz, 1H).

### b) 5-{[3-(aminocarbonyl)-7-(3,5-dimethyl-4-isoxazolyl)-4-quinolinyl]amino}-2-hydroxybenzoic acid, trifluoroacetate salt

To a suspension of 5-{[3-(aminocarbonyl)-7-bromo-4-quinolinyl]amino}-2-hydroxybenzoic acid (50 mg, 0.124 mmol), (3,5-dimethyl-4-isoxazolyl)boronic acid (35.0 mg, 0.249 mmol) and tetrakis(triphenylphosphine)palladium(0) (7.18 mg, 6.22 μmol) in 1,4-dioxane (6 mL) and water (2.000 mL) was added potassium carbonate (34.4 mg, 0.249 mmol). The reaction mixture was stirred at 100° C. for 2 hours. After cooling, the mixture was purifed through preparative HPLC (YMC 75×30 mm column, 0.1% TFA in water and 0.1% TFA in acetonitrile) to afford the title compound (30 mg, 0.056 mmol, 45.3% yield) as a yellow solid. ¹H NMR (400 MHz, DMSO-d6) δ ppm 11.31 (br. s., 1H), 8.97 (s, 1H), 8.28 (br. s., 1H), 7.83-7.92 (m, 2H), 7.74 (br. s., 1H), 7.53 (d, J=2.5 Hz, 1H), 7.42-7.49 (m, 1H), 7.25 (dd, J=8.8, 2.8 Hz, 1H), 6.86 (d, J=8.8 Hz, 1H), 2.48 (s, 3H), 2.29 (s, 3H).

### Example 37

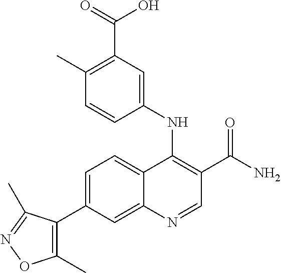 [SMILES: Cc1ccc(Nc2c(C(N)=O)cnc3cc(-c4c(C)noc4C)ccc23)cc1C(=O)O]

### 5-{[3-(aminocarbonyl)-7-(3,5-dimethyl-4-isoxazolyl)-4-quinolinyl]amino}-2-methylbenzoic acid, trifluoroacetate salt

To a suspension of 4-chloro-7-(3,5-dimethyl-4-isoxazolyl)-3-quinolinecarboxamide (example 86a, 25 mg, 0.083 mmol) in acetic acid (10 mL) was added 5-amino-2-methylbenzoic acid (12.52 mg, 0.083 mmol). The reaction mixture was stirred at 80° C. for 2 hours. After cooling, the reaction was purifed through preparative HPLC (YMC 75×30 mm column, 0.1% TFA in water and 0.1% TFA in acetonitrile) to afford the title compound (13 mg, 0.025 mmol, 29.6% yield) as a yellow solid. ¹H NMR (400 MHz, DMSO-d6) δ ppm 13.05 (br. s., 1H), 11.76 (br. s., 1H), 9.00 (s, 1H), 8.28 (br. s., 1H), 8.17 (d, J=8.6 Hz, 1H), 7.98 (d, J=1.3 Hz, 1H), 7.83 (br. s., 1H), 7.74 (s, 1H), 7.69 (d, J=8.1 Hz, 1H), 7.36 (s, 2H), 2.55 (s, 3H), 2.52 (br. s., 3H), 2.33 (s, 3H).

### Example 38

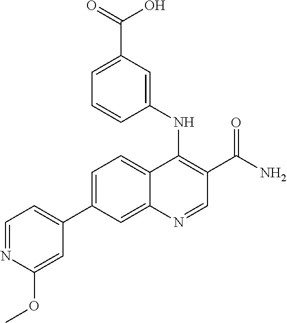 [SMILES: COc1cc(-c2ccc3c(Nc4cccc(C(=O)O)c4)c(C(N)=O)cnc3c2)ccn1]

### 3-({3-(aminocarbonyl)-7-[2-(methyloxy)-4-pyridinyl]-4-quinolinyl}amino)benzoic acid

### a) ethyl 3-({3-(aminocarbonyl)-7-[2-(methyloxy)-4-pyridinyl]-4-quinolinyl}amino)benzoate

A solution of ethyl 3-{[3-(aminocarbonyl)-7-bromo-4-quinolinyl]amino}benzoate (example 6a, 300 mg, 0.724 mmol), 2-methoxypyridinyl-4-boronic acid (332 mg, 2.173 mmol), potassium carbonate (300 mg, 2.173 mmol), and tetrakis(triphenylphosphine)palladium (0) (37.7 mg, 0.033 mmol) in 1,4-dioxane (2.0 ml) and water (0.667 ml) was heated to 120° C. for 20 min. in a Biotage Initiator® microwave synthesizer. Upon cooling, the reaction mixture was diluted with water and extracted with ethyl acetate twice. The combined organic portions were dried (MgSO₄) and concentrated. Following purification via flash column chromatography (0-10% methanol in dichloromethane) the desired fractions were concentrated and the residue triturated in ethyl ether to obtain the title compound (0.130 g, 41%) as a yellow solid. ¹H NMR (400 MHz, DMSO-d₆) ppm 10.32 (s, 1H), 9.02 (s, 1H), 8.29 (d, J=1.8 Hz, 1H), 8.25 (br. s., 1H), 7.93 (d, J=8.8 Hz, 1H), 7.83 (d, J=7.1 Hz, 1H), 7.80 (dd, J=9.0, 1.9 Hz, 1H), 7.69 (br. s., 1H), 7.52-7.61 (m, 2H), 7.38 (t, J=7.8 Hz, 1H), 7.21 (dd, J=8.1, 1.5 Hz, 1H), 6.85 (d, J=2.0 Hz, 1H), 6.76 (dd, J=7.1, 2.0 Hz, 1H), 4.27 (q, J=7.1 Hz, 2H), 1.99 (s, 3H), 1.27 (t, J=7.1 Hz, 3H).

### b) 3-({3-(aminocarbonyl)-7-[2-(methyloxy)-4-pyridinyl]-4-quinolinyl}amino)benzoic acid

To a suspension of ethyl 3-({3-(aminocarbonyl)-7-[2-(methyloxy)-4-pyridinyl]-4-quinolinyl}amino)benzoate (130 mg, 0.294 mmol) in ethanol (2.0 mL) was added 1N aqueous sodium hydroxide (2 ml, 2.000 mmol). After stirring 2 h at ambient temperature, the reaction mixture was concentrated to an aqueous residue, which was then neutralized with 6N aqueous hydrochloric acid. The resulting precipitate was filtered, washed with water, and dried in vacuo to obtain the title compound (0.060 g, 49%) as a bright yellow solid. ¹H NMR (400 MHz, DMSO-d₆) ppm 11.60 (br. s., 1H), 9.03 (s, 1H), 8.30-8.41 (m, 2H), 8.24 (d, J=8.8 Hz, 1H), 7.94 (dd, J=9.1, 1.5 Hz, 1H), 7.89 (d, J=7.1 Hz, 1H), 7.76-7.84 (m, 2H), 7.75 (br. s., 1H), 7.40-7.58 (m, 2H), 6.85 (d, J=2.0 Hz, 1H), 6.68 (dd, J=7.2, 2.1 Hz, 1H), 3.48 (br. s., 3H).

### Example 39

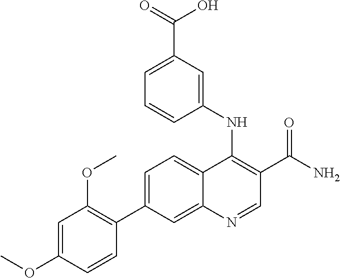 [SMILES: COc1ccc(-c2ccc3c(Nc4cccc(C(=O)O)c4)c(C(N)=O)cnc3c2)c(OC)c1]

### 3-({3-(aminocarbonyl)-7-[2,4-bis(methyloxy)phenyl]-4-quinolinyl}amino)benzoic acid

### a) ethyl 3-({3-(aminocarbonyl)-7-[2,4-bis(methyloxy)phenyl]-4-quinolinyl}amino)benzoate

A solution of ethyl 3-{[3-(aminocarbonyl)-7-bromo-4-quinolinyl]amino}benzoate (example 6a, 280 mg, 0.676 mmol), 2,4-dimethoxyphenylboronic acid (369 mg, 2.028 mmol), potassium carbonate (280 mg, 2.028 mmol), and tetrakis(triphenylphosphine)palladium(0) (35.1 mg, 0.030 mmol) in 1,4-dioxane (2.0 ml) and water (0.667 ml) was heated to 120° C. for 20 min. in a Biotage Initiator® microwave synthesizer. Upon cooling, the reaction mixture was diluted with water and extracted with ethyl acetate twice. The combined organic portions were dried (MgSO₄) and concentrated. Following purification via flash column chromatography (0-10% methanol in dichloromethane) the desired fractions were concentrated and the residue triturated in ethyl ether to obtain the title compound (0.270 g, 85%) as a pale beige solid. ¹H NMR (400 MHz, DMSO-d₆) ppm 10.33 (s, 1H), 8.99 (s, 1H), 8.24 (br. s., 1H), 8.03 (d, J=1.5 Hz, 1H), 7.79 (d, J=8.8 Hz, 1H), 7.67 (br. s., 1H), 7.52-7.62 (m, 3H), 7.33-7.42 (m, 2H), 7.20 (dd, J=8.1, 1.5 Hz, 1H), 6.71 (d, J=2.5 Hz, 1H), 6.67 (dd, J=8.5, 2.4 Hz, 1H), 4.27 (q, J=7.1 Hz, 2H), 3.83 (s, 3H), 3.80 (s, 3H), 1.27 (t, J=7.1 Hz, 3H).

### b) 3-({3-(aminocarbonyl)-7-[2,4-bis(methyloxy)phenyl]-4-quinolinyl}amino)benzoic acid

To a suspension of ethyl 3-({3-(aminocarbonyl)-7-[2,4-bis(methyloxy)phenyl]-4-quinolinyl}amino)benzoate (270 mg, 0.573 mmol) in ethanol (3.0 mL) was added 1N aqueous sodium hydroxide (3 ml, 3.00 mmol). After stirring overnight at ambient temperature, the reaction mixture was concentrated to an aqueous residue, which was then neutralized with 6N aqueous hydrochloric acid. The resulting precipitate was filtered, washed with water, and dried in vacuo to obtain the title compound (0.134 g, 53%) as a bright yellow solid. ¹H NMR (400 MHz, DMSO-d₆) ppm 13.17 (br. s., 1H), 11.85 (br. s., 1H), 9.00 (s, 1H), 8.31 (br. s., 1H), 8.17 (d, J=1.5 Hz, 1H), 8.10 (d, J=9.1 Hz, 1H), 7.81-7.93 (m, 2H), 7.79 (br. s., 1H), 7.75 (dd, J=8.8, 1.5 Hz, 1H), 7.54 (d, J=5.1 Hz, 2H), 7.44 (d, J=8.6 Hz, 1H), 6.76 (d, J=2.3 Hz, 1H), 6.71 (dd, J=8.6, 2.5 Hz, 1H), 3.84 (s, 3H), 3.84 (s, 3H).

### Example 40

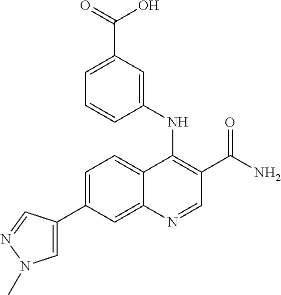 [SMILES: Cn1cc(-c2ccc3c(Nc4cccc(C(=O)O)c4)c(C(N)=O)cnc3c2)cn1]

### 3-{[3-(aminocarbonyl)-7-(1-methyl-1H-pyrazol-4-yl)-4-quinolinyl]amino}benzoic acid, bistrifluoroacetate salt

A solution of ethyl 3-{[3-(aminocarbonyl)-7-bromo-4-quinolinyl]amino}benzoate (example 6a, 100 mg, 0.241 mmol), 1-methylpyrazole-4-boronic acid (151 mg, 0.724 mmol), potassium carbonate (100 mg, 0.724 mmol), and tetrakis(triphenylphosphine)palladium(0) (12.55 mg, 10.86 μmol) in 1,4-dioxane (2.0 ml) and water (2 ml) was heated to 120° C. for 20 min. in a Biotage Initiator® microwave synthesizer. Upon cooling, the reaction mixture was diluted with water and extracted with ethyl acetate. The organic portion was concetnrated and the residue diluted with ethanol (2.0 mL) and treated with sodium hydroxide (1N aq solution) (1.629 ml, 1.629 mmol). Following stirring overnight at room temperature, the ethanol was concentrated, the residue diluted with water, then adjusted to pH˜4 using 1N hydrochloric acid. The resulting solid was purified using Reverse Phase HPLC (0.1% TFA in acetonitrile and water) to obtain the title compound (0.004 g, 3%) as a yellow solid. ¹H NMR (400 MHz, DMSO-d₆) ppm 13.17 (br. s., 1H), 11.64 (br. s., 1H), 8.92 (s, 1H), 8.42 (s, 1H), 8.09 (s, 1H), 7.98-8.06 (m, 2H), 7.66-7.92 (m, 4H), 7.38-7.61 (m, 2H), 3.91 (s, 3H).

### Example 41

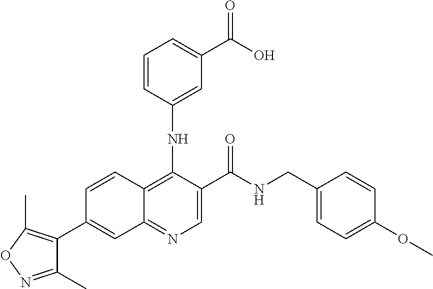 [SMILES: COc1ccc(CNC(=O)c2cnc3cc(-c4c(C)noc4C)ccc3c2Nc2cccc(C(=O)O)c2)cc1]

### 3-({7-(3,5-dimethyl-4-isoxazolyl)-3-[({[4-(methyloxy)phenyl]methyl}amino)carbonyl]-4-quinolinyl}amino)benzoic acid

### a) ethyl 3-({7-(3,5-dimethyl-4-isoxazolyl)-3-[({[4-(methyloxy)phenyl]methyl}amino)carbonyl]-4-quinolinyl}amino)benzoate

Benzotriazol-1-yloxy-trispyrrolidinophosphonium hexafluorophosphate (130 mg, 0.250 mmol) was added to a stirred solution of 7-(3,5-dimethyl-4-isoxazolyl)-4-({3-[(ethyloxy)carbonyl]phenyl}amino)-3-quinolinecarboxylic acid (example 13c, 72 mg, 0.167 mmol), 4-methoxybenzylamine (0.033 mL, 0.250 mmol), and triethylamine (0.070 mL, 0.501 mmol) in dichloromethane (2 mL) at room temperature and the mixture stirred for 18 h. 1M aqueous hydrochloric acid (10 mL) was added and the mixture extracted with dichloromethane. The extracts were washed with brine, dried (MgSO4) and evaporated under reduced pressure. The residue was chromatographed (silica gel, 20-100% ethyl acetate/hexane) to give the title compound (0.151 g, 89%) as a white solid. ¹H NMR (400 MHz, METHANOL-d₄) ppm 1.40 (t, J=7.07 Hz, 3H) 2.38 (s, 3H) 2.56 (s, 3H) 3.80 (s, 3H) 4.05 (s, 2H) 4.39 (q, J=7.24 Hz, 2H) 6.85-6.92 (m, 2H) 7.13-7.22 (m, 2H) 7.53-7.61 (m, 2H) 7.75 (dd, J=9.09, 1.77 Hz, 1H) 7.94-7.98 (m, 2H) 7.99-8.04 (m, 1H) 8.47 (d, J=8.84 Hz, 1H) 8.80 (s, 1H).

### b) 3-({7-(3,5-dimethyl-4-isoxazolyl)-3-[({[4-(methyloxy)phenyl]methyl}amino)carbonyl]-4-quinolinyl}amino)benzoic acid

Sodium hydroxide (0.039 mL, 0.234 mmol) was added to a stirred solution of ethyl 3-({7-(3,5-dimethyl-4-isoxazolyl)-3-[({[4-(methyloxy)phenyl]methyl}amino)carbonyl]-4-quinolinyl}amino)benzoate (43 mg, 0.078 mmol) in ethanol (2 mL) at room temperature. After one hour, the temperature was raised to 50° C. After an additional hour, 1N HCl was added dropwise until neutral pH was obtained. The solution was filtered and purified by HPLC (5-80% Acetonitrile/water w/0.1% TFA @ 10 min gradient) to give the title compound (22 mg, 0.042 mmol, 53.9% yield) as a white powder. ¹H NMR (METHANOL-d₄): 8.79 (s, 1H), 8.47 (d, J=9.1 Hz, 1H), 8.04 (td, J=4.5, 1.6 Hz, 1H), 7.90-8.00 (m, 2H), 7.77 (dd, J=8.8, 1.8 Hz, 1H), 7.57-7.64 (m, 2H), 7.20 (d, J=8.6 Hz, 2H), 6.90 (d, J=8.6 Hz, 2H), 4.04 (s, 1H), 3.81 (s, 3H), 2.56 (s, 3H), 2.39 (s, 3H).

### Example 42

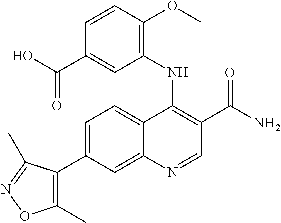 [SMILES: COc1ccc(C(=O)O)cc1Nc1c(C(N)=O)cnc2cc(-c3c(C)noc3C)ccc12]

### 3-{[3-(aminocarbonyl)-7-(3,5-dimethyl-4-isoxazolyl)-4-quinolinyl]amino}-4-(methyloxy)benzoic acid, trifluoroacetate salt

### a) 3-{[3-(aminocarbonyl)-7-bromo-4-quinolinyl]amino}-4-(methyloxy)benzoic acid

To a suspension of 7-bromo-4-chloro-3-quinolinecarboxamide (example 1a, 100 mg, 0.350 mmol) in acetic acid (10 mL) was added 3-amino-4-(methyloxy)benzoic acid (58.5 mg, 0.350 mmol). The reaction mixture was stirred at 100° C. for 2 hours. After cooling, the reaction was quenched with water, the precipitate was collected and dried in the air to afford the title compound (105 mg, 0.252 mmol, 72.0% yield) as a brown solid. ¹H NMR (400 MHz, DMSO-d6) δ ppm 12.15 (br. s., 1H), 9.09 (s, 1H), 8.45 (br. s., 1H), 8.29 (d, J=1.8 Hz, 1H), 7.94 (dd, J=8.7, 2.1 Hz, 2H),7.89 (d, J=10.1 Hz, 1H), 7.80 (s, 1H), 7.73 (dd, J=9.2, 1.9 Hz, 1H), 7.27 (d, J=8.6 Hz, 1H), 3.80 (s, 3H).

### b) 3-{[3-(aminocarbonyl)-7-(3,5-dimethyl-4-isoxazolyl)-4-quinolinyl]amino}-4-(methyloxy)benzoic acid, trifluoroacetate salt

To a suspension of 3-{[3-(aminocarbonyl)-7-bromo-4-quinolinyl]amino}-4-(methyloxy)benzoic acid (100 mg, 0.240 mmol), (3,5-dimethyl-4-isoxazolyl)boronic acid (67.7 mg, 0.481 mmol) and tetrakis(triphenylphosphine)palladium(0) (13.88 mg, 0.012 mmol) in 1,4-dioxane (6 mL) and water (2.000 mL) was added potassium carbonate (66.4 mg, 0.481 mmol). The reaction mixture was stirred at 100° C. for 2 hours. After cooling, the mixture was purifed through preparative HPLC (YMC 75×30 mm column, 0.1% TFA in water and 0.1% TFA in acetonitrile) to afford the title compound (65 mg, 0.119 mmol, 49.5% yield) as a yellow solid. ¹H NMR (400 MHz, DMSO-d6) δ ppm 12.84 (br. s., 1H), 11.90 (br. s., 1H), 8.99 (s, 1H), 8.30 (br. s., 1H), 8.06 (d, J=8.6 Hz, 1H), 7.96 (d, J=1.8 Hz, 1H), 7.84 (br. s., 1H), 7.64 (dd, J=9.0, 1.4 Hz, 1H), 7.60 (d, J=2.8 Hz, 1H), 7.47 (dd, J=8.8, 2.8 Hz, 1H), 7.20 (d, J=8.8 Hz, 1H), 3.86 (s, 3H), 2.51 (s, 3H), 2.32 (s, 3H).

### Example 43

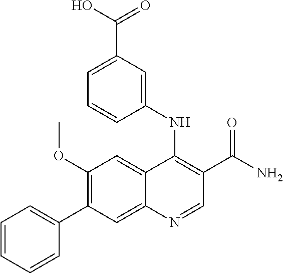 [SMILES: COc1cc2c(Nc3cccc(C(=O)O)c3)c(C(N)=O)cnc2cc1-c1ccccc1]

### 3-{[3-(aminocarbonyl)-6-(methyloxy)-7-phenyl-4-quinolinyl]amino}benzoic acid

### a) methyl 3-{[3-(aminocarbonyl)-7-chloro-6-(methyloxy)-4-quinolinyl]amino}benzoate

A mixture of 4,7-dichloro-6-(methyloxy)-3-quinolinecarboxamide (example 9d, 1.43 g, 5.27 mmol), methyl 3-aminobenzoate (1.04 g, 6.88 mmol) and acetic acid (20 mL) was heated at 50° C. for 1 h. On cooling to room temperature, a precipitate separated and was filtered off, washed with acetic acid and dried to give the title compound (1.93 g, 95%) as a yellow solid. ¹H NMR (400 MHz, DMSO-d₆) ppm 3.86 (s, 3H) 3.87 (s, 3H) 7.47-7.63 (m, 3H) 7.79-7.85 (m, 2H) 7.91 (s, 1H) 8.14 (s, 1H) 8.20 (s, 1H) 8.87 (s, 1H) 11.31 (br. s., 1H).

### b) methyl 3-{[3-(aminocarbonyl)-6-(methyloxy)-7-phenyl-4-quinolinyl]amino}benzoate

A mixture of methyl 3-{[3-(aminocarbonyl)-7-chloro-6-(methyloxy)-4-quinolinyl]amino}benzoate (0.154 g, 0.400 mmol), phenylboronic acid (0.195 g, 1.60 mmol), bis(tri-t-butylphosphine)palladium(0) (0.020 g, 0.040 mmol) and potassium carbonate (0.276 g, 2.00 mmol) in 1,4-dioxane (3 mL) and water (1 mL) was stirred at 120° C. in a microwave synthesiser for 0.5 h, then cooled and filtered. The filtrate was concentrated under reduced pressure, then diluted with water and acidified to pH 2 with 1M aqueous hydrochloric acid. The precipitate was filtered off, washed with water and dried, then chromatographed (silica gel, 1-10% methanol/dichloromethane) to give the title compound (0.110 g, 64%) as a yellow powder. ¹H NMR (400 MHz, DMSO-d₆) ppm 3.55 (s, 3H) 3.83 (s, 3H) 7.20-7.24 (m, 1H) 7.26 (s, 1H) 7.38-7.51 (m, 4H) 7.55-7.66 (m, 5H) 7.87 (s, 1H) 8.17 (br. s., 1H) 8.87 (s, 1H) 10.13 (s, 1H).

### c) 3-{[3-(aminocarbonyl)-6-(methyloxy)-7-phenyl-4-quinolinyl]amino}benzoic acid

1M aqueous sodium hydroxide (2.00 mL, 2.00 mmol) was added to a solution of methyl 3-{[3-(aminocarbonyl)-6-(methyloxy)-7-phenyl-4-quinolinyl]amino}benzoate

(0.093 g, 0.218 mmol) in ethanol (5 mL) and the mixture stirred at room temperature for 3 h, then diluted with water (30 mL). The pH was adjusted to 2 with 1M aqueous hydrochloric acid and the precipitate filtered off, washed with water and dried to give the title compound (0.074 g, 82%) as a pale yellow solid. ¹H NMR (400 MHz, DMSO-d₆) ppm 3.55 (s, 3H) 7.23-7.29 (m, 1H) 7.30 (s, 1H) 7.37-7.52 (m, 4H) 7.57-7.63 (m, 4H) 7.65 (br. s., 1H) 7.87 (s, 1H) 8.20 (br. s., 1H) 8.88 (s, 1H) 10.35 (br. s., 1H) 12.98 (br. s., 1H).

### Example 44

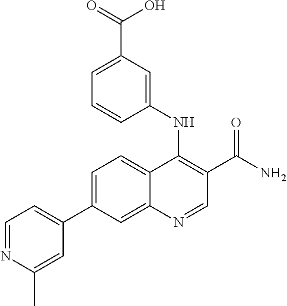 [SMILES: Cc1cc(-c2ccc3c(Nc4cccc(C(=O)O)c4)c(C(N)=O)cnc3c2)ccn1]

### 3-{[3-(aminocarbonyl)-7-(2-methyl-4-pyridinyl)-4-quinolinyl]amino}benzoic acid

### a) ethyl 3-{[3-(aminocarbonyl)-7-(2-methyl-4-pyridinyl)-4-quinolinyl]amino}benzoate

A solution of ethyl 3-{[3-(aminocarbonyl)-7-chloro-4-quinolinyl]amino}benzoate (example 14b, 143 mg, 0.387 mmol), 2-methyl-4-(4,4,5,5-tetramethyl-1,3,2-dioxaborolan-2-yl)pyridine (254 mg, 1.160 mmol), potassium carbonate (160 mg, 1.160 mmol), and tetrakis(triphenylphosphine)palladium(0) (20.11 mg, 0.017 mmol) in 1,4-dioxane (2.0 ml) and water (0.667 ml) was heated to 120° C. for 20 min. in a Biotage Initiator® microwave synthesizer. Poor reaction profile was observed. The mixture was re-submitted to 150° C. for 20 minutes. Upon cooling, the reaction mixture was diluted with water and extracted with ethyl acetate twice. The combined organic portions were dried (MgSO4) and concentrated. Following purification via flash column chromatography (10% methanol in dichloromethane) the desired fractions were concentrated and the residue triturated in ethyl ether to obtain the title compound (0.064 g, 39%) as a yellow solid. ¹H NMR (400 MHz, DMSO-d₆) ppm 10.91 (br. s., 1H), 9.03 (s, 1H), 8.66 (d, J=5.3 Hz, 1H), 8.43 (s, 1H), 8.24 (br. s., 1H), 8.11-8.19 (m, 1H), 7.99 (dd, J=8.5, 1.1 Hz, 1H), 7.92 (br. s., 1H), 7.82 (br. s., 1H), 7.67-7.75 (m, 3H), 7.47 (t, J=8.1 Hz, 1H), 7.37 (d, J=9.3 Hz, 1H), 4.30 (q, J=7.2 Hz, 2H), 3.17 (s, 3H), 1.30 (t, J=7.2 Hz, 3H).

### b) 3-{[3-(aminocarbonyl)-7-(2-methyl-4-pyridinyl)-4-quinolinyl]amino}benzoic acid

To a suspension of ethyl 3-{[3-(aminocarbonyl)-7-(2-methyl-4-pyridinyl)-4-quinolinyl]amino}benzoate (60 mg, 0.141 mmol) in ethanol (5.0 mL) was added 1N aqueous sodium hydroxide (2 ml, 2.000 mmol). After stirring 3 hr at ambient temperature, the reaction mixture was concentrated to an aqueous residue, which was then neutralized with 6N aqueous hydrochloric acid. The resulting precipitate was filtered, washed with water, and dried in vacuo to obtain the title compound (0.020 g, 36%) as a yellow solid. ¹H NMR (400 MHz, DMSO-d₆) ppm 13.04 (br. s., 1H), 10.81 (s, 1H), 9.04 (s, 1H), 8.63 (d, J=5.3 Hz, 1H), 8.41 (s, 1H), 8.28 (br. s., 1H), 8.08 (d, J=8.8 Hz, 1H), 7.96 (d, J=8.8 Hz, 1H), 7.89 (br. s., 1H), 7.78 (d, J=4.0 Hz, 1H), 7.74 (br. s., 1H), 7.68 (d, J=7.6 Hz, 1H), 7.63 (br. s., 1H), 7.45 (t, J=7.8 Hz, 1H), 7.29-7.39 (m, 1H), 2.60 (s, 3H).

### Example 45

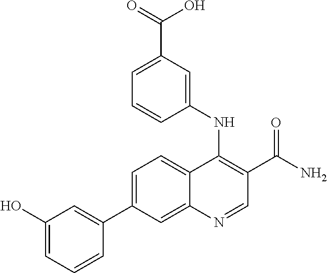 [SMILES: NC(=O)c1cnc2cc(-c3cccc(O)c3)ccc2c1Nc1cccc(C(=O)O)c1]

### 3-{[3-(aminocarbonyl)-7-(3-hydroxyphenyl)-4-quinolinyl]amino}benzoic acid

A solution of 3-({3-(aminocarbonyl)-7-[3-(methyloxy)phenyl]-4-quinolinyl}amino)benzoic acid (example 35, 98 mg, 0.237 mmol) in dichloromethane (10 mL) was treated with boron tribromide (1M solution in dichloromethane) (1.304 mL, 1.304 mmol) at room temperature overnight. The reaction mixture was poured into water, filtered and washed with water. The obtained solid was purified using Reverse Phase HPLC (ODS, 0.1% TFA in acetontirle and water) to obtain the title compound (0.029 g, 31%) as a bright yellow solid. ¹H NMR (400 MHz, DMSO-d₆) ppm 11.32 (br. s., 1H), 9.76 (br. s., 1H), 9.00 (s, 1H), 8.26 (br. s., 1H), 8.15 (d, J=1.8 Hz, 1H), 8.11 (d, J=8.8 Hz, 1H), 7.86 (dd, J=9.0, 1.4 Hz, 1H), 7.77 (d, J=7.6 Hz, 1H), 7.73 (br. s., 2H), 7.47-7.53 (m, 1H), 7.41-7.46 (m, 1H), 7.31-7.38 (m, 1H), 7.25 (d, J=8.1 Hz, 1H), 7.18 (t, J=1.9 Hz, 1H), 6.89 (dd, J=8.1, 1.5 Hz, 1H).

### Example 46

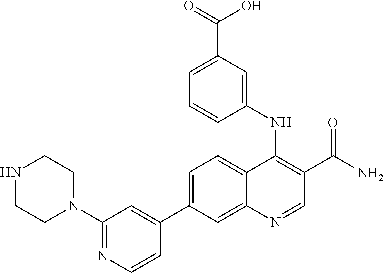 [SMILES: NC(=O)c1cnc2cc(-c3ccnc(N4CCNCC4)c3)ccc2c1Nc1cccc(C(=O)O)c1]

### 3-({3-(aminocarbonyl)-7-[2-(1-piperazinyl)-4-pyridinyl]-4-quinolinyl}amino)benzoic acid, trifluoroacetate salt

A solution of ethyl 3-{[3-(aminocarbonyl)-7-bromo-4-quinolinyl]amino}benzoate (example 6a, 70 mg, 0.169 mmol), 2-(1-piperazinyl)-pyridinyl-4-boronic acid (105 mg, 0.507 mmol), potassium carbonate (283 mg, 2.048 mmol), and tetrakis(triphenylphosphine)palladium(0) (8.79 mg, 7.60 μmol) in 1,4-dioxane (2.0 ml) and water (0.667 mL) was heated to 100° C. for 20 min. in a Biotage Initiator® microwave synthesizer. Upon cooling, the reaction mixture was concentrated in vacuo, diluted with ethanol (2 ml) and then treated with sodium hydroxide (1M aq solution) (2 ml, 2.000 mmol). Following stirring overnight at room temperature, the ethanol was removed in vacuo, the residue diluted with water and extracted using ethyl acetate. The aqueous portion was then adjusted to pH˜4 using 1N hydrochloric acid. The solution was purified using Reverse Phase (0.1% TFA in acetonitrile and water) to obtain the title compound (0.039 g, 40%) as a bright yellow solid. ¹H NMR (400 MHz, DMSO-d₆) ppm 11.87 (br. s., 1H), 9.05 (s, 1H), 8.96 (br. s., 2H), 8.43 (d, J=1.5 Hz, 1H), 8.33 (br. s., 1H), 8.34 (d, J=5.3 Hz, 1H), 8.23 (d, J=9.1 Hz, 1H), 8.08 (dd, J=9.1, 1.5 Hz, 1H), 7.84-7.91 (m, 2H), 7.81 (br. s., 1H), 7.57 (d, J=5.1 Hz, 2H), 7.37 (s, 1H), 7.19 (d, J=5.3 Hz, 1H), 3.81-3.90 (m, 4H), 3.15-3.30 (m, 4H).

### Example 47

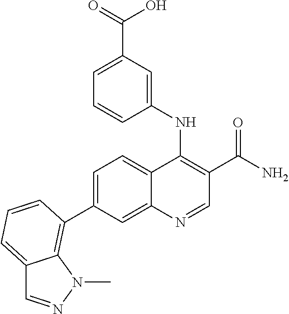 [SMILES: Cn1ncc2cccc(-c3ccc4c(Nc5cccc(C(=O)O)c5)c(C(N)=O)cnc4c3)c21]

### 3-{[3-(aminocarbonyl)-7-(1-methyl-1H-indazol-7-yl)-4-quinolinyl]amino}benzoic acid, trifluoroacetate salt

A solution of ethyl 3-{[3-(aminocarbonyl)-7-bromo-4-quinolinyl]amino}benzoate (example 6a, 106 mg, 0.256 mmol), (1-methyl-11H-indazol-7-yl)boronic acid (113 mg, 0.640 mmol), potassium carbonate (106 mg, 0.768 mmol), and tetrakis(triphenylphosphine)palladium(0) (13.31 mg, 0.012 mmol) in 1,4-dioxane (2.0 ml) and water (0.667 ml) was heated to 100° C. for 20 min. in a Biotage Initiator® microwave synthesizer. Upon cooling, the reaction mixture was concentrated in vacuo, diluted with ethanol (2 ml) and then treated with sodium hydroxide (1M aq solution) (2 ml, 2.000 mmol). Following stirring overnight at room temperature, the ethanol was removed in vacuo, the residue diluted with water and extracted using ethyl acetate. The aqueous portion was then adjusted to pH˜4 using 1N hydrochloric acid. The resulting solid was purified using Reverse phase (0.1% TFA in acetonitrile and water) to obtain the title compound (0.067 g, 48%) as a yellow solid. ¹H NMR (400 MHz, DMSO-d₆) ppm 13.12 (br. s., 1H), 11.51 (br. s., 1H), 9.03 (s, 1H), 8.22-8.28 (m, J=7.2, 7.2 Hz, 2H), 8.21 (s, 1H), 8.05 (d, J=1.8 Hz, 1H), 7.91 (dd, J=8.1, 1.0 Hz, 1H), 7.72-7.83 (m, 4H), 7.53 (d, J=4.8 Hz, 2H), 7.39 (dd, J=7.1, 1.0 Hz, 1H), 7.28 (dd, J=7.8, 7.1 Hz, 1H), 3.60 (s, 3H).

### Example 48

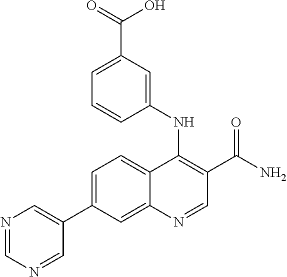 [SMILES: NC(=O)c1cnc2cc(-c3cncnc3)ccc2c1Nc1cccc(C(=O)O)c1]

### 3-{[3-(aminocarbonyl)-7-(5-pyrimidinyl)-4-quinolinyl]amino}benzoic acid

### a) ethyl 3-{[3-(aminocarbonyl)-7-(5-pyrimidinyl)-4-quinolinyl]amino}benzoate

A solution of ethyl 3-{[3-(aminocarbonyl)-7-bromo-4-quinolinyl]amino}benzoate (example 6a, 100 mg, 0.241 mmol), pyrimidine-5-boronic acid (90 mg, 0.724 mmol), potassium carbonate (100 mg, 0.724 mmol), and tetrakis(triphenylphosphine)palladium(0) (20 mg, 0.017 mmol) in 1,4-dioxane (2.0 ml) and water (0.667 ml) was heated to 100° C. for 20 min. in a Biotage Initiator® microwave synthesizer. Upon cooling, the reaction mixture was diluted with water, extracted with ethyl acetate, dried (magnesium sulfate) and concentrated in vacuo. The residue was purified via flash column chromatography (10% methanol in dichloromethane) and the desired fractions were collected and washed with ether to furnish the title compound (0.066 g, 66%) as a yellow solid. ¹H NMR (400 MHz, DMSO-d₆) ppm 10.35 (s, 1H), 9.34 (s, 2H), 9.25 (s, 1H), 9.04 (s, 1H), 8.46 (d, J=1.8 Hz, 1H), 8.27 (br. s., 1H), 7.95-8.02 (m, 1H), 7.89-7.94 (m, 1H), 7.70 (br. s., 1H), 7.55-7.63 (m, 2H), 7.39 (t, J=8.2 Hz, 1H), 7.19-7.26 (m, 1H), 4.27 (q, 2H), 1.28 (t, J=7.1 Hz, 3H).

### b) 3-{[3-(aminocarbonyl)-7-(5-pyrimidinyl)-4-quinolinyl]amino}benzoic acid

To a suspension of ethyl 3-{[3-(aminocarbonyl)-7-(5-pyrimidinyl)-4-quinolinyl]amino}benzoate (66 mg, 0.160 mmol) in ethanol (2.0 mL) was added 1N aqueous sodium hydroxide (2 ml, 2.000 mmol). After stirring 30 min. at ambient temperature, the reaction was concentrated to an aq residue, then quenched with 6N aqueous hydrochloric acid and the resulting precipitate was filtered, washed with water, and dried in vacuo to obtain the title compound (56 mg, 0.145 mmol, 91% yield) as a yellow solid. ¹H NMR (400 MHz, DMSO-d₆) ppm 10.56 (br. s., 1H), 9.33 (s, 2H), 9.25 (s, 1H), 9.06 (s, 1H), 8.45 (d, J=1.5 Hz, 1H), 8.33 (br. s., 1H), 7.97-8.07 (m, 1H), 7.91 (dd, J=8.8, 1.8 Hz, 1H), 7.72 (br. s., 1H), 7.62 (d, 1H), 7.56 (s, 1H), 7.40 (t, J=7.8 Hz, 1H), 7.27 (dd, J=8.1, 1.5 Hz, 1H).

### Example 49

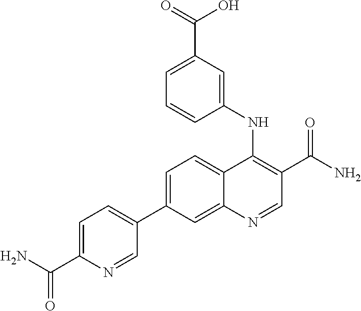 [SMILES: NC(=O)c1ccc(-c2ccc3c(Nc4cccc(C(=O)O)c4)c(C(N)=O)cnc3c2)cn1]

### 3-{3-aminocarbonyl)-7-[6-(aminocarbonylyl)-3-pyridinyl]-4-quinolinyl}amino)benzoic acid, trifluoroacetate salt

### a) ethyl 3-{[3-(aminocarbonyl)-7-(6-cyano-3-pyridinyl)-4-quinolinyl]amino}benzoate

A solution of ethyl 3-{[3-(aminocarbonyl)-7-bromo-4-quinolinyl]amino}benzoate (example 6a, 100 mg, 0.241 mmol), 2-cyanopyridine-5-boronic acid pinacol ester (107 mg, 0.724 mmol), potassium carbonate (100 mg, 0.724 mmol), and tetrakis(triphenylphosphine)palladium(0) (12.55 mg, 10.86 μmol) in 1,4-dioxane (3 mL) and water (1.000 mL) was heated to 100° C. for 20 min. in a Biotage Initiator® microwave synthesizer. Upon cooling, the reaction mixture was diluted with water and extracted with ethyl acetate. The organic portion was dried over magnesium sulfate, filtered and concentrated. The residue was purified via flash column chromatography (0-100% ethyl acetate in hexanes, then 10% methanol in ethyl acetate) to give the title compound (0.100 g, 95%). ¹H NMR (400 MHz, DMSO-d₆) ppm 10.35 (s, 1H), 9.31 (dd, J=2.3, 0.8 Hz, 1H), 9.05 (s, 1H), 8.57 (dd, J=8.1, 2.3 Hz, 1H), 8.49 (d, J=2.0 Hz, 1H), 8.27 (s, 1H), 8.20 (dd, J=8.2, 0.6 Hz, 1H), 7.98-8.04 (m, 1H), 7.88-7.96 (m, 1H), 7.71 (s, 1H), 7.57-7.65 (m, 2H), 7.40 (t, J=8.1 Hz, 1H), 7.23 (dt, J=7.6, 1.4 Hz, 1H), 4.28 (q, 2H), 1.28 (t, J=7.1 Hz, 3H).

### b) 3-({3-(aminocarbonyl)-7-[6-(aminocarbonyl)-3-pyridinyl]-4-quinolinyl}amino)benzoic acid, trifluoroacetate salt

To a suspension of ethyl 3-{[3-(aminocarbonyl)-7-(6-cyano-3-pyridinyl)-4-quinolinyl]amino}benzoate (100 mg, 0.229 mmol) in ethanol (5.0 mL) was added 1N aqueous sodium hydroxide (1.000 ml, 1.000 mmol). After stirring 30 min. at ambient temperature, the reaction was concentrated to an aqueous residue, which was neutralized using 6N hydrochloric acid. The solution was purified using Reverse Phase HPLC (0.1% TFA in acetonitrile and water) to obtain the title compound (0.024 g, 19%) as a yellow solid. ¹H NMR (400 MHz, DMSO-d₆) ppm 13.12 (br. s., 1H), 11.37 (br. s., 1H), 8.46 (dd, J=8.1, 2.3 Hz, 1H), 8.36 (d, J=1.5 Hz, 1H), 8.28 (br. s., 1H), 8.19 (m, 3H), 8.05 (d, J=9.9 Hz, 1H), 7.68-7.84 (m, 4H), 7.48-7.55 (m, 1H), 7.40-7.48 (m, 1H).

### Example 50

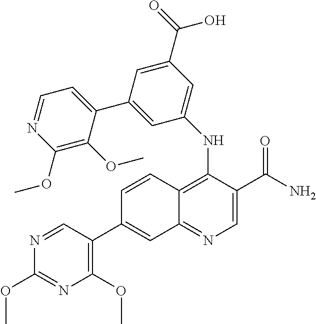 [SMILES: COc1ncc(-c2ccc3c(Nc4cc(C(=O)O)cc(-c5ccnc(OC)c5OC)c4)c(C(N)=O)cnc3c2)c(OC)n1]

### 3-({3-(aminocarbonyl)-7-[2,4-bis(methyloxy)-5-pyrimidinyl]-4-quinolinyl}amino)-5-[2,3-bis(methyloxy)-4-pyridinyl]benzoic acid

### a) 3-amino-5-[2,3-bis(methyloxy)-4-pyridinyl]benzoic acid

A suspension of 3-amino-5-bromobenzoic acid (200 mg, 0.926 mmol), (2,3-dimethoxypyridin-4-yl)boronic acid (186 mg, 1.018 mmol) and potassium carbonate (256 mg, 1.852 mmol) in 1,4-dioxane (6 mL) and water (2 mL) was degassed by bubbling with Ar for few min. Tetrakis(triphenylphosphine)palladium(0) (53.5 mg, 0.046 mmol) was added. The reaction mixture was sealed and heated in a Biotage Initiator microwave reactor at 100° C. for 30 min. According to UPLC-MS the conversion was incomplete and some small content of starting 3-Amino-5-bromobenzoic acid was present but still the reaction was stopped. The reaction mixture was diluted with water and extracted two times with EtOAc (some less polar impurities were present in organic layer while product remained in water layer). The water layer was acidified with 6N HCl (pH˜5-6) and extracted with EtOAc. The collected organic layers were dried over sodium sulfate and evaporated to obtain 184 mg of crudeproduct (area %=80.15) containing starting acid as a main impurity (area %=15.13). The product was recrystallized from MeOH to obtain the title compound (0.077 g, 30%) as an off-white solid, MS (ES+) m/z: 275 [M+H]⁺, and slightly impure product (0.07 g, 23%) after evaporation of mother liquour.

### b) 3-({3-(aminocarbonyl)-7-[2,4-bis(methyloxy)-5-pyrimidinyl]-4-quinolinyl}amino)-5-[2,3-bis(methyloxy)-4-pyridinyl]benzoic acid

To a suspension of 3-amino-5-[2,3-bis(methyloxy)-4-pyridinyl]benzoic acid (71 mg, 0.259 mmol) in acetic acid (10 mL), 7-[2,4-bis(methyloxy)-5-pyrimidinyl]-4-chloro-3-quinolinecarboxamide (example 1b, 89 mg, 0.259 mmol) was added. The reaction mixture (suspension, reactants were not dissolved completely) was stirred at room temperature overnight. After 17 h according to UPLC-MS the conversion to desired product was incomplete but still the reaction was stopped. The volume of the reaction mixture was reduced by evaporating, the water was added and the precipitate was filtered, washed with water and dried to afford the title compound (0.080 g, 51%). MS (ES+) m/e 583 [M+H]⁺.

### Example 51

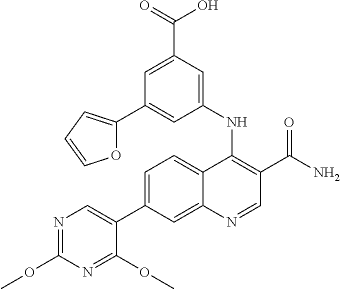 [SMILES: COc1ncc(-c2ccc3c(Nc4cc(C(=O)O)cc(-c5ccco5)c4)c(C(N)=O)cnc3c2)c(OC)n1]

### 3-({3-(aminocarbonyl)-7-[2,4-bis(methyloxy)-5-pyrimidinyl]-4-quuinolinyl}amino)-5-(2-furanyl)benzoic acid, trifluoroacetate salt

### a) 3-amino-5-(2-furanyl)benzoic acid

To a suspension of 3-amino-5-bromobenzoic acid (1 g, 4.63 mmol), 2-furanylboronic acid (0.518 g, 4.63 mmol) and potassium carbonate (1.279 g, 9.26 mmol) in 1,4-dioxane (12 mL) and water (4.00 mL) was added tetrakis(triphenylphosphine)palladium(0) (0.267 g, 0.231 mmol). The mixture was heated to 100° C. overnight. After cooling, the mixture was diluted with water, acidified with 6N HCl and extracted with ethyl acetate. The organic layer was dried over MgSO₄, filtered and concentrated under reduced pressure. The precipitate was collected and washed with methylene chloride and ether to afford the title compound (350 mg, 1.722 mmol, 37.2% yield) as a brown solid. 1H NMR (400 MHz, DMSO-d6) δ ppm 12.77 (br. s., 1H), 7.73 (d, J=1.0 Hz, 1H), 7.41 (t, J=1.5 Hz, 1H), 7.11 (d, J=1.5 Hz, 2H), 6.84 (d, J=3.3 Hz, 1H), 6.58 (dd, J=3.4, 1.9 Hz, 1H), 5.48 (br. s., 2H). MS (ES+) m/e 204 [M+H]+.

### b) 3-({3-(aminocarbonyl)-7-[2,4-bis(methyloxy)-5-pyrimidinyl]-4-quinolinyl}amino)-5-(2-furanyl)benzoic acid, trifluoroacetate salt

To a suspension of 7-[2,4-bis(methyloxy)-5-pyrimidinyl]-4-chloro-3-quinolinecarboxamide (example 1b, 50 mg, 0.145 mmol) in acetic acid (10 mL) was added 3-amino-5-(2-furanyl)benzoic acid (29.5 mg, 0.145 mmol). The reaction mixture was stirred at room temperature overnight and was purified through preparative HPLC (YMC 75×30 mm column, 0.1% TFA in water and 0.1% TFA in acetonitrile) to afford 3-({3-(aminocarbonyl)-7-[2,4-bis(methyloxy)-5-pyrimidinyl]-4-quinolinyl}amino)-5-(2-furanyl)benzoic acid (40 mg, 0.064 mmol, 44.1% yield) as a yellow solid. ¹H NMR (400 MHz, MeOD) δ ppm 8.94 (s, 1H), 8.55 (s, 1H), 8.32 (t, J=1.5 Hz, 1H), 8.15-8.22 (m, 2H), 7.83-7.92 (m, 3H), 7.63 (d, J=1.3 Hz, 1H), 6.94 (d, J=3.5 Hz, 1H), 6.57 (dd, J=3.4, 1.9 Hz, 1H), 4.11 (s, 3H), 4.08 (s, 3H). MS (ES+) m/e 512 [M+H]+

### Example 52

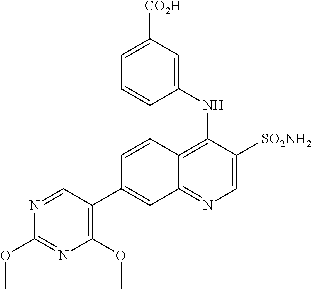 [SMILES: COc1ncc(-c2ccc3c(Nc4cccc(C(=O)O)c4)c(S(N)(=O)=O)cnc3c2)c(OC)n1]

### 3-({3-(aminosulfonyl)-7-[2,4-bis(methyloxy)-5-pyrimidinyl]-4-quinolinyl}amino)benzoic acid

### a) 7-bromo-4-hydroxy-3-quinolinesulfonyl chloride

A solution of 7-bromo-4-quinolinol (1.00 g, 4.46 mmol) in chlorosulfonic acid (10 mL) was heated at 100° C. for 18 h, then cooled and poured carefully onto ice. The solid was filtered off, washed with water and dried to give the title compound (1.33 g, 85% pure, 79%) as a solid, containing a little (˜12%) of the corresponding sulfonic acid by NMR. ¹H NMR (400 MHz, DMSO-d₆) ppm 7.71 (dd, J=8.72, 1.89 Hz, 1H) 8.04 (d, J=1.52 Hz, 1H) 8.16 (d, J=8.84 Hz, 1H) 8.78 (s, 1H).

### b) 7-bromo-4-chloro-3-quinolinesulfonamide

A mixture of 7-bromo-4-hydroxy-3-quinolinesulfonyl chloride (1.32 g, 4.34 mmol) and phosphorus oxychloride (10 mL) was stirred under reflux for 3 h, then cooled. The solvent was removed under reduced pressure and the residue azeotroped twice with toluene. The flask was cooled in iced water and 0.5M ammonia in dioxane (87 mL) added rapidly with stirring. The mixture was stirred 0.25 h at room temperature, then the solvent removed under reduced pressure. The residue was slurried in water (100 mL). The solid was filtered off, washed with water and dried to give the title compound (1.18 g, 94% pure, 84%) as a cream solid. ¹H NMR (400 MHz, DMSO-d₆) ppm 8.05 (dd, J=9.09, 1.77 Hz, 1H) 8.07 (s, 2H) 8.37 (d, J=8.84 Hz, 1H) 8.45 (d, J=1.77 Hz, 1H) 9.30 (s, 1H).

### c) ethyl 3-{[3-(aminosulfonyl)-7-bromo-4-quinolinyl]amino}benzoate

A mixture of 7-bromo-4-chloro-3-quinolinesulfonamide (0.321 g, 1.00 mmol), ethyl 3-aminobenzoate (0.247 g, 1.50 mmol) and acetic acid (6 mL) was stirred in a heating block set at 50° C. for 2 h, then cooled. Ether (10 mL) was added and the mixture stirred 0.25 h. The precipitate was filtered off, washed with ether and dried to give the title compound (0.379 g, 93% pure, 84%) as a yellow solid. ¹H NMR (400 MHz, DMSO-d₆) ppm 1.30 (t, J=7.07 Hz, 3H) 4.30 (q, J=7.07 Hz, 2H) 7.23-7.27 (m, 1H) 7.45 (t, J=7.83 Hz, 1H) 7.52 (d, J=9.09 Hz, 1H) 7.60 (dd, J=9.09, 1.77 Hz, 1H) 7.69 (s, 1H) 7.73 (d, J=7.83 Hz, 1H) 8.02 (br. s., 2H) 8.29 (d, J=1.77 Hz, 1H) 8.94 (br. s., 1H) 9.15 (s, 1H).

### d) 3-({3-(aminosulfonyl)-7-[2,4-bis(methyloxy)-5-pyrimidinyl]-4-quinolinyl}amino)benzoic acid

A mixture of ethyl 3-{[3-(aminosulfonyl)-7-bromo-4-quinolinyl]amino}benzoate (0.130 g, 0.289 mmol), [2,4-bis(methyloxy)-5-pyrimidinyl]boronic acid (0.106 g, 0.577 mmol), potassium carbonate (0.160 g, 1.16 mmol), tetrakis(triphenylphosphine)palladium(0) (0.017 g, 0.014 mmol), 1,4-dioxane (1.5 mL) and water (0.58 mL) was stirred at 150° C. in a microwave reactor for 0.5 h, then cooled. The supernatant liquor was chromatographed (silica gel, 2-9% methanol/dichloromethane) to give the intermediate ester (0.079 g). The ester was slurried in methanol (6 mL) and 1M aqueous sodium hydroxide (1.50 mL, 1.50 mmol) added. The mixture was stirred at room temperature for 24 h. The methanol was removed under reduced pressure and water (10 mL) added. The solution was filtered, then acidified to pH 3 with 1M aqueous hydrochloric acid. The precipitate was filtered off, washed with water and dried to give the title compound (0.063 g, 45%) as a yellow solid. ¹H NMR (400 MHz, DMSO-d₆) ppm 3.97 (s, 3H) 3.98 (s, 3H) 7.15 (dd, J=8.08, 1.52 Hz, 1H) 7.40 (t, J=7.83 Hz, 1H) 7.51 (t, J=1.77 Hz, 1H) 7.62 (d, J=7.58 Hz, 1H) 7.66 (d, J=1.01 Hz, 2H) 7.90 (s, 2H) 8.25 (s, 1H) 8.41 (s, 1H) 8.59 (s, 1H) 9.14 (s, 1H) 13.01 (br. s., 1H).

### Example 53

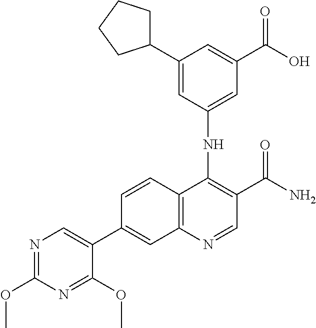 [SMILES: COc1ncc(-c2ccc3c(Nc4cc(C(=O)O)cc(C5CCCC5)c4)c(C(N)=O)cnc3c2)c(OC)n1]

### 3-({3-(aminocarbonyl)-7-[2,4-bis(methyloxy)-5-pyrimidinyl]-4-quinolinyl}amino)-5-cyclopentylbenzoic acid

### a) 3-amino-5-(1-cyclopenten-1-yl)benzoic acid

A 5 mL microwave vial was charged with 3-amino-5-bromobenzoic acid (200 mg, 0.926 mmol), cyclopenten-1-ylboronic acid (207 mg, 1.852 mmol), tetrakis(triphenylphosphine)palladium(0) (53.5 mg, 0.046 mmol), and potassium carbonate (256 mg, 1.852 mmol) in 1,4-dioxane (3 mL)/water (1.000 mL). The sealed vial was irradiated in a Biotage microwave synthesizer at 145° C. for 25 minutes. Purification was achieved using a reverse-phase HPLC (10-90% acetonitrile/water gradient with 0.1% TFA) to give the title compound (209 mg, 0.659 mmol, 71.2% yield) as a semiclear solid. ¹H NMR (DMSO-d₆) 7.41 (s, 1H), 7.21-7.30 (m, 1H), 7.08 (s, 1H), 6.25 (t, J=1.9 Hz, 1H), 2.57-2.69 (m, 2H), 2.48 (d, J=2.5 Hz, 2H), 1.86-2.06 (m, 2H).

### b) 3-amino-5-cyclopentylbenzoic acid

10% Pd/C (3.93 mg, 0.037 mmol) was added to a stirred solution of 3-amino-5-(1-cyclopenten-1-yl)benzoic acid (150 mg, 0.738 mmol) in ethanol (4 mL) under nitrogen. The flask was then purged with hydrogen and the mixture stirred at rt overnight. The catalyst was then filtered and solvent concentrated to give the title compound (139 mg, 0.677 mmol, 92% yield) as a clear glassy solid. MS (ES+) m/e 206 [M+H]⁺.

### c) 3-({3-(aminocarbonyl)-7-[2,4-bis(methyloxy)-5-pyrimidinyl]-4-quinolinyl}amino)-5-cyclopentylbenzoic acid

3-amino-5-cyclopentylbenzoic acid (72.0 mg, 0.351 mmol) was added to a stirred solution of 7-[2,4-bis(methyloxy)-5-pyrimidinyl]-4-chloro-3-quinolinecarboxamide (example 1b, 110 mg, 0.319 mmol) in acetic acid (2 mL) at rt. The reaction was stirred over the weekend before being purified by HPLC (10-70% acetonitrile/water w/01.% TFA) to give the title compound (43 mg, 0.069 mmol, 21.47% yield) as a yellow solid. ¹H NMR (METHANOL-d₄) 8.97 (s, 1H), 8.53 (s, 1H), 8.20 (s, 1H), 7.98 (d, J=9.1 Hz, 1H), 7.93 (s, 1H), 7.80 (t, J=1.8 Hz, 1H), 7.75 (dd, J=9.1, 1.8 Hz, 1H), 7.48 (s, 1H), 4.12 (s, 3H), 4.09 (s, 3H), 3.33 (dt, J=3.3, 1.6 Hz, 1H), 2.06-2.21 (m, 2H), 1.78-1.89 (m, 2H), 1.73 (dd, J=7.7, 4.2 Hz, 2H), 1.54-1.67 (m, 2H).

### Example 54

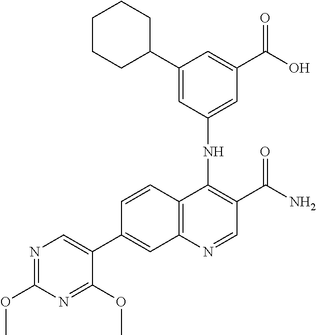 [SMILES: COc1ncc(-c2ccc3c(Nc4cc(C(=O)O)cc(C5CCCCC5)c4)c(C(N)=O)cnc3c2)c(OC)n1]

### 3-({3-(aminocarbonyl)-7-[2,4-bis(methyloxy)-5-pyrimidinyl]-4-quinolinyl}amino)-5-cyclohexylbenzoic acid

### a) 3-amino-5-(1-cyclohexen-1-yl)benzoic acid

A 5 mL microwave vial was charged with 3-amino-5-bromobenzoic acid (200 mg, 0.926 mmol), cyclohexen-1-ylboronic acid (233 mg, 1.852 mmol), tetrakis(triphenylphosphine)palladium(0) (53.5 mg, 0.046 mmol), and potassium carbonate (256 mg, 1.852 mmol) in 1,4-dioxane (3 mL)/water (1.000 mL). The sealed vial was irradiated in a Biotage microwave synthesizer at 145° C. for 25 minutes. The mixture was filtered and acidified with aqueous hydrochloric acid, and the resulting precipitate filtered. The eluent was concentrated to give the crude product. Compound was advanced to the next step with no purification. ¹H NMR (DMSO-d₆): 7.41 (s, 1H), 7.21 (s, 1H), 7.08-7.16 (m, 1H), 6.89 (t, J=1.9 Hz, 1H), 6.01-6.14 (m, 1H), 2.31 (dd, 2H), 2.17 (dd, 2H), 1.95-2.07 (m, 2H), 1.51 (d, J=2.5 Hz, 2H).

### b) 3-amino-5-cyclohexylbenzoic acid

10% palladium-on-charcoal (2.89 mg, 0.027 mmol) was added to a stirred solution of 3-amino-5-(1-cyclohexen-1-yl)benzoic acid (118 mg, 0.543 mmol) in ethanol (3 mL) under nitrogen. The flask was then purged with hydrogen and the mixture stirred at rt overnight. The catalyst was then filtered and solvent concentrated to give the title compound (60 mg, 0.274 mmol, 50.4% yield) as a clear glassy solid. ¹H NMR (METHANOL-d₄): 7.89 (s, 1H), 7.73-7.83 (m, 1H), 7.41 (t, J=1.8 Hz, 1H), 2.65 (ddd, J=11.4, 8.7, 2.7 Hz, 1H), 1.90 (dd, J=6.2, 2.7 Hz, 4H), 1.79 (d, J=12.6 Hz, 1H), 1.42-1.54 (m, 4H), 1.27-1.39 (m, 1H).

### c) 3-({3-(aminocarbonyl)-7-[2,4-bis(methyloxy)-5-pyrimidinyl]-4-quinolinyl}amino)-5-cyclohexylbenzoic acid

3-amino-5-cyclohexylbenzoic acid (60 mg, 0.276 mmol) was added to a stirred solution of 7-[2,4-bis(methyloxy)-5-pyrimidinyl]-4-chloro-3-quinolinecarboxamide (example 1b, 95 mg, 0.276 mmol) in acetic acid (2 mL) at rt.

The reaction was stirred over the weekend before being purified by HPLC (10-70% acetonitrile/water w/01. % TFA) to give the title compound (34 mg, 0.053 mmol, 19.23% yield) as a yellow solid. ¹H NMR (METHANOL-d₄): 8.96 (s, 1H), 8.54 (s, 1H), 8.19 (s, 1H), 8.00 (d, J=9.1 Hz, 1H), 7.93 (s, 1H), 7.83 (t, J=1.8 Hz, 1H), 7.78 (dd, J=9.1, 1.5 Hz, 1H), 7.40-7.50 (m, 1H), 4.13 (s, 3H), 4.09 (s, 3H), 2.54-2.75 (m, 1H), 1.82-2.00 (m, 4H), 1.77 (d, J=12.4 Hz, 1H), 1.40-1.53 (m, 4H), 1.23-1.37 (m, 1H).

### Example 55

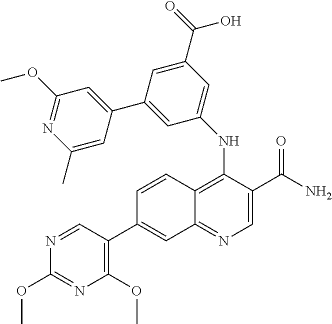 [SMILES: COc1cc(-c2cc(Nc3c(C(N)=O)cnc4cc(-c5cnc(OC)nc5OC)ccc34)cc(C(=O)O)c2)cc(C)n1]

### 3-({3-(aminocarbonyl)-7-[2,4-bis(methyloxy)-5-pyrimidinyl]-4-quinolinyl}amino)-5-[2-methyl-6-(methyloxy)-4-pyridinyl]benzoic acid

### a) 3-amino-5-[2-methyl-6-(methyloxy)-4-pyridinyl]benzoic acid

A suspension of 3-amino-5-bromobenzoic acid (200 mg, 0.926 mmol), 6-methoxy-2-picoline-4-boronic acid, pinacol ester (254 mg, 1.018 mmol) and potassium carbonate (384 mg, 2.78 mmol) in 1,4-dioxane (9 mL) and water (3 mL) was degassed by bubbling with Ar for few min. Tetrakis(triphenylphosphine)palladium(0) (53.5 mg, 0.046 mmol) was added. The reaction mixture was sealed and heated in a Biotage Initiator microwave reactor at 110° C. for 35 min. According to UPLC-MS the conversion was complete. The reaction mixture was diluted with water and extracted two times with EtOAc (some less polar impurities were present in organic layer while product remained in water layer). The water layer was acidified with 6N HCl (pH˜5-6) and extracted with EtOAc. The collected organic layers were dried over Na₂SO₄ and evaporated to obtain the title compound (0.174 g, 96% pure, 70%) which was pure enough for use in the next step. MS (ES+) m/e 259 [M+H]⁺.

### b) 3-({3-(aminocarbonyl)-7-[2,4-bis(methyloxy)-5-pyrimidinyl]-4-quinonyl}amino)-5-[2-methyl-6-(methyloxy)-4-pyridinyl]benzoic acid

To a suspension of 3-amino-5-[2-methyl-6-(methyloxy)-4-pyridinyl]benzoic acid (74.9 mg, 0.290 mmol) in acetic acid (11 mL), 7-[2,4-bis(methyloxy)-5-pyrimidinyl]-4-chloro-3-quinolinecarboxamide (example 1b, 100 mg, 0.290 mmol) was added. The reaction mixture (suspension, reactants were not dissolved completely) was stirred at RT and monitored by UPLC-MS. According to UPLC-MS beside desired product (MH+=567.4), a large quantity of a 4-hydroxyquinoline side product was observed in the reaction mixture. After 21 h, water was added to the reaction mixture resulting in the clear solution. After several attempts of adjusting pH values and extraction with EtOAc, product was partially extracted from water to organic phase. The collected organic layers were dried over Na₂SO₄ and evaporated to obtain an impure product, which was purified by reverse phase HPLC (ODS, aqueous 10 mM ammonium bicarbonate/acetonitrile) to obtain the title compound (0.022 g, 13%). MS (ES+) m/e 567 [M+H]⁺.

### Example 56

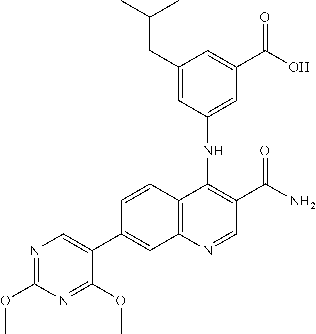 [SMILES: COc1ncc(-c2ccc3c(Nc4cc(CC(C)C)cc(C(=O)O)c4)c(C(N)=O)cnc3c2)c(OC)n1]

### 3-({3-(aminocarbonyl)-7-[2,4-bis(methyloxy)-5-pyrimidinyl]-4-quinolinyl}amino)-5-(2-methylpropyl)benzoic acid, acetate salt

### a) 3-amino-5-(2-methyl-1-propen-1-yl)benzoic acid

A 5 mL microwave vial was charged with 3-amino-5-bromobenzoic acid (200 mg, 0.926 mmol), (2-methyl-1-propen-1-yl)boronic acid (185 mg, 1.852 mmol), tetrakis(triphenylphosphine)palladium(0) (53.5 mg, 0.046 mmol), and potassium carbonate (256 mg, 1.852 mmol) in 1,4-dioxane (3 mL)/water (1.000 mL). The sealed vial was irradiated in a Biotage microwave synthesizer at 145° C. for 25 minutes. Purification was performed by reverse phase HPLC (10-90% acetonitrile/water w/0.1% TFA) to give the title compound (178 mg, 0.931 mmol, 101% yield) as a tan solid. ¹H NMR (METHANOL-d₄): 7.91 (s, 1H), 7.84 (s, 1H), 7.46 (s, 1H), 6.33 (s, 1H), 1.95 (d, J=1.0 Hz, 3H), 1.90 (d, 3H).

### b) 3-amino-5-(2-methylpropyl)benzoic acid

10% palladium-on-charcoal (2.476 mg, 0.023 mmol) was added to a stirred solution of 3-amino-5-(2-methyl-1-propen-1-yl)benzoic acid (89 mg, 0.465 mmol) in methanol (3 mL) under nitrogen. The flask was then purged with hydrogen and the mixture stirred at rt overnight. The catalyst was then filtered and the solvent concentrated to afford the title compound (70 mg, 0.362 mmol, 78% yield). No purification was performed. 1H NMR (METHANOL-d4): 7.67-7.88 (m, 2H), 7.34 (t, J=1.8 Hz, 1H), 2.60 (d, J=7.3 Hz, 2H), 1.83-1.99 (m, 1H), 0.94 (d, J=6.6 Hz, 6H).

### c) 3-({3-(aminocarbonyl)-7-[2,4-bis(methyloxy)-5-pyrimidinyl]-4-quinolinyl}amino)-5-(2-methylpropyl)benzoic acid, acetate salt

3-amino-5-(2-methylpropyl)benzoic acid (70.6 mg, 0.365 mmol) was added to a stirred solution of 7-[2,4-bis(methyloxy)-5-pyrimidinyl]-4-chloro-3-quinolinecarboxamide (example 1b, 63 mg, 0.183 mmol) in acetic acid (2 mL) at rt. The reaction was stirred for 48 hours, then the precipitate was filtered, washed with acetic acid, and dried in vacuo to give the title compound (69 mg, 0.123 mmol, 67.2% yield) as a pale yellow solid. MS (ES+) m/e 502 [M+H]⁺.

### Example 57

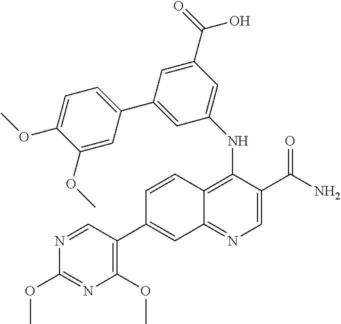 [SMILES: COc1ncc(-c2ccc3c(Nc4cc(C(=O)O)cc(-c5ccc(OC)c(OC)c5)c4)c(C(N)=O)cnc3c2)c(OC)n1]

### 5-({3-(aminocarbonyl)-7-[2,4-bis(methyloxy)-5-pyrimidinyl]-4-quinolinyl}amino)-3′,4′-bis(methyloxy)-3-biphenylcarboxylic acid

### a) 5-amino-3′,4′-bis(methyloxy)-3-biphenylcarboxylic acid

A suspension of 3-amino-5-bromobenzoic acid (200 mg, 0.926 mmol), 3,4-dimethoxyphenylboronic acid (185 mg, 1.018 mmol) and potassium carbonate (384 mg, 2.78 mmol) in 1,4-dioxane (9 mL) and water (3 mL) was degassed by bubbling with Ar for few min. Tetrakis(triphenylphosphine)palladium(0) (53.5 mg, 0.046 mmol) was added. The reaction mixture was sealed and heated in Biotage Initiator microwave reactor at 110° C. for 35 min. According to UPLC-MS, conversion was complete. The reaction mixture was diluted with water and extracted two times with EtOAc (some less polar impurities were present in organic layer while product remained in water layer). The water layer was acidified with 6N HCl (pH˜5-6) and cooled. The resulting precipitate was filtered, washed with water and dried to obtain the title compound (0.157 g, 60%) as an off-white solid. MS (ES+) m/e 274 [M+H]⁺.

### b) 5-({3-(aminocarbonyl)-7-[2,4-bis(methyloxy)-5-pyrimidinyl]-4-quinolinyl}amino)-3′,4′-bis(methyloxy)-3-biphenylcarboxylic acid

To a suspension of 5-amino-3′,4′-bis(methyloxy)-3-biphenylcarboxylic acid (79 mg, 0.290 mmol) in acetic acid (10 mL), 7-[2,4-bis(methyloxy)-5-pyrimidinyl]-4-chloro-3-quinolinecarboxamide (example 1b, 100 mg, 0.290 mmol) was added. The reaction mixture (suspension, reactants were not dissolved completely) was stirred at room temperature overnight. After 18 h according to UPLC-MS, beside minor quanitities of starting material, mainly desired product was observed in the reaction mixture. Prolongation of reaction for additional 1 h at 80° C. gave no significant change, so the reaction was stopped. The volume of the reaction mixture was reduced by evaporating, the water was added and the precipitate was filtered, washed with water and dried to afford the title compound (0.038 g, 95% pure, 21%) as a yellow solid. MS (ES+) m/e 582 [M+H]⁺.

### Example 58

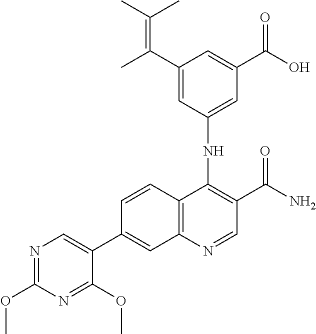 [SMILES: COc1ncc(-c2ccc3c(Nc4cc(C(=O)O)cc(C(C)=C(C)C)c4)c(C(N)=O)cnc3c2)c(OC)n1]

### 3-({3-(aminocarbonyl)-7-[2,4-bis(methyloxy)-5-pyrimidinyl]-4-quinolinyl}amino)-5-(1,2-dimethyl-1-propen-1-yl)benzoic acid, acetate salt

### a) 3-amino-5-(1,2-dimethyl-1-propen-1-yl)benzoic acid

A 5 mL microwave vial was charged with 3-amino-5-bromobenzoic acid (250 mg, 1.157 mmol), (1,2-dimethyl-1-propen-1-yl)boronic acid (264 mg, 2.314 mmol), tetrakis(triphenylphosphine)palladium(0) (66.9 mg, 0.058 mmol), and potassium carbonate (320 mg, 2.314 mmol) in 1,4-dioxane (3 mL)/water (1.000 mL). The sealed vial was irradiated in a Biotage microwave synthesizer at 145° C. for 25 minutes. Purification was performed by reverse-phase HPLC (10-90% acetonitrile/water w/0.1% TFA) to give the title compound (242 mg, 1.179 mmol, 102% yield) as a tan solid. ¹H NMR (METHANOL-d₄): 7.89 (s, 1H), 7.81 (s, 1H), 7.38 (s, 1H), 1.98 (s, 3H), 1.85 (s, 3H), 1.59 (d, 3H).

### b) 3-({3-(aminocarbonyl)-7-[2,4-bis(methyloxy)-5-pyrimidinyl]-4-quinolinyl}amino)-5-(1,2-dimethyl-1-propen-1-yl)benzoic acid, acetate salt

3-amino-5-(1,2-dimethyl-1-propen-1-yl)benzoic acid (121 mg, 0.590 mmol) was added to a stirred solution of 7-[2,4-bis(methyloxy)-5-pyrimidinyl]-4-chloro-3-quinolinecarboxamide (example 1b, 100 mg, 0.290 mmol) in acetic acid (2 mL) at rt. The reaction was stirred for 48 hours, then the precipitate was filtered, washed with acetic acid, and dried in vacuo to give the title compound (60 mg, 0.105 mmol, 36.1% yield) as a pale yellow solid. ¹H NMR (DMSO-d₆): 8.99 (s, 1H), 8.63 (s, 1H), 8.31 (br. s., 1H), 8.22 (d, J=1.5 Hz, 1H), 8.12 (d, J=8.8 Hz, 1H), 7.75-7.89 (m, 2H), 7.69 (s, 1H), 7.54 (s, 1H), 7.19 (s, 1H), 4.01 (s, 3H), 3.99 (s, 3H), 1.91 (s, 3H), 1.76 (s, 3H), 1.54 (d, J=1.3 Hz, 3H).

### Example 59

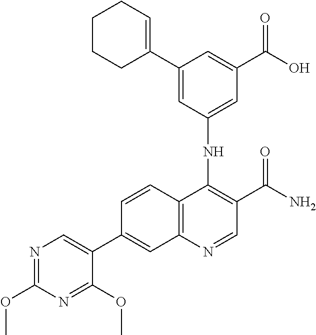 [SMILES: COc1ncc(-c2ccc3c(Nc4cc(C(=O)O)cc(C5=CCCCC5)c4)c(C(N)=O)cnc3c2)c(OC)n1]

### 3-({3-(aminocarbonyl)-7-[2,4-bis(methyloxy)-5-pyrimidinyl]-4-quinolinyl}amino)-5-(1-cyclohexen-1-yl)benzoic acid, trifluoroacetate salt

3-amino-5-(1-cyclohexen-1-yl)benzoic acid (example 54a, 69.3 mg, 0.319 mmol) was added to a stirred solution of 7-[2,4-bis(methyloxy)-5-pyrimidinyl]-4-chloro-3-quinolinecarboxamide (example 1b, 100 mg, 0.290 mmol) in acetic acid (3 mL) at rt. The reaction was stirred for two days before being purified twice by reverse-phase HPLC (10-90% acetonitrile/water w/0.1% TFA, 10 min. gradient) to give the title compound (22.5 mg, 0.035 mmol, 12.13% yield) as an offwhite solid. ¹H NMR (METHANOL-d₄): 8.95 (s, 1H), 8.57 (s, 1H), 8.19 (d, J=1.5 Hz, 1H), 8.07 (s, 2H), 7.77-7.87 (m, 2H), 7.61 (s, 1H), 6.22-6.35 (m, 1H), 4.14 (s, 3H), 4.10 (s, 3H), 2.44 (d, J=1.5 Hz, 2H), 2.19-2.30 (m, 2H), 1.76-1.88 (m, 2H), 1.60-1.76 (m, 2H).

### Example 60

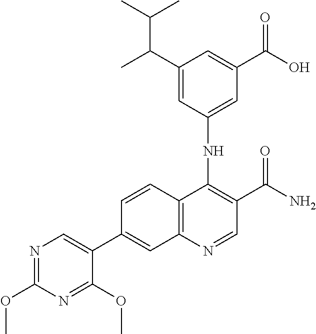 [SMILES: COc1ncc(-c2ccc3c(Nc4cc(C(=O)O)cc(C(C)C(C)C)c4)c(C(N)=O)cnc3c2)c(OC)n1]

### 3-({3-(aminocarbonyl)-7-[2,4-bis(methyloxy)-5-pyrimidinyl]-4-quinolinyl}amino)-5-(1,2-dimethylpropyl)benzoic acid, acetate salt

### a) 3-amino-5-(1,2-dimethylpropyl)benzoic acid

10% palladium-on-charcoal (3.14 mg, 0.029 mmol) was added to a stirred solution of 3-amino-5-(1,2-dimethyl-1-propen-1-yl)benzoic acid (example 58a, 121 mg, 0.590 mmol) in ethanol (3 mL) under nitrogen. The flask was then purged with hydrogen and the mixture stirred at rt overnight. The catalyst was then filtered and the solvent removed. HPLC purification (10-90% Acetonitrile/water w/0.1% TFA) afforded the title compound (76 mg, 0.367 mmol, 62.2% yield) as a clear glassy solid. ¹H NMR (METHANOL-d₄): 7.70 (s, 1H), 7.67-7.69 (m, 1H), 2.42-2.60 (m, 1H), 1.69-1.89 (m, 1H), 1.27 (d, J=7.1 Hz, 3H), 0.98 (d, J=6.6 Hz, 3H), 0.77 (d, J=6.6 Hz, 3H).

### b) 3-({3-(aminocarbonyl)-7-[2,4-bis(methyloxy)-5-pyrimidinyl]-4-quinolinyl}amino)-5-(1,2-dimethylpropyl)benzoic acid

3-amino-5-(1,2-dimethylpropyl)benzoic acid (78 mg, 0.377 mmol) was added to a stirred solution of 7-[2,4-bis(methyloxy)-5-pyrimidinyl]-4-chloro-3-quinolinecarboxamide (example 1b, 65 mg, 0.189 mmol) in acetic acid (2 mL) at rt. The reaction was stirred for 48 hours, then the precipitate was filtered, washed with acetic acid, and dried in vacuo to give the title compound (25 mg, 0.043 mmol, 23.04% yield) as a pale yellow solid. ¹H NMR (DMSO-d₆): 9.00 (s, 1H), 8.58-8.70 (m, 1H), 8.26-8.38 (m, 1H), 8.16-8.26 (m, 1H), 7.95-8.11 (m, 1H), 7.71-7.90 (m, 2H), 7.58-7.71 (m, 2H), 7.22-7.36 (m, 1H), 4.01 (s, 3H), 3.99 (s, 3H), 1.67-1.78 (m, 1H), 1.17 (d, 3H), 0.88 (d, 3H), 0.71 (d, 3H).

### Example 61

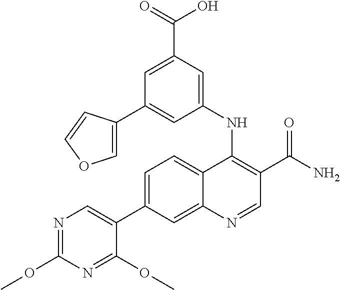 [SMILES: COc1ncc(-c2ccc3c(Nc4cc(C(=O)O)cc(-c5ccoc5)c4)c(C(N)=O)cnc3c2)c(OC)n1]

### 3-({3-(aminocarbonyl)-7-[2,4-bis(methyloxy)-5-pyrimidinyl]-4-quinolinyl}amino)-5-(3-furanyl)benzoic acid, trifluoroacetate salt

### a) 3-amino-5-(3-furanyl)benzoic acid

To a suspension of 3-amino-5-bromobenzoic acid (1 g, 4.63 mmol), 3-furanylboronic acid (0.518 g, 4.63 mmol) and potassium carbonate (1.279 g, 9.26 mmol) in 1,4-dioxane (12 mL) and water (4.00 mL) was added tetrakis(triphenylphosphine)palladium(0) (0.267 g, 0.231 mmol). The mixture was heated to 100° C. overnight. After cooling, the mixture was diluted with water, acidified with 6N HCl and extracted with ethyl acetate. The organic layer was dried over MgSO₄, filtered and concentrated under reduced pressure. The precipitate was collected and washed with methylene chloride and ether to afford 3-amino-5-(3-furanyl)benzoic acid (550 mg, 2.71 mmol, 58.5% yield) as a brown solid. 1H NMR (400 MHz, DMSO-d6) δ ppm 12.67 (br. s., 1H), 8.10 (s, 1H), 7.74 (t, J=1.6 Hz, 1H), 7.29 (t, J=1.5 Hz, 1H), 7.07-7.11 (m, 1H), 6.97 (t, J=1.9 Hz, 1H), 6.80-6.84 (m, 1H), 5.38 (br. s., 2H). MS (ES+) m/e 204 [M+H]+

### b) 3-({3-(aminocarbonyl)-7-[2,4-bis(methoxy)-5-pyrimidinyl]-4-quinolinyl}amino)-5-(3-furanyl)benzoic acid, trifluoroacetate salt

To a suspension of 7-[2,4-bis(methyloxy)-5-pyrimidinyl]-4-chloro-3-quinolinecarboxamide (example 1b, 50 mg, 0.145 mmol) in acetic acid (10 mL) was added 3-amino-5-(3-furanyl)benzoic acid (29.5 mg, 0.145 mmol). The reaction mixture was stirred at room temperature overnight and was purified through preparative HPLC (YMC 75×30 mm column, 0.1% TFA in water and 0.1% TFA in acetonitrile) to afford 3-({3-(aminocarbonyl)-7-[2,4-bis(methyloxy)-5-pyrimidinyl]-4-quinolinyl}amino)-5-(3-furanyl)benzoic acid (18 mg, 0.029 mmol, 19.84% yield) as a yellow solid. 1H NMR (400 MHz, MeOD) δ ppm 8.94 (s, 1H), 8.54 (s, 1H), 8.15-8.23 (m, 3H), 8.03 (s, 1H), 7.82-7.88 (m, 2H), 7.75-7.79 (m, 1H), 7.59-7.63 (m, 1H), 6.87 (d, J=1.0 Hz, 1H), 4.11 (s, 3H), 4.08 (s, 3H). MS (ES+) m/e 512 [M+H]+

### Example 62

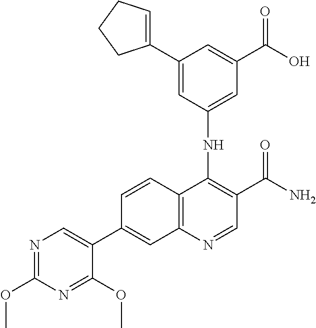 [SMILES: COc1ncc(-c2ccc3c(Nc4cc(C(=O)O)cc(C5=CCCC5)c4)c(C(N)=O)cnc3c2)c(OC)n1]

### 3-({3-(aminocarbonyl)-7-[2,4-bis(methyloxy)-5-pyrimidinyl]-4-quinolinyl}amino)-5-(1-cyclopenten-1-yl)benzoic acid

3-amino-5-(1-cyclopenten-1-yl)benzoic acid (example 53a, 64.8 mg, 0.319 mmol) was added to a stirred solution of 7-[2,4-bis(methyloxy)-5-pyrimidinyl]-4-chloro-3-quinolinecarboxamide (example 1b, 100 mg, 0.290 mmol) in acetic acid (2 mL) at 70° C.

The reaction was stirred overnight and purified by reverse-phase HPLC (10-70% acetonitrile/water w/01. % TFA) to give the title compound (9 mg, 0.018 mmol, 6.07% yield) as a yellow solid. ¹H NMR (METHANOL-d₄): 8.95 (s, 1H), 8.58 (br. s., 1H), 8.19 (d, J=1.8 Hz, 1H), 8.11 (s, 2H), 7.79-7.91 (m, 2H), 7.68 (t, J=1.9 Hz, 1H), 6.40 (t, J=1.9 Hz, 1H), 4.13 (s, 3H), 4.10 (s, 3H), 2.71-2.79 (m, 2H), 2.58 (td, J=7.4, 2.4 Hz, 2H), 2.07 (quin, 2H).

### Example 63

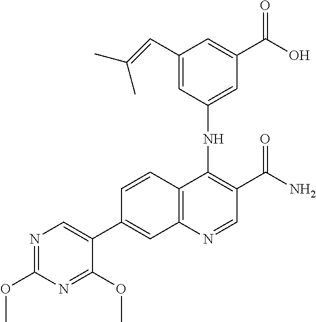 [SMILES: COc1ncc(-c2ccc3c(Nc4cc(C=C(C)C)cc(C(=O)O)c4)c(C(N)=O)cnc3c2)c(OC)n1]

### 3-({3-(aminocarbonyl)-7-[2,4-bis(methyloxy)-5-pyrimidinyl]-4-quinolinyl}amino)-5-(2-methyl-1-propen-1-yl)benzoic acid

3-amino-5-(2-methyl-1-propen-1-yl)benzoic acid (example 56a, 47.7 mg, 0.249 mmol) was added to a stirred solution of 7-[2,4-bis(methyloxy)-5-pyrimidinyl]-4-chloro-3-quinolinecarboxamide (example 1b, 43 mg, 0.125 mmol) in acetic acid (2 mL) at rt. The reaction was stirred for 48 hours, then the precipitate was filtered, washed with acetic acid, and dried in vacuo to give the title compound (27 mg, 0.048 mmol, 38.7% yield) as a pale yellow solid. ¹H NMR (METHANOL-d₄): 8.96 (s, 1H), 8.56 (br. s., 1H), 8.19 (s, 1H), 8.06 (d, J=9.3 Hz, 1H), 7.92 (s, 1H), 7.74-7.87 (m, 2H), 7.44 (s, 1H), 6.34 (br. s., 1H), 4.13 (s, 3H), 4.09 (s, 3H), 1.94 (s, 3H), 1.87 (s, 3H).

### Example 64

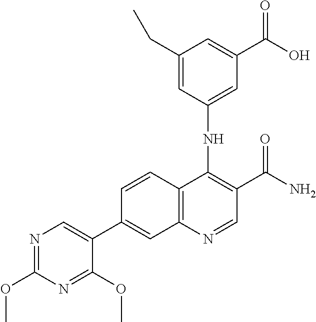 [SMILES: CCc1cc(Nc2c(C(N)=O)cnc3cc(-c4cnc(OC)nc4OC)ccc23)cc(C(=O)O)c1]

### 3-({3-(aminocarbonyl)-7-[2,4-bis(methyloxy)-5-pyrimidinyl]-4-quinolinyl}amino)-5-ethylbenzoic acid, trifluoroacetate salt

### a) 3-amino-5-ethenylbenzoic acid

A 5 mL microwave vial was charged with 3-amino-5-bromobenzoic acid (200 mg, 0.926 mmol), 2,4,6-trivinylcyclotriboroxane pyridine complex (446 mg, 1.852 mmol), tetrakis(triphenylphosphine)palladium(0) (53.5 mg, 0.046 mmol), and potassium carbonate (256 mg, 1.852 mmol) in 1,4-dioxane (3 mL)/water (1.000 mL). The sealed vial was irradiated in a Biotage microwave synthesizer at 145° C. for 25 minutes. The mixture was filtered and acidified with aqueous hydrochloric acid, and the resulting precipitate filtered. The eluent was concentrated to give the crude product. The compound was advanced to the next step with no purification. ¹H NMR (DMSO-d6): 7.38 (s, 1H), 7.27 (s, 1H), 7.06 (s, 1H), 6.71 (dd, J=17.7, 10.9 Hz, 1H), 5.77 (d, J=17.4 Hz, 1H), 5.29 (d, 1H).

### b) 3-amino-5-ethylbenzoic acid

10% palladium-on-charcoal (2.364 mg, 0.022 mmol) was added to a stirred solution of 3-amino-5-ethenylbenzoic acid (72.5 mg, 0.444 mmol) in ethanol (3 mL) under nitrogen. The flask was then purged with hydrogen and the mixture stirred at rt overnight. The catalyst was then filtered and the mixture concentrated to give the title compound (38 mg, 0.230 mmol, 51.8% yield) as a clear glassy solid. ¹H NMR (METHANOL-d₄): 7.89 (s, 1H), 7.79 (s, 1H), 7.40 (s, 1H), 2.77 (q, J=7.6 Hz, 2H), 1.29 (t, J=7.6 Hz, 3H).

### c) 3-({3-(aminocarbonyl)-7-[2,4-bis(methyloxy)-5-pyrimidinyl]-4-quinolinyl}amino)-5-ethylbenzoic acid, trifluoroacetate salt

3-amino-5-ethylbenzoic acid (37.4 mg, 0.226 mmol) was added to a stirred solution of 7-[2,4-bis(methyloxy)-5-pyrimidinyl]-4-chloro-3-quinolinecarboxamide (example 1b, 65 mg, 0.189 mmol) in acetic acid (2 mL) at rt. The reaction was stirred for 48 hours, then the precipitate was filtered, washed with acetic acid, and dried in vacuo to give the title compound (6 mg, 10.21 μmol, 5.42% yield) as a pale yellow solid. ¹H NMR (METHANOL-d₄): 8.96 (s, 1H), 8.48 (s, 1H), 8.15 (d, J=1.8 Hz, 1H), 7.93 (d, J=8.8 Hz, 1H), 7.66 (s, 1H), 7.51-7.64 (m, 2H), 7.16 (d, J=1.8 Hz, 1H), 4.11 (s, 3H), 4.08 (s, 3H), 2.67 (q, J=7.7 Hz, 2H), 1.24 (t, J=7.6 Hz, 3H).

### Example 65

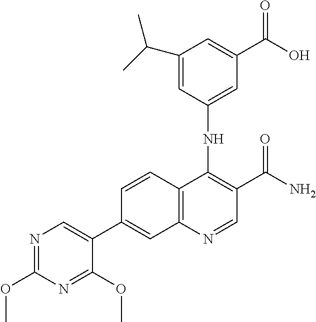 [SMILES: COc1ncc(-c2ccc3c(Nc4cc(C(=O)O)cc(C(C)C)c4)c(C(N)=O)cnc3c2)c(OC)n1]

### 3-({3-(aminocarbonyl)-7-[2,4-bis(methyloxy)-5-pyrimidinyl]-4-quinolinyl}amino)-5-(1-methylethyl)benzoic acid, acetate salt

### a) 3-amino-5-(1-methylethenyl)benzoic acid

A 5 mL microwave vial was charged with 3-amino-5-bromobenzoic acid (200 mg, 0.926 mmol), 4,4,5,5-tetramethyl-2-(1-methylethenyl)-1,3,2-dioxaborolane (0.348 mL, 1.852 mmol), tetrakis(triphenylphosphine)palladium(0) (53.5 mg, 0.046 mmol), and potassium carbonate (256 mg, 1.852 mmol) in 1,4-dioxane (3 mL)/water (1.000 mL). The sealed vial was irradiated in a Biotage microwave synthesizer at 145° C. for 25 minutes. The mixture was then filtered, concentrated, and purified by reverse-phase HPLC (10-90% acetonitrile/water gradient with 0.1% TFA to give the title compound (165 mg, 0.931 mmol, 101% yield) as a white solid. ¹H NMR (DMSO-d₆): 7.40 (s, 1H), 7.27 (s, 1H), 7.09 (d, J=2.0 Hz, 1H), 5.38 (s, 1H), 5.13 (m, J=1.4 Hz, 1H), 2.09 (s, 3H).

### b) 3-amino-5-(1-methylethyl)benzoic acid

10% palladium-on-charcoal (2.462 mg, 0.023 mmol) was added to a stirred solution of 3-amino-5-(1-methylethenyl)benzoic acid (82 mg, 0.463 mmol) in ethanol (3 mL) under nitrogen. The flask was then purged with hydrogen and the mixture stirred at rt overnight. The catalyst was then filtered and solvent concentrated to give the title compound (74 mg, 0.413 mmol, 89% yield) as a clear glassy solid. ¹H NMR (400 MHz, METHANOL-d₄) ppm 1.31 (d, J=7.07 Hz, 6H) 2.98-3.11 (m, 1H) 7.46 (s, 1H) 7.79-7.83 (m, 1H) 7.93 (s, 1H). MS (ES+) m/e 180 [M+H]⁺.

### c) 3-({3-(aminocarbonyl)-7-[2,4-bis(methyloxy)-5-pyrimidinyl]-4-quinolinyl}amino)-5-(1-methylethyl)benzoic acid, acetate salt

3-amino-5-(1-methylethyl)benzoic acid (73.8 mg, 0.412 mmol) was added to a stirred solution of 7-[2,4-bis(methyloxy)-5-pyrimidinyl]-4-chloro-3-quinolinecarboxamide (example 1b, 71 mg, 0.206 mmol) in acetic acid (2 mL) at rt. The reaction was stirred for 48 hours, then the precipitate was filtered, washed with acetic acid, and dried in vacuo to give the title compound (111 mg, 0.203 mmol, 98% yield) as a pale yellow solid. MS (ES+) m/e 488 [M+H]⁺.

### Example 66

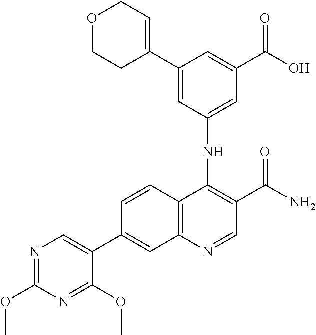 [SMILES: COc1ncc(-c2ccc3c(Nc4cc(C(=O)O)cc(C5=CCOCC5)c4)c(C(N)=O)cnc3c2)c(OC)n1]

### 3-({3-(aminocarbonyl)-7-[2,4-bis(methyloxy)-5-pyrimidinyl]-4-quinolinyl}amino)-5-(3,6-dihydro-2H-pyran-4-yl)benzoic acid

### a) 3-amino-5-(3,6-dihydro-2H-pyran-4-yl)benzoic acid

A 5 mL microwave vial was charged with 3-amino-5-bromobenzoic acid (250 mg, 1.157 mmol), 4-(4,4,5,5-tetramethyl-1,3,2-dioxaborolan-2-yl)-3,6-dihydro-2H-pyran (486 mg, 2.314 mmol), tetrakis(triphenylphosphine)palladium(0) (66.9 mg, 0.058 mmol), and potassium carbonate (320 mg, 2.314 mmol) in 1,4-dioxane (3 mL)/water (1.000 mL). The sealed vial was irradiated in a Biotage microwave synthesizer at 145° C. for 25 minutes. Purification was performed by reverse-phase HPLC (10-90% acetonitrile/water w/0.1% TFA) to give the title compound (211 mg, 0.962 mmol, 83% yield) as a tan solid. ¹H NMR (METHANOL-d₄): 8.15 (t, J=1.5 Hz, 1H), 7.86-7.96 (m, 1H), 7.65 (t, J=1.9 Hz, 1H), 6.40 (dt, J=3.0, 1.5 Hz, 1H), 4.26-4.39 (m, 2H), 3.96 (t, J=5.4 Hz, 2H), 2.53-2.62 (m, 2H).

### b) 3-({3-(aminocarbonyl)-7-[2,4-bis(methyloxy)-5-pyrimidinyl]-4-quinolinyl}amino)-5-(3,6-dihydro-2H-pyran-4-yl)benzoic acid

3-amino-5-(3,6-dihydro-2H-pyran-4-yl)benzoic acid (102 mg, 0.464 mmol) was added to a stirred solution of 7-[2,4-bis(methyloxy)-5-pyrimidinyl]-4-chloro-3-quinolinecarboxamide (example 1b, 80 mg, 0.232 mmol) in acetic acid (2 mL) at rt. The reaction was stirred for 48 hours, then the precipitate was filtered, washed with acetic acid, and dried in vacuo to give the title compound (106 mg, 0.180 mmol, 78% yield) as a pale yellow solid. ¹H NMR (DMSO-d₆): 8.98 (s, 1H), 8.67 (s, 1H), 8.27 (d, J=1.5 Hz, 3H), 7.88 (s, 2H), 7.73 (br. s., 2H), 7.60 (s, 1H), 6.39 (br. s., 1H), 4.23 (d, J=2.8 Hz, 2H), 4.03 (s, 3H), 4.00 (s, 3H), 3.82 (t, J=5.4 Hz, 2H), 2.45 (d, 2H).

### Example 67

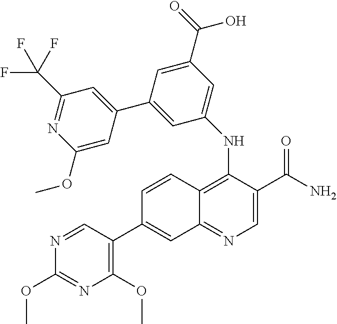 [SMILES: COc1cc(-c2cc(Nc3c(C(N)=O)cnc4cc(-c5cnc(OC)nc5OC)ccc34)cc(C(=O)O)c2)cc(C(F)(F)F)n1]

### 3-({3-(aminocarbonyl)-7-[2,4-bis(methyloxy)-5-pyrimidinyl]-4-quinolinyl}amino)-5-[2-(methyloxy)-6-(trifluoromethyl)-4-pyridinyl]benzoic acid

### a) 3-amino-5-[2-(methyloxy)-6-(trifluoromethyl)-4-pyridinyl]benzoic acid

A suspension of 3-amino-5-bromobenzoic acid (200 mg, 0.926 mmol), 2-methoxy-6-trifluoromethylpyridine-4-boronic acid, pinacol ester (309 mg, 1.018 mmol) and potassium carbonate (384 mg, 2.78 mmol) in 1,4-dioxane (9 mL) and water (3 mL) was degassed by bubbling with Ar for few min.

Tetrakis(triphenylphosphine)palladium(0) (53.5 mg, 0.046 mmol) was added. The reaction mixture was sealed and heated in Biotage Initiator microwave reactor at 110° C. for 35 min. According to UPLC-MS, conversion was complete. The reaction mixture was diluted with water and extracted two times with EtOAc (some less polar impurities were present in organic layer while product remained in water layer). The water layer was acidified with 6N HCl (pH˜5-6) and extracted with EtOAc. The collected organic layers were dried over Na₂SO₄ and evaporated to obtain the title compound (0.194 g, 65%), pure enough according to LC-MS for use in the next step. MS (ES+) m/e 313 [M+H]⁺.

### b) 3-({3-(aminocarbonyl)-7-[2,4-bis(methyloxy)-5-pyrimidinyl]-4-quinolinyl}amino)-5-[2-(methyloxy)-6-(trifluoromethyl)-4-pyridinyl]benzoic acid

To a suspension of 3-amino-5-[2-(methyloxy)-6-(trifluoromethyl)-4-pyridinyl]benzoic acid (91 mg, 0.290 mmol) in acetic acid (7 mL), 7-[2,4-bis(methyloxy)-5-pyrimidinyl]-4-chloro-3-quinolinecarboxamide (example 1b, 100 mg, 0.290 mmol) was added. The reaction mixture (suspension, reactants were not dissolved completely) was stirred at 80° C. for 2 h. According to UPLC-MS, beside desired product (MH+=621.5), some quantity of starting acid and large quantity of 4-hydroxy-quinoline side product were observed in the reaction mixture. Additional quantity of 7-[2,4-bis(methyloxy)-5-pyrimidinyl]-4-chloro-3-quinolinecarboxamide (20 mg, 0.058 mmol) was added to the reaction mixture and reaction continued for 2 h at 80° C. After that time UPLC-MS showed no significant improvement of yield, so the reaction was stopped. The volume of the reaction mixture was reduced by evaporating, the water was added and the precipitate was filtered, washed with water and dried to afford an impure product, which was purified by reverse phase HPLC (ODS, aqueous 10 mM ammonium bicarbonate/acetonitrile) to obtain the title compound (0.035 g, 19%). MS (ES+) m/e 621 [M+H]⁺.

### Example 68

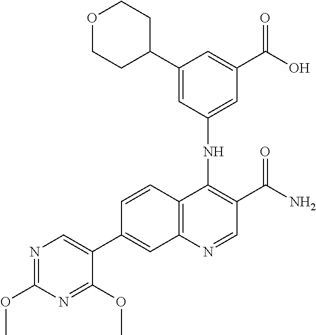 [SMILES: COc1ncc(-c2ccc3c(Nc4cc(C(=O)O)cc(C5CCOCC5)c4)c(C(N)=O)cnc3c2)c(OC)n1]

### 3-({3-(aminocarbonyl)-7-[2,4-bis(methyloxy)-5-pyrimidinyl]-4-quinolinyl}amino)-5-(tetrahydro-2H-pyran-4-yl)benzoic acid

### a) 3-amino-5-(tetrahydro-2H-pyran-4-yl)benzoic acid

10% palladium-on-charcoal (24.27 mg, 0.228 mmol) was added to a stirred solution of 3-amino-5-(3,6-dihydro-2H-pyran-4-yl)benzoic acid (example 66a, 100 mg, 0.456 mmol) in ethanol (3 mL) under nitrogen. The flask was then purged with hydrogen and the mixture stirred at rt overnight. The catalyst was then filtered and solvent removed to give the title compound (80 mg, 0.362 mmol, 79% yield) as a clear glassy solid. ¹H NMR (METHANOL-d₄): 7.81 (s, 1H), 7.78 (s, 1H), 7.46 (br. s., 1H), 4.07 (d, J=10.9 Hz, 2H), 3.52-3.67 (m, 2H), 2.68 (s, 1H), 1.72-1.93 (m, 4H).

### b) 3-({3-(aminocarbonyl)-7-[2,4-bis(methyloxy)-5-pyrimidinyl]-4-quinolinyl}amino)-5-(tetrahydro-2H-pyran-4-yl)benzoic acid

3-amino-5-(tetrahydro-2H-pyran-4-yl)benzoic acid (25.4 mg, 0.115 mmol) was added to a stirred solution of 7-[2,4-bis(methyloxy)-5-pyrimidinyl]-4-chloro-3-quinolinecarboxamide (example 1b, 22 mg, 0.064 mmol) in acetic acid (2 mL) at rt. The reaction was stirred for 48 hours, then the precipitate was filtered, washed with acetic acid, and dried in vacuo to give the title compound (6.6 mg, 0.012 mmol, 19.53% yield) as a pale yellow solid. ¹H NMR (METHANOL-d₄): 8.95 (s, 1H), 8.57 (s, 1H), 8.19 (d, J=1.8 Hz, 1H), 8.08 (d, J=8.8 Hz, 1H), 7.97 (s, 1H), 7.86-7.90 (m, 1H), 7.83 (dd, J=9.1, 1.8 Hz, 1H), 7.51 (t, J=1.8 Hz, 1H), 4.13 (s, 3H), 4.10 (s, 3H), 4.05 (d, J=9.3 Hz, 2H), 3.51-3.67 (m, 2H), 2.86-3.04 (m, 1H), 1.83 (dd, J=9.3, 3.5 Hz, 4H).

### Example 69

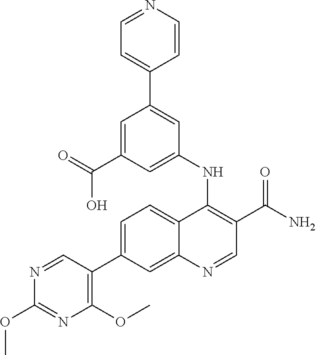 [SMILES: COc1ncc(-c2ccc3c(Nc4cc(C(=O)O)cc(-c5ccncc5)c4)c(C(N)=O)cnc3c2)c(OC)n1]

### 3-({3-(aminocarbonyl)-7-[2,4-bis(methyloxy)-5-primidinyl]-4-quiuinolinyl}amino)-5-(4-pyridinyl)benzoic acid

### a) 3-amino-5-(4-pyridinyl)benzoic acid

A suspension of 3-amino-5-bromobenzoic acid (200 mg, 0.926 mmol), 4-pyridinylboronic acid (171 mg, 1.389 mmol) and potassium carbonate (384 mg, 2.78 mmol) in 1,4-dioxane (9 mL) and water (3 mL) was degassed with argon for 2-5 min, then tetrakis(triphenylphosphine)palladium(0) (53.5 mg, 0.046 mmol) was added and mixture heated by microwave (Biotage Initiator Sixty) at 110° C. for 35 min. UPLC showed the peak with the expected mass. The solution was decanted from the black precipitate, water was added and extracted once with ethyl acetate to remove the majority of the impurities. The aqueous layer was acidified with 6N HCl to pH ca. 5-6 and product extracted once with ethyl acetate, but then left standing at rt for 15 min and in the fridge for 1 h whereupon the product crystallized out. It was filtered off and dried to give the title compound (80 mg, 40%) as yellow crystals. MS (ES+) m/e 215 [M+H]⁺.

### b) 3-({3-(aminocarbonyl)-7-[2,4-bis(methyloxy)-5-pyrimidinyl]-4-quinolinyl}amino)-5-(4-pyridinyl)benzoic acid

A suspension of 3-amino-5-(4-pyridinyl)benzoic acid (65.2 mg, 0.305 mmol) and 7-[2,4-bis(methyloxy)-5-pyrimidinyl]-4-chloro-3-quinolinecarboxamide (example 1b, 70 mg, 0.203 mmol) in acetic acid (12 mL) was stirred at rt overnight. No reaction occurred, except the hydrolysis to produce the 4-hydroxy derivative by UPLC (m/z=325 (M−H) in ES−). The reaction mixture was heated to 105° C. for 30 min, and UPLC showed the reactants were consumed. The peak of the expected product appeared (m/z=523 (MH+) in ES+, m/z=521 (M−H) in ES−). The reaction mixture was cooled to rt and then for a short time at 4° C., whereupon the product crystallized out. It was filtered off, washed with ether and dried to give the title compound (35 mg, 31%) as a yellow solid. MS (ES+) m/e 523 [M+H]⁺.

### Example 70

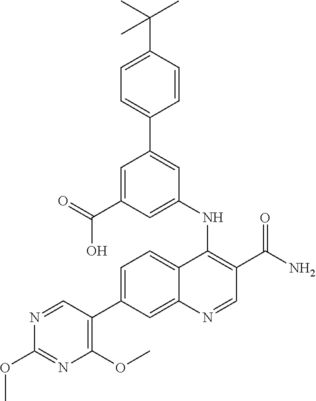 [SMILES: COc1ncc(-c2ccc3c(Nc4cc(C(=O)O)cc(-c5ccc(C(C)(C)C)cc5)c4)c(C(N)=O)cnc3c2)c(OC)n1]

### 5-({3-(aminocarbonyl)-7-[2,4-bis(methyloxy)-5-pyrimidinyl]-4-quinolinyl}amino)-4′-(1,1-dimethylethyl)-3-biphenylcarboxylic acid

### a) 5-amino-4′-(1,1-dimethylethyl)-3-biphenylcarboxylic acid

A suspension of 3-amino-5-bromobenzoic acid (200 mg, 0.926 mmol), [4-(1,1-dimethylethyl)phenyl]boronic acid (247 mg, 1.389 mmol) and potassium carbonate (384 mg, 2.78 mmol) in 1,4-dioxane (9 mL) and water (3 mL) was degassed with argon for 2-5 min, then tetrakis(triphenylphosphine)palladium(0) (53.5 mg, 0.046 mmol) was added and mixture heated by microwave (Biotage Initiator Sixty) at 100° C. for 30 min. TLC showed a minor quantity of starting 3-amino-5-bromobenzoic acid left unreacted. The solution was decanted from the black precipitate, water was added and the mixture extracted twice with ethyl acetate. The solution was evaporated till dryness, then hexane and ether (few drops) were added, whereupon the product crystallized out and was filtered off to give the title compound (135 mg, 88% pure, 48%) as a light yellow solid. MS (ES+) m/e 270 [M+H]⁺.

### b) 5-({3-(aminocarbonyl)-7-[2,4-bis(methyloxy)-5-pyrimidinyl]-4-quinolinyl}amino)-4′-(1,1-dimethylethyl)-3-biphenylcarboxylic acid

A suspension of 5-amino-4′-(1,1-dimethylethyl)-3-biphenylcarboxylic acid (82 mg, 0.305 mmol) and 7-[2,4-bis(methyloxy)-5-pyrimidinyl]-4-chloro-3-quinolinecarboxamide (example 1b, 70 mg, 0.203 mmol) in acetic acid (12 mL) was stirred at 80° C. for 15 min, and UPLC showed the reactants were consumed. The peak of the expected product appeared at Rt=1.04 min (m/z=578 (MH+) in ES+, m/z=576 (M−H) in ES−). The reaction mixture was cooled to rt and then for a short time at 4° C., but no crystallization occurred. The solution was concentrated in vacuo to volume ca 1 mL. Ether was added (2-3 mL) whereupon the product crystallized out. It was filtered off, washed with ether and dried to give the title compound (61 mg, 88% pure, 46%) as a yellow solid. MS (ES+) m/e 578 [M+H]⁺.

### Example 71

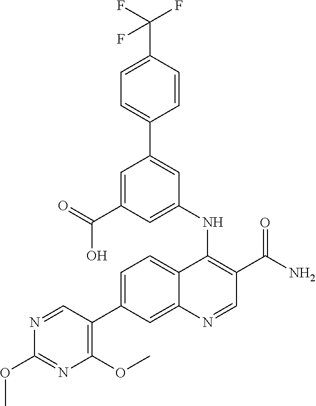 [SMILES: COc1ncc(-c2ccc3c(Nc4cc(C(=O)O)cc(-c5ccc(C(F)(F)F)cc5)c4)c(C(N)=O)cnc3c2)c(OC)n1]

### 5-({3-(aminocarbonyl)-7-[2,4-bis(methyloxy)-5-pyrimidinyl]-4-quinolinyl}amino)-4′-(trifluoromethyl)-3-biphenylcarboxylic acid

### a) 5-amino-4′-(trifluoromethyl)-3-biphenylcarboxylic acid

A solution of 3-amino-5-bromobenzoic acid (200 mg, 0.926 mmol), [4-(trifluoromethyl)phenyl]boronic acid (176 mg, 0.926 mmol) and potassium carbonate (384 mg, 2.78 mmol) in 1,4-dioxane (9 mL) and water (3 mL) was degassed with argon for 2-3 min, then tetrakis(triphenylphosphine)palladium(0) (53.5 mg, 0.046 mmol) was added and mixture heated by microwave (Biotage Initiator Sixty) at 100° C. for 30 min. TLC showed a negligible quantity of starting 3-amino-5-bromobenzoic acid left unreacted. The solution was decanted from the black precipitate, water was added and the mixture extracted twice with ethyl acetate. Solvent was evaporated to dryness, then ether (few drops) and hexane were added, whereupon the product crystallized out. This suspension was allowed to stand at 4° C. for 2.5 days, then the product was filtered off to give the title compound (108 mg, 92% pure, 38%) as a light yellow solid. MS (ES+) m/e 280 [M+H]⁺.

### b) 5-({3-(aminocarbonyl)-7-[2,4-bis(methyloxy)-5-pyrimidinyl]-4-quinolinyl}amino)-4′-(trifluoromethyl)-3-biphenylcarboxylic acid

A suspension of 5-amino-4′-(trifluoromethyl)-3-biphenylcarboxylic acid (77 mg, 0.274 mmol) and 7-[2,4-bis(methyloxy)-5-pyrimidinyl]-4-chloro-3-quinolinecarboxamide (example 1b, 70 mg, 0.203 mmol) in acetic acid (10 mL) was stirred at 75° C. for 45 min, and UPLC showed the reactants were consumed. The reaction mixture was cooled to rt, whereupon the product crystallized out. It was filtered off, washed with ether and dried to give the title compound (46 mg, 93% pure, 36%) as a light yellow solid. MS (ES+) m/e 590 [M+H]⁺.

### Example 72

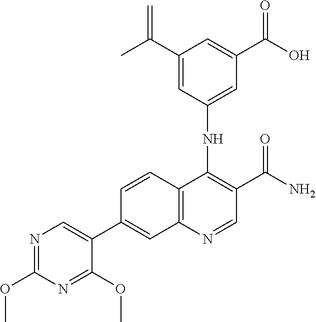 [SMILES: C=C(C)c1cc(Nc2c(C(N)=O)cnc3cc(-c4cnc(OC)nc4OC)ccc23)cc(C(=O)O)c1]

### 3-({3-(aminocarbonyl)-7-[2,4-bis(methyloxy)-5-pyrimidinyl]-4-quinolinyl}amino)-5-(1-methylethenyl)benzoic acid, acetate salt

3-amino-5-(1-methylethenyl)benzoic acid (example 65a, 82 mg, 0.464 mmol) was added to a stirred solution of 7-[2,4-bis(methyloxy)-5-pyrimidinyl]-4-chloro-3-quinolinecarboxamide (example 1b, 80 mg, 0.232 mmol) in acetic acid (3 mL) at rt. The reaction was stirred for 48 hours, then the precipitate was filtered, washed with acetic acid, and dried in vacuo to give the title compound (103 mg, 0.189 mmol, 81% yield) as a pale yellow solid. MS (ES+) m/e 485 [M+H]⁺.

### Example 73

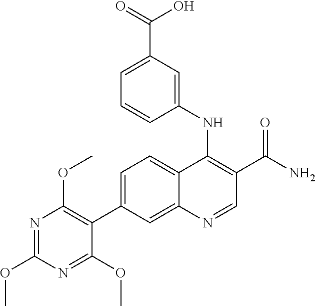 [SMILES: COc1nc(OC)c(-c2ccc3c(Nc4cccc(C(=O)O)c4)c(C(N)=O)cnc3c2)c(OC)n1]

### 3-({3-(aminocarbonyl)-7-[2,4,6-tris(methyloxy)-5-pyrimidinyl]-4-quinolinyl}amino)benzoic acid

A solution of ethyl 3-({3-(aminocarbonyl)-7-bromo-4-quinolinyl}amino)benzoate (example 6a, 250 mg, 0.603 mmol), bis(pinacolato)diboron (230 mg, 0.905 mmol), potassium acetate (178 mg, 1.810 mmol), and tetrakis(triphenylphosphine)palladium(0) (31.4 mg, 0.027 mmol) in 1,4-dioxane (5 mL) was heated to 110° C. for 3 h in an oil bath. Upon cooling, the reaction mixture was treated with 5-bromo-2,4,6-tris(methyloxy)pyrimidine (150 mg, 0.603 mmol) followed by sodium bicarbonate (sat aq solution) (2 mL, 0.603 mmol). The solution was bubbled through with a nitrogen current. After 5 minutes, the solution was treated with additional tetrakis(triphenylphosphine)palladium(0) (31.4 mg, 0.027 mmol) and heated to 110° C. overnight. Upon cooling the contents were partitioned between water and ethyl acetate. The organic portion was dried over sodium sulfate, filtered and concentrated to obtain a solid which was dissolved into ethanol and treated with sodium hydroxide (1N aq solution) (0.5 mL, 0.500 mmol). Following stirring at ambient temperature for 2 h, the reaction mixture was left unstirred overnight at room temperature. The mixture was then concentrated to an aqueous residue, which was purified using Reverse Phase HPLC (0.1% TFA in acetonitrile and water) to obtain the title compound (0.043 g, 15%) as a yellow solid. ¹H NMR (400 MHz, DMSO-d₆) ppm 13.12 (br. s., 1H), 11.63 (br. s., 1H), 8.96 (s, 1H), 8.26 (br. s., 1H), 8.05 (d, J=8.6 Hz, 1H), 7.99 (s, 1H), 7.71-7.87 (m, 3H), 7.60 (d, J=8.6 Hz, 1H), 7.42-7.57 (m, 2H), 3.98 (s, 3H), 3.89 (s, 6H). MS (ES+) m/e 476 [M+H]⁺.

### Example 74

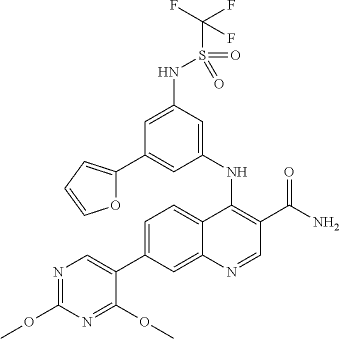 [SMILES: COc1ncc(-c2ccc3c(Nc4cc(NS(=O)(=O)C(F)(F)F)cc(-c5ccco5)c4)c(C(N)=O)cnc3c2)c(OC)n1]

### 7-[2,4-bis(methyloxy)-5-pyrimidinyl]-4-[(3-(2-furanyl)-5-{[(trifluoromethyl)sulfonyl]amino}phenyl)amino]-3-quinolinecarboxamide, trifluoroacetate salt

### a) (3-bromo-5-nitrophenyl)amine

To a solution of 1-bromo-3,5-dinitrobenzene (3 g, 12.15 mmol) in ethanol (30 mL) was added pyridine (4.91 mL, 60.7 mmol). The mixture was refluxed for half hour before ammonium sulfide (12.41 g, 36.4 mmol) in 12 mL water was added via addition funnel over 1 hour. After the addition was complete, the reflux was continued for 0.5 h. After cooling, the mixture was poured into ice water. The precipitate was collected, washed with water and dried to afford (3-bromo-5-nitrophenyl)amine (2.1 g, 9.68 mmol, 80% yield) as a yellow solid. 1H NMR (400 MHz, MeOD) δ ppm 7.51 (t, J=1.9 Hz, 1H), 7.43 (t, J=2.0 Hz, 1H), 7.14 (t, J=1.9 Hz, 1H).

### b) [3-(2-furanyl)-5-nitrophenyl]amine

To a suspension of (3-bromo-5-nitrophenyl)amine (1 g, 4.61 mmol), 2-furanylboronic acid (1.031 g, 9.22 mmol) and potassium carbonate (1.274 g, 9.22 mmol) in 1,4-dioxane (12 mL) and water (4.00 mL) was added tetrakis(triphenylphosphine)palladium(0) (0.532 g, 0.461 mmol). The mixture was heated to 100° C. overnight. After cooling, the mixture was diluted with water, acidified with 6N HCl and extracted with ethyl acetate. The organic layer was dried over MgSO₄, filtered and concentrated under reduced pressure. The crude was purified through flash chromatography (0-100% ethyl acetate in hexanes) to afford [3-(2-furanyl)-5-nitrophenyl]amine (320 mg, 1.567 mmol, 34.0% yield) as a brown solid. 1H NMR (400 MHz, CHLOROFORM-d) δ ppm 7.88 (d, J=1.3 Hz, 1H), 7.50 (s, 1H), 7.36-7.40 (m, 1H), 7.26 (d, J=1.5 Hz, 1H), 6.75 (d, J=3.3 Hz, 1H), 6.49-6.54 (m, 1H).

### c) 1,1,1-trifluoro-N-[3-(2-furanyl)-5-nitrophenyl]methanesulfonamide

To a solution of [3-(2-furanyl)-5-nitrophenyl]amine (320 mg, 1.567 mmol) and triethylamine (0.655 mL, 4.70 mmol) in dichloromethane (20 mL) cooled in ice water was added trifluoromethanesulfonic anhydride (0.397 mL, 2.351 mmol). The reaction was kept stirring for 2 hours at room temperature and concentrated under reduced pressure. The residual was dissolved in methanol, and sodium hydroxide (6.0N in water) (1.306 mL, 7.84 mmol) was added. The mixture was kept stirring for half hour at room temperature and was purified preparative HPLC (YMC 20×50 mm column, 0.1% TFA in water and 0.1% TFA in acetonitrile) to afford 1,1,1-trifluoro-N-[3-(2-furanyl)-5-nitrophenyl]methanesulfonamide (35 mg, 0.104 mmol, 6.64% yield) as a brown tar. MS (ES+) m/e 337 [M+H]⁺.

### d) 7-[2,4-bis(methyloxy)-5-pyrimidinyl]-4-[(3-(2-furanyl)-5-{[(trifluoromethyl)sulfonyl]amino}phenyl)amino]-3-quinolinecarboxamide, trifluoroacetate salt

To a suspension of 1,1,1-trifluoro-N-[3-(2-furanyl)-5-nitrophenyl]methanesulfonamide (34.1 mg, 0.102 mmol) in acetic acid (10 mL) was added palladium hydroxide on carbon (14.26 mg, 0.102 mmol). The reaction mixture was hydrogenated at room temperature under balloon for half hour, then filtered. 7-[2,4-bis(methyloxy)-5-pyrimidinyl]-4-chloro-3-quinolinecarboxamide (example 1b, 35 mg, 0.102 mmol) was added to the filtrate. The mixture was kept stirring at room temperature overnight and purified preparative HPLC (YMC 20×50 mm column, 0.1% TFA in water and 0.1% TFA in acetonitrile) to afford 7-[2,4-bis(methyloxy)-5-pyrimidinyl]-4-[(3-(2-furanyl)-5-{[(trifluoromethyl)sulfonyl]amino}phenyl)amino]-3-quinolinecarboxamide (12 mg, 0.016 mmol, 16.22% yield) as a yellow solid. ¹H NMR (400 MHz, MeOD) ppm 8.99 (s, 1H), 8.51 (s, 1H), 8.17 (d, J=1.5 Hz, 1H), 8.05 (d, J=9.1 Hz, 1H), 7.77 (dd, J=9.1, 1.8 Hz, 1H), 7.61 (d, J=1.3 Hz, 1H), 7.55 (s, 2H), 7.15 (t, J=2.0 Hz, 1H), 6.87 (d, J=3.5 Hz, 1H), 6.55 (dd, J=3.4, 1.9 Hz, 1H), 4.10 (s, 3H), 4.07 (s, 3H). MS (ES+) m/e 615 [M+H]⁺.

### Example 75

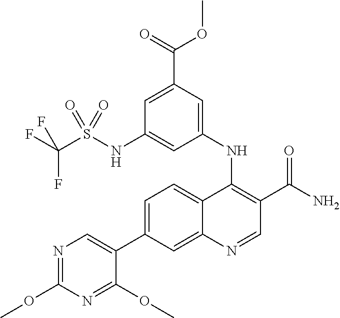 [SMILES: COC(=O)c1cc(Nc2c(C(N)=O)cnc3cc(-c4cnc(OC)nc4OC)ccc23)cc(NS(=O)(=O)C(F)(F)F)c1]

### methyl 3-({3-(aminocarbonyl)-7-[2,4-bis(methyloxy)-5-pyrimidinyl]-4-quinolinyl}amino)-5-{[(trifluoromethyl)sulfonyl]amino}benzoate

### a) methyl 3-nitro-5-{[(trifluoromethyl)sulfonyl]amino}benzoate

To a solution of methyl 3-amino-5-nitrobenzoate (500 mg, 2.55 mmol) and triethylamine (0.533 mL, 3.82 mmol) in dichloromethane (10 mL) cooled at 0° C. was added trifluoromethanesulfonic anhydride (1.292 mL, 7.65 mmol). The mixture was kept stirring for one hour at room temperature and was concentrated under reduced pressure. The residue was dissolved in methanol (10.00 mL), and sodium hydroxide (2.124 mL, 12.74 mmol) was added. The reaction was kept stirring for half hour at room temperature and purified by preparative HPLC (YMC 75×30 mm column, 0.1% TFA in water and 0.1% TFA in acetonitrile) to afford methyl 3-nitro-5-{[(trifluoromethyl)sulfonyl]amino}benzoate (210 mg, 0.640 mmol, 25.1% yield) as an off white solid. ¹H NMR (400 MHz, MeOD) δ ppm 8.63-8.66 (m, 1H), 8.35 (t, J=2.1 Hz, 1H), 8.23-8.27 (m, 1H), 3.99 (s, 3H). MS (ES+) m/e 329[M+H]⁺.

### b) methyl 3-({3-(aminocarbonyl)-7-[2,4-bis(methyloxy)-5-pyrimidinyl]-4-quinolinyl}amino)-5-{[(trifluoromethyl)sulfonyl]amino}benzoate

To a suspension of methyl 3-nitro-5-{[(trifluoromethyl)sulfonyl]amino}benzoate (190 mg, 0.580 mmol) in acetic acid (10 mL) was added wet palladium on carbon (6.17 mg, 0.058 mmol). The reaction was hydrogenated on Parr shaker under 40 psi hydrogen for 3 hours. The catalyst was removed by filtration, and 7-[2,4-bis(methyloxy)-5-pyrimidinyl]-4-chloro-3-quinolinecarboxamide (example 1b, 200 mg, 0.580 mmol) was added to the filtrate. The reaction mixture was kept stirring overnight and was purified by preparative HPLC (YMC 75×30 mm column, 0.1% TFA in water and 0.1% TFA in acetonitrile) to afford methyl 3-({3-(aminocarbonyl)-7-[2,4-bis(methyloxy)-5-pyrimidinyl]-4-quinolinyl}amino)-5-{[(trifluoromethyl)sulfonyl]amino}benzoate (102 mg, 0.142 mmol, 24.40% yield) as a yellow solid. ¹H NMR (400 MHz, MeOD) δ ppm 8.98 (s, 1H), 8.54 (s, 1H), 8.20 (d, J=1.5 Hz, 1H), 8.09 (d, J=9.1 Hz, 1H), 7.81-7.90 (m, 3H), 7.47-7.53 (m, 1H), 4.12 (s, 3H), 4.08 (s, 3H), 3.92 (s, 3H). MS (ES+) m/e 607 [M+H]⁺.

### Example 76

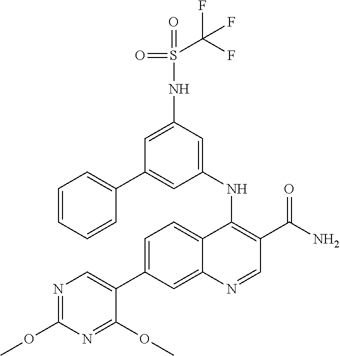 [SMILES: COc1ncc(-c2ccc3c(Nc4cc(NS(=O)(=O)C(F)(F)F)cc(-c5ccccc5)c4)c(C(N)=O)cnc3c2)c(OC)n1]

### 7-[2,4-bis(methyloxy)-5-pyrimidinyl]-4-[(5-{[(trifluoromethyl)sulfonyl]amino}-3-biphenylyl)amino]-3-quinolinecarboxamide, trifluoroacetate salt

### a) 5-nitro-3-biphenylamine

To a suspension of (3-bromo-5-nitrophenyl)amine (example 74a, 1 g, 4.61 mmol), phenylboronic acid (1.124 g, 9.22 mmol) and potassium carbonate (1.274 g, 9.22 mmol) in 1,4-dioxane (12 mL) and water (4.00 mL) was added tetrakis(triphenylphosphine)palladium(0) (0.532 g, 0.461 mmol). The mixture was heated to 100° C. overnight. After cooling, the mixture was diluted with water, acidified with 6N HCl and extracted with ethyl acetate. The organic layer was dried over MgSO₄, filtered, concentrated under reduced pressure and purified via flash chromatography (0-10% methanol in methylene chloride) to afford (5-nitro-3-biphenylyl)amine (560 mg, 2.61 mmol, 56.7% yield) as a brown solid. ¹H NMR (400 MHz, CHLOROFORM-d) δ ppm 7.85 (s, 1H), 7.60 (d, J=7.1 Hz, 2H), 7.39-7.53 (m, 4H), 7.19 (s, 1H). MS (ES+) m/e 215 [M+H]⁺.

### b) 1,1,1-trifluoro-N-(5-nitro-3-biphenylyl)methanesulfonamide

To a solution of 5-nitro-3-biphenylamine (100 mg, 0.467 mmol) in dichloromethane (20 mL) was added triethylamine (0.195 mL, 1.400 mmol) and trifluoromethanesulfonic anhydride (0.118 mL, 0.700 mmol) at 0° C. After the mixture was kept stirring for 2 hours, the reaction was quenched with water. The organic layer was collected, concentrated under reduced pressure and dissolved in methanol (10 mL), then sodium hydroxide (6.0 N in water) (93 mg, 2.334 mmol) added. The mixture was kept stirring for another half hour and quenched with water. The reaction was extracted with methylene chloride. The organic layer was separated, dried over MgSO₄, concentrated under reduced pressure and purified through preparative HPLC (YMC 75×30 mm column, 0.1% TFA in water and 0.1% TFA in acetonitrile) to afford 1,1,1-trifluoro-N-(5-nitro-3-biphenylyl)methanesulfonamide (50 mg, 0.144 mmol, 30.9% yield) as a brown oil. MS (ES+) m/e 347 [M+H]⁺.

### c) 7-[2,4-bis(methyloxy)-5-pyrimidinyl]-4-[(5-{[(trifluoromethyl)sulfonyl]amino}-3-biphenylyl)amino]-3-quinolinecarboxamide, trifluoroacetate salt

To a solution of 1,1,1-trifluoro-N-(5-nitro-3-biphenylyl)methanesulfonamide (50 mg, 0.144 mmol) in acetic acid (15 mL) was added palladium on carbon (1.537 mg, 0.014 mmol). The mixture was hydrogenated via hydrogen balloon at room temperature for 2 hours, then was filtered. 7-[2,4-bis(methyloxy)-5-pyrimidinyl]-4-chloro-3-quinolinecarboxamide (example 1b, 49.8 mg, 0.144 mmol) was added to the filtrate. The mixture was kept stirring at room temperature for 4 days and was purified through preparative HPLC (YMC 75×30 mm column, 0.1% TFA in water and 0.1% TFA in acetonitrile) to afford the title compound (0.014 g, 13%) as a yellow solid. ¹H NMR (400 MHz, MeOD) δ ppm 9.01 (s, 1H), 8.54 (s, 1H), 8.20 (d, J=1.5 Hz, 1H), 8.11 (d, J=9.1 Hz, 1H), 7.82 (dd, J=9.1, 1.8 Hz, 1H), 7.61-7.65 (m, 2H), 7.53-7.55 (m, 1H), 7.52 (t, J=1.8 Hz, 1H), 7.45-7.51 (m, 2H), 7.39-7.45 (m, 1H), 7.32 (t, J=2.0 Hz, 1H), 4.12 (s, 3H), 4.09 (s, 3H). MS (ES+) m/e 625 [M+H]⁺.

### Example 77

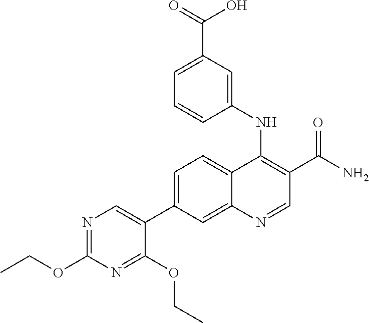 [SMILES: CCOc1ncc(-c2ccc3c(Nc4cccc(C(=O)O)c4)c(C(N)=O)cnc3c2)c(OCC)n1]

### 3-({3-(aminocarbonyl)-7-[2,4-bis(ethyloxy)-5-pyrimidinyl]-4-quinolinyl}amino)benzoic acid

### a) 5-bromo-2,4-diethoxypyrimidine

Sodium ethoxide (21% wt in ethanol) (1.819 mL, 4.87 mmol) was added to a slurry of 5-bromo-2,4-dichloropyrimidine (500 mg, 2.194 mmol) in ethanol (10 mL) and the mixture stirred at reflux under nitrogen for 2 h. After cooling down, the mixture was concentrated under vacuum and washed with water. The obtained solid was washed with water and filtered to obtain the title compound as a granular white solid. ¹H NMR (400 MHz, DMSO-d₆) ppm 8.47 (s, 1H), 4.42 (q, J=7.1 Hz, 2H), 4.31 (q, J=7.1 Hz, 2H), 1.33-1.37 (m, 3H), 1.28-1.33 (m, 3H). MS (ES+) m/e 247/249 [M+H]⁺.

### b) 3-({3-(aminocarbonyl)-7-[2,4-bis(ethyloxy)-5-pyrimidinyl]-4-quinolinyl}amino)benzoic acid

A solution of ethyl 3-{[3-(aminocarbonyl)-7-bromo-4-quinolinyl]amino}benzoate (example 6a, 150 mg, 0.362 mmol), bis(pinacolato)diboron (138 mg, 0.543 mmol), potassium acetate (178 mg, 1.810 mmol), and tetrakis(triphenylphosphine)palladium(0) (18.83 mg, 0.016 mmol) in 1,4-dioxane (10 ml) was heated to 115° C. for 3 h in an oil bath. Upon cooling, the reaction mixture was treated with 5-bromo-2,4-diethoxypyrimidine (89 mg, 0.362 mmol) followed by sodium bicarbonate (sat aq solution) (2 mL, 0.362 mmol). The solution was bubbled through with a nitrogen current. After 5 minutes, the solution was treated with additional tetrakis(triphenylphosphine)palladium(0) (18.83 mg, 0.016 mmol) and heated to 115° C. overnight. Upon cooling the contents were partitioned between water and ethyl acetate. The organic portion was concentrated and the residue treated with ethanol (10 mL) and sodium hydroxide (1N aq solution) (2 mL, 2.000 mmol). After stirring overnight at room temperature, the solution was concentrated, then dissolved into acetonitrile and purified using reverse-phase HPLC (0.1% TFA in acetonitrile and water) to obtain the title compound (0.079 g, 46%) as a pale yellow solid. ¹H NMR (400 MHz, DMSO-d₆) ppm 13.13 (br. s., 1H), 11.52 (br. s., 1H), 8.98 (s, 1H), 8.63 (s, 1H), 8.25 (br. s., 1H), 8.23 (d, J=1.5 Hz, 1H), 8.06-8.18 (m, 1H), 7.68-7.91 (m, 4H), 7.39-7.58 (m, 2H), 4.49 (q, J=7.1 Hz, 2H), 4.42 (q, J=7.1 Hz, 2H), 1.37 (t, J=7.1 Hz, 3H), 1.36 (t, J=7.1 Hz, 3H). MS (ES+) m/e 474 [M+H]⁺.

### Example 78

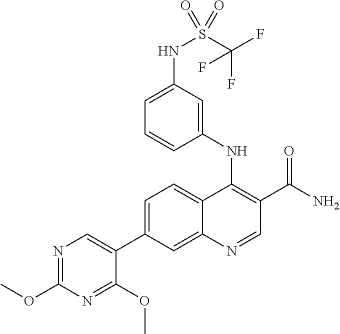 [SMILES: COc1ncc(-c2ccc3c(Nc4cccc(NS(=O)(=O)C(F)(F)F)c4)c(C(N)=O)cnc3c2)c(OC)n1]

### 7-[2,4-bis(methyloxy)-5-pyrimidinyl]-4-[(3-{[(trifluoromethyl)sulfonyl]amino}phenyl)amino]-3-quinolinecarboxamide, trifluoroacetate salt

To a suspension of 7-[2,4-bis(methyloxy)-5-pyrimidinyl]-4-chloro-3-quinolinecarboxamide (example 1b, 50 mg, 0.145 mmol) in acetic acid (10 mL) was added N-(3-aminophenyl)-1,1,1-trifluoromethanesulfonamide (34.8 mg, 0.145 mmol). The reaction mixture was stirred at room temperature overnight and was purified through preparative HPLC (YMC 75×30 mm column, 0.1% TFA in water and 0.1% TFA in acetonitrile) to afford the title compound (26 mg, 0.039 mmol, 27.1% yield) as a yellow solid. ¹H NMR (400 MHz, DMSO-d6) δ ppm 12.03 (br. s., 1H), 9.03 (s, 1H), 8.62 (s, 1H), 8.36 (br. s., 1H), 8.20 (d, J=1.5 Hz, 1H), 7.97 (d, J=9.3 Hz, 1H), 7.91 (br. s., 1H), 7.78 (dd, J=9.1, 1.3 Hz, 1H), 7.35-7.43 (m, 1H), 7.07-7.17 (m, 3H), 4.01 (s, 3H), 3.99 (s, 3H). MS (ES+) m/e 549 [M+H]⁺.

### Example 79

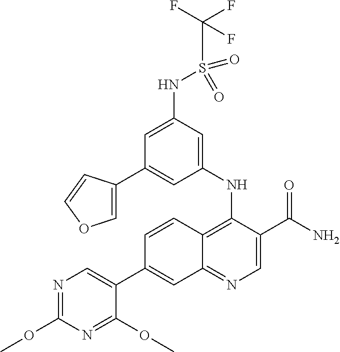 [SMILES: COc1ncc(-c2ccc3c(Nc4cc(NS(=O)(=O)C(F)(F)F)cc(-c5ccoc5)c4)c(C(N)=O)cnc3c2)c(OC)n1]

### 7-[2,4-bis(methyloxy)-5-pyrimidinyl]-4-[(3-(3-furanyl)-5-{[(trifluoromethyl)sulfonyl]amino}phenyl)amino]-3-quinolinecarboxamide

### a) [3-(3-furanyl)-5-nitrophenyl]amine

To a suspension of (3-bromo-5-nitrophenyl)amine (example 74a, 1 g, 4.61 mmol), 3-furanylboronic acid (0.516 g, 4.61 mmol) and potassium carbonate (1.274 g, 9.22 mmol) in 1,4-dioxane (12 mL) and water (4.00 mL) was added tetrakis(triphenylphosphine)palladium(0) (0.532 g, 0.461 mmol). The mixture was heated to 100° C. overnight. After cooling, the mixture was diluted with water, acidified with 6N HCl and extracted with ethyl acetate. The organic layer was dried over MgSO₄, filtered and concentrated under reduced pressure. The crude was purified through flash chromatography (0-100% ethyl acetate in hexanes) to afford [3-(3-furanyl)-5-nitrophenyl]amine (700 mg, 3.43 mmol, 74.4% yield) as a brown solid. ¹H NMR (400 MHz, CHLOROFORM-d) δ ppm 7.79 (s, 1H), 7.71 (s, 1H), 7.51 (s, 1H), 7.40 (s, 1H), 7.06 (s, 1H), 6.70 (s, 1H), 4.06 (br. s., 2H). MS (ES+) m/e 205 [M+H]⁺.

### b) 1,1,1-trifluoro-N-[3-(3-furanyl)-5-nitrophenyl]methanesulfonamide

To a solution of [3-(3-furanyl)-5-nitrophenyl]amine (500 mg, 2.449 mmol) in dichloromethane (20 mL) was added triethylamine (1.024 mL, 7.35 mmol) and trifluoromethanesulfonic anhydride (0.455 mL, 2.69 mmol) at 0° C. After the reaction was kept stirring for 2 hours, it was quenched with water. The organic layer was collected, dried over MgSO₄, filtered, concentrated under reduced pressure and washed with methanol to afford a grey solid. The solid was disolved in dichloromethane and methanol (1:1) and sodium hydroxide (6.0M in water) (0.408 mL, 2.449 mmol) was added. The mixture was kept stirring for another half hour, diluted with water and extracted with dichloromethane. The organic layer was removed. The aqueous layer was acidified and extracted with ethyl acetate. The organic layer was collected, dried over MgSO₄, filtered and concentrated under reduced pressure to afford 1,1,1-trifluoro-N-[3-(3-furanyl)-5-nitrophenyl]methanesulfonamide (250 mg, 0.744 mmol, 30.4% yield) as a brown solid. ¹H NMR (400 MHz, DMSO-d6) δ ppm 8.45 (s, 1H), 8.23 (d, J=1.5 Hz, 1H), 7.92 (t, J=2.0 Hz, 1H), 7.83 (t, J=1.6 Hz, 1H), 7.78 (t, J=1.8 Hz, 1H), 7.05-7.09 (m, 1H). MS (ES+) m/e 337 [M+H]⁺.

### c) 7-[2,4-bis(methyloxy)-5-pyrimidinyl]-4-[(3-(3-furanyl)-5-{[(trifluoromethyl)sulfonyl]amino}phenyl)amino]-3-quinolinecarboxamide

To a solution of 1,1,1-trifluoro-N-[3-(3-furanyl)-5-nitrophenyl]methanesulfonamide (100 mg, 0.297 mmol) in acetic acid (10 mL) was added palladium on carbon (3.16 mg, 0.030 mmol). The mixture was hydrogenated via hydrogen balloon at room temperature for 1 hour, then was filtered. 7-[2,4-bis(methyloxy)-5-pyrimidinyl]-4-chloro-3-quinolinecarboxamide (example 1b, 80 mg, 0.232 mmol) was added to the solution. The mixture was kept stirring overnight. The solvent was removed under reduced pressure. The resulting solid was washed with methanol methylene chloride and ether to afford 7-[2,4-bis(methyloxy)-5-pyrimidinyl]-4-[(3-(3-furanyl)-5-{[(trifluoromethyl)sulfonyl]amino}phenyl)amino]-3-quinolinecarboxamide (75 mg, 0.122 mmol, 52.6% yield) as an off white solid. ¹H NMR (400 MHz, DMSO-d6) δ ppm 12.17 (br. s., 1H), 9.13 (s, 1H), 8.64 (s, 1H), 8.55 (br. s., 1H), 8.40 (s, 1H), 8.18-8.25 (m, 2H), 7.88 (br. s., 1H), 7.85 (dd, J=9.1, 1.5 Hz, 1H), 7.77 (t, J=1.8 Hz, 1H), 7.43 (s, 1H), 7.37 (s, 1H), 7.13 (s, 1H), 6.91 (d, J=1.0 Hz, 1H), 4.02 (s, 3H), 3.99 (s, 3H). MS (ES+) m/e 615 [M+H]⁺.

### Example 80

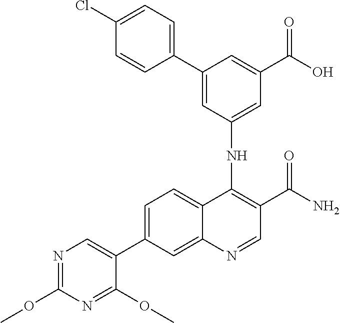 [SMILES: COc1ncc(-c2ccc3c(Nc4cc(C(=O)O)cc(-c5ccc(Cl)cc5)c4)c(C(N)=O)cnc3c2)c(OC)n1]

### 5-({3-(aminocarbonyl)-7-[2,4-bis(methyloxy)-5-pyrimidinyl]-4-quinolinyl}amino)-4′-chloro-3-biphenylcarboxylic acid

### a) 5-amino-4′-chloro-3-biphenylcarboxylic acid

To a solution of 3-amino-5-bromobenzoic acid (100 mg, 0.463 mmol) and (4-chlorophenyl)boronic acid (72.4 mg, 0.463 mmol) in a mixture of 1,4-dioxane (6 mL) and water (2.000 mL), tetrakis(triphenylphosphine)palladium(0) (26.7 mg, 0.023 mmol) and potassium carbonate (128 mg, 0.926 mmol) were added and the mixture stirred at 80° C. in a microwave reactor for 30 mins. After that time, LCMS showed complete conversion to desired product. The reaction mixture was diluted with water, acidified to pH 4 and extracted with ethyl acetate. The organic layer was dried over sodium sulfate and evaporated affording the title compound (70 mg, 0.198 mmol, 42.7% yield). MS (ES+) m/e 248 [M+H]⁺.

### b) 5-({3-(aminocarbonyl)-7-[2,4-bis(methyloxy)-5-pyrimidinyl]-4-quinolinyl}amino)-4′-chloro-3-biphenylcarboxylic acid

5-amino-4′-chloro-3-biphenylcarboxylic acid (57.5 mg, 0.232 mmol) and 7-[2,4-bis(methyloxy)-5-pyrimidinyl]-4-chloro-3-quinolinecarboxamide (example 1b, 80 mg, 0.232 mmol) were suspended in acetic acid (7 mL) and stirred at RT overnight. After that time, LCMS indicated complete conversion of starting material. The reaction mixture was diluted with water and the obtained precipitate was filtered and dried affording the title compound (30 mg, 0.050 mmol, 21.63% yield). MS (ES+) m/e 556 [M+H]⁺.

### Example 81

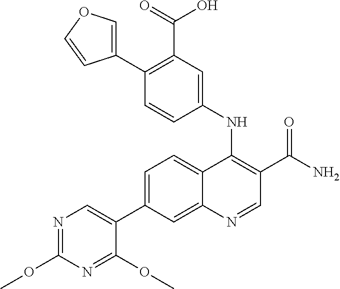 [SMILES: COc1ncc(-c2ccc3c(Nc4ccc(-c5ccoc5)c(C(=O)O)c4)c(C(N)=O)cnc3c2)c(OC)n1]

### 5-({3-(aminocarbonyl)-7-[2,4-bis(methyloxy)-5-pyrimidinyl]-4-quinolinyl}amino)-2-(3-furanyl)benzoic acid, trifluoroacetate salt

### a) methyl 5-amino-2-(3-furanyl)benzoate

To a suspension of methyl 5-amino-2-bromobenzoate (200 mg, 0.869 mmol), 3-furanylboronic acid (97 mg, 0.869 mmol) and potassium carbonate (240 mg, 1.739 mmol) in 1,4-dioxane (12 mL) and water (4.00 mL) was added tetrakis(triphenylphosphine)palladium(0) (50.2 mg, 0.043 mmol). The mixture was heated to 100° C. overnight. After cooling, the mixture was diluted with water, acidified with 6N HCl and extracted with ethyl acetate. The organic layer was dried over MgSO₄, filtered and concentrated under reduced pressure. The precipitate was collected and washed with methylene chloride and ether to afford the title compound (100 mg, 0.460 mmol, 53.0% yield) as a brown oil. ¹H NMR (400 MHz, CHLOROFORM-d) δ ppm 7.48 (s, 1H), 7.42-7.44 (m, 1H), 7.18 (d, J=8.1 Hz, 1H), 7.07 (d, J=2.8 Hz, 1H), 6.79 (dd, J=8.3, 2.5 Hz, 1H), 6.44 (s, 1H), 3.80 (br. s., 2H), 3.78 (s, 3H). MS (ES+) m/e 218 [M+H]⁺.

### b) methyl 5-({3-(aminocarbonyl)-7-[2,4-bis(methyloxy)-5-pyrimidinyl]-4-quinolinyl}amino)-2-(3-furanyl)benzoate trifluoroacetate salt

To a suspension of 7-[2,4-bis(methyloxy)-5-pyrimidinyl]-4-chloro-3-quinolinecarboxamide (example 1b, 150 mg, 0.435 mmol) in acetic acid (10 mL) was added methyl 5-amino-2-(3-furanyl)benzoate (95 mg, 0.435 mmol). The reaction mixture was stirred room temperature overnight and was purified through preparative HPLC (YMC 75×30 mm column, 0.1% TFA in water and 0.1% TFA in acetonitrile) to afford the title compound (100 mg, 0.156 mmol, 35.9% yield) as a yellow solid. ¹H NMR (400 MHz, DMSO-d6) δ ppm 8.16 (s, 1H), 7.75 (s, 1H), 7.38 (d, J=1.5 Hz, 1H), 7.33 (d, J=9.3 Hz, 1H), 7.04 (dd, J=9.1, 1.8 Hz, 1H),6.93 (s, 1H), 6.88 (d, J=2.3 Hz, 1H), 6.76-6.81 (m, 2H), 6.67-6.72 (m, 1H), 5.77 (d, J=1.0 Hz, 1H), 3.33 (s, 3H), 3.29 (s, 3H), 3.00 (s, 3H). MS (ES+) m/e 526[M+H]⁺.

### c) 5-({3-(aminocarbonyl)-7-[2,4-bis(methyloxy)-5-pyrimidinyl]-4-quinolinyl}amino)-2-(3-furanyl)benzoic acid, trifluoroacetate salt

To methyl 5-({3-(aminocarbonyl)-7-[2,4-bis(methyloxy)-5-pyrimidinyl]-4-quinolinyl}amino)-2-(3-furanyl)benzoate (50 mg, 0.095 mmol) in methanol (15 mL) was added sodium hydroxide (6.0N in water) (0.016 mL, 0.095 mmol). The reaction mixture was stirred at 50° C. overnight. After cooling, the reaction was purified through preparative HPLC (YMC 75×30 mm column, 0.1% TFA in water and 0.1% TFA in acetonitrile) to afford the title compound (15 mg, 0.024 mmol, 25.2% yield) as a yellow solid. ¹H NMR (400 MHz, DMSO-d6) δ ppm 13.12 (br. s., 1H), 11.33 (br. s., 1H), 9.02 (s, 1H), 8.64 (s, 1H), 8.31 (br. s., 1H), 8.22 (d, J=1.0 Hz, 1H),8.11 (d, J=8.3 Hz, 1H), 7.80-7.89 (m, 3H), 7.72 (s, 1H), 7.46 (d, J=8.6 Hz, 2H), 7.30 (d, J=8.1 Hz, 1H), 6.64 (s, 1H), 4.02 (s, 3H), 3.99 (s, 3H). MS (ES+) m/e 512 [M+H]⁺.

### Example 82

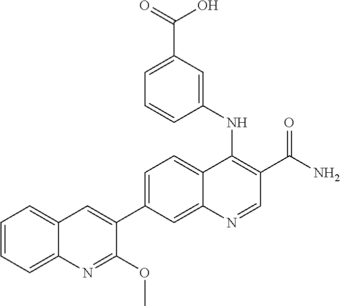 [SMILES: COc1nc2ccccc2cc1-c1ccc2c(Nc3cccc(C(=O)O)c3)c(C(N)=O)cnc2c1]

### 3-{[3′-(aminocarbonyl)-2-(methyloxy)-3,7′-biquinolin-4′-yl]amino}benzoic acid

A solution of ethyl 3-{[3-(aminocarbonyl)-7-bromo-4-quinolinyl]amino}benzoate (example 6a, 150 mg, 0.362 mmol), 2-methoxy-3-quinolineboronic acid (110 mg, 0.543 mmol), sodium bicarbonate (sat aq) (2 mL, 0.362 mmol), and tetrakis(triphenylphosphine)palladium(0) (18.83 mg, 0.016 mmol) in 1,4-dioxane (2.0 ml) was heated to 100° C. for 20 min. in an oil bath overnight. Upon cooling, the reaction mixture was concentrated in vacuo. The residue was purified via flash column chromatography (0-100% ethyl acetate in hexanes) to obtain the intermediate ester (MS (ES+) m/e 493 [M+H]⁺, 80% by LCMS) contaminated with triphenylphosphine oxide

The column was then flushed with 10% methanol in ethyl acetate to obtain a mixture of the title compound (MS (ES+) m/e 465 [M+H]+) and the intermediate ester. The two fractions were collected and dissolved into ethanol (5 mL), then treated wiith sodium hydroxide (1N aq solution) (2 mL, 2.000 mmol) at room temperature. After stirring for 3 h the mixture was concentrated in vacuo and dissolved into water. A precipitate formed upon adding acetonitrile, therefore the solid was filtered. The solid was dispersed in water, neutralized using 1M aq hydrochloric acid and filtered to obtain the title compound as a pale yellow solid. ¹H NMR (400 MHz, DMSO-d₆) ppm 12.98 (br. s., 1H), 10.49 (br. s., 1H), 9.05 (s, 1H), 8.49 (s, 1H), 8.29 (br. s., 1H), 8.28 (d, J=1.8 Hz, 1H), 7.99 (dd, J=8.2, 0.9 Hz, 1H), 7.91 (d, J=8.8 Hz, 1H), 7.84 (d, J=8.1 Hz, 1H), 7.68-7.77 (m, 3H), 7.61 (d, J=8.1 Hz, 1H), 7.56 (s, 1H), 7.49 (ddd, J=8.1, 6.9, 1.0 Hz, 1H), 7.41 (t, J=7.8 Hz, 1H), 7.27 (d, J=7.6 Hz, 1H), 4.05 (s, 3H). MS (ES+) m/e 465 [M+H]⁺.

### Example 83

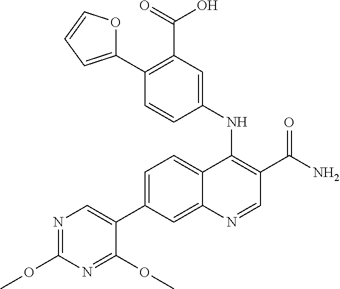 [SMILES: COc1ncc(-c2ccc3c(Nc4ccc(-c5ccco5)c(C(=O)O)c4)c(C(N)=O)cnc3c2)c(OC)n1]

### 5-({3-(aminocarbonyl)-7-[2,4-bis(methyloxy)-5-pyrimidinyl]-4-quinolinyl}amino)-2-(2-furanyl)benzoic acid

### a) methyl 5-amino-2-(2-furanyl)benzoate

To a suspension of methyl 5-amino-2-bromobenzoate (250 mg, 1.087 mmol), 2-furanylboronic acid (122 mg, 1.087 mmol) and potassium carbonate (300 mg, 2.173 mmol) in 1,4-dioxane (12 mL) and water (4.00 mL) was added tetrakis(triphenylphosphine)palladium(0) (62.8 mg, 0.054 mmol). The mixture was heated to 100° C. overnight. After cooling, the mixture was diluted with water, acidified with 6N HCl and extracted with ethyl acetate. The organic layer was dried over MgSO₄, filtered and concentrated under reduced pressure. The precipitate was collected and washed with methylene chloride and ether to afford methyl 5-amino-2-(2-furanyl)benzoate (135 mg, 0.621 mmol, 57.2% yield) as a brown oil. ¹H NMR (400 MHz, CHLOROFORM-d) δppm 7.44 (d, J=1.0 Hz, 1H), 7.42 (d, J=8.3 Hz, 1H), 7.03 (d, J=2.5 Hz, 1H), 6.86 (dd, J=8.5, 2.4 Hz, 1H), 6.46 (dd, J=3.3, 1.8 Hz, 1H), 6.40-6.43 (m, 1H), 3.83 (s, 3H). MS (ES+) m/e 218 [M+H]⁺.

### b) methyl 5-({3-(aminocarbonyl)-7-[2,4-bis(methyloxy)-5-pyrimidinyl]-4-quinolinyl}amino)-2-(2-furanyl)benzoate trifluoroacetate

To a suspension of 7-[2,4-bis(methyloxy)-5-pyrimidinyl]-4-chloro-3-quinolinecarboxamide (example 1b, 200 mg, 0.580 mmol) in acetic acid (10 mL) was added methyl 5-amino-2-(2-furanyl)benzoate (126 mg, 0.580 mmol). The reaction mixture was stirred at room temperature overnight. The reaction was purified through preparative HPLC (YMC 75×30 mm column, 0.1% TFA in water and 0.1% TFA in acetonitrile) to afford the title compound (105 mg, 0.164 mmol, 28.3% yield) as a yellow solid. ¹H NMR (400 MHz, MeOD) δ ppm 8.98 (s, 1H), 8.56 (s, 1H), 8.20 (d, J=1.5 Hz, 1H), 8.14 (d, J=9.1 Hz, 1H), 7.86 (dd, J=9.1, 1.8 Hz, 1H), 7.79 (d, J=8.6 Hz, 1H), 7.64 (d, J=1.0 Hz, 1H), 7.59 (d, J=2.3 Hz, 1H), 7.51 (dd, J=8.3, 2.3 Hz, 1H), 6.74 (d, J=3.5 Hz, 1H), 6.58 (dd, J=3.4, 1.9 Hz, 1H), 4.13 (s, 3H), 4.09 (s, 3H), 3.85 (s, 3H). MS (ES+) m/e 526 [M+H]⁺.

### c) 5-({3-(aminocarbonyl)-7-[2,4-bis(methyloxy)-5-pyrimidinyl]-4-quinolinyl}amino)-2-(2-furanyl)benzoic acid

To a solution of methyl 5-({3-(aminocarbonyl)-7-[2,4-bis(methyloxy)-5-pyrimidinyl]-4-quinolinyl}amino)-2-(2-furanyl)benzoate (50 mg, 0.095 mmol) in methanol (5 ml) was added sodium hydroxide (6.0 N in water) (0.095 ml, 0.571 mmol). The reaction mixture was stirred at 60° C. overnight. After cooling, the mixture was purified through preparative HPLC (YMC 75×30 mm column, 0.1% TFA in water and 0.1% TFA in acetonitrile) to afford the title compound (25 mg, 0.049 mmol, 51.4% yield) as a brown solid. ¹H NMR (400 MHz, DMSO-d6) δ ppm 13.18 (br. s., 1H), 11.54 (br. s., 1H), 9.02 (s, 1H), 8.65 (s, 1H), 8.32 (br. s., 1H), 8.25 (d, J=1.5 Hz, 1H),8.18 (d, J=9.1 Hz, 1H), 8.14-8.21 (m, 1H), 7.83-7.92 (m, 2H), 7.77 (d, J=1.0 Hz, 1H), 7.67 (d, J=8.6 Hz, 1H), 7.48 (d, J=2.0 Hz, 1H), 7.37 (dd, J=8.5, 1.9 Hz, 1H), 6.78 (d, J=3.0 Hz, 1H), 6.61 (dd, J=3.4, 1.9 Hz, 1H), 4.02 (s, 3H), 3.99 (s, 3H). MS (ES+) m/e 512 [M+H]⁺.

### Example 84

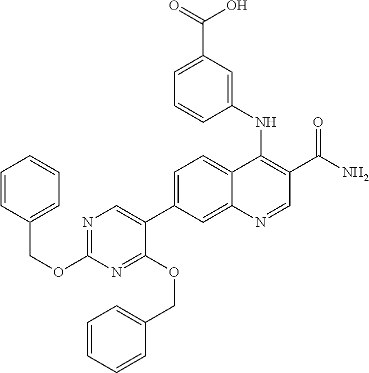 [SMILES: NC(=O)c1cnc2cc(-c3cnc(OCc4ccccc4)nc3OCc3ccccc3)ccc2c1Nc1cccc(C(=O)O)c1]

### 3-[(3-(aminocarbonyl)-7-{2,4-bis[(phenylmethyl)oxy]-5-pyrimidinyl}-4-quinolinyl)amino]benzoic acid

A solution of ethyl 3-{[3-(aminocarbonyl)-7-bromo-4-quinolinyl]amino}benzoate (example 6a, 100 mg, 0.241 mmol), 2,4-bis(benzyloxy)pyrimidine-5-boronic acid (122 mg, 0.362 mmol) and tetrakis(triphenylphosphine)palladium(0) (12.55 mg, 10.86 μmol) in 1,4-dioxane (5 ml) and sodium bicarbonate (saturated aqueous solution) (2 mL, 0.241 mmol) was heated to 115° C. in an oil bath overnight. Upon cooling, the reaction mixture was diluted with ethyl acetate and washed with water. The organic portion was dried over sodium sulfate and concentrated. A portion of the residue was purified using flash chromatography (0-100% ethyl acetate in hexanes) to obtain the intermediate ester as a pale yellow solid. The rest of the material was treated with sodium hydroxide (1N aq solution) (1 mL, 1.000 mmol) as a solution in ethanol (5 mL). After stirring at room temperature overnight, the contents were purified using reverse phase HPLC (0.1% TFA in acetonitrile and water) to obtain the title compound (0.004 g, 3%) as a yellow solid. ¹H NMR (400 MHz, DMSO-d₆) ppm 13.16 (br. s., 1H), 11.49 (s, 1H), 8.96 (s, 1H), 8.67 (s, 1H), 8.23 (br. s., 1H), 8.21 (d, J=1.5 Hz, 1H), 8.07-8.18 (m, 1H), 7.68-7.93 (m, 4H), 7.43-7.52 (m, 6H), 7.31-7.42 (m, 5H), 5.53 (s, 2H), 5.48 (s, 2H). MS (ES+) m/e 598 [M+H]⁺.

### Example 85

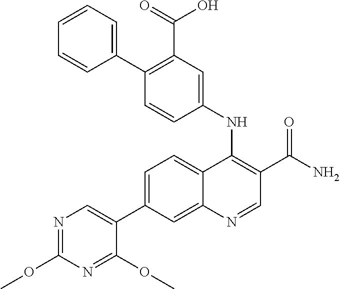 [SMILES: COc1ncc(-c2ccc3c(Nc4ccc(-c5ccccc5)c(C(=O)O)c4)c(C(N)=O)cnc3c2)c(OC)n1]

### 4-({3-(aminocarbonyl)-7-[2,4-bis(methyloxy)-5-pyrimidinyl]-4-quinolinyl}amino)-2-biphenylcarboxylic acid, trifluoroacetate salt

### a) methyl 4-amino-2-biphenylcarboxylate

To a suspension of methyl 5-amino-2-bromobenzoate (300 mg, 1.304 mmol), phenylboronic acid (175 mg, 1.434 mmol) and potassium carbonate (360 mg, 2.61 mmol) in 1,4-dioxane (12 mL) and water (4.00 mL) was added tetrakis(triphenylphosphine)palladium(0) (75 mg, 0.065 mmol). The mixture was heated to 100° C. overnight. After cooling, the mixture was diluted with water, acidified with 6N HCl and extracted with ethyl acetate. The organic layer was dried over MgSO₄, filtered and concentrated under reduced pressure. The precipitate was collected and washed with methylene chloride and ether to afford methyl 4-amino-2-biphenylcarboxylate (250 mg, 1.100 mmol, 84% yield) as a brown solid. ¹H NMR (400 MHz, CHLOROFORM-d) δ ppm 7.36-7.42 (m, 2H), 7.28-7.35 (m, 3H), 7.20 (d, J=8.3 Hz, 1H), 7.14 (d, J=2.3 Hz, 1H), 6.85 (dd, J=8.3, 2.5 Hz, 1H), 3.64 (s, 3H). MS (ES+) m/e 228 [M+H]⁺.

### b) methyl 4-({3-(aminocarbonyl)-7-[2,4-bis(methyloxy)-5-pyrimidinyl]-4-quinolinyl}amino)-2-biphenylcarboxylate

To a suspension of 7-[2,4-bis(methyloxy)-5-pyrimidinyl]-4-chloro-3-quinolinecarboxamide (example 1b, 250 mg, 0.725 mmol) in acetic acid (10 mL) was added methyl 4-amino-2-biphenylcarboxylate (165 mg, 0.725 mmol). The reaction mixture was stirred at room temperature overnight. The precipitate was collected, washed with ether and dried under reduced pressure to afford methyl 4-({3-(aminocarbonyl)-7-[2,4-bis(methyloxy)-5-pyrimidinyl]-4-quinolinyl}amino)-2-biphenylcarboxylate (145 mg, 0.271 mmol, 37.3% yield) as an off white solid. ¹H NMR (400 MHz, DMSO-d6) δ ppm 11.80 (br. s., 1H), 9.05 (s, 1H), 8.68 (s, 1H), 8.36 (br. s., 1H), 8.28-8.33 (m, 2H), 7.94 (dd, J=9.1, 1.3 Hz, 1H), 7.87 (br. s., 1H), 7.69 (s, 1H), 7.43-7.53 (m, 4H), 7.37-7.42 (m, 1H), 7.31-7.35 (m, 2H), 4.03 (s, 3H), 4.00 (s, 3H), 3.60 (s, 3H). MS (ES+) m/e 536 [M+H]⁺.

### c) 4-({3-(aminocarbonyl)-7-[2,4-bis(methyloxy)-5-pyrimidinyl]-4-quinolinyl}amino)-2-biphenylcarboxylic acid, trifluoroacetate salt

To a suspension of methyl 4-({3-(aminocarbonyl)-7-[2,4-bis(methyloxy)-5-pyrimidinyl]-4-quinolinyl}amino)-2-biphenylcarboxylate (60 mg, 0.112 mmol) in methanol (10 mL) was added sodium hydroxide (6.0 N in water) (0.093 mL, 0.560 mmol). The reaction mixture was stirred at 60° C. for overnight. After cooling, the reaction was purified through preparative HPLC (YMC 75×30 mm column, 0.1% TFA in water and 0.1% TFA in acetonitrile) to afford the title compound (20 mg, 0.031 mmol, 28.1% yield) as an off white solid. ¹H NMR (400 MHz, MeOD) δ ppm 8.98 (s, 1H), 8.57 (s, 1H), 8.20 (d, J=1.8 Hz, 1H), 8.18 (d, J=9.1 Hz, 1H), 7.88 (dd, J=9.1, 1.8 Hz, 1H), 7.81 (d, J=2.0 Hz, 1H), 7.55-7.58 (m, 1H), 7.50-7.54 (m, 1H), 7.37-7.46 (m, 5H), 4.14 (s, 3H), 4.10 (s, 3H). MS (ES+) m/e 522 [M+H]⁺.

### Example 86

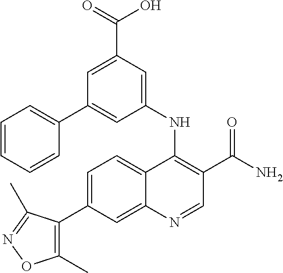 [SMILES: Cc1noc(C)c1-c1ccc2c(Nc3cc(C(=O)O)cc(-c4ccccc4)c3)c(C(N)=O)cnc2c1]

### 5-{[3-(aminocarbonyl)-7-(3,5-dimethyl-4-isoxazolyl)-4-quinolinyl]amino}-3-biphenylcarboxylic acid, trifluoroacetate salt

### a) 4-chloro-7-(3,5-dimethyl-4-isoxazolyl)-3-quinolinecarboxamide

To a mixture of 7-bromo-4-chloro-3-quinolinecarboxamide (example 1a, 2 g, 7.00 mmol), (3,5-dimethyl-4-isoxazolyl)boronic acid (1.974 g, 14.01 mmol) and tetrakis(triphenylphosphine)palladium(0) (0.405 g, 0.350 mmol) in 1,4-dioxane (12 mL) and water (4.00 mL) was added potassium carbonate (1.936 g, 14.01 mmol). The mixture was heated at 80° C. overnight. After cooling, the reaction was quenched with water and acidified with 1N HCl. The precipitate was collected, washed with water and ether, and dried in the air to afford the title compound (1.5 g, 4.97 mmol, 71.0% yield) as a yellow solid. ¹H NMR (400 MHz, DMSO-d6) δ ppm 8.92 (s, 1H), 8.37 (d, J=8.6 Hz, 1H), 8.21 (br. s., 1H), 8.13 (s, 1H), 8.00 (br. s., 1H), 7.87 (dd, J=8.7, 1.6 Hz, 1H), 2.51 (s, 3H), 2.33 (s, 3H). MS (ES+) m/e 302 [M+H]⁺.

### b) 5-amino-3-biphenylcarboxylic acid

To a suspension of 3-amino-5-bromobenzoic acid (300 mg, 1.389 mmol), phenylboronic acid (339 mg, 2.78 mmol) and potassium carbonate (384 mg, 2.78 mmol) in 1,4-dioxane (12 mL) and water (4.00 mL) was added tetrakis(triphenylphosphine)palladium(0) (80 mg, 0.069 mmol). The mixture was heated to 100° C. overnight. After cooling, the mixture was diluted with water, acidified with 6N hydrochloric acid and extracted with ethyl acetate. The organic layer was dried over MgSO₄, filtered and concentrated under reduced pressure. The precipitate was collected and washed with methylene chloride and ether to afford 5-amino-3-biphenylcarboxylic acid (150 mg, 0.703 mmol, 50.7% yield) as an off-white solid. ¹H NMR (400 MHz, DMSO-d6) δ ppm 12.72 (br. s., 1H), 7.54-7.59 (m, 2H), 7.42-7.49 (m, 2H), 7.33-7.38 (m, 1H), 7.33 (t, J=1.5 Hz, 1H),7.17-7.20 (m, 1H), 7.04 (t, J=1.9 Hz, 1H), 5.46 (br. s., 2H). MS (ES+) m/e 214 [M+H]⁺.

### c) 5-{[3-(aminocarbonyl)-7-(3,5-dimethyl-4-isoxazolyl)-4-quinolinyl]amino}-3-biphenylcarboxylic acid, trifluoroacetate salt

To a suspension of 4-chloro-7-(3,5-dimethyl-4-isoxazolyl)-3-quinolinecarboxamide (50 mg, 0.166 mmol) in acetic acid (10 mL) was added 5-amino-3-biphenylcarboxylic acid (35.3 mg, 0.166 mmol). The reaction mixture was stirred at room temperature overnight, then quenched with water. The precipitate was collected and dried in the air to afford the title compound (25 mg, 0.042 mmol, 25.5% yield) as a yellow solid. ¹H NMR (400 MHz, DMSO-d6) δ ppm 11.74 (br. s., 1H), 9.02 (s, 1H), 8.44 (d, J=8.8 Hz, 1H), 8.27 (br. s., 1H), 8.08-8.11 (m, 1H), 8.06 (d, J=1.5 Hz, 1H), 7.86 (d, J=1.5 Hz, 1H), 7.75-7.83 (m, 3H), 7.69-7.74 (m, 2H), 7.46-7.53 (m, 2H), 7.39-7.45 (m, 1H), 2.54 (s, 3H), 2.35 (s, 3H). MS (ES+) m/e 479 [M+H]⁺.

### Example 87

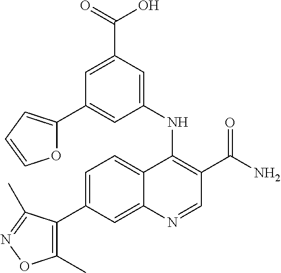 [SMILES: Cc1noc(C)c1-c1ccc2c(Nc3cc(C(=O)O)cc(-c4ccco4)c3)c(C(N)=O)cnc2c1]

### 3-{[3-(aminocarbonyl)-7-(3,5-dimethyl-4-isoxazolyl)-4-quinolinyl]amino}-5-(2-furanyl)benzoic acid, trifluoroacetate salt

To a suspension of 4-chloro-7-(3,5-dimethyl-4-isoxazolyl)-3-quinolinecarboxamide (example 86a, 50 mg, 0.166 mmol) in acetic acid (10 mL) was added 3-amino-5-(2-furanyl)benzoic acid (example 51a, 33.7 mg, 0.166 mmol). The reaction mixture was stirred at room temperature overnight and was purified through preparative HPLC (YMC 75×30 mm column, 0.1% TFA in water and 0.1% TFA in acetonitrile) to afford the title compound (16 mg, 0.027 mmol, 16.58% yield) as a yellow solid. ¹H NMR (400 MHz, DMSO-d6) δ ppm 8.97 (s, 1H), 8.29 (d, J=1.5 Hz, 1H), 8.21 (d, J=8.8 Hz, 1H), 7.90 (d, J=1.5 Hz, 1H), 7.86 (t, J=1.8 Hz, 1H), 7.83 (d, J=1.5 Hz, 1H), 7.57-7.65 (m, 2H), 6.92 (d, J=3.3 Hz, 1H), 6.56 (dd, J=3.5, 1.8 Hz, 1H), 2.52 (s, 3H), 2.35 (s, 3H). MS (ES+) m/e 469 [M+H]⁺.

### Example 88

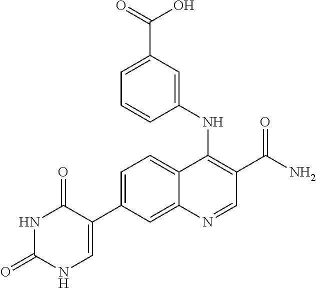 [SMILES: NC(=O)c1cnc2cc(-c3c[nH]c(=O)[nH]c3=O)ccc2c1Nc1cccc(C(=O)O)c1]

### 3-{[3-(aminocarbonyl)-7-(2,4-dioxo-1,2,3,4-tetrahydro-5-pyrimidinyl)-4-quinolinyl]amino}benzoic acid, trifluoroacetate salt

### a) 3-{[3-(aminocarbonyl)-7-bromo-4-quinolinyl]amino}benzoic acid

To a suspension of ethyl 3-{[3-(aminocarbonyl)-7-bromo-4-quinolinyl]amino}benzoate (example 6a, 1 g, 2.414 mmol) in ethanol (20.0 mL) was added 1N aqueous sodium hydroxide (10 ml, 10.00 mmol). After stirring overnight at ambient temperature, the reaction was concentrated to an aqueous residue, which was neutralized using 6N aqueous hydrochloric acid and the resulting precipitate was filtered, washed with water, and dried in vacuo to obtain the title compound (822 mg, 2.128 mmol, 88% yield) as a yellow solid. MS (ES+) m/e 386/388 [M+H]⁺.

### b) 3-{[3-(aminocarbonyl)-7-(2,4-dioxo-1,2,3,4-tetrahydro-5-pyrimidinyl)-4-quinolinyl]amino}benzoic acid, trifluoroacetate salt

A solution of 3-{[3-(aminocarbonyl)-7-bromo-4-quinolinyl]amino}benzoic acid (100 mg, 0.259 mmol), uracil-5-boronic acid (117 mg, 0.751 mmol), potassium carbonate (179 mg, 1.295 mmol), and tetrakis(triphenylphosphine)palladium(0) (30 mg, 0.026 mmol) in 1,4-dioxane (2.0 ml) and water (0.667 ml) was heated to 100° C. for 20 min. in a Biotage Initiator® microwave synthesizer. Upon cooling, the reaction mixture was concentrated to the aqueous residue and purified using Reverse Phase HPLC (0.1% TFA in water and acetonitrile) to obtain the title compound (0.017 g, 12%) as an off white solid. ¹H NMR (400 MHz, DMSO-d₆) ppm 13.18 (br. s., 1H), 11.70 (br. s., 1H), 11.61 (d, J=6.1 Hz, 1H), 11.50 (d, J=1.5 Hz, 1H), 8.93 (s, 1H), 8.55 (d, J=1.5 Hz, 1H), 8.28 (br. s., 1H), 8.11 (d, J=6.3 Hz, 1H), 8.03 (d, J=9.1 Hz, 1H), 7.79-7.87 (m, 3H), 7.77 (br. s., 1H), 7.46-7.56 (m, 2H). MS (ES+) m/e 418 [M+H]⁺.

### Example 89

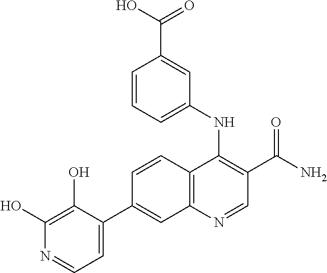 [SMILES: NC(=O)c1cnc2cc(-c3ccnc(O)c3O)ccc2c1Nc1cccc(C(=O)O)c1]

### 3-{[3-(aminocarbonyl)-7-(2,3-dihydroxy-4-pyridinyl)-4-quinolinyl]amino}benzoic acid

To a suspension of ethyl 3-({3-(aminocarbonyl)-7-[2,3-bis(methyloxy)-4-pyridinyl]-4-quinolinyl}amino)benzoate (example 102a, 196 mg, 0.415 mmol) in dichloromethane (8.4 mL) cooled at 00° C., 1M boron tribromide in dichloromethane (2.9 mL, 2.90 mmol) was added dropwise and allowed to equilibrate to room temperature. The reaction mixture was stirred at rt for 4.5 h when according to UPLC-MS beside some small quantity of starting material major product was acid and minor product was ester. The reaction mixture was portionwise poured into ice-water and the yellowish precipitate was filtered, washed with water and dichloromethane and dried to afford the crude product, which was purified by reverse phase HPLC (ODS, aqueous 10 mM ammonium bicarbonate/acetonitrile) to obtain the title compound (0.082 g, 47%). MS (ES+) m/e 417 [M+H]⁺.

### Example 90

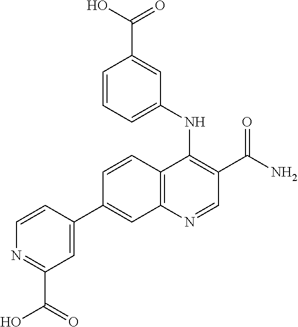 [SMILES: NC(=O)c1cnc2cc(-c3ccnc(C(=O)O)c3)ccc2c1Nc1cccc(C(=O)O)c1]

### 4-{3-(aminocarbonyl)-4-[(3-carboxyphenyl)amino]-7-quinolinyl}-2-pyridinecarboxylic acid

### a) ethyl 3-{[3-(aminocarbonyl)-7-(2-cyano-4-pyridinyl)-4-quinolinyl]amino}benzoate

A solution of ethyl 3-{[3-(aminocarbonyl)-7-bromo-4-quinolinyl]amino}benzoate (example 6a, 150 mg, 0.362 mmol), 2-cyanopyridine-4-boronic acid, pinacol ester (92 mg, 0.398 mmol) and potassium carbonate (150 mg, 1.086 mmol) in 1,4-dioxane (4.5 mL) and water (1.5 mL) was degassed by bubbling with Ar for few min. Tetrakis(triphenylphosphine)palladium(0) (20.92 mg, 0.018 mmol) was added. The reaction mixture was sealed and heated in a Biotage Initiator microwave reactor at 100° C. for 35 min. According to UPLC-MS, the conversion was incomplete. Additional quantities of 2-cyanopyridine-4-boronic acid, pinacol ester (50 mg, 0.217 mmol) and tetrakis(triphenylphosphine)palladium(0) (10 mg, 8.65 μmol) were added. After additional heating in the microwave at 100° C. for 30 min, then at 120° C. for 20 min, the mixture was cooled, diluted with water and extracted two times with EtOAc. The collected organic layers were washed with water, dried over Na₂SO₄ and evaporated to obtain crude product, which was purified by silica gel column chromatography (dichloromethane/methanol, 15:1 Supelco, 20 g) to afford the title compound (55.7 mg, 34%), MS (ES+) m/e 438 [M+H]⁺; and ethyl 3-({3-(aminocarbonyl)-7-[2-(aminocarbonyl)-4-pyridinyl]-4-quinolinyl}amino)benzoate (17 mg, 90% pure, 9%), MS (ES+) m/e 456 [M+H]⁺.

### b) 4-{3-(aminocarbonyl)-4-[(3-carboxyphenyl)amino]-7-quinolinyl}-2-pyridinecarboxylic acid

To a suspension of ethyl 3-{[3-(aminocarbonyl)-7-(2-cyano-4-pyridinyl)-4-quinolinyl]amino}benzoate (50 mg, 0.114 mmol) in ethanol (1.7 mL), 1M sodium hydroxide (1.7 mL, 1.700 mmol) was added. (Note: the colour of the reaction mixture changed from intensive yellow to intensive orange-red after adding of NaOH). The reaction mixture was stirred at RT for 24 h, then at 80° C. for 4 h. The reaction mixture was concentrated to an aqueous residue which was then neutralized with 6M HCl. The resulting precipitate was filtered, washed with water and dried, then purified by reverse phase HPLC (ODS, aqueous 10 mM ammonium bicarbonate/acetonitrile) to obtain the title compound (7.7 mg, 15%). MS (ES+) m/e 429 [M+H]⁺.

### Example 91

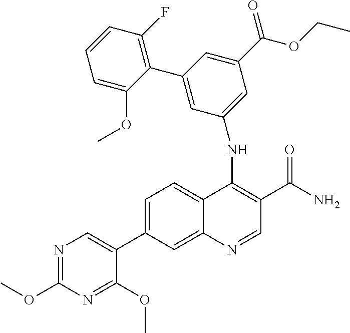 [SMILES: CCOC(=O)c1cc(Nc2c(C(N)=O)cnc3cc(-c4cnc(OC)nc4OC)ccc23)cc(-c2c(F)cccc2OC)c1]

### ethyl 5-({3-(aminocarbonyl)-7-[2,4-bis(methyloxy)-5-pyrimidinyl]-4-quinolinyl}amino)-2′-fluoro-6′-(methyloxy)-3-biphenylcarboxylate

### a) ethyl 5-amino-2′-fluoro-6′-(methyloxy)-3-biphenylcarboxylate

Ethyl 3-amino-5-bromobenzoate (100 mg, 0.410 mmol), [2-fluoro-6-(methyloxy)phenyl]boronic acid (84 mg, 0.492 mmol), tetrakis(triphenylphosphine)palladium(0) (23.67 mg, 0.020 mmol) and potassium carbonate (56.6 mg, 0.410 mmol) were dissolved in 1,4-dioxane (4 mL) and water (1.333 mL). The reaction mixture was heated in a microwave reactor at 180° C. for 30 minutes. After cooling, water (10 ml) was added and reaction mixture extracted with ethyl acetate (3×10 mL). The organic layers were combined, filtered through a phase separator and evaporated under reduced pressure to obtain the title compound (0.112 g, 94%) as a yellow-green solid. MS (ES+) m/e 290 [M+H]⁺.

### b) ethyl 5-({3-(aminocarbonyl)-7-[2,4-bis(methyloxy)-5-pyrimidinyl]-4-quinolinyl}amino)-2′-fluoro-6′-(methyloxy)-3-biphenylcarboxylate

To a suspension of 7-[2,4-bis(methyloxy)-5-pyrimidinyl]-4-chloro-3-quinolinecarboxamide (example 1b, 112 mg, 0.324 mmol) in acetic acid (5 ml, 87 mmol), ethyl 5-amino-2′-fluoro-6′-(methyloxy)-3-biphenylcarboxylate (78 mg, 0.270 mmol) was added. The reaction mixture was stirred at room temperature overnight. Ethyl acetate (10 ml) and water (10 ml) were added to the clear yellowish reaction mixture and pH was adjusted to 8 with 2N aqueous sodium hydroxide. The preciptated product was filtered and dried with ether to give the title compound (0.172 g, 83% pure, 89%). MS (ES+) m/e 598 [M+H]⁺.

### Example 92

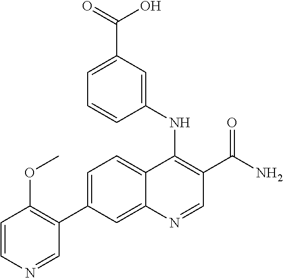 [SMILES: COc1ccncc1-c1ccc2c(Nc3cccc(C(=O)O)c3)c(C(N)=O)cnc2c1]

### 3-({3-(aminocarbonyl)-7-[4-(methyloxy)-3-pyridinyl]-4-quinolinyl}amino)benzoic acid

A solution of ethyl 3-{[3-(aminocarbonyl)-7-bromo-4-quinolinyl]amino}benzoate (example 6a, 115 mg, 0.278 mmol), 4-methoxypyridine-3-boronic acid hydrate (127 mg, 0.833 mmol), potassium carbonate (115 mg, 0.833 mmol), and tetrakis(triphenylphosphine)palladium(0) (35 mg, 0.030 mmol) in 1,4-dioxane (2.0 ml) and water (0.667 ml) was heated to 100° C. for 20 min. in a Biotage Initiator® microwave synthesizer. Upon cooling, the reaction mixture was diluted in water and extracted with ethyl acetate. The organic portion was dried over sodium sulfate, filtered and concentrated. The obtained residue was dissolved in ethanol (2 mL) and treated with sodium hydroxide (1N aq solution) (2 ml, 2.000 mmol). Following stirring at room temperature overnight, the reaction mixture was concentrated under reduced pressure to an aqueous residue, which was extracted using ethyl acetate. The aqueous portion was neutralized with a few drops of 6N hydrochloric, upon which a solid appeared. This solid was filtered and washed with water to obtain the title compound (0.035 g, 30%) as a light yellow powder. ¹H NMR (400 MHz, DMSO-d₆) ppm 11.65 (br. s., 1H), 9.01 (s, 1H), 8.82 (s, 1H), 8.80 (d, J=6.6 Hz, 1H), 8.27 (br. s., 1H), 8.19-8.25 (m, 2H), 7.81-7.88 (m, 3H), 7.78 (br. s., 1H), 7.62 (d, J=6.6 Hz, 1H), 7.49-7.58 (m, 2H), 4.05 (s, 3H). MS (ES+) m/e 415 [M+H]⁺.

### Example 93

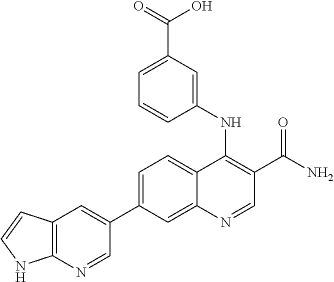 [SMILES: NC(=O)c1cnc2cc(-c3cnc4[nH]ccc4c3)ccc2c1Nc1cccc(C(=O)O)c1]

### 3-{[3-(aminocarbonyl)-7-(1H-pyrrolo[2,3-b]pyridin-5-yl)-4-quinolinyl]amino}benzoic acid, trifluoroacetate salt

A solution of ethyl 3-{[3-(aminocarbonyl)-7-bromo-4-quinolinyl]amino}benzoate (example 6a, 100 mg, 0.241 mmol), 7-azaindole-5-boronic acid pinacol ester (177 mg, 0.724 mmol), potassium carbonate (100 mg, 0.724 mmol), and tetrakis(triphenylphosphine)palladium(0) (13.95 mg, 0.012 mmol) in 1,4-dioxane (2.0 ml) and water (0.667 ml) was heated to 100° C. for 20 min. in a Biotage Initiator® microwave synthesizer. Upon cooling, the reaction mixture was diluted with water and extracted with ethyl acetate. The organic portion was concentrated under vacuo and the obtained residue was treated with ethanol (2 mL), followed by sodium hydroxide (1N aq solution) (2 ml, 2.000 mmol). After stirring at ambient temperature overnight, the reaction mixture was concentrated to an aqueous residue and extracted with ethyl acetate twice. The aqueous portion was neutralized using 6N hydrochloric acid and the precipitate filtered and purified using Reverse Phase HPLC (0.1% TFA in water and acetonitrile) to obtain the title compound (0.049 g, 38%) as a pale yellow solid. ¹H NMR (400 MHz, DMSO-d₆) ppm 13.17 (br. s., 1H), 11.92 (br. s., 1H), 11.65 (br. s., 1H), 8.99 (s, 1H), 8.71 (d, J=2.3 Hz, 1H), 8.45 (d, J=2.0 Hz, 1H), 8.27 (br. s., 1H), 8.26 (d, J=1.8 Hz, 1H), 8.22 (d, J=9.1 Hz, 1H), 8.08 (dd, J=9.0, 1.6 Hz, 1H), 7.81-7.87 (m, 2H), 7.77 (br. s., 1H), 7.58-7.63 (m, 1H), 7.51-7.57 (m, 2H), 6.59 (dd, J=3.4, 1.9 Hz, 1H). MS (ES+) m/e 424 [M+H]⁺.

### Example 94

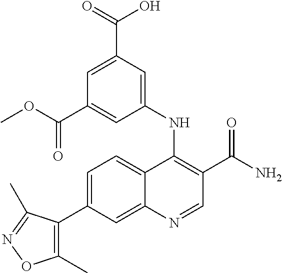 [SMILES: COC(=O)c1cc(Nc2c(C(N)=O)cnc3cc(-c4c(C)noc4C)ccc23)cc(C(=O)O)c1]

### 3-{[3-(aminocarbonyl)-7-(3,5-dimethyl-4-isoxazolyl)-4-quinolinyl]amino}-5-[(methyloxy)carbonyl]benzoic acid, trifluoroacetate salt

To a suspension of 4-chloro-7-(3,5-dimethyl-4-isoxazolyl)-3-quinolinecarboxamide (example 86a, 50 mg, 0.166 mmol) in acetic acid (10 mL) was added 3-amino-5-[(methyloxy)carbonyl]benzoic acid (32.3 mg, 0.166 mmol). The reaction mixture was stirred at room temperature overnight and was purified through preparative HPLC (YMC 75×30 mm column, 0.1% TFA in water and 0.1% TFA in acetonitrile) to afford the title compound (35 mg, 0.061 mmol, 36.8% yield) as a yellow solid. ¹H NMR (400 MHz, DMSO-d6) δ ppm 13.43 (br. s., 1H), 11.41 (br. s., 1H), 8.98 (s, 1H), 8.45 (d, J=8.8 Hz, 1H), 8.33 (t, J=1.5 Hz, 1H), 8.20 (br.s., 1H), 8.06 (d, J=1.8 Hz, 1H), 8.02-8.05 (m, 2H), 7.82 (dd, J=9.0, 1.6 Hz, 1H), 7.66 (s, 1H), 3.90 (s, 3H), 2.55 (s, 3H), 2.35 (s, 3H). MS (ES+) m/e 461 [M+H]⁺.

### Example 95

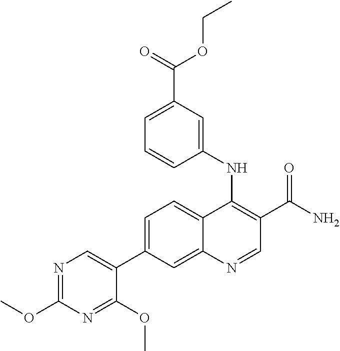 [SMILES: CCOC(=O)c1cccc(Nc2c(C(N)=O)cnc3cc(-c4cnc(OC)nc4OC)ccc23)c1]

### ethyl 3-({3-(aminocarbonyl)-7-[2,4-bis(methyloxy)-5-pyrimidinyl]-4-quinolinyl}amino)benzoate

A solution of ethyl 3-({3-(aminocarbonyl)-7-bromo-4-quinolinyl}amino)benzoate (example 6a, 300 mg, 0.724 mmol), 2,4-dimethoxy-5-pyrimidinylboronic acid (400 mg, 2.173 mmol), sodium bicarbonate (saturated aqueous solution) (1 mL, 0.724 mmol), and tetrakis(triphenylphosphine)palladium(0) (37.7 mg, 0.033 mmol) in 1,4-dioxane (2.0 ml) and water (0.667 mL) was heated to 100° C. for 20 min. in a Biotage Initiator® microwave synthesizer. Upon cooling, the reaction mixture was diluted with water and extracted twice with ethyl acetate. The aqueous portion contained a solid, which was filtered and washed with plenty of water to obtain the title compound (0.180 g, 53%) as dark beige solid. ¹H NMR (400 MHz, DMSO-d₆) ppm 10.34 (s, 1H), 9.01 (s, 1H), 8.57 (s, 1H), 8.25 (br. s., 1H), 8.17 (d, J=1.8 Hz, 1H), 7.85 (d, J=9.1 Hz, 1H), 7.68 (br. s., 1H), 7.63 (dd, J=8.8, 1.8 Hz, 1H), 7.55-7.61 (m, 2H), 7.39 (t, J=7.8 Hz, 1H), 7.20 (dd, J=7.8, 2.3 Hz, 1H), 4.27 (q, J=7.1 Hz, 2H), 3.98 (s, 3H), 3.97 (s, 3H), 1.28 (t, J=7.1 Hz, 3H). MS (ES+) m/e 474 [M+H]⁺.

### Example 96

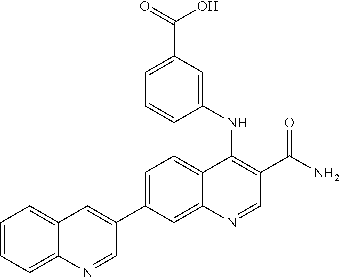 [SMILES: NC(=O)c1cnc2cc(-c3cnc4ccccc4c3)ccc2c1Nc1cccc(C(=O)O)c1]

### 3-{[3′-(aminocarbonyl)-3,7′-biquinolin-4′-yl]amino}benzoic acid

### a) ethyl 3-{[3′-(aminocarbonyl)-3,7′-biquinolin-4′-yl]amino}benzoate

A solution of ethyl 3-{[3-(aminocarbonyl)-7-bromo-4-quinolinyl]amino}benzoate (example 6a, 200 mg, 0.483 mmol), 3-quinolineboronic acid (125 mg, 0.724 mmol), sodium bicarbonate (saturated aqueous) (2 mL, 0.483 mmol), and tetrakis(triphenylphosphine)palladium(0) (25.1 mg, 0.022 mmol) in 1,4-dioxane (2.0 ml) was heated in a sealed tube to 100° C. overnight. Upon cooling, the reaction mixture was diluted with ethyl acetate, then washed with water. The organic portion was isolated and a precipitate was obtained upon standing still for several minutes. This precipitate was filtered, washed with ethyl ether and dried in vacuo to obtain the title compound (0.124 g, 56%) as a tanned yellow solid. ¹H NMR (400 MHz, DMSO-d₆) ppm 10.27 (br. s., 1H), 9.38 (d, J=2.3 Hz, 1H), 8.99 (s, 1H), 8.85 (s, 1H), 8.45 (br. s., 1H), 8.27 (br. s., 1H), 8.10 (d, J=8.8 Hz, 1H), 8.07 (d, J=8.8 Hz, 1H), 8.00 (br. s., 1H), 7.97 (br. s., 1H), 7.81 (ddd, J=8.5, 6.9, 1.3 Hz, 1H), 7.64-7.71 (m, 1H), 7.51-7.62 (m, 3H), 7.39 (t, J=7.8 Hz, 1H), 7.22 (dd, J=4.9, 2.7 Hz, 1H), 4.25 (q, J=7.2 Hz, 2H), 1.25 (t, J=7.2 Hz, 3H). MS (ES+) m/e 463 [M+H]⁺.

### b) 3-{[3′-(aminocarbonyl)-3,7′-biquinolin-4′-yl]amino}benzoic acid

To a suspension of ethyl 3-{[3′-(aminocarbonyl)-3,7′-biquinolin-4′-yl]amino}benzoate (100 mg, 0.216 mmol) in ethanol (5.0 mL) was added 1N aqueous sodium hydroxide (1 ml, 1.000 mmol). After stirring overnight at ambient temperature, the reaction was concentrated in vacuo and treated with 6N hydrochloric acid. A yellow precipitate was filtered and dried in vacuo to obtain the title compound (0.020 g, 21%) as a yellow solid. ¹H NMR (400 MHz, DMSO-d₆) ppm 11.46 (br. s., 1H), 9.40 (d, J=2.3 Hz, 1H), 9.04 (s, 1H), 8.90 (d, J=2.3 Hz, 1H), 8.48 (d, J=1.8 Hz, 1H), 8.33 (br. s., 1H), 8.21-8.28 (m, 1H), 8.16 (d, J=1.5 Hz, 1H), 8.12 (t, J=8.3 Hz, 2H), 7.85 (ddd, J=8.3, 6.8, 1.5 Hz, 1H), 7.74-7.82 (m, 3H), 7.71 (td, J=7.5, 1.1 Hz, 1H), 7.45-7.57 (m, 2H). MS (ES+) m/e 435 [M+H]⁺.

### Example 97

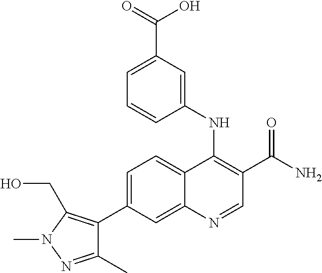 [SMILES: Cc1nn(C)c(CO)c1-c1ccc2c(Nc3cccc(C(=O)O)c3)c(C(N)=O)cnc2c1]

### 3-({3-(aminocarbonyl)-7-[5-(hydroxymethyl)-1,3-dimethyl-1H-pyrazol-4-yl]-4-quinolinyl}amino)benzoic acid

### a) (4-bromo-1,3-dimethyl-1H-pyrazol-5-yl)methanol

To a solution of (1,3-dimethyl-1H-pyrazole-5-yl)methanol (315 mg, 2.497 mmol) in ethanol (10 mL) was added sodium bicarbonate (262 mg, 3.12 mmol) and bromine (0.154 mL, 3.00 mmol). Following stirring for 1 h at room temperature, the reaction mixture was concentrated in vacuo. The residue was washed with sodium hydrogencarbonate and extracted with ethyl acetate. The organic portion was dried over sodium sulfate, filtered and concentrated to obtain the title compound (0.300 g, 59%) as a white solid. ¹H NMR (400 MHz, DMSO-d₆) ppm 5.32 (t, J=5.4 Hz, 1H), 4.43 (d, J=5.4 Hz, 2H), 3.77 (s, 3H), 2.08 (s, 3H). MS (ES+) m/e 205/207 [M+H]⁺.

### b) 3-({3-(aminocarbonyl)-7-[5-(hydroxymethyl)-1,3-dimethyl-1H-pyrazol-4-yl]-4-quinolinyl}amino)benzoic acid

A solution of ethyl 3-{[3-(aminocarbonyl)-7-bromo-4-quinolinyl]amino}benzoate (example 6a, 261 mg, 0.630 mmol), bis(pinacolato)diboron (240 mg, 0.945 mmol), tetrakis(triphenylphosphine)palladium(0) (32.8 mg, 0.028 mmol) and potassium acetate (186 mg, 1.890 mmol) in 1,4-dioxane (2.0 ml) was heated to 110° C. overnight in an oil bath. Upon cooling, the reaction mixture was treated with (4-bromo-1,3-dimethyl-1H-pyrazol-5-yl)methanol (100 mg, 0.488 mmol) followed by sodium bicarbonate (saturated aqueous solution) (2 mL, 0.603 mmol). The solution was bubbled through with a nitrogen current. After 5 minutes, the solution was treated with additional tetrakis(triphenylphosphine)palladium(0) (32.8 mg, 0.028 mmol) and heated to 110° C. overnight. After cooling, the contents were partitioned between water and ethyl acetate. The organic portion was dried over sodium sulfate, filtered and concentrated to obtain a solid which was dissolved in ethanol (5 ml) and treated with sodium hydroxide (1N aq solution) (0.5 mL, 0.125 mmol). After stirring overnight at room temperature, the mixture was concentrated to an aqueous residue, which was purified using Reverse Phase HPLC (0.1% TFA in acetonitrile and water) to give the title compound (0.050 g, 18%). ¹H NMR (400 MHz, DMSO-d₆) ppm 13.19 (br. s., 1H), 11.86 (br. s., 1H), 8.97 (s, 1H), 8.26 (br. s., 1H), 8.15 (d, J=9.1 Hz, 1H), 7.94 (d, J=1.8 Hz, 1H), 7.82-7.90 (m, 2H), 7.74-7.81 (m, 1H), 7.67 (dd, J=9.0, 1.4 Hz, 1H), 7.56 (d, J=5.1 Hz, 2H), 4.48 (s, 2H), 3.85 (s, 3H), 2.26 (s, 3H). MS (ES+) m/e 432 [M+H]⁺.

### Example 98

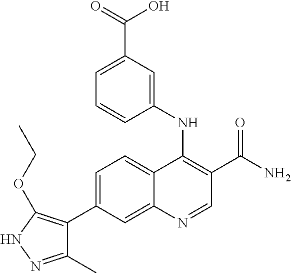 [SMILES: CCOc1[nH]nc(C)c1-c1ccc2c(Nc3cccc(C(=O)O)c3)c(C(N)=O)cnc2c1]

### 3-({3-(aminocarbonyl)-7-[5-(ethyloxy)-3-methyl-1H-pyrazol-4-yl]-4-quinolinyl}amino)benzoic acid

### a) t-butyl 3-{[3-(aminocarbonyl)-7-bromo-4-quinolinyl]amino}benzoate

A mixture of 7-bromo-4-chloro-3-quinolinecarboxamide (example 1a, 540 mg, 1.891 mmol) and t-butyl-3-amino benzoate (416 mg, 2.153 mmol) in 1,4-dioxane (10 mL) was heated to 80° C. for 2 h in an oil bath. Upon cooling, a solid precipitated out and was filtered, then washed with ethyl ether and dried to obtain the title compound (0.640 g, 77%) as a pale yellow solid. ¹H NMR (400 MHz, DMSO-d₆) ppm 11.50 (br. s., 1H), 8.98 (s, 1H), 8.23 (br. s., 1H), 8.22 (d, J=1.8 Hz, 1H), 8.13 (d, J=8.3 Hz, 1H), 7.81 (dd, J=9.1, 1.5 Hz, 1H), 7.71-7.78 (m, 3H), 7.45-7.53 (m, 1H), 7.39-7.45 (m, 1H), 1.54 (s, 9H). MS (ES+) m/e 442/444 [M+H]⁺.

### b) 4-bromo-3-(ethyloxy)-5-methyl-1H-pyrazole

To a solution of 3-(ethyloxy)-5-methyl-1H-pyrazole (200 mg, 1.585 mmol) in ethanol (10 mL) was added bromine (0.098 mL, 1.902 mmol). Following stirring for 1 h at room temperature, the reaction mixture was concentrated in vacuo, the residue was washed with sodium hydrogencarbonate and extracted with dichloromethane. The organic portion was dried over Na₂SO₄, filtered and concentrated to obtain the title compound (0.159 g, 49%) as a yellow solid. ¹H NMR (400 MHz, DMSO-d₆) ppm 12.02 (br. s., 1H), 4.14 (q, J=7.0 Hz, 2H), 2.12 (s, 3H), 1.28 (t, J=7.1 Hz, 3H). MS (ES+) m/e

205/207 [M+H]⁺.

### c) 3-({3-(aminocarbonyl)-7-[5-(ethyloxy)-3-methyl-1H-pyrazol-4-yl]-4-quinolinyl}amino)benzoic acid

A solution of t-butyl 3-{[3-(aminocarbonyl)-7-bromo-4-quinolinyl]amino}benzoate (200 mg, 0.452 mmol), bis(pinacolato) diboron (172 mg, 0.678 mmol), potassium acetate (133 mg, 1.357 mmol) and tetrakis(triphenylphosphine)palladium(0) (23.51 mg, 0.020 mmol) in 1,4-dioxane (5 ml) was heated to 115° C. for 3 h in an oil bath. Upon cooling, the reaction mixture was treated with 4-bromo-3-(ethyloxy)-5-methyl-1H-pyrazole (93 mg, 0.452 mmol) followed by sodium bicarbonate (saturated aq solution) (2 mL, 0.452 mmol). The solution was bubbled through with a nitrogen current. After 5 minutes, the solution was treated with additional tetrakis(triphenylphosphine)palladium(0) (23.51 mg, 0.020 mmol) and heated to 115° C. overnight. Upon cooling the contents were partitioned between water and ethyl acetate. The organic portion was dried over sodium sulfate, filtered and concentrated to obtain a solid which was dissolved into dichloromethane (5.00 mL), then treated with trifluoroacetic acid (1.2 mL, 15.58 mmol) at room temperature. After stirring overnight, the solution was concentrated, then dissolved into acetonitrile and purified using Reverse Phase HPLC (0.1% TFA in acetonitrile and water) to obtain the title compound (0.008 g, 4%). MS (ES+) m/e 432 [M+H]⁺.

### Example 99

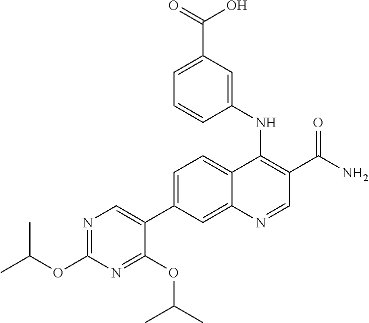 [SMILES: CC(C)Oc1ncc(-c2ccc3c(Nc4cccc(C(=O)O)c4)c(C(N)=O)cnc3c2)c(OC(C)C)n1]

### 3-[(3-(aminocarbonyl)-7-{2,4-bis[(1-methylethyl)oxy]-5-pyrimidinyl}-4-quinolinyl)amino]benzoic acid

### a) 5-bromo-2,4-di(isopropoxy)pyrimidine

Sodium isopropoxide (270 mg, 3.29 mmol) was added to a slurry of 5-bromo-2,4-dichloropyrimidine (300 mg, 1.317 mmol) in isopropanol (5 mL) and the mixture stirred at reflux for 2 h. The mixture was then cooled to room temperature, evaporated to dryness under reduced pressure and carried on directly through the next step. MS (ES+) m/e 275/277 [M+H]⁺.

### b) 3-[(3-(aminocarbonyl)-7-{2,4-bis[(1-methylethyl)oxy]-5-pyrimidinyl}-4-quinolinyl)amino]benzoic acid

A solution of ethyl 3-{[3-(aminocarbonyl)-7-bromo-4-quinolinyl]amino}benzoate (example 6a, 200 mg, 0.483 mmol), bis(pinacolato)diboron (153 mg, 0.603 mmol), potassium acetate (237 mg, 2.414 mmol), and tetrakis(triphenylphosphine)palladium(0) (25.1 mg, 0.022 mmol) in 1,4-dioxane (5 mL) was heated to 115° C. for 3 h in an oil bath. Upon cooling, the reaction mixture was treated with 5-bromo-2,4-di(isopropoxy)pyrimidine (133 mg, 0.483 mmol) followed by sodium bicarbonate (sat aq solution) (2 mL, 0.362 mmol). The solution was bubbled through with a nitrogen current. After 5 minutes, the solution was treated with additional tetrakis(triphenylphosphine)palladium(0) (18.83 mg, 0.016 mmol) and heated to 115° C. overnight. Upon cooling the contents were partitioned between water and ethyl acetate. The organic portion was concentrated and the residue treated with ethanol (10 mL) and sodium hydroxide (1N aq solution) (2 mL, 2.000 mmol). After stirring overnight at room temperature, the solution was concentrated, then dissolved into acetonitrile and purified using Reverse Phase HPLC (0.1% TFA in acetonitrile and water) to obtain the title compound (0.004 g, 2%) as a pale yellow solid. ¹H NMR (400 MHz, DMSO-d₆) ppm 13.25 (br. s., 1H), 11.82 (br. s., 1H), 8.98 (s, 1H), 8.63 (s, 1H), 8.26 (d, J=1.5 Hz, 2H), 8.20 (d, J=9.1 Hz, 1H), 7.84-7.93 (m, 3H), 7.80 (br. s., 1H), 7.54-7.60 (m, 2H), 5.44 (quin, J=6.2 Hz, 1H), 5.26 (quin, J=6.2 Hz, 1H), 1.38 (d, J=2.5 Hz, 6H), 1.36 (d, J=2.5 Hz, 6H). MS (ES+) m/e 502 [M+H]⁺.

### Example 100

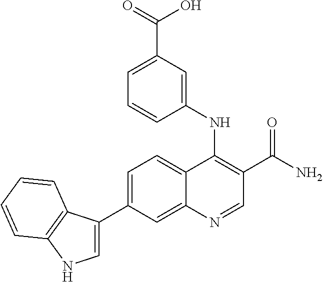 [SMILES: NC(=O)c1cnc2cc(-c3c[nH]c4ccccc34)ccc2c1Nc1cccc(C(=O)O)c1]

### 3-{[3-(aminocarbonyl)-7-(1H-indol-3-yl)-4-quinolinyl]amino}benzoic acid, trifluoroacetate salt

A solution of ethyl 3-{[3-(aminocarbonyl)-7-bromo-4-quinolinyl]amino}benzoate (example 6a, 100 mg, 0.241 mmol), 1-(phenylsulfonyl)-3-indoleboronic acid (278 mg, 0.724 mmol), potassium carbonate (100 mg, 0.724 mmol), and tetrakis(triphenylphosphine)palladium(0) (13.95 mg, 0.012 mmol) in 1,4-dioxane (2.0 ml) and water (0.667 mL) was heated to 100° C. for 30 min. in a Biotage Initiator® microwave synthesizer. Upon cooling, the reaction mixture was diluted with water and extracted with ethyl acetate. The organic portion was concentrated under vacuum and the obtained residue was treated with ethanol (10 ml), followed by sodium hydroxide (1N aq solution) (5 mL, 5.00 mmol). After stirring at ambient temperature overnight, the reaction mixture was concentrated to an aqueous residue and extracted with ethyl acetate twice. The aqueous portion was neutralized using 6N hydrochloric acid and the precipitate filtered and purified using Reverse Phase HPLC (0.1% TFA in water and acetonitrile) to obtain the title compound (0.035 g, 27%) as an orange solid. ¹H NMR (400 MHz, DMSO-d₆) ppm 11.93 (d, J=2.0 Hz, 1H), 11.90 (s, 1H), 9.00 (s, 1H), 8.41 (s, 1H), 8.30 (br. s., 1H), 8.22 (d, J=2.8 Hz, 1H), 8.09-8.15 (m, 1H), 7.98-8.08 (m, 2H), 7.85-7.93 (m, 2H), 7.81 (br. s., 1H), 7.56-7.62 (m, 2H), 7.50-7.56 (m, 1H), 7.20-7.30 (m, 2H). MS (ES+) m/e 423 [M+H]⁺.

### Example 101

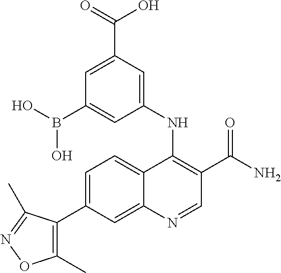 [SMILES: Cc1noc(C)c1-c1ccc2c(Nc3cc(B(O)O)cc(C(=O)O)c3)c(C(N)=O)cnc2c1]

### 3-{[3-(aminocarbonyl)-7-(3,5-dimethyl-4-isoxazolyl)-4-quinolinyl]amino}-5-(dihydroxyboranyl)benzoic acid

To a suspension of 4-chloro-7-(3,5-dimethyl-4-isoxazolyl)-3-quinolinecarboxamide (example 86a, 50 mg, 0.166 mmol) in acetic acid (10 mL) was added 3-amino-5-(dihydroxyboranyl)benzoic acid (30.0 mg, 0.166 mmol). The reaction mixture was stirred at room temperature overnight. The precipitate was collected, washed with ether and acetone, and dried under reduced pressure to afford the title compound (45 mg, 0.101 mmol, 60.9% yield) as a yellow solid. ¹H NMR (400 MHz, MeOD) δ ppm 8.98 (s, 1H), 8.30 (br. s., 1H), 8.05 (d, J=8.8 Hz, 1H), 7.97 (br. s., 1H), 7.91 (d, J=1.3 Hz, 1H), 7.81 (br. s., 1H), 7.53 (d, J=8.8 Hz, 1H), 2.52 (s, 3H), 2.34 (s, 3H). MS (ES+) m/e 447 [M+H]⁺.

### Example 102

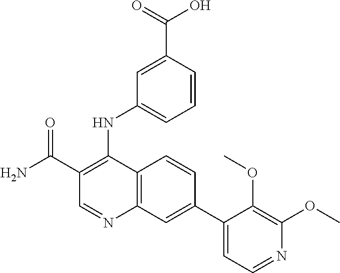 [SMILES: COc1nccc(-c2ccc3c(Nc4cccc(C(=O)O)c4)c(C(N)=O)cnc3c2)c1OC]

### 3-({3-(aminocarbonyl)-7-[2,3-bis(methyloxy)-4-pyridinyl]-4-quinolinyl}amino)benzoic acid

### a) ethyl 3-({3-(aminocarbonyl)-7-[2,3-bis(methyloxy)-4-pyridinyl]-4-quinolinyl}amino)benzoate

A solution of ethyl 3-{[3-(aminocarbonyl)-7-bromo-4-quinolinyl]amino}benzoate (example 6a, 150 mg, 0.362 mmol), (2,3-dimethoxypyridin-4-yl)boronic acid (72.9 mg, 0.398 mmol) and potassium carbonate (150 mg, 1.086 mmol) in 1,4-dioxane (4.5 mL) and water (1.5 mL) was degassed by bubbling with Ar for few min.

Tetrakis(triphenylphosphine)palladium(0) (20.92 mg, 0.018 mmol) was added. The reaction mixture was sealed and heated in a Biotage Initiator microwave reactor at 100° C. for 35 min. According to UPLC-MS, the conversion was complete. The reaction mixture was diluted with water and extracted two times with EtOAc. The collected organic layers were washed with water, dried over Na₂SO₄ and evaporated to obtain the crude product which was purified by column chromatography (silica gel, dichloromethane/methanol, 15:1 Supelco, 20 g) to afford the title compound (140 mg, 80%) as a yellow solid. MS (ES+) m/e 473 [M+H]⁺.

### b) 3-({3-(aminocarbonyl)-7-[2,3-bis(methyloxy)-4-pyridinyl]-4-quinolinyl}amino)benzoic acid

To a suspension of ethyl 3-({3-(aminocarbonyl)-7-[2,3-bis(methyloxy)-4-pyridinyl]-4-quinolinyl}amino)benzoate (112 mg, 0.237 mmol) in ethanol (2.42 mL), 1M solution of sodium hydroxide (2.42 mL, 2.420 mmol) was added (Note: the colour of the reaction mixture changed from intensive yellow to intensive orange-red after adding NaOH). The reaction mixture was stirred at RT. According to UPLC-MS, after stirring at RT for 4 h, the conversion was complete. The reaction mixture was concentrated to an aqueous residue which was then neutralized with 6M HCl. The resulting precipitate was filtered, washed with water and dried to obtain the title compound (102.9 mg, 94%) as a yellow solid. MS (ES+) m/e 445 [M+H]⁺.

### Example 103

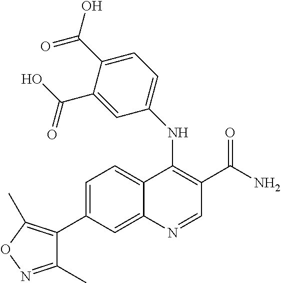 [SMILES: Cc1noc(C)c1-c1ccc2c(Nc3ccc(C(=O)O)c(C(=O)O)c3)c(C(N)=O)cnc2c1]

### 4-{[3-(aminocarbonyl)-7-(3,5-dimethyl-4-isoxazolyl)-4-quinolinyl]amino}-1,2-benzenedicarboxylic acid

### a) dimethyl 4-{[3-(aminocarbonyl)-7-(3,5-dimethyl-4-isoxazolyl)-4-quinolinyl]amino}-1,2-benzenedicarboxylate

To a solution of dimethyl 4-nitro-1,2-benzenedicarboxylate (200 mg, 0.836 mmol) in acetic acid (15 mL) was added palladium-on-carbon (8.90 mg, 0.084 mmol). The mixture was hydrogenated under a balloon at room temperature for 3 hours. After filtration, 4-chloro-7-(3,5-dimethyl-4-isoxazolyl)-3-quinolinecarboxamide (example 86a, 252 mg, 0.836 mmol) was added to the filtrate. The reaction mixture was stirred at room temperature overnight and was purified through preparative HPLC (YMC 75×30 mm column, 0.1% TFA in water and 0.1% TFA in acetonitrile) to afford the title compound (85 mg, 0.144 mmol, 17.27% yield) as a yellow solid. ¹H NMR (400 MHz, MeOD) δ ppm 9.02 (s, 1H), 8.20 (d, J=9.1 Hz, 1H), 7.96 (d, J=1.5 Hz, 1H), 7.85 (d, J=8.3 Hz, 1H), 7.67 (dd, J=9.0, 1.6 Hz, 1H), 7.57 (d, J=2.3 Hz, 1H), 7.49 (dd, J=8.3, 2.3 Hz, 1H), 3.89 (s, 3H), 3.87 (s, 3H), 2.54 (s, 3H), 2.36 (s, 3H). MS (ES+) m/e 475[M+H]⁺.

### b) 4-{[3-(aminocarbonyl)-7-(3,5-dimethyl-4-isoxazolyl)-4-quinolinyl]amino}-1,2-benzenedicarboxylic acid

To a solution of dimethyl 4-{[3-(aminocarbonyl)-7-(3,5-dimethyl-4-isoxazolyl)-4-quinolinyl]amino}-1,2-benzenedicarboxylate (50 mg, 0.105 mmol) in methanol (10 mL) was added sodium hydroxide (6.0 N in water) (0.088 mL, 0.527 mmol). The mixture was kept stirring for 2 hours, then acidified. The precipitate was collected, washed with water and dried under reduced pressure to afford the title compound (20 mg, 0.045 mmol, 42.5% yield) as a yellow solid. ¹H NMR (400 MHz, DMSO-d6) δ ppm 10.30 (br. s., 1H), 9.03 (s, 1H), 8.22 (br. s., 1H), 8.00-8.06 (m, 2H), 7.73 (br. s., 1H), 7.68 (d, J=8.6 Hz, 1H), 7.61 (dd, J=8.8, 1.5 Hz, 1H), 7.20 (br. s., 1H), 7.01 (dd, J=8.3, 2.3 Hz, 1H), 2.50 (s, 3H), 2.32 (s, 3H). MS (ES+) m/e 447 [M+H]⁺.

### Example 104

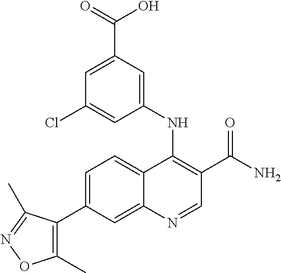 [SMILES: Cc1noc(C)c1-c1ccc2c(Nc3cc(Cl)cc(C(=O)O)c3)c(C(N)=O)cnc2c1]

### 3-{[3-(aminocarbonyl)-7-(3,5-dimethyl-4-isoxazolyl)-4-quinolinyl]amino}-5-chlorobenzoic acid, trifluoroacetate salt

To a suspension of 4-chloro-7-(3,5-dimethyl-4-isoxazolyl)-3-quinolinecarboxamide (example 86a, 50 mg, 0.166 mmol) in acetic acid (10 mL) was added 3-amino-5-chlorobenzoic acid (28.4 mg, 0.166 mmol). The reaction mixture was stirred at room temperature overnight and was purified through preparative HPLC (YMC 75×30 mm column, 0.1% TFA in water and 0.1% TFA in acetonitrile) to afford the title compound (28 mg, 0.051 mmol, 30.7% yield) as a yellow solid. ¹H NMR (400 MHz, DMSO-d6) δ ppm 13.48 (br. s., 1H), 11.16 (br. s., 1H), 9.00 (s, 1H), 8.35 (d, J=8.8 Hz, 1H), 8.22 (br. s., 1H), 8.04 (d, J=1.5 Hz, 1H), 7.79 (dd, J=8.8, 1.3 Hz, 1H), 7.75 (br. s., 1H), 7.70 (d, J=1.5 Hz, 2H), 7.54 (s, 1H), 2.54 (s, 3H), 2.35 (s, 3H). MS (ES+) m/e 437 [M+H]⁺.

### Example 105

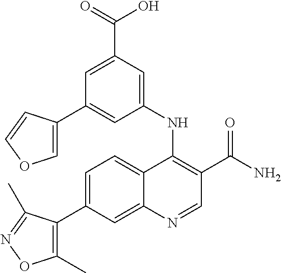 [SMILES: Cc1noc(C)c1-c1ccc2c(Nc3cc(C(=O)O)cc(-c4ccoc4)c3)c(C(N)=O)cnc2c1]

### 3-{[3-(aminocarbonyl)-7-(3,5-dimethyl-4-isoxazolyl)-4-quinolinyl]amino}-5-(3-furanyl)benzoic acid, trifluoroacetate salt

To a suspension of 4-chloro-7-(3,5-dimethyl-4-isoxazolyl)-3-quinolinecarboxamide (example 86a, 50 mg, 0.166 mmol) in acetic acid (10 mL) was added 3-amino-5-(3-furanyl)benzoic acid (example 61a, 33.7 mg, 0.166 mmol). The reaction mixture was stirred at room temperature overnight and was purified through preparative HPLC (YMC 75×30 mm column, 0.1% TFA in water and 0.1% TFA in acetonitrile) to afford the title compound (35 mg, 0.060 mmol, 36.3% yield) as a yellow solid. ¹H NMR (400 MHz, MeOD) δ ppm 8.97 (s, 1H), 8.21 (d, J=9.1 Hz, 1H), 8.19 (t, J=1.5 Hz, 1H), 8.03 (s, 1H), 7.90 (d, J=1.5 Hz, 1H), 7.84-7.87 (m, 1H), 7.78 (t, J=1.9 Hz, 1H), 7.59-7.65 (m, 2H), 6.87 (m, 1H), 2.52 (s, 3H), 2.35 (s, 3H). MS (ES+) m/e 469 [M+H]⁺.

### Example 106

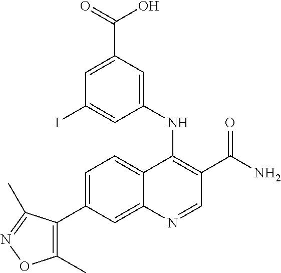 [SMILES: Cc1noc(C)c1-c1ccc2c(Nc3cc(I)cc(C(=O)O)c3)c(C(N)=O)cnc2c1]

### 3-{[3-(aminocarbonyl)-7-(3,5-dimethyl-4-isoxazolyl)-4-quinolinyl]amino}-5-iodobenzoic acid

### a) methyl 3-{[3-(aminocarbonyl)-7-(3,5-dimethyl-4-isoxazolyl)-4-quinolinyl]amino}-5-iodobenzoate trifluoroacetate

To a suspension of 4-chloro-7-(3,5-dimethyl-4-isoxazolyl)-3-quinolinecarboxamide (example 86a, 50 mg, 0.166 mmol) in acetic acid (10 mL) was added methyl 3-amino-5-iodobenzoate (45.9 mg, 0.166 mmol). The reaction mixture was stirred at room temperature overnight and was purified through preparative HPLC (YMC 75×30 mm column, 0.1% TFA in water and 0.1% TFA in acetonitrile) to afford the title compound (50 mg, 0.076 mmol, 46.0% yield) as a yellow solid. ¹H NMR (400 MHz, DMSO-d6) ppm 11.28 (br. s., 1H), 8.99 (s, 1H), 8.40 (d, J=8.6 Hz, 1H), 8.21 (br. s., 1H), 8.06 (s, 1H), 8.03 (s, 1H), 7.86 (s, 1H), 7.79-7.84 (m, 2H), 7.74 (s, 1H), 3.87 (s, 3H), 2.55 (s, 3H), 2.35 (s, 3H). MS (ES+) m/e 543 [M+H]⁺.

### b) 3-{[3-(aminocarbonyl)-7-(3,5-dimethyl-4-isoxazolyl)-4-quinolinyl]amino}-5-iodobenzoic acid

To a solution of methyl 3-{[3-(aminocarbonyl)-7-(3,5-dimethyl-4-isoxazolyl)-4-quinolinyl]amino}-5-iodobenzoate (30 mg, 0.055 mmol) in methanol (10 mL) was added sodium hydroxide (6.0 N in water) (11.06 mg, 0.277 mmol). The mixture was kept stirred at room temperature overnight, diluted with water and neatralized with 6N HCl. The precipiatate was collected and dried under reduced pressure to afford the title compound (22 mg, 0.042 mmol, 75% yield) as an off white solid. ¹H NMR (400 MHz, DMSO-d6) δ ppm 13.37 (br. s., 1H), 11.28 (br. s., 1H), 9.01 (s, 1H), 8.36 (d, J=8.8 Hz, 1H), 8.26 (br. s., 1H), 8.09 (d, J=1.5 Hz, 1H), 8.02 (s, 1H), 7.82 (s, 1H), 7.72-7.79 (m, 3H), 2.54 (s, 3H), 2.35 (s, 3H). MS (ES+) m/e 529 [M+H]⁺.

### Example 107

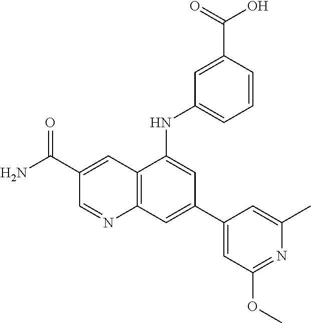 [SMILES: COc1cc(-c2cc(Nc3cccc(C(=O)O)c3)c3cc(C(N)=O)cnc3c2)cc(C)n1]

### 3-({3-(aminocarbonyl)-7-[2-methyl-6-(methyloxy)-4-pyridinyl]-4-quinolinyl}amino)benzoic acid

### a) ethyl 3-({3-(aminocarbonyl)-7-[2-methyl-6-(methyloxy)-4-pyridinyl]-4-quinolinyl}amino)benzoate

A solution of ethyl 3-{[3-(aminocarbonyl)-7-bromo-4-quinolinyl]amino}benzoate (example 6a, 150 mg, 0.362 mmol), 6-methoxy-2-picoline-4-boronic acid, pinacol ester (90 mg, 0.362 mmol) and potassium carbonate (100 mg, 0.724 mmol) in 1,4-dioxane (4.5 mL) and water (1.5 mL) was degassed by bubbling with Ar for few min.

Tetrakis(triphenylphosphine)palladium(0) (20.92 mg, 0.018 mmol) was added. The reaction mixture was sealed and heated in a Biotage Initiator microwave reactor at 80° C. for 30 min. According to UPLC-MS, the reaction was incomplete. Additional quantities of 6-methoxy-2-picoline-4-boronic acid, pinacol ester (20 mg, 0.080 mmol), potassium carbonate (50.0 mg, 0.362 mmol) and tetrakis(triphenylphosphine)palladium(0) (10.46 mg, 9.05 μmol) were added. After additional heating under MW at 100° C. for 20 min, according to UPLC-MS the conversion was complete. The reaction mixture was diluted with water and extracted two times with EtOAc. The collected organic layers were washed with water, dried over Na₂SO₄ and evaporated to obtain crude product, which was purified by column chromatography (silica gel, dichloromethane/methanol, 15:1 Supelco, 20 g) to afford the title compound (144.6 mg, 84%) as a yellow solid. MS (ES+) m/e 457 [M+H]⁺.

### b) 3-({3-(aminocarbonyl)-7-[2-methyl-6-(methyloxy)-4-pyridinyl]-4-quinolinyl}amino)benzoic acid

To a suspension of ethyl 3-({3-(aminocarbonyl)-7-[2-methyl-6-(methyloxy)-4-pyridinyl]-4-quinolinyl}amino)benzoate (131 mg, 0.287 mmol) in ethanol (3 mL), 1M sodium hydroxide (3 mL, 3.00 mmol) was added

(Note: the colour of the reaction mixture changed from intensive yellow to intensive orange-red after adding of NaOH). The reaction mixture was stirred at RT for 4 h, the reaction was complete by UPLC-MS. The reaction mixture was concentrated to an aqueous residue which was then neutralized with 6M HCl. The resulting precipitate was filtered, washed with water and dried to obtain the title compound (99.5 mg, 77%) as a yellow solid. MS (ES+) m/e 429 [M+H]⁺.

### Example 108

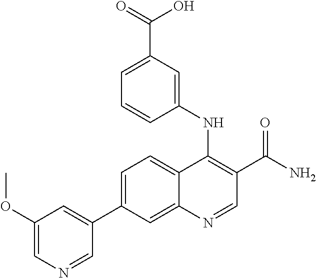 [SMILES: COc1cncc(-c2ccc3c(Nc4cccc(C(=O)O)c4)c(C(N)=O)cnc3c2)c1]

### 3-({3-(aminocarbonyl)-7-[5-(methyloxy)-3-pyridinyl]-4-quinolinyl}amino)benzoic acid, trifluoroacetate salt

A solution of ethyl 3-{[3-(aminocarbonyl)-7-bromo-4-quinolinyl]amino}benzoate (example 6a, 100 mg, 0.241 mmol), 5-methoxypyridine-3-boronic acid, pinacol ester (170 mg, 0.724 mmol), potassium carbonate (100 mg, 0.724 mmol), and tetrakis(triphenylphosphine)palladium(0) (12.55 mg, 10.86 μmol) in 1,4-dioxane (3 mL) and water (1.000 mL) was heated to 100° C. for 20 min in a Biotage Initiator® microwave synthesizer. Upon cooling, the reaction mixture was diluted with water and extracted with ethyl acetate. The organic portion was dried over magnesium sulfate, filtered and concentrated. The residue was purified via flash column chromatography (0-100% ethyl acetate in hexanes, then 10% methanol in ethyl acetate) to obtain a yellow solid (140 mg, MS (ES+) m/e 443 [M+H]⁺). Sodium hydroxide (1M aq solution) (3 mL, 3.00 mmol) was added to a slurry of this solid in ethanol (5 mL). After stirring at room temperature overnight, the mixture was concentrated to an aqueous residue and neutralized with a few drops of 6N hydrochloric acid. The resulting solid was purified using Reverse Phase HPLC (0.1% TFA in acetonitrile and water) to obtain the title compound (0.040 g, 31%) as a yellow solid. ¹H NMR (400 MHz, DMSO-d₆) ppm 11.92 (br. s., 1H), 9.02 (s, 1H), 8.69 (s, 1H), 8.46 (br. s., 1H), 8.39 (d, J=1.5 Hz, 1H), 8.31 (br. s., 1H), 8.27 (d, J=9.1 Hz, 1H), 8.11 (dd, J=9.1, 1.5 Hz, 1H), 7.84-7.95 (m, 3H), 7.80 (br. s., 1H), 7.58 (d, J=1.5 Hz, 1H), 7.57 (d, J=1.8 Hz, 1H), 3.96 (s, 3H). MS (ES+) m/e 415 [M+H]⁺.

### Example 109

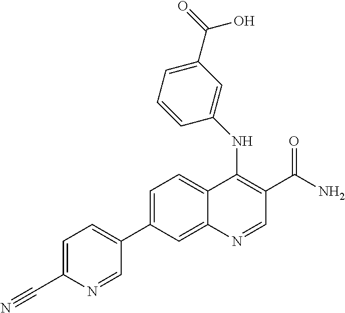 [SMILES: N#Cc1ccc(-c2ccc3c(Nc4cccc(C(=O)O)c4)c(C(N)=O)cnc3c2)cn1]

### 3-{[3-(aminocarbonyl)-7-(6-cyano-3-pyridinyl)-4-quinolinyl]amino}benzoic acid, bis(trifluoroacetate) salt

A solution of 3-{[3-(aminocarbonyl)-7-bromo-4-quinolinyl]amino}benzoic acid (example 88a, 100 mg, 0.259 mmol), 2-cyano-5-pyrimidineboronic acid pinacol ester (115 mg, 0.777 mmol), potassium carbonate (179 mg, 1.295 mmol), and tetrakis(triphenylphosphine)palladium(0) (30 mg, 0.026 mmol) in 1,4-dioxane (2.0 ml) and water (0.667 ml) was heated to 100° C. for 20 min in a Biotage Initiator® microwave synthesizer. Upon cooling, the reaction mixture was concentrated to an aqueous residue, which was purified via Reverse Phase HPLC (0.1% TFA in acetonitrile and water) to obtain the title compound (0.043 g, 26%) as an off white solid. ¹H NMR (400 MHz, DMSO-d₆) ppm 11.98 (br. s., 1H), 9.69 (d, J=1.8 Hz, 1H), 9.46 (s, 1H), 8.97 (dd, J=8.2, 2.4 Hz, 1H), 8.87 (d, J=1.5 Hz, 1H), 8.73 (br. s., 1H), 8.63-8.71 (m, 2H), 8.51 (dd, J=9.0, 1.6 Hz, 1H), 8.19 (br. s., 1H), 7.83-8.01 (m, 2H). MS (ES+) m/e 410 [M+H]⁺.

### Example 110

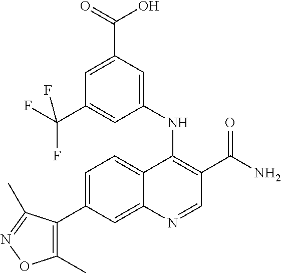 [SMILES: Cc1noc(C)c1-c1ccc2c(Nc3cc(C(=O)O)cc(C(F)(F)F)c3)c(C(N)=O)cnc2c1]

### 3-{[3-(aminocarbonyl)-7-(3,5-dimethyl-4-isoxazolyl)-4-quinolinyl]amino}-5-(trifluoromethyl)benzoic acid, trifluoroacetate salt

To a suspension of 4-chloro-7-(3,5-dimethyl-4-isoxazolyl)-3-quinolinecarboxamide (example 86a, 50 mg, 0.166 mmol) in acetic acid (10 mL) was added 3-amino-5-(trifluoromethyl)benzoic acid (34.0 mg, 0.166 mmol). The reaction mixture was stirred at room temperature overnight and was purified through preparative HPLC (YMC 75×30 mm column 0.1% TFA in water and 0.1% TFA in acetonitrile) to afford the title compound (15 mg, 0.026 mmol, 15.49% yield) as a yellow solid. ¹H NMR (400 MHz, DMSO-d6) δ ppm 10.49 (br. s., 1H), 8.97 (s, 1H), 8.23 (d, J=8.6 Hz, 1H), 8.18 (br. s., 1H), 8.02 (d, J=1.3 Hz, 1H), 7.82 (br.s., 1H), 7.80 (s, 1H), 7.68 (d, J=8.8 Hz, 2H), 7.65 (br. s., 1H), 7.62 (br. s., 1H), 2.51 (s, 3H), 2.32 (s, 3H). MS (ES+) m/e 471 [M+H]⁺.

### Example 111

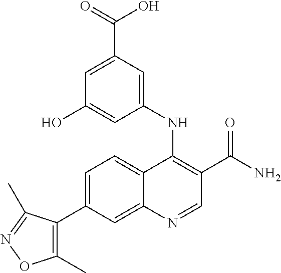 [SMILES: Cc1noc(C)c1-c1ccc2c(Nc3cc(O)cc(C(=O)O)c3)c(C(N)=O)cnc2c1]

### 3-{[3-(aminocarbonyl)-7-(3,5-dimethyl-4-isoxazolyl)-4-quinolinyl]amino}-5-hydroxybenzoic acid

To a suspension of 4-chloro-7-(3,5-dimethyl-4-isoxazolyl)-3-quinolinecarboxamide (example 86a, 50 mg, 0.166 mmol) in acetic acid (10 mL) was added 3-amino-5-hydroxybenzoic acid (25.4 mg, 0.166 mmol). The reaction mixture was stirred at room temperature over night. The precipitate was collected, washed with ether and dried under reduced pressure to afford the title compound (54 mg, 0.129 mmol, 78% yield) as an off white solid. ¹H NMR (400 MHz, MeOD) δ ppm 8.99 (s, 1H), 8.08 (d, J=8.8 Hz, 1H), 7.88 (d, J=1.5 Hz, 1H), 7.58 (dd, J=9.0, 1.6 Hz, 1H), 7.50-7.53 (m, 1H), 7.47 (d, J=1.0 Hz, 2H), 7.43-7.45 (m, 1H), 6.98-7.05 (m, 2H), 2.52 (s, 3H), 2.35 (s, 3H). MS (ES+) m/e 419 [M+H]⁺.

### Example 112

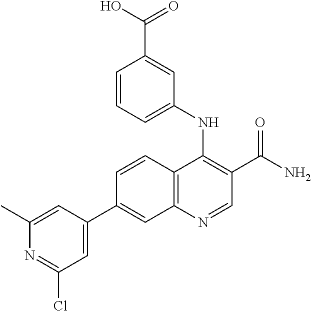 [SMILES: Cc1cc(-c2ccc3c(Nc4cccc(C(=O)O)c4)c(C(N)=O)cnc3c2)cc(Cl)n1]

### 3-{[3-(aminocarbonyl)-7-(2-chloro-6-methyl-4-pyridinyl)-4-quinolinyl]amino}benzoic acid

### a) ethyl 3-{[3-(aminocarbonyl)-7-(2-chloro-6-methyl-4-pyridinyl)-4-quinolinyl]amino}benzoate

A solution of ethyl 3-{[3-(aminocarbonyl)-7-bromo-4-quinolinyl]amino}benzoate (example 6a, 150 mg, 0.362 mmol), 2-chloro-6-methylpyridine-4-boronic acid, pinacol ester (101 mg, 0.398 mmol) and potassium carbonate (150 mg, 1.086 mmol) in 1,4-dioxane (4.5 mL) and water (1.5 mL) was degassed by bubbling with Ar for few min.

Tetrakis(triphenylphosphine)palladium(0) (20.92 mg, 0.018 mmol) was added. The reaction mixture was sealed and heated in a Biotage Initiator microwave reactor at 100° C. for 30 min. According to UPLC-MS, the conversion was almost complete and the reaction was stopped. The reaction mixture was diluted with water and extracted two times with EtOAc. The collected organic layers were washed with water, dried over Na₂SO₄ and evaporated to obtain the crude product, which was purified by column chromatography (silica gel, dichloromethane/methanol, 15:1 Supelco, 20 g) to afford the title compound (120 mg, 86% pure, 62%) as a yellow solid, which was used in the next step without further purification. MS (ES+) m/e 461 [M+H]⁺.

### b) 3-{[3-(aminocarbonyl)-7-(2-chloro-6-methyl-4-pyridinyl)-4-quinolinyl]amino}benzoic acid

To a suspension of ethyl 3-{[3-(aminocarbonyl)-7-(2-chloro-6-methyl-4-pyridinyl)-4-quinolinyl]amino}benzoate (103 mg, 0.223 mmol) in ethanol (2.335 mL), 1M sodium hydroxide (2.335 mL, 2.335 mmol) was added (Note: the colour of the reaction mixture changed from intensive yellow to intensive orange-red after adding of NaOH). The reaction mixture was stirred at RT for 4 h, whereupon the conversion was almost complete according to UPLC-MS and the reaction was stopped. The reaction mixture was concentrated to an aqueous residue which was then neutralized with 6M HCl. The resulting precipitate was filtered, washed with water and dried to obtain slightly impure product, which was purified by reverse phase HPLC (ODS, aqueous 10 mM ammonium bicarbonate/acetonitrile) to obtain the title compound (65 mg, 66%). MS (ES+) m/e 433 [M+H]⁺.

### Example 113

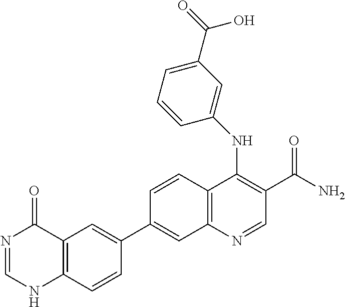 [SMILES: NC(=O)c1cnc2cc(-c3ccc4[nH]cnc(=O)c4c3)ccc2c1Nc1cccc(C(=O)O)c1]

### 3-{[3-(aminocarbonyl)-7-(4-oxo-1,4-dihydro-6-quinazolinyl)-4-quinolinyl]amino}benzoic acid

### a) ethyl 3-{[3-(aminocarbonyl)-7-(4-oxo-1,4-dihydro-6-quinazolinyl)-4-quinolinyl]amino}benzoate

A solution of ethyl 3-{[3-(aminocarbonyl)-7-bromo-4-quinolinyl]amino}benzoate (example 6a, 200 mg, 0.483 mmol), (4-oxo-1,4-dihydro-6-quinazolinyl)boronic acid (459 mg, 2.414 mmol), potassium carbonate (200 mg, 1.448 mmol) and tetrakis(triphenylphosphine)palladium(0) (167 mg, 0.145 mmol) in 1,4-dioxane (8 mL) and water (2 ml) was heated to 100° C. for 20 min in a Biotage Initiator microwave synthesizer. The reaction mixture was checked by UPLC-MS and the desired product was observed. Upon cooling, the reaction mixture was diluted with water (20 ml) and extracted twice with ethyl-acetate (2×20 ml). The combined organic layers were dried over Na₂SO₄ and concentrated to yield a yellowish solid which was chromatographed (10 g silica column, 0-8% dichloromethane/methanol) to give the title compound (55 mg, 23%) as a yellow solid. MS (ES+) m/e 480 [M+H]⁺.

### b) 3-{[3-(aminocarbonyl)-7-(4-oxo-1,4-dihydro-6-quinazolinyl)-4-quinolinyl]amino}benzoic acid

A solution of ethyl 3-{[3-(aminocarbonyl)-7-(4-oxo-1,4-dihydro-6-quinazolinyl)-4-quinolinyl]amino}benzoate (35 mg, 0.073 mmol) in sodium hydroxide (1.022 mL, 1.022 mmol) and ethanol (1 mL) was heated to 80° C. for 1 hr. The reaction mixture was checked by UPLC-MS after 1 hour and the desired product was observed. Upon cooling, the ethanol was evaporated, the pH of the reaction mixture was adjusted with 1N hydrochloric acid down to pH 3 but no precipitate was observed. The pH was adjusted to neutral, the aqueous solution was lyophilized and the crude yellow substance was purified by mass-directed preparative HPLC to give the title compound (3.7 mg, 11%) as a yellow solid. MS (ES+) m/e 452 [M+H]⁺.

### Example 114

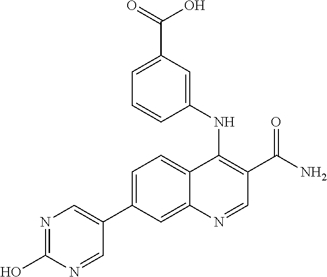 [SMILES: NC(=O)c1cnc2cc(-c3cnc(O)nc3)ccc2c1Nc1cccc(C(=O)O)c1]

### 3-{[3-(aminocarbonyl)-7-(2-oxo-1,2-dihydro-5-pyrimidinyl)-4-quinolinyl]amino}benzoic acid, bis(trifluoroacetate) salt

A solution of ethyl 3-{[3-(aminocarbonyl)-7-bromo-4-quinolinyl]amino}benzoate (example 6a, 100 mg, 0.241 mmol), 2-methoxypyrimidine-5-boronic acid (111 mg, 0.724 mmol), potassium carnbonate (100 mg, 0.724 mmol), and tetrakis(triphenylphosphine)palladium(0) (13.95 mg, 0.012 mmol) in 1,4-dioxane (2.0 ml) and water (0.667 ml) was heated to 100° C. for 30 min. in a Biotage Initiator® microwave synthesizer. Upon cooling, the reaction mixture was diluted with water and extracted with ethyl acetate. The organic portion was concentrated under vacuum and the obtained residue was diluted with ethanol (10 ml), followed by sodium hydroxide (1N aq solution) (5 mL, 5.00 mmol). After stirring at ambient temperature overnight, the mixture was examined via LCMS to reveal a mixture of the originally desired methoxypyrimidine carboxylic acid, methyl ester and the title compound. Conversion to the title compound was achieved by additional sodium hydroxide (1N aq solution) (5 ml, 5.00 mmol) and gently heating the mixture to 80° C. over a period of 2 h. After that time, the reaction had progressed to ˜70% completion and it was concentrated to the aqueous residue, followed by purification via Reverse Phase HPLC (0.1% TFA in acetonitrile and water) to obtain the title compound (0.017 g, 11%) as a yellow solid. ¹H NMR (400 MHz, DMSO-d₆) ppm 12.94 (br. s., 1H), 10.56 (br. s., 1H), 9.01 (s, 1H), 8.85 (br. s., 1H), 8.27 (br. s., 1H), 8.24 (d, J=1.8 Hz, 1H), 7.82-7.90 (m, 1H), 7.75 (dd, J=9.0, 1.6 Hz, 1H), 7.70 (br. s., 1H), 7.61 (d, J=7.8 Hz, 1H), 7.53 (s, 1H), 7.40 (t, J=7.8 Hz, 1H), 7.26 (dd, J=7.6, 1.5 Hz, 1H). MS (ES+) m/e 402 [M+H]⁺.

### Example 115

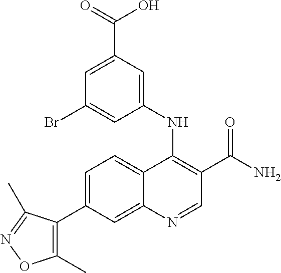 [SMILES: Cc1noc(C)c1-c1ccc2c(Nc3cc(Br)cc(C(=O)O)c3)c(C(N)=O)cnc2c1]

### 3-{[3-(aminocarbonyl)-7-(3,5-dimethyl-4-isoxazolyl)-4-quinolinyl]amino}-5-bromobenzoic acid, trifluoroacetate salt

To a suspension of 4-chloro-7-(3,5-dimethyl-4-isoxazolyl)-3-quinolinecarboxamide (example 86a, 50 mg, 0.166 mmol) in acetic acid (10 mL) was added 3-amino-5-bromobenzoic acid (35.8 mg, 0.166 mmol). The reaction mixture was stirred at room temperature overnight and was purified through preparative HPLC (YMC 75×30 mm column, 0.1% TFA in water and 0.1% TFA in acetonitrile) to afford the title compound (45 mg, 0.076 mmol, 45.6% yield) as a yellow solid. ¹H NMR (400 MHz, DMSO-d6) δ ppm 13.45 (br. s., 1H), 11.14 (br. s., 1H), 8.99 (s, 1H), 8.35 (d, J=8.8 Hz, 1H), 8.22 (br. s., 1H), 8.03 (d, J=1.5 Hz, 1H), 7.82 (br. s., 1H), 7.78 (dd, J=9.0, 1.4 Hz, 1H), 7.71-7.76 (m, 2H), 7.66 (s, 1H), 2.53 (s, 3H), 2.34 (s, 3H). MS (ES+) m/e 481 [M+H]⁺.

### Example 116

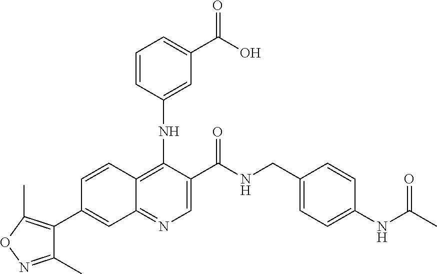 [SMILES: CC(=O)Nc1ccc(CNC(=O)c2cnc3cc(-c4c(C)noc4C)ccc3c2Nc2cccc(C(=O)O)c2)cc1]

### 3-{[3-[({[4-(acetylamino)phenyl]methyl}amino)carbonyl]-7-(3,5-dimethyl-4-isoxazolyl)-4-quinolinyl]amino}benzoic acid

### a) ethyl 3-{[3-[({[4-(acetylamino)phenyl]methyl}amino)carbonyl]-7-(3,5-dimethyl-4-isoxazolyl)-4-quinolinyl]amino}benzoate

PyBOP (226 mg, 0.435 mmol) was added to a stirred solution of 7-(3,5-dimethyl-4-isoxazolyl)-4-({3-[(ethyloxy)carbonyl]phenyl}amino)-3-quinolinecarboxylic acid (example 13c, 125 mg, 0.290 mmol), 4-acetamidobenzylamine (57.1 mg, 0.348 mmol) and triethylamine (0.121 mL, 0.869 mmol) in dichloromethane (3 mL) at room temperature, and the mixture stirred for 2 h. The solution was filtered and purified by HPLC (10-70% acetonitrile/water with 0.1% TFA) to give the title compound (132 mg, 0.229 mmol, 79% yield) as a pale yellow solid. MS (ES+) m/e 578 [M+H]⁺.

### b) 3-{[3-[({[4-(acetylamino)phenyl]methyl}amino)carbonyl]-7-(3,5-dimethyl-4-isoxazolyl)-4-quinolinyl]amino}benzoic acid

6M aqueous sodium hydroxide (0.152 mL, 0.914 mmol) was added to a stirred solution of ethyl 3-{[3-[({[4-(acetylamino)phenyl]methyl}amino)carbonyl]-7-(3,5-dimethyl-4-isoxazolyl)-4-quinolinyl]amino}benzoate (132 mg, 0.229 mmol) in ethanol (3 mL) at 60° C. overnight. HPLC purification (10-70% acetonitrile/water with 0.1% TFA @ 8 min gradient) afforded the title compound (64 mg, 0.116 mmol, 51.0% yield) as a white solid. ¹H NMR (METHANOL-d₄): 8.81 (s, 1H), 8.46 (d, J=8.8 Hz, 1H), 8.05 (td, J=4.4, 1.5 Hz, 1H), 7.98 (d, J=1.3 Hz, 1H), 7.92 (d, J=1.5 Hz, 1H), 7.76 (dd, J=8.8, 1.8 Hz, 1H), 7.56-7.62 (m, 2H), 7.49-7.56 (m, 2H), 7.24 (d, J=8.6 Hz, 2H), 4.08 (s, 2H), 2.56 (s, 3H), 2.39 (s, 3H), 2.14 (s, 3H).

### Example 117

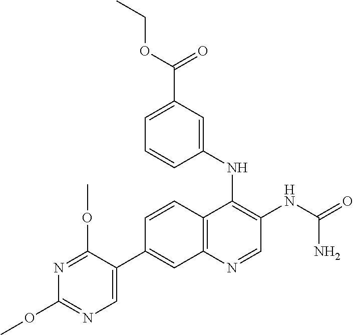 [SMILES: CCOC(=O)c1cccc(Nc2c(NC(N)=O)cnc3cc(-c4cnc(OC)nc4OC)ccc23)c1]

### ethyl 3-({3-[(aminocarbonyl)amino]-7-[2,4-bis(methyloxy)-5-pyrimidinyl]-4-quinolinyl}amino)benzoate

### a) 7-bromo-3-nitro-4-quinolinol

A stirred suspension of 7-bromo-4-quinolinol (2 g, 8.93 mmol) in propionic acid (46.8 ml, 625 mmol) was heated to 110° C. After nitric acid (0.545 ml, 11.60 mmol) was added, in portions over 5 min, the resulting suspension was stirred at 110° C. overnight. According to UPLC, conversion was complete. The reaction was cooled to RT and the solid was collected by filtration. The filter cake was washed with ice cold ethanol until the washings were nearly colourless. The product was dried under vacuum to afford the title compound (1.6 g, 94% pure, 63% yield) as an off-white powder. MS (ES+) m/e 271 [M+H]⁺. The product was used in a next step without purification.

### b) ethyl 3-[(7-bromo-3-nitro-4-quinolinyl)amino]benzoate

To 7-bromo-3-nitro-4-quinolinol (1.6 g, 5.95 mmol) was added phosphorus oxychloride (6 ml, 64.4 mmol). After stirring at reflux (120° C.) overnight, UPLC showed complete conversion into the desired chloro derivative (m/z=288.9 [M+H]⁺). The reaction mixture was concentrated under vacuum. The residue was mixed with ethyl 3-aminobenzoate (0.982 g, 5.95 mmol) and the mixture was suspended in 1,4-dioxane (10 mL) and N,N-dimethylformamide (2.00 ml), then vigorously shaken in an ultrasound bath whereupon a yellow precipitate formed. According to UPLC, conversion into desired product was complete. The precipitate was collected by filtration and washed with diethyl-ether to afford the title compound (3 g, 72% pure, 87% yield) as a yellow solid. MS (ES+) m/e 418 [M+H]⁺.

### c) ethyl 3-({7-[2,4-bis(methyloxy)-5-pyrimidinyl]-3-nitro-4-quinolinyl}amino)benzoate

A suspension of ethyl 3-[(7-bromo-3-nitro-4-quinolinyl)amino]benzoate (1.26 g, 3.03 mmol),2,4-bis(methyloxy)-5-(4,4,5,5-tetramethyl-1,3,2-dioxaborolan-2-yl)pyrimidine (0.806 g, 3.03 mmol), tetrakis(triphenylphosphine)palladium(0) (0.175 g, 0.151 mmol) and potassium carbonate (0.837 g, 6.05 mmol) in a mixture of 1,4-dioxane (12 mL) and water (4.00 mL) was heated under microwave conditions (Biotage Initiator) at 80° C. for 20 min. According to UPLC, conversion was complete. The layers were separated. The organic layer was additionally extracted with water (2×20 ml) and then dried using phase separator and evaporated in vacuo to afford crude product that was used in the next step without further purification. MS (ES+) m/e 476 [M+H]⁺.

### d) ethyl 3-({3-amino-7-[2,4-bis(methyloxy)-5-pyrimidinyl]-4-quinolinyl}amino)benzoate

A suspension of ethyl 3-({7-[2,4-bis(methyloxy)-5-pyrimidinyl]-3-nitro-4-quinolinyl}amino)benzoate (1.35 g, 2.84 mmol) and tin(II) chloride dihydrate (3.20 g, 14.20 mmol) in ethanol (20 mL) was heated at 80° C. for 2 h. The reaction was cooled to RT, poured into ice and the pH was adjusted to >10 using saturated aqueous sodium carbonate. After 10 min, to the resulting suspension was added dichloromethane (100 ml). The resulting two-layer suspension was filtered. The layers from the liqour were separated and the aqueous layer was additionally extracted with dichloromethane (2×80 ml). The combined organic layers were dried over a phase separator and evaporated in vacuo to afford crude product which was purified by chromatography (silicagel 50 g column, 0-30% ethyl acetate/cyclohexane to afford the title compound (320 mg, 86% pure, 21.76% yield) as a yellow solid. Product was used in the next step without further purification. MS (ES+) m/e 446 [M+H]⁺.

### e) ethyl 3-({3-[(aminocarbonyl)amino]-7-[2,4-bis(methyloxy)-5-pyrimidinyl]-4-quinolinyl}amino)benzoate

To a solution of ethyl 3-({3-amino-7-[2,4-bis(methyloxy)-5-pyrimidinyl]-4-quinolinyl}amino)benzoate (80 mg, 0.180 mmol) in dry N,N-dimethylformamide (3 mL) was added bis(1,1-dimethylethyl) [(Z)-(methylthio)methylylidene]biscarbamate (104 mg, 0.359 mmol), triethylamine (0.150 mL, 1.078 mmol) and finally mercury trifluoroacetate (153 mg, 0.359 mmol). The resulting solution was stirred at room temperature overnight. According to UPLC, conversion was complete. The resulting suspension was diluted with dichloromethane (50 ml) and sat aq sodium bicarbonate (25 ml). The organic layer was washed with water (30 ml), then brine (30 ml). A precipitate was removed by filtration (inorganics) and the organic layer was dried by phase separator to afford the Boc intermediate (110 mg) m/z=589.2 [M+H]⁺. The Boc derivative was dissolved in trifluoroacetic acid (3 mL, 38.9 mmol) and stirred at RT for 2 h. According to UPLC conversion was complete. Solvent was evaporated and the residue was dissolved in 2N aqueous sodium hydroxide (20 ml), then extracted with dichloromethane (3×30 ml). The combined organic layers were washed with water (2×20 ml), dried by phase separator and evaporated in vacuo to afford the crude product which was purified by chromatography (silicagel, 10 g column, 1-4% methanol/dichloromethane) to afford impure product (41 mg). The material was purified by prep HPLC to afford the title compound (18 mg, 0.034 mmol, 18.82% yield) as a white solid. ¹H NMR (300 MHz, DMSO-d₆) d ppm 1.19-1.32 (m, 3H) 3.88-4.04 (m, 6H) 4.25 (q, J=7.14 Hz, 2H) 6.29 (s, 2H) 6.69 (dt, J=7.58, 1.35 Hz, 1H) 7.18-7.30 (m, 1H) 7.30-7.38 (m, 2H) 7.66 (dd, J=8.80, 1.83 Hz, 1H) 7.82 (d, J=8.71 Hz, 1H) 8.14 (d, J=1.57 Hz, 1H) 8.35 (s, 1H) 8.47-8.60 (m, 2H) 9.49 (s, 1H).

### Example 118

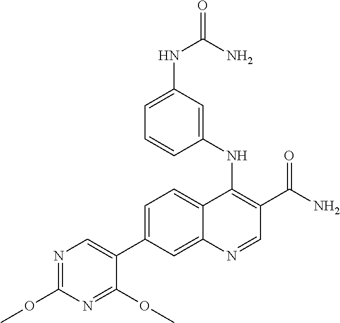 [SMILES: COc1ncc(-c2ccc3c(Nc4cccc(NC(N)=O)c4)c(C(N)=O)cnc3c2)c(OC)n1]

### 4-({3-[(aminocarbony)amino]phenyl}amino)-7-[2,4-bis(methyloxy)-5-pyrimidinyl]-3-quinolinecarboxamide, hydrochloride salt

To a suspension of 7-[2,4-bis(methyloxy)-5-pyrimidinyl]-4-chloro-3-quinolinecarboxamide (example 1b, 50 mg, 0.145 mmol) in acetic acid (10 mL) was added N-(3-aminophenyl)urea (21.92 mg, 0.145 mmol). The reaction mixture was stirred at room temperature overnight. The precipitate was collected, washed with ether and dried under reduced pressure to afford the title compound (34 mg, 0.069 mmol, 47.3% yield) as a yellow solid. ¹H NMR (400 MHz, DMSO-d6) δ ppm 12.44 (br. s., 1H), 9.11 (s, 1H), 8.92 (s, 1H), 8.61 (s, 1H), 8.50 (br. s., 1H), 8.25 (d, J=1.5 Hz, 1H), 7.98 (br. s., 1H), 7.89 (d, J=8.8 Hz, 1H), 7.74 (dd, J=9.1, 1.8 Hz, 1H), 7.56 (s, 1H), 7.29 (d, J=5.1 Hz, 2H), 6.80-6.88 (m, 1H), 5.99 (br. s., 2H), 4.00 (s, 3H), 3.98 (s, 3H). MS (ES+) m/e 460 [M+H]⁺.

### Example 119

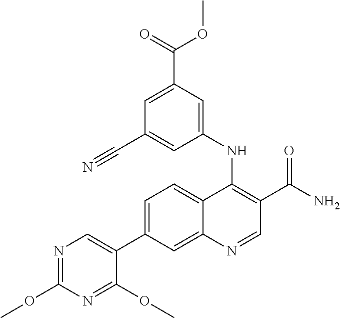 [SMILES: COC(=O)c1cc(C#N)cc(Nc2c(C(N)=O)cnc3cc(-c4cnc(OC)nc4OC)ccc23)c1]

### methyl 3-({3-(aminocarbonyl)-7-[2,4-bis(methyloxy)-5-pyrimidinyl]-4-quinolinyl}amino)-5-cyanobenzoate, trifluoroacetate salt

To a suspension of 7-[2,4-bis(methyloxy)-5-pyrimidinyl]-4-chloro-3-quinolinecarboxamide (example 1b, 100 mg, 0.290 mmol) in acetic acid (10 mL) was added methyl 3-amino-5-cyanobenzoate (51.1 mg, 0.290 mmol). The reaction mixture was stirred at room temperature overnight and was purified through preparative HPLC (YMC 75×30 mm column, 0.1% TFA in water and 0.1% TFA in acetonitrile) to afford the title compound (40 mg, 0.067 mmol, 23.04% yield) as an off white solid. ¹H NMR (400 MHz, DMSO-d6) δ ppm 11.16 (br. s., 1H), 8.98 (s, 1H), 8.69 (s, 1H), 8.41 (d, J=9.1 Hz, 1H), 8.29 (d, J=1.5 Hz, 1H), 8.21 (br. s., 1H), 8.14 (s, 1H), 8.08 (s, 1H), 8.00 (dd, J=9.0, 1.4 Hz, 1H), 7.92 (s, 1H), 7.70 (s, 1H), 4.04 (s, 3H), 4.00 (s, 3H), 3.90 (s, 3H). MS (ES+) m/e 485 [M+H]⁺.

### Example 120

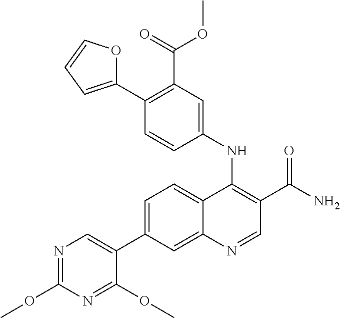 [SMILES: COC(=O)c1cc(Nc2c(C(N)=O)cnc3cc(-c4cnc(OC)nc4OC)ccc23)ccc1-c1ccco1]

### methyl 5-({3-(aminocarbonyl)-7-[2,4-bis(methyloxy)-5-pyrimidinyl]-4-quinolinyl}amino)-2-(2-furanyl)benzoate, trifluoroacetate salt

Preparation described in example 83b.

### Example 121

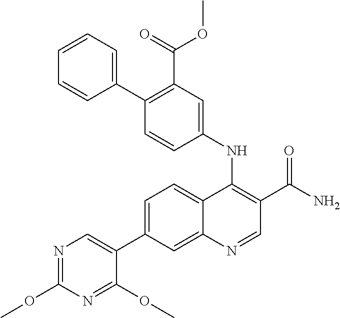 [SMILES: COC(=O)c1cc(Nc2c(C(N)=O)cnc3cc(-c4cnc(OC)nc4OC)ccc23)ccc1-c1ccccc1]

### methyl 4-({3-(aminocarbonyl)-7-[2,4-bis(methyloxy)-5-pyrimidinyl]-4-quinolinyl}amino)-2-biphenylcarboxylate

Preparation described in example 85b.

### Example 122

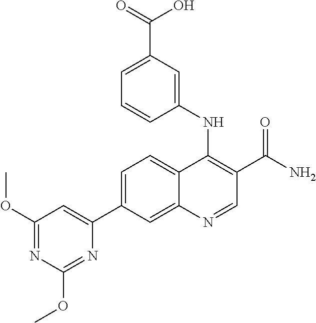 [SMILES: COc1cc(-c2ccc3c(Nc4cccc(C(=O)O)c4)c(C(N)=O)cnc3c2)nc(OC)n1]

### 3-({3-(aminocarbonyl)-7-[2,6-bis(methyloxy)-4-pyrimidinyl]-4-quinolinyl}amino)benzoic acid

A solution of ethyl 3-{[3-(aminocarbonyl)-7-bromo-4-quinolinyl]amino}benzoate (example 6a, 200 mg, 0.483 mmol), bis(pinacolato)diboron (135 mg, 0.531 mmol), potassium acetate (104 mg, 1.062 mmol) and tetrakis(triphenylphosphine)palladium(0) (25.1 mg, 0.022 mmol) in 1,4-dioxane (5.0 ml) was heated to 115° C. for 3 h in an oil bath.

Upon cooling, the reaction mixture was treated with 6-chloro-2,4-dimethoxy-pyrimidine (93 mg, 0.531 mmol) followed by sodium bicarbonate (sat aq solution) (2 mL, 0.483 mmol). The solution was bubbled through with a nitrogen current. After 5 minutes, the solution was treated with additional tetrakis(triphenylphosphine)palladium(0) (25.1 mg, 0.022 mmol) and heated to 115° C. overnight. Upon cooling the contents were partitioned between water and ethyl acetate. The organic portion was dried over sodium sulfate, filtered and concentrated. The residue was treated with ethanol (5 mL) followed by sodium hydroxide (1N aq solution) (1 mL, 1.000 mmol) and stirred for 3 h. The contents were concentrated to an aqueous residue and purified using Reverse Phase HPLC (0.1% TFA in acetonitrile and water) to obtain the title compound (0.017 g, 8%) as a pale yellow solid. ¹H NMR (400 MHz, DMSO-d₆) ppm 13.13 (br. s., 1H), 11.47 (br. s., 1H), 9.03 (s, 1H), 8.83 (d, J=1.8 Hz, 1H), 8.32 (dd, J=9.0, 1.4 Hz, 1H), 8.19-8.29 (m, 2H), 7.81 (d, J=7.3 Hz, 1H), 7.73-7.79 (m, 2H), 7.45-7.56 (m, 2H), 7.38 (s, 1H), 4.05 (s, 3H), 3.98 (s, 3H). MS (ES+) m/e 446 [M+H]⁺.

### Example 123

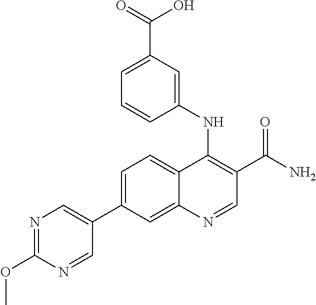 [SMILES: COc1ncc(-c2ccc3c(Nc4cccc(C(=O)O)c4)c(C(N)=O)cnc3c2)cn1]

### 3-({3-(aminocarbonyl)-7-[2-(methyloxy)-5-pyrimidinyl]-4-quinolinyl}amino)benzoic acid, bis(trifluoroacetate) salt

A solution of 3-{[3-(aminocarbonyl)-7-bromo-4-quinolinyl]amino}benzoic acid (example 88a, 100 mg, 0.259 mmol), 2-methoxypyrimidine-5-boronic acid (120 mg, 0.777 mmol), potassium carbonate (179 mg, 1.295 mmol), and tetrakis(triphenylphosphine)palladium(0) (13.46 mg, 0.012 mmol) in 1,4-dioxane (2.0 ml) and water (0.667 ml) was heated to 100° C. for 20 min. in a Biotage Initiator® microwave synthesizer. Upon cooling, the reaction mixture was concentrated in vacuo. The residue was dissolved into water and DMSO and purified via Reverse Phase HPLC (0.1% TFA in acetonitrile and water) to obtain the title compound (0.124 g, 74%) as a white solid. ¹H NMR (400 MHz, DMSO-d₆) ppm 11.50 (br. s., 1H), 9.13 (s, 2H), 8.99 (s, 1H), 8.28 (d, J=1.3 Hz, 2H), 8.19 (d, J=8.8 Hz, 1H), 8.02 (dd, J=9.0, 1.6 Hz, 1H), 7.81 (d, J=7.6 Hz, 1H), 7.72-7.79 (m, 2H), 7.50-7.57 (m, 1H), 7.44-7.50 (m, 1H), 4.01 (s, 3H). MS (ES+) m/e 416 [M+H]⁺.

### Example 124

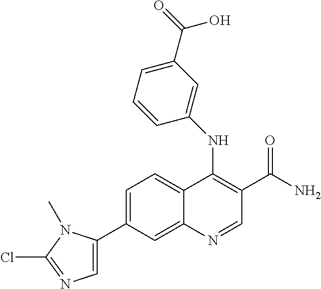 [SMILES: Cn1c(-c2ccc3c(Nc4cccc(C(=O)O)c4)c(C(N)=O)cnc3c2)cnc1Cl]

### 3-{[3-(aminocarbonyl)-7-(2-chloro-1-methyl-1H-imidazol-5-yl)-4-quinolinyl]amino}benzoic acid

### a) ethyl 3-{[3-(aminocarbonyl)-7-(2-chloro-1-methyl-1H-imidazol-5-yl)-4-quinolinyl]amino}benzoate

A solution of ethyl 3-{[3-(aminocarbonyl)-7-bromo-4-quinolinyl]amino}benzoate (example 6a, 100 mg, 0.241 mmol), 2-chloro-1-methyl-5-(4,4,5,5-tetramethyl-1,3,2-dioxaborolan-2-yl)-1H-imidazole (58.5 mg, 0.241 mmol), potassium carbonate (100 mg, 0.724 mmol) and tetrakis(triphenylphosphine)palladium(0) (11.16 mg, 9.66 μmol) in 1,4-dioxane (3 ml) and water (1 ml) was heated to 100° C. for 20 min in a Biotage Initiator microwave synthesizer. The reaction mixture was checked by UPLC-MS, and desired product was observed. Upon cooling, the reaction mixture was diluted with water (20 ml) and extracted twice with ethyl-acetate (2×20 ml). The combined organic layers were dried over Na₂SO₄ and concentrated to yield a yellowish solid which was purified by chromatography (10 g silica column, 0-8% methanol/dichloromethane) to give the title compound (46 mg, 36%) as a yellow solid. MS (ES+) m/e 450 [M+H]⁺.

### b) 3-{[3-(aminocarbonyl)-7-(2-chloro-1-methyl-1H-imidazol-5-yl)-4-quinolinyl]amino}benzoic acid

To a solution of ethyl 3-{[3-(aminocarbonyl)-7-(2-chloro-1-methyl-1H-imidazol-5-yl)-4-quinolinyl]amino}benzoate (45 mg, 0.100 mmol) in ethanol (3 ml), aqueous sodium hydroxide (1.400 ml, 1.400 mmol) was added. The reaction mixture was stirred at 22° C. for 4 hours and checked by UPLC-MS. The desired product was obtained. The pH of the reaction mixture was adjusted to 7 at which point a yellow precipitate was formed. The precipitate was filtered under vacuum and washed with diethyl ether a few times, then dried under vacuum overnight to give the title compound (0.023 g, 53%). MS (ES+) m/e 422 [M+H]⁺.

### Example 125

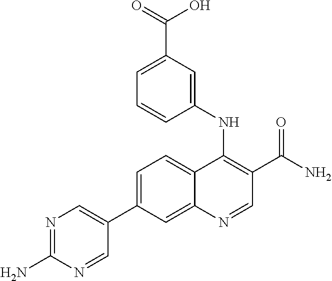 [SMILES: NC(=O)c1cnc2cc(-c3cnc(N)nc3)ccc2c1Nc1cccc(C(=O)O)c1]

### 3-{[3-(aminocarbonyl)-7-(2-amino-5-pyrimidinyl)-4-quinolinyl]amino}benzoic acid

A solution of ethyl 3-{[3-(aminocarbonyl)-7-bromo-4-quinolinyl]amino}benzoate (example 6a, 100 mg, 0.241 mmol), 2-aminopyrimidine-5-boronic acid (160 mg, 0.724 mmol), potassium carbonate (100 mg, 0.724 mmol), and tetrakis(triphenylphosphine)palladium(0) (12.55 mg, 10.86 μmol) in 1,4-dioxane (2.0 ml) and water (3 mL) was heated to 120° C. for 20 min. in a Biotage Initiator® microwave synthesizer. Upon cooling, the reaction mixture was diluted with water and extracted with ethyl acetate, then concentrated in vacuo. The residue was dissolved into ethanol (0.667 ml) and treated with sodium hydroxide (1N aq solution) (2.0 ml, 2.000 mmol). Stirring continued for 2 h after which time the solution was concentrated under reduced pressure to remove most of the ethanol. The aqueous residue was then extracted with ethyl acetate and neutralized with 1N hydrochloric acid. The precipitate was filtered and washed with water to obtain the title compound (36 mg, 37%) as a yellow solid. 1H NMR (400 MHz, DMSO-d6) ppm 13.19 (br. s., 1H), 11.80 (br. s., 1H), 8.96 (s, 1H), 8.79 (s, 2H), 8.29 (br. s., 1H), 8.11-8.19 (m, 2H), 7.99 (dd, J=9.1, 1.5 Hz, 1H), 7.84-7.89 (m, 1H), 7.84 (s, 1H), 7.77 (br. s., 1H), 7.55 (d, J=5.6 Hz, 2H), 7.19 (br. s., 2H). MS (ES+) m/e 401 [M+H]⁺.

### Example 126

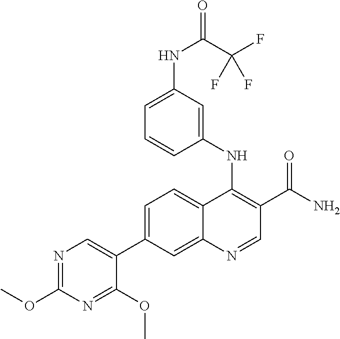 [SMILES: COc1ncc(-c2ccc3c(Nc4cccc(NC(=O)C(F)(F)F)c4)c(C(N)=O)cnc3c2)c(OC)n1]

### 7-[2,4-bis(methyloxy)-5-pyrimidinyl]-4-({3-[(trifluoroacetyl)amino]phenyl}amino)-3-quinolinecarboxamide, trifluoroacetate salt

To a suspension of 7-[2,4-bis(methyloxy)-5-pyrimidinyl]-4-chloro-3-quinolinecarboxamide (example 1b, 50 mg, 0.145 mmol) in acetic acid (10 mL) was added N-(3-aminophenyl)-2,2,2-trifluoroacetamide (29.6 mg, 0.145 mmol). The reaction mixture was stirred at room temperature overnight and was purified through preparative HPLC (YMC 75×30 mm column, 0.1% TFA in water and 0.1% TFA in acetonitrile) to afford the title compound (13 mg, 0.021 mmol, 14.31% yield) as a yellow solid. ¹H NMR (400 MHz, DMSO-d6) 5 ppm 11.84 (br. s., 1H), 11.38 (s, 1H), 9.03 (s, 1H), 8.63 (s, 1H), 8.33 (br. s., 1H), 8.22 (d, J=1.5 Hz, 1H), 8.04 (d, J=9.1 Hz, 1H), 7.87 (br. s., 1H), 7.80 (dd, J=9.1, 1.5 Hz, 1H), 7.69 (br. s., 1H), 7.58 (d, J=8.3 Hz, 1H), 7.44 (t, J=8.1 Hz, 1H), 7.11 (d, J=7.8 Hz, 1H), 4.01 (s, 3H), 3.98 (s, 3H). MS (ES+) m/e 513 [M+H]⁺.

### Example 127

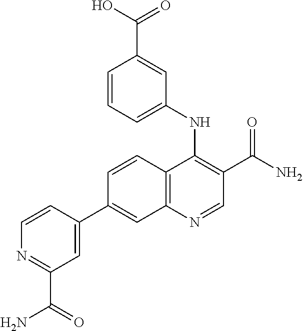 [SMILES: NC(=O)c1cc(-c2ccc3c(Nc4cccc(C(=O)O)c4)c(C(N)=O)cnc3c2)ccn1]

### 3-({3-(aminocarbonl)-7-[2-(aminocarbonyl)-4-pidinyl]-4-quiuinolinyl}amino)benzoic acid

To a suspension of ethyl 3-({3-(aminocarbonyl)-7-[2-(aminocarbonyl)-4-pyridinyl]-4-quinolinyl}amino)benzoate (from example 90a, 16.5 mg, 0.036 mmol) in ethanol (1 mL), 1M sodium hydroxide (1 mL, 1.000 mmol) was added (Note: the colour of the reaction mixture changed from intensive yellow to intensive orange-red after adding of NaOH).

The reaction mixture was stirred 4 h at RT when, according to UPLC-MS, the conversion was complete. The reaction mixture was concentrated to an aqueous residue which was then neutralized with 6M HCl. The resulting precipitate was filtered, washed with water and dried to obtain the title compound (12.9 mg, 93% pure, 77%) as a yellow solid. MS (ES+) m/e 428 [M+H]⁺.

### Example 128

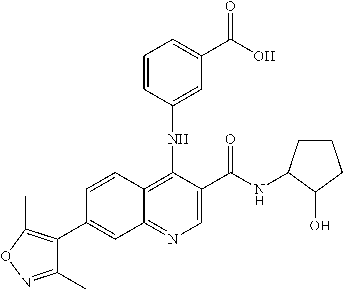 [SMILES: Cc1noc(C)c1-c1ccc2c(Nc3cccc(C(=O)O)c3)c(C(=O)NC3CCCC3O)cnc2c1]

### 3-[(7-(3,5-dimethyl-4-isoxazolyl)-3-{[(2-hydroxycyclopentyl)amino]carbonyl}-4-quinolinyl)amino]benzoic acid

a) (+,−) 2-aminocyclopentanol

A 25 mL microwave vial was charged with epoxycyclopentane (1 g, 11.89 mmol) in concentrated ammonium hydroxide (0.463 ml, 11.89 mmol). The vial was sealed and heated to 75° C. in an oil bath overnight. The vial was cooled to rt, NaOH pellets added to liberate the amine alcohol, and diethyl ether was added. The resulting oil layer was collected and the amino alcohol dissolved in dilute hydrochloric acid. Drying under reduced pressure afforded the title compound (1.636 g, 11.89 mmol, 100% yield) as a crude HCl salt, used without further purification.

### b) ethyl 3-[(7-(3,5-dimethyl-4-isoxazolyl)-3-{[(2-hydroxycyclopentyl)amino]carbonyl}-4-quinolinyl)amino]benzoate

PyBOP (226 mg, 0.435 mmol) was added to a stirred solution of 7-(3,5-dimethyl-4-isoxazolyl)-4-({3-[(ethyloxy)carbonyl]phenyl}amino)-3-quinolinecarboxylic acid (example 13c, 125 mg, 0.290 mmol), (+,−) 2-aminocyclopentanol (47.6 mg, 0.348 mmol), and triethylamine (0.121 mL, 0.869 mmol) in dichloromethane (3 mL) at room temperature, and the mixture stirred overnight. Purification by reverse-phase HPLC (10-70% acetonitrile/water+0.1% TFA) afforded the title compound (87 mg, 0.169 mmol, 58.4% yield) as a yellow solid. MS (ES+) m/e 515 [M+H]⁺.

### c) 3-[(7-(3,5-dimethyl-4-isoxazolyl)-3-{[(2-hydroxycyclopentyl)amino]carbonyl}-4-quinolinyl)amino]benzoic acid

6N sodium hydroxide (0.028 mL, 0.167 mmol) was added to a stirred solution of ethyl 3-[(7-(3,5-dimethyl-4-isoxazolyl)-3-{[(2-hydroxycyclopentyl)amino]carbonyl}-4-quinolinyl)amino]benzoate (43 mg, 0.084 mmol) in ethanol (1 mL) at room temperature, and the mixture stirred overnight. Purification by reverse-phase HPLC twice (10-70% MeCN/Water w/01. % TFA) afforded the title compound (6.5 mg, 0.013 mmol, 15.99% yield, ˜90% purity). ¹H NMR (METHANOL-d₄): 8.85 (s, 1H), 8.51 (d, J=8.8 Hz, 1H), 8.05 (dt, J=7.3, 1.5 Hz, 1H), 7.91-8.01 (m, 2H), 7.79 (dd, J=8.8, 1.5 Hz, 1H), 7.52-7.67 (m, 2H), 3.86 (d, J=5.8 Hz, 1H), 3.62 (br. s., 1H), 2.57 (s, 3H), 2.40 (s, 3H), 1.85-1.96 (m, 2H), 1.66-1.81 (m, 2H), 1.33-1.47 (m, 2H).

### Example 129

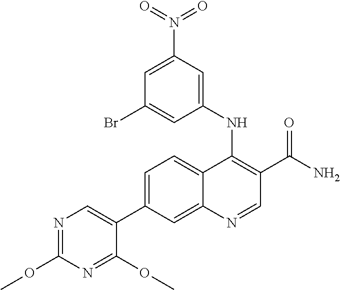 [SMILES: COc1ncc(-c2ccc3c(Nc4cc(Br)cc([N+](=O)[O-])c4)c(C(N)=O)cnc3c2)c(OC)n1]

### 7-[2,4-bis(methyloxy)-5-pyrimidinyl]-4-[(3-bromo-5-nitrophenyl)amino]-3-quinolinecarboxamide, trifluoroacetate salt

To a suspension of 7-[2,4-bis(methyloxy)-5-pyrimidinyl]-4-chloro-3-quinolinecarboxamide (example 1b, 200 mg, 0.580 mmol) in acetic acid (10 mL) was added (3-bromo-5-nitrophenyl)amine (example 74a, 126 mg, 0.580 mmol). The reaction mixture was stirred at room temperature overnight and was purified through preparative HPLC (YMC 75×30 mm column, 0.1% TFA in water and 0.1% TFA in acetonitrile) to afford the title compound (55 mg, 0.086 mmol, 14.83% yield) as an off white solid. ¹H NMR (400 MHz, DMSO-d6) δ ppm 10.83 (br. s., 1H), 8.99 (s, 1H), 8.68 (s, 1H), 8.34 (d, J=8.8 Hz, 1H), 8.29 (d, J=1.5 Hz, 1H), 8.23 (s, 1H), 8.10 (s, 1H), 7.94-7.99 (m, 2H), 7.74 (d, J=5.8 Hz, 2H), 4.03 (s, 3H), 4.00 (s, 3H). MS (ES+) m/e 525/527 [M+H]⁺.

### Example 130

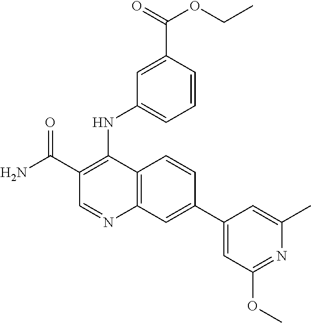 [SMILES: CCOC(=O)c1cccc(Nc2c(C(N)=O)cnc3cc(-c4cc(C)nc(OC)c4)ccc23)c1]

### ethyl 3-({3-(aminocarbonyl)-7-[2-methyl-6-(methyloxy)-4-pyridinyl]-4-quinolinyl}amino)benzoate

Preparation described in Example 107a.

### Example 131

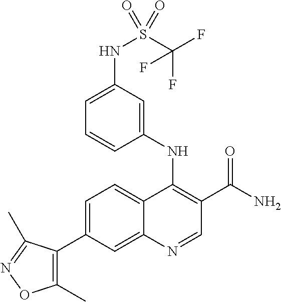 [SMILES: Cc1noc(C)c1-c1ccc2c(Nc3cccc(NS(=O)(=O)C(F)(F)F)c3)c(C(N)=O)cnc2c1]

### 7-(3,5-dimethyl-4-isoxazolyl)-4-[(3-{[(trifluoromethyl)sulfonyl]amino}phenyl)amino]-3-quinolinecarboxamide, trifluoroacetate salt

To a suspension of 4-chloro-7-(3,5-dimethyl-4-isoxazolyl)-3-quinolinecarboxamide (example 86a, 25 mg, 0.083 mmol) in acetic acid (10 mL) was added N-(3-aminophenyl)-1,1,1-trifluoromethanesulfonamide (19.90 mg, 0.083 mmol). The reaction mixture was stirred at room temperature overnight and was purified through preparative HPLC (YMC 75×30 mm column, 0.1% TFA in water and 0.1% TFA in acetonitrile) to afford the title compound (12 mg, 0.019 mmol, 23.38% yield) as a yellow solid. ¹H NMR (400 MHz, MeOD) δ ppm 9.02 (s, 1H), 7.93 (d, J=9.1 Hz, 1H), 7.86 (d, J=1.5 Hz, 1H), 7.45-7.55 (m, 2H), 7.25-7.33 (m, 3H), 2.49 (s, 3H), 2.32 (s, 3H). MS (ES+) m/e 506 [M+H]⁺.

### Example 132

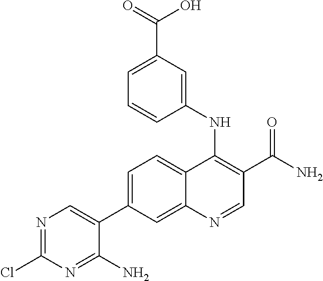 [SMILES: NC(=O)c1cnc2cc(-c3cnc(Cl)nc3N)ccc2c1Nc1cccc(C(=O)O)c1]

### 3-{[3-(aminocarbonyl)-7-(4-amino-2-chloro-5-pyrimidinyl)-4-quinolinyl]amino}benzoic acid, trifluoroacetate salt

A solution of ethyl 3-{[3-(aminocarbonyl)-7-bromo-4-quinolinyl]amino}benzoate (example 6a, 100 mg, 0.241 mmol), bis(pinacolato)diboron (92 mg, 0.362 mmol), potassium acetate (71.1 mg, 0.724 mmol), and tetrakis(triphenylphosphine)palladium(0) (12.55 mg, 10.86 μmol) in 1,4-dioxane (2.0 ml) was heated to 120° C. overnight in an oil bath. Upon cooling, the reaction mixture was treated with 5-bromo-2-chloro-4-pyrimidinamine (50.3 mg, 0.241 mmol) and sat aq sodium hydrogencarbonate (1 mL), then bubbled through with a nitrogen current (syringe in solution) for 5 minutes. The vial was capped, sealed and heated to 115° C. over 2 h using an oil bath. Upon cooling, the mixture was diluted with water and extracted with ethyl acetate twice. The organic portions were combined and concentrated to a solid, to which ethanol (2 ml) was added. After adding sodium hydroxide (1N aq solution) (0.5 ml, 0.500 mmol), the solution was stirred for 2 h at room temperature. The homogeneous solution was concentrated to an aqueous residue and purified using Reverse Phase HPLC (0.1% TFA in acetonitrile and water) to obtain the title compound as a white solid MS (ES+) m/e 435 [M+H]⁺.

### Example 133

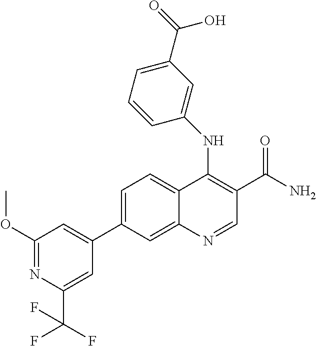 [SMILES: COc1cc(-c2ccc3c(Nc4cccc(C(=O)O)c4)c(C(N)=O)cnc3c2)cc(C(F)(F)F)n1]

### 3-({3-(aminocarbonyl)-7-[2-(methyloxy)-6-(trifluoromethyl)-4-pyridinyl]-4-quinolinyl}amino)benzoic acid

### a) ethyl 3-({3-(aminocarbonyl)-7-[2-(methyloxy)-6-(trifluoromethyl)-4-pyridinyl]-4-quinolinyl}amino)benzoate

A solution of ethyl 3-{[3-(aminocarbonyl)-7-bromo-4-quinolinyl]amino}benzoate (example 6a, 200 mg, 0.483 mmol), 2-(methyloxy)-4-(4,4,5,5-tetramethyl-1,3,2-dioxaborolan-2-yl)-6-(trifluoromethyl)pyridine (146 mg, 0.483 mmol) and tetrakis(triphenylphosphine)palladium(0) (27.9 mg, 0.024 mmol) in 1,4-dioxane (4 mL) and water (1 mL) was heated to 80° C. for 30 min in a Biotage Initiator microwave synthesizer. The reaction mixture was checked by UPLC-MS after 30 min and the desired product was observed. The reaction was stopped. Water was added and the mixture extracted 3×(20 ml) with ethyl-acetate. The combined organic extracts were dried over Na₂SO₄, filtered and evaporated to yield a yellow solid which was purified by using a silicagel column (10 g, 1-5% methanol/dichloromethane) to give the title compound (0.164 g, 73% pure, 49%) as a yellow solid. MS (ES+) m/e 511 [M+H]⁺.

### b) 3-({3-(aminocarbonyl)-7-[2-(methyloxy)-6-(trifluoromethyl)-4-pyridinyl]-4-quinolinyl}amino)benzoic acid

To a solution of ethyl 3-({3-(aminocarbonyl)-7-[2-(methyloxy)-6-(trifluoromethyl)-4-pyridinyl]-4-quinolinyl}amino)benzoate (164 mg, 0.321 mmol) in ethanol (4.5 ml), aqueous sodium hydroxide (4.50 ml, 4.50 mmol) was added and the mixture heated to 80° C. for 1.5 h in a Biotage Initiator microwave synthesizer. The reaction mixture was checked by UPLC-MS after 1.5 h and desired product was observed. The reaction was stopped. Ethanol was evaporated under reduced pressure and the pH was adjusted with 2M hydrochloric acid. The brownish precipitate was isolated by filtration and dried under vacuum. The sample was purified by mass-directed preparative HPLC to give the title compound (35 mg, 22%) as a yellow solid. MS (ES+) m/e 483 [M+H]⁺.

### Example 134

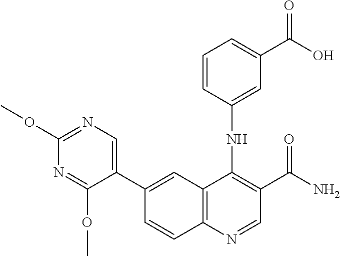 [SMILES: COc1ncc(-c2ccc3ncc(C(N)=O)c(Nc4cccc(C(=O)O)c4)c3c2)c(OC)n1]

### 3-({3-(aminocarbonyl)-6-[2,4-bis(methyloxy)-5-pyrimidinyl]-4-quinolinyl}amino)benzoic acid

### a) ethyl 6-bromo-4-hydroxy-3-quinolinecarboxylate

4-bromoaniline (2 g, 11.63 mmol) was added to neat diethyl [(ethyloxy)methylidene]propanedioate (2.446 ml, 12.21 mmol) and heated to 120° C. for 40 minutes. The ethanol was removed under reduced pressure, and the title compound crystallized in the flask as beige crystals. The crystals were then directly added portionwise to refluxing diphenyl ether, and were stirred for 1 hour. The solution was then cooled to rt and the resulting precipitate filtered to give the title compound (1.87 g, 6.32 mmol, 54.3% yield). MS (ES+) m/e 296 [M+H]⁺. The material was taken directly to the next step without further purification.

### b) 6-bromo-4-hydroxy-3-quinolinecarboxylic acid

6N sodium hydroxide (3.16 mL, 18.95 mmol) was added to a stirred solution of ethyl 6-bromo-4-hydroxy-3-quinolinecarboxylate (1.87 g, 6.32 mmol) in ethanol (40 mL) at 50° C. After two hours, the temperature was raised to 55° C. and reaction stirred overnight. A thick white precipitate formed, which was filtered and washed with ethanol to give the title compound (1.22 g, 4.55 mmol, 72.1% yield). ¹H NMR (DMSO-d₆): 13.57 (br. s., 1H), 8.96 (s, 1H), 8.38 (d, J=2.0 Hz, 1H), 8.06 (dd, J=8.8, 2.3 Hz, 1H), 7.80 (d, J=8.8 Hz, 1H).

### c) 6-bromo-4-chloro-3-quinolinecarboxamide

A 50 mL flask was charged with 6-bromo-4-hydroxy-3-quinolinecarboxylic acid (1.22 g, 4.55 mmol) and phosphorus oxychloride (7 mL, 75 mmol). The reaction was stirred at reflux. The mixture was concentrated to dryness, and redissolved in 1,4-dioxane (5 mL). The mixture was then cooled to 0° C. and ammonia was bubbled into the solution. After disappearance of starting material in the LCMS, the reaction was concentrated, filtered, and the solid washed with water, methanol, and ether to give the title compound (0.138 g, 11%). MS (ES+) m/e 285 [M+H]⁺.

### d) 3-{[3-(aminocarbonyl)-6-bromo-4-quinolinyl]amino}benzoic acid

3-aminobenzoic acid (72.8 mg, 0.532 mmol) was added to a stirred solution of 6-bromo-4-chloro-3-quinolinecarboxamide (138 mg, 0.483 mmol) in acetic acid (3 mL) at 60° C. The reaction was stirred for 3 hours before the mixture was cooled to r.t. The resulting precipitate was then filtered and washed with acetic acid to give the title compound (207 mg), which contained ˜25% of an unknown impurity. ¹H NMR (DMSO-d₆): 11.51 (br. s., 1H), 8.94 (s, 2H), 8.09-8.23 (m, 2H), 7.97-8.11 (m, 2H), 7.77-7.92 (m, 3H), 7.41-7.60 (m, 2H).

### e) 3-({3-(aminocarbonyl)-6-[2,4-bis(methyloxy)-5-pyrimidinyl]-4-quinolinyl}amino)benzoic acid

3-{[3-(aminocarbonyl)-6-bromo-4-quinolinyl]amino}benzoic acid (200 mg, 0.518 mmol), 2,4-dimethoxypyrimidine-5-boronic acid (143 mg, 0.777 mmol), tetrakis(triphenylphosphine)palladium(0) (29.9 mg, 0.026 mmol), and potassium carbonate (107 mg, 0.777 mmol) in 1,4-dioxane (3 mL)/water (1.500 mL) were added to a sealed microwave tube and irradiated to 120° C. for 30 minutes. 15 mL of water were then added and a thick precipitate formed. The precipitate was filtered and washed with water, ether and ethanol to give a cream solid of −87% purity. Additional purification by reverse-phase HPLC (C18, acetonitrile/300 mM aqueous ammonium formate) afforded the title compound (0.004 g, 1.8%). ¹H NMR (METHANOL-d₄): 8.96 (s, 1H), 8.07 (s, 1H), 8.02 (s, 1H), 7.97-8.01 (m, 2H), 7.78-7.84 (m, 1H), 7.69-7.75 (m, 1H), 7.45 (t, J=8.0 Hz, 1H), 7.28-7.35 (m, 1H), 4.02 (s, 3H), 3.94 (s, 3H).

### Example 135

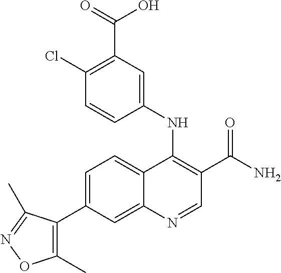 [SMILES: Cc1noc(C)c1-c1ccc2c(Nc3ccc(Cl)c(C(=O)O)c3)c(C(N)=O)cnc2c1]

### 5-{[3-(aminocarbonyl)-7-(3,5-dimethyl-4-isoxazolyl)-4-quinolinyl]amino}-2-chlorobenzoic acid, trifluoroacetate salt

To a suspension of 4-chloro-7-(3,5-dimethyl-4-isoxazolyl)-3-quinolinecarboxamide (example 86a, 50 mg, 0.166 mmol) in acetic acid (10 mL) was added 5-amino-2-chlorobenzoic acid (28.4 mg, 0.166 mmol). The reaction mixture was stirred at room temperature overnight and was purified through preparative HPLC (YMC 75×30 mm column, 0.1% TFA in water and 0.1% TFA in acetonitrile) to afford the title compound (35 mg, 0.064 mmol, 38.3% yield) as a yellow solid. ¹H NMR (400 MHz, DMSO-d6) δ ppm 13.57 (br. s., 1H), 11.41 (br. s., 1H), 9.01 (s, 1H), 8.29 (d, J=8.8 Hz, 1H), 8.24 (br. s., 1H), 8.03 (d, J=1.3 Hz, 1H), 7.80 (br. s., 1H), 7.76 (dd, J=9.0, 1.4 Hz, 1H), 7.68 (d, J=2.8 Hz, 1H), 7.55 (d, J=8.6 Hz, 1H), 7.39 (dd, J=8.6, 2.8 Hz, 1H), 2.53 (s, 3H), 2.34 (s, 3H). MS (ES+) m/e 437 [M+H]⁺.

### Example 136

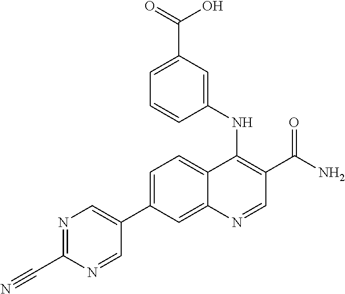 [SMILES: N#Cc1ncc(-c2ccc3c(Nc4cccc(C(=O)O)c4)c(C(N)=O)cnc3c2)cn1]

### 3-{[3-(aminocarbonyl)-7-(2-cyano-5-pyrimidinyl)-4-quinolinyl]amino}benzoic acid, trifluoroacetate salt

A solution of 3-{[3-(aminocarbonyl)-7-bromo-4-quinolinyl]amino}benzoic acid (example 88a, 100 mg, 0.259 mmol), 2-cyanopyrimidine-5-boronic acid (116 mg, 0.777 mmol), potassium carbonate (179 mg, 1.295 mmol), and tetrakis(triphenylphosphine)palladium(0) (13.46 mg, 0.012 mmol) in 1,4-dioxane (2.0 ml) and water (0.667 ml) was heated to 100° C. for 20 min. in a Biotage Initiator® microwave synthesizer. Upon cooling, the reaction mixture was concentrated in vacuo. The residue was dissolved into water and DMSO and purified via Reverse Phase HPLC (0.1% TFA in acetonitrile and water) to obtain the title compound (0.016 g, 12%) as a white solid. ¹H NMR (400 MHz, DMSO-d₆) ppm 13.11 (br. s., 1H), 11.27 (br. s., 1H), 9.56 (s, 2H), 9.03 (s, 1H), 8.48 (d, J=1.5 Hz, 1H), 8.20-8.34 (m, 2H), 8.11 (dd, J=8.97, 1.39 Hz, 1H), 7.67-7.84 (m, 3H), 7.47-7.54 (m, 1H), 7.40-7.48 (m, 1H). MS (ES+) m/e 411 [M+H]⁺.

### Example 137

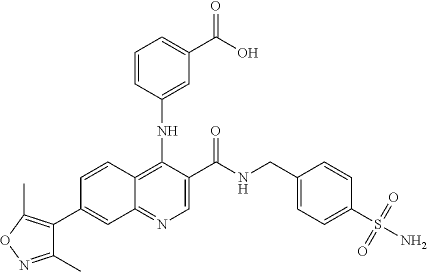 [SMILES: Cc1noc(C)c1-c1ccc2c(Nc3cccc(C(=O)O)c3)c(C(=O)NCc3ccc(S(N)(=O)=O)cc3)cnc2c1]

### 3-{[3-[({[4-(aminosulfonyl)phenyl]methyl}amino)carbonyl]-7-(3,5-dimethyl-4-isoxazolyl)-4-quinolinyl]amino}benzoic acid

### a) ethyl 3-{[3-[({[4-(aminosulfonyl)phenyl]methyl}amino)carbonyl]-7-(3,5-dimethyl-4-isoxazolyl)-4-quinolinyl]amino}benzoate

PyBOP (226 mg, 0.435 mmol) was added to a stirred solution of 7-(3,5-dimethyl-4-isoxazolyl)-4-({3-[(ethyloxy)carbonyl]phenyl}amino)-3-quinolinecarboxylic acid (example 13c, 125 mg, 0.290 mmol), 4-aminomethylbenzenesulfonamide hydrochloride (77 mg, 0.348 mmol), and triethylamine (0.121 mL, 0.869 mmol) in dichloromethane (3 mL) at room temperature, and the mixture stirred for 2 h. The solution was filtered and purified by HPLC (10-80% acetonitrile/water with 0.1% TFA) to give the title compound (41 mg, 0.068 mmol, 23.60% yield) as a pale yellow solid. ¹H NMR (METHANOL-d₄): 8.89 (s, 2H), 8.44 (d, J=8.8 Hz, 1H), 7.93-8.04 (m, 4H), 7.81-7.89 (m, 2H), 7.75 (dd, J=9.0, 1.6 Hz, 1H), 7.53-7.61 (m, 2H), 7.43 (d, J=8.6 Hz, 2H), 4.39 (q, J=7.2 Hz, 2H), 4.21 (s, 2H), 2.56 (s, 3H), 2.39 (s, 3H), 1.40 (t, J=7.1 Hz, 3H).

### b) 3-{[3-[({[4-(aminosulfonyl)phenyl]methyl}amino)carbonyl]-7-(3,5-dimethyl-4-isoxazolyl)-4-quinolinyl]amino}benzoic acid

6M aqueous sodium hydroxide (0.023 mL, 0.137 mmol) was added to a stirred solution of ethyl 3-{[3-[({[4-(aminosulfonyl)phenyl]methyl}amino)carbonyl]-7-(3,5-dimethyl-4-isoxazolyl)-4-quinolinyl]amino}benzoate (41 mg, 0.068 mmol) in ethanol (2 mL) at 60° C. overnight. Purification by reverse-phase preparative HPLC (10-70% acetonitrile/water, w/0.1% TFA) afforded the title compound (16 mg, 0.028 mmol, 40.9% yield) as a white powder. ¹H NMR (METHANOL-d₄): 8.87 (br. s., 1H), 8.44 (d, J=8.8 Hz, 1H), 7.99-8.04 (m, 1H), 7.96 (d, J=8.1 Hz, 2H), 7.87 (d, J=8.3 Hz, 2H), 7.75 (dd, J=8.8, 1.0 Hz, 1H), 7.55-7.60 (m, 2H), 7.46 (d, J=8.3 Hz, 2H), 4.22 (s, 2H), 2.56 (s, 3H), 2.39 (s, 3H).

### Example 138

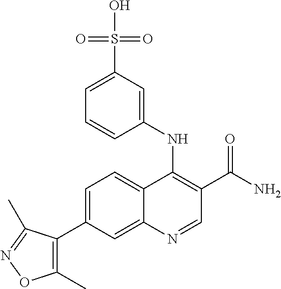 [SMILES: Cc1noc(C)c1-c1ccc2c(Nc3cccc(S(=O)(=O)O)c3)c(C(N)=O)cnc2c1]

### 3-{[3-(aminocarbonyl)-7-(3,5-dimethyl-4-isoxazolyl)-4-quinolinyl]amino}benzenesulfonic acid, trifluoroacetate salt

To a suspension of 4-chloro-7-(3,5-dimethyl-4-isoxazolyl)-3-quinolinecarboxamide (example 86a, 50 mg, 0.166 mmol) in acetic acid (10 mL) was added 3-aminobenzenesulfonic acid (28.7 mg, 0.166 mmol). The reaction mixture was stirred at room temperature overnight and was purified through preparative HPLC (YMC 75×30 mm column, 0.1% TFA in water and 0.1% TFA in acetonitrile) to afford the title compound (4 mg, 7.24 μmol, 4.37% yield) as a yellow solid. ¹H NMR (400 MHz, DMSO-d6) δ ppm 12.41 (br. s., 1H), 9.05 (s, 1H), 8.39 (br. s., 1H), 8.03 (d, J=9.1 Hz, 1H), 7.97 (br. s., 1H), 7.94 (d, J=1.8 Hz, 1H), 7.65 (dd, J=9.1, 1.8 Hz, 1H), 7.56-7.60 (m, 2H), 7.43 (t, J=8.0 Hz, 1H), 7.26-7.34 (m, 1H), 2.52 (s, 3H), 2.32 (s, 3H). MS (ES+) m/e 439 [M+H]⁺.

### Example 139

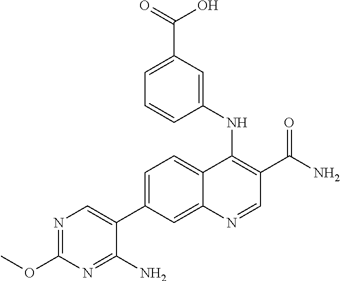 [SMILES: COc1ncc(-c2ccc3c(Nc4cccc(C(=O)O)c4)c(C(N)=O)cnc3c2)c(N)n1]

### 3-({3-(aminocarbonyl)-7-[4-amino-2-(methyloxy)-5-pyrimidinyl]-4-quinolinyl}amino)benzoic acid, trifluoroacetate salt

A solution of ethyl 3-{[3-(aminocarbonyl)-7-bromo-4-quinolinyl]amino}benzoate (example 6a, 200 mg, 0.483 mmol), bis(pinacolato)diboron (184 mg, 0.724 mmol), tetrakis(triphenylphosphine)palladium(0) (25.1 mg, 0.022 mmol) and potassium acetate (142 mg, 1.448 mmol) in 1,4-dioxane (2.0 ml) was heated to 100° C. for 20 min. in an oil bath. Upon cooling, the reaction mixture was treated with 5-bromo-2-(methyloxy)-4-pyrimidinamine (99 mg, 0.483 mmol) and sat aq sodium hydrogencarbonate (1 mL), then bubbled through with a nitrogen current (syringe in solution) for 5 minutes. The vial was capped, sealed and heated to 115° C. over 2 h using an oil bath. Upon cooling, the mixture was diluted with water and extracted with ethyl acetate twice. The organic portions were combined and concentrated to a solid, to which ethanol (2 ml) was added. After adding sodium hydroxide (1N aqueous solution) (0.5 ml, 0.500 mmol), the solution was stirred for 2 h at room temperature. The homogeneous solution was concentrated to an aqueous residue and purified using Reverse Phase HPLC (0.1% TFA in acetonitrile and water) to give the title compound (0.134 g, 51%). ¹H NMR (400 MHz, DMSO-d₆) ppm 11.62 (br. s., 1H), 8.98 (s, 1H), 8.20-8.32 (m, 2H), 8.11 (s, 1H), 8.00 (d, J=1.5 Hz, 1H), 7.85 (d, J=7.3 Hz, 1H), 7.81 (s, 1H), 7.76 (br. s., 1H), 7.68 (d, J=10.1 Hz, 1H), 7.44-7.58 (m, 3H), 3.90 (s, 3H). MS (ES+) m/e 431 [M+H]⁺.

### Example 140

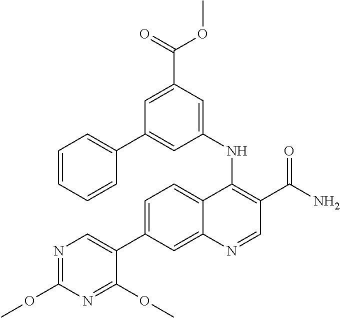 [SMILES: COC(=O)c1cc(Nc2c(C(N)=O)cnc3cc(-c4cnc(OC)nc4OC)ccc23)cc(-c2ccccc2)c1]

### methyl 5-({3-(aminocarbonyl)-7-[2,4-bis(methyloxy)-5-pyrimidinyl]-4-quinolinyl}amino)-3-biphenylcarboxylate

### a) methyl 5-amino-3-biphenylcarboxylate

To a suspension of methyl 3-amino-5-bromobenzoate (1.0 g, 4.35 mmol), phenylboronic acid (0.636 g, 5.22 mmol) and potassium carbonate (1.201 g, 8.69 mmol) in 1,4-dioxane (12 mL) and water (4.00 mL) was added tetrakis(triphenylphosphine)palladium(0) (0.251 g, 0.217 mmol). The mixture was heated to 100° C. overnight. After cooling, the mixture was diluted with water, acidified with 6N HCl and extracted with ethyl acetate. The organic layer was dried over MgSO₄, filtered, concentrated under reduced pressure and purified through flash chromatography to afford the title compound (750 mg, 3.30 mmol, 76% yield) as a colorless oil. ¹H NMR (400 MHz, CHLOROFORM-d) δ ppm 7.71 (t, J=1.5 Hz, 1H), 7.59-7.61 (m, 1H), 7.56-7.59 (m, 1H), 7.41-7.47 (m, 2H), 7.36-7.40 (m, 2H), 7.12-7.15 (m, 1H), 3.92 (s, 3H). MS (ES+) m/e 228 [M+H]⁺.

### b) methyl 5-({3-(aminocarbonyl)-7-[2,4-bis(methyloxy)-5-pyrimidinyl]-4-quinolinyl}amino)-3-biphenylcarboxylate

To a suspension of 7-[2,4-bis(methyloxy)-5-pyrimidinyl]-4-chloro-3-quinolinecarboxamide (example 1b, 100 mg, 0.290 mmol) in acetic acid (10 mL) was added methyl 5-amino-3-biphenylcarboxylate (65.9 mg, 0.290 mmol). The reaction mixture was stirred at room temperature overnight, quenched with water and extracted with methylene chloride. The organic layer was collected, dried over MgSO₄, filtered, concentrated under reduced pressure and purified through flash chromatography (0-10% methanol in methylene chloride) to afford the title compound (80 mg, 0.149 mmol, 51.5% yield) as a yellow solid. ¹H NMR (400 MHz, DMSO-d6) δ ppm 10.22 (s, 1H), 8.98 (s, 1H), 8.59 (s, 1H), 8.21 (br. s., 1H), 8.18 (d, J=1.5 Hz, 1H), 8.03 (d, J=8.8 Hz, 1H),7.79 (s, 1H), 7.69 (dd, J=9.0, 1.6 Hz, 1H), 7.65 (br. s., 1H), 7.64 (s, 1H), 7.62 (s, 1H), 7.54 (s, 1H), 7.48 (s, 1H), 7.42-7.47 (m, 2H), 7.35-7.41 (m, 1H), 3.98 (s, 3H), 3.97 (s, 3H), 3.84 (s, 3H). MS (ES+) m/e 536 [M+H]⁺.

### Example 141

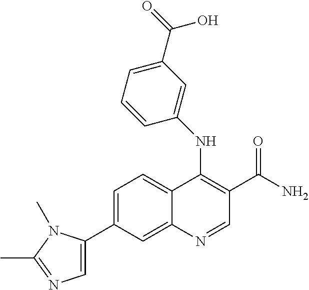 [SMILES: Cc1ncc(-c2ccc3c(Nc4cccc(C(=O)O)c4)c(C(N)=O)cnc3c2)n1C]

### 3-{[3-(aminocarbonyl)-7-(1,2-dimethyl-1H-imidazol-5-yl)-4-quinolinyl]amino}benzoic acid

### a) ethyl 3-{[3-(aminocarbonyl)-7-(1,2-dimethyl-1H-imidazol-5-yl)-4-quinolinyl]amino}benzoate

A solution of ethyl 3-{[3-(aminocarbonyl)-7-bromo-4-quinolinyl]amino}benzoate (example 6a, 400 mg, 0.966 mmol), (1,2-dimethyl-1H-imidazol-5-yl)boronic acid (405 mg, 2.90 mmol) and tetrakis(triphenylphosphine)palladium(0) (84 mg, 0.072 mmol) in dimethyl sulfoxide (8 ml) was heated to 100° C. for 30 min in a Biotage Initiator microwave synthesizer. The reaction mixture was checked by UPLC-MS after 30 min and 35% of desired product was observed. The reaction was stopped. Water was added and the mixture extracted with ethyl-acetate (3×20 ml). The combined organic extracts were dried over Na₂SO₄, filtered and evaporated to yield a yellow solid which was purified by chromatography (silicagel column (10 g), 1-5% dichloromethane/methanol) to yield the title compound (111 mg, 26%) as a yellow solid. MS (ES+) m/e 430 [M+H]⁺.

### b) 3-{[3-(aminocarbonyl)-7-(1,2-dimethyl-1H-imidazol-5-yl)-4-quinolinyl]amino}benzoic acid

To a solution of ethyl 3-{[3-(aminocarbonyl)-7-(1,2-dimethyl-1H-imidazol-5-yl)-4-quinolinyl]amino}benzoate (111 mg, 0.258 mmol) in ethanol (8 mL), aqueous sodium hydroxide (3.61 mL, 3.61 mmol) was added. The reaction mixture was stirred at 80° C. for 1 hour and checked by UPLC-MS. Desired product was obtained. The ethanol was removed in vacuo. The pH of reaction mixture adjusted down to 3, but a precipitate was not formed. By washing with dichloromethane, product was not extracted into the organic phase. The water layer was lyophilized and 94 mg of yellow solid was obtained, which was purified by preparative HPLC to give the title compound (8 mg, 8%) as a yellow solid. MS (ES+) m/e 402 [M+H]⁺.

### Example 142

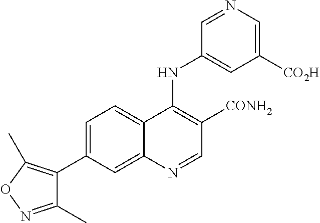 [SMILES: Cc1noc(C)c1-c1ccc2c(Nc3cncc(C(=O)O)c3)c(C(N)=O)cnc2c1]

### 5-{[3-(aminocarbonyl)-7-(3,5-dimethyl-4-isoxazolyl)-4-quinolinyl]amino}-3-pyridinecarboxylic acid

### a) methyl 5-{[3-(aminocarbonyl)-7-(3,5-dimethyl-4-isoxazolyl)-4-quinolinyl]amino}-3-pyridinecarboxylate

4-chloro-7-(3,5-dimethyl-4-isoxazolyl)-3-quinolinecarboxamide (example 86a, 150 mg, 0.497 mmol) and methyl 5-amino-3-pyridinecarboxylate (151 mg, 0.994 mmol) were dissolved in acetic acid (2.260 mL) and stirred at RT overnight. After that time, LCMS showed about 50% of conversion to the desired product. The reaction mixture was diluted with water and the pH was adjusted to 7.5. The mixture was extracted with dichloromethane. The organic layer was dried over sodium sulfate, filtered and evaporated affording crude methyl 5-{[3-(aminocarbonyl)-7-(3,5-dimethyl-4-isoxazolyl)-4-quinolinyl]amino}-3-pyridinecarboxylate (150 mg, 0.162 mmol, 32.5% yield), which was used in a next step without further purification. MS (ES+) m/e 418 [M+H]⁺.

### b) 5-{[3-(aminocarbonyl)-7-(3,5-dimethyl-4-isoxazolyl)-4-quinolinyl]amino}-3-pyridinecarboxylic acid

methyl 5-{[3-(aminocarbonyl)-7-(3,5-dimethyl-4-isoxazolyl)-4-quinolinyl]amino}-3-pyridinecarboxylate (100 mg, 0.240 mmol) was dissolved in methanol and 1N sodium hydroxide (0.240 mL, 0.240 mmol) was added. The mixture was stirred in a microwave reactor at 100° C. for 10 mins. After that time, the LCMS showed complete conversion. The solvent was evaporated and the crude product purified by mass-directed preparative HPLC yielding the title compound (15 mg, 92% pure, 14%). MS (ES+) m/e 404 [M+H]⁺.

### Example 143

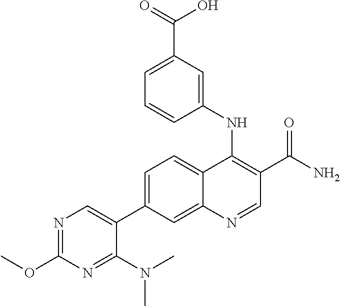 [SMILES: COc1ncc(-c2ccc3c(Nc4cccc(C(=O)O)c4)c(C(N)=O)cnc3c2)c(N(C)C)n1]

### 3-({3-(aminocarbonyl)-7-[4-(dimethylamino)-2-(methyloxy)-5-pyrimidinyl]-4-quinolinyl}amino)benzoic acid, trifluoroacetate salt

A solution of ethyl 3-{[3-(aminocarbonyl)-7-bromo-4-quinolinyl]amino}benzoate (example 6a, 200 mg, 0.483 mmol), bis(pinacolato)diboron (184 mg, 0.724 mmol), potassium acetate (142 mg, 1.448 mmol) and tetrakis(triphenylphosphine)palladium(0) (25.1 mg, 0.022 mmol) in 1,4-dioxane (5 ml) was heated to 115° C. for 3 h in an oil bath. Upon cooling, the reaction mixture was treated with 5-bromo-4-dimethylamino-2-(methyloxy)-pyrimidinamine (112 mg, 0.483 mmol) followed by sodium bicarbonate (sat aq solution) (2 mL, 0.483 mmol). The solution was bubbled through with a nitrogen current. After 5 minutes, the solution was treated with additional tetrakis(triphenylphosphine)palladium(0) (25.1 mg, 0.022 mmol) and heated to 115° C. overnight. Upon cooling, the contents were partitioned between water and ethyl acetate. The organic portion was dried over sodium sulfate, filtered and concentrated. The residue was purified via flash chromatography (10% methanol in dichloromethane) to obtain the crude intermediate ester as a yellow solid. A portion of this yellow solid was treated with sodium hydroxide (1N aqueous solution) (1 mL, 1.000 mmol) as a slurry in ethanol (2 mL). After stirring overnight at room temperature, the contents were purified using Reverse Phase HPLC (0.1% TFA in acetonitrile and water) to obtain the title compound (0.120 g, 43%) as a thick yellow oil. ¹H NMR (400 MHz, DMSO-d₆) ppm 11.83 (br. s., 1H), 8.98 (s, 1H), 8.27 (br. s., 1H), 8.21 (d, J=9.1 Hz, 1H), 8.19 (s, 1H), 7.96 (d, J=1.8 Hz, 1H), 7.84-7.89 (m, 1H), 7.83 (s, 1H), 7.77 (br. s., 1H), 7.63 (dd, J=9.1, 1.8 Hz, 1H), 7.52-7.58 (m, 2H), 3.97 (s, 3H), 2.88 (s, 6H). MS (ES+) m/e 459 [M+H]⁺.

### Example 144

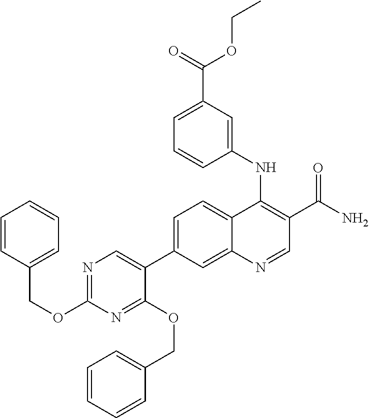 [SMILES: CCOC(=O)c1cccc(Nc2c(C(N)=O)cnc3cc(-c4cnc(OCc5ccccc5)nc4OCc4ccccc4)ccc23)c1]

### ethyl 3-[(3-(aminocarbonyl)-7-{2,4-bis[(phenylmethyl)oxy]-5-pyrimidinyl}-4-quinolinyl)amino]benzoate

A solution of ethyl 3-{[3-(aminocarbonyl)-7-bromo-4-quinolinyl]amino}benzoate (example 6a, 100 mg, 0.241 mmol), 2,4-bis(benzyloxy)pyrimidine-5-boronic acid (122 mg, 0.362 mmol) and tetrakis(triphenylphosphine)palladium(0) (12.55 mg, 10.86 μmol) in 1,4-dioxane (5 ml) and sodium bicarbonate (saturated aqueous solution) (2 mL, 0.241 mmol) was heated to 115° C. in an oil bath overnight. Upon cooling, the reaction mixture was diluted with ethyl acetate and washed with water. The organic portion was dried over sodium sulfate and concentrated. A portion of the residue was purified using flash chromatography (0-100% ethyl acetate in hexanes) to obtain the title compound (0.010 g, 7%) as a pale yellow solid. The rest of the material was treated with sodium hydroxide (1N aq solution) (1 mL, 1.000 mmol) as a solution in ethanol (5 mL). After stirring at room temperature overnight, the contents were purified using reverse phase HPLC (0.1% TFA in acetonitrile and water) to obtain the title compound (0.004 g, 3%) as a yellow solid. MS (ES+) m/e 626 [M+H]⁺.

### Example 145

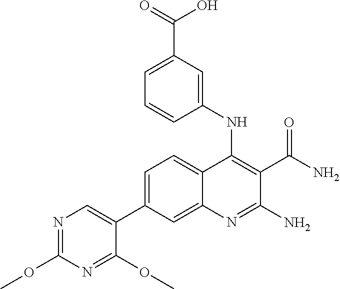 [SMILES: COc1ncc(-c2ccc3c(Nc4cccc(C(=O)O)c4)c(C(N)=O)c(N)nc3c2)c(OC)n1]

### 3-({2-amino-3-(aminocarbonyl)-7-[2,4-bis(methyloxy)-5-pyrimidinyl]-4-quinolinyl}amino)benzoic acid

### a) [(2-amino-4-bromophenyl)(hydroxy)methylidene]propanedinitrile

A solution of 7-bromo-2H-3,1-benzoxazine-2,4(1H)-dione (2.42 g, 10.0 mmol) was added to a stirred solution of malononitrile (0.727 g, 11.0 mmol) and triethylamine (1.53 mL, 11.0 mmol) in N,N-dimethylformamide (10 mL) and the mixture stirred at 60° C. under nitrogen for 1.5 h, before cooling. The mixture was poured into water (200 mL) and acidified to pH 1 with 6 M aqueous hydrochloric acid. The solid was filtered off, washed repeatedly with water and dried to give the title compound (2.34 g, 89%) as a dark green solid. ¹H NMR (400 MHz, DMSO-d₆) ppm 6.75 (dd, J=8.34, 1.77 Hz, 1H) 6.94 (d, J=1.77 Hz, 1H) 7.29 (d, J=8.34 Hz, 1H).

### b) 2-amino-7-bromo-4-hydroxy-3-quinolinecarbonitrile

A solution of [(2-amino-4-bromophenyl)(hydroxy)methylidene]propanedinitrile (2.08 g, 7.88 mmol) in N,N-dimethylformamide (40 mL) was boiled for 1 minute, then cooled to room temperature. The mixture was diluted with water (40 mL). The precipitate was filtered off, washed with water and dried to give the title compound (1.65 g, 79%) as a tan solid. ¹H NMR (400 MHz, DMSO-d₆) ppm 7.35 (s, 2H) 7.40 (dd, J=8.46, 1.89 Hz, 1H) 7.60 (d, J=1.77 Hz, 1H) 7.84 (d, J=8.59 Hz, 1H) 11.23 (br. s., 1H).

### c) 2-amino-7-bromo-4-chloro-3-quinolinecarboxamide

A mixture of 2-amino-7-bromo-4-hydroxy-3-quinolinecarbonitrile (0.197 g, 0.746 mmol) and phosphorus oxychloride (1 mL) was stirred at 110° C. for 5 h, then cooled and poured into iced water (10 mL). The stirred mixture was neutralised with ice-cooling using 5M aqueous sodium hydroxide. The solid was filtered off, washed with water and dried. LCMS showed mainly phosphorylated materials, with ˜15-20% product. The solid (0.140 g) was dissolved in 90% aqueous sulfuric acid (2 mL). The solution was stirred at 100° C. (mantle temperature) for 2 h, then cooled and poured into ice (30 mL). The mixture was made basic with 5M aqueous sodium hydroxide solution. The precipitate was filtered off, washed with water and dried to give the title compound (0.095 g, 42%) as a brown solid. ¹H NMR (400 MHz, DMSO-d₆) ppm 6.61 (br. s., 2H) 7.46 (dd, J=8.84, 2.02 Hz, 1H) 7.69 (d, J=2.02 Hz, 1H) 7.87 (d, J=8.84 Hz, 1H) 7.92 (br. s., 1H) 8.21 (s, 1H).

### d) ethyl 3-{[2-amino-3-(aminocarbonyl)-7-bromo-4-quinolinyl]amino}benzoate

A mixture of 2-amino-7-bromo-4-chloro-3-quinolinecarboxamide (0.093 g, 0.309 mmol), ethyl 3-aminobenzoate (0.102 g, 0.619 mmol) and acetic acid (1 mL) was stirred in a heating block set at 50° C. for 2 h, then cooled. The mixture was diluted with ether (1 mL). The solid was filtered off, washed with ether and dried to give the title compound (0.066 g, 50%) as a mustard yellow solid. ¹H NMR (400 MHz, DMSO-d₆) ppm 1.33 (t, J=7.07 Hz, 3H) 4.33 (q, J=7.07 Hz, 2H) 7.40-7.49 (m, 2H) 7.66-7.75 (m, 2H) 7.75-7.82 (m, 3H) 7.86 (d, J=2.02 Hz, 1H) 8.42 (d, J=8.34 Hz, 1H) 9.85 (br. s., 1H) 13.24 (br. s., 1H).

### e) 3-({2-amino-3-(aminocarbonyl)-7-[2,4-bis(methyloxy)-5-pyrimidinyl]-4-quinolinyl}amino)benzoic acid

A mixture of ethyl 3-{[2-amino-3-(aminocarbonyl)-7-bromo-4-quinolinyl]amino}benzoate (0.063 g, 0.147 mmol), [2,4-bis(methyloxy)-5-pyrimidinyl]boronic acid (0.054 g, 0.294 mmol), potassium carbonate (0.061 g, 0.440 mmol), bis(tri-t-butylphosphine)palladium(0) (0.008 g, 0.015 mmol), 1,4-dioxane (1 mL) and water (0.22 mL) was stirred at 150° C. in a microwave reactor for 0.5 h, then cooled. Acetic acid (0.060 g) was added and the mixture chromatographed (silica gel, 2-10% methanol/dichloromethane). The purified ester (LCMS:N16020-58-A1) was dissolved in ethanol (5 mL). 1M aqueous sodium hydroxide (1.5 mL, 1.5 mmol) was added and the mixture stirred for 18 h. The ethanol was removed under reduced pressure and the mixture diluted with water (3 mL) and adjusted to pH 4 with 1M aqueous hydrochloric acid. The precipitate was filtered off, washed with water and dried to give the title compound (0.010 g, 15%) as a orange-brown solid. ¹H NMR (400 MHz, DMSO-d₆) ppm 3.97 (s, 3H) 3.98 (s, 3H) 6.40 (br. s., 2H) 7.07 (dd, J=8.08, 1.52 Hz, 1H) 7.27 (t, J=7.83 Hz, 1H) 7.33 (dd, J=8.59, 1.52 Hz, 1H) 7.40 (d, J=7.58 Hz, 1H) 7.49 (br. s., 1H) 7.51 (s, 1H) 7.62-7.69 (m, 2H) 7.95 (d, J=8.59 Hz, 1H) 8.48 (s, 1H) 8.51 (s, 1H). ¹H NMR (400 MHz, DMSO-d₆+TFA) ppm 3.99 (s, 3H) 4.02 (s, 2H) 7.39-7.46 (m, 2H) 7.49 (br. s., 1H) 7.69-7.83 (m, 6H) 7.86 (d, J=1.52 Hz, 1H) 8.54 (d, J=8.84 Hz, 1H) 8.62 (s, 1H) 9.83 (s, 1H) 13.03 (br. s., 1H).

### Example 146

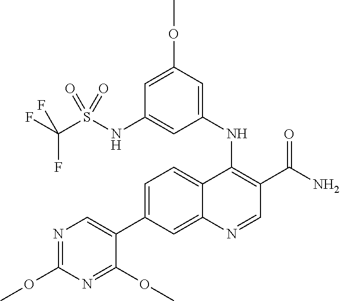 [SMILES: COc1cc(Nc2c(C(N)=O)cnc3cc(-c4cnc(OC)nc4OC)ccc23)cc(NS(=O)(=O)C(F)(F)F)c1]

### 7-[2,4-bis(methyloxy)-5-pyrimidinyl]-4-[(3-(methyloxy)-5-{[(trifluoromethyl)sulfonyl]amino}phenyl)amino]-3-quinolinecarboxamide

### a) 1,1,1-trifluoro-N-[3-(methyloxy)-5-nitrophenyl]methanesulfonamide

To a solution of [3-(methyloxy)-5-nitrophenyl]amine (100 mg, 0.595 mmol) and triethylamine (0.249 mL, 1.784 mmol) in dichloromethane (20 mL) cooled at 0° C. was added trifluoromethanesulfonic anhydride (0.201 mL, 1.189 mmol). After the reaction was kept stirring for 2 h at room temperature, it was quenched with water. The organic layer was collected, dried over MgSO₄, filtered, concentrated under reduced pressure and washed with methanol to afford a grey solid. The solid was dissolved in dichloromethane and methanol (1:1) and sodium hydroxide (6.0M in water) (0.099 mL, 0.595 mmol) was added. The mixture was kept stirring for another 0.5 h, diluted with water and extracted with dichloromethane. The organic layer was removed. The aqueous layer was acidified and extracted with ethyl acetate. The organic layer was collected, dried over MgSO₄, filtered and concentrated under reduced pressure to afford the title compound (95 mg, 0.316 mmol, 53.2% yield) as a brown solid. The product was used without further purification. MS (ES+) m/e 301 [M+H]⁺.

### b) 7-[2,4-bis(methyloxy)-5-pyrimidinyl]-4-[(3-(methyloxy)-5-{[(trifluoromethyl)sulfonyl]amino}phenyl)amino]-3-quinolinecarboxamide

To a suspension of 1,1,1-trifluoro-N-[3-(methyloxy)-5-nitrophenyl]methanesulfonamide (87 mg, 0.290 mmol) in acetic acid (10 mL) was added palladium-on-carbon, wet (3.09 mg, 0.029 mmol). The reaction was hydrogenated on a Parr shaker at 40 psi hydrogen for 3 h. The catalyst was removed by filtration, and 7-[2,4-bis(methyloxy)-5-pyrimidinyl]-4-chloro-3-quinolinecarboxamide (example 1b, 100 mg, 0.290 mmol) was added to the filtrate. The reaction mixture was kept stirring overnight and was purified by preparative HPLC (YMC 75×30 mm column, 0.1% TFA in water and 0.1% TFA in acetonitrile) to afford the title compound (25 mg, 0.036 mmol, 12.45% yield) as a yellow solid. ¹H NMR (400 MHz, MeOD) δ ppm 8.99 (s, 1H), 8.52 (s, 1H), 8.15 (d, J=1.5 Hz, 1H), 7.97 (d, J=8.8 Hz, 1H), 7.75 (dd, J=9.1, 1.8 Hz, 1H), 6.85-6.87 (m, 1H), 6.84 (qd, J=2.1, 1.9 Hz, 2H), 4.11 (s, 3H), 4.08 (s, 3H), 3.82 (s, 3H). MS (ES+) m/e 579 [M+H]⁺.

### Example 147

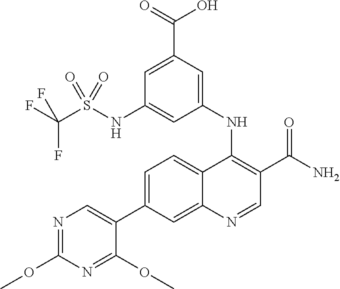 [SMILES: COc1ncc(-c2ccc3c(Nc4cc(NS(=O)(=O)C(F)(F)F)cc(C(=O)O)c4)c(C(N)=O)cnc3c2)c(OC)n1]

### 3-({3-(aminocarbonyl)-7-[2,4-bis(methyloxy)-5-pyrimidinyl]-4-quinolinyl}amino)-5-{[(trifluoromethyl)sulfonyl]amino}benzoic acid

To a solution of methyl 3-({3-(aminocarbonyl)-7-[2,4-bis(methyloxy)-5-pyrimidinyl]-4-quinolinyl}amino)-5-{[(trifluoromethyl)sulfonyl]amino}benzoate (example 75, 60 mg, 0.099 mmol) in methanol (10 mL) was added sodium hydroxide (6.0 N) (0.082 mL, 0.495 mmol). The reaction mixture was stirred at 50° C. overnight and was purified by preparative HPLC (YMC 75×30 mm column, 0.1% TFA in water and 0.1% TFA in acetonitrile) to afford the title compound (48 mg, 0.068 mmol, 68.7% yield) as a yellow solid. ¹H NMR (400 MHz, MeOD) δ ppm 8.98 (s, 1H), 8.53 (s, 1H), 8.20 (d, J=1.5 Hz, 1H), 8.07 (d, J=9.1 Hz, 1H), 7.87-7.90 (m, 1H), 7.81-7.86 (m, 2H), 7.50 (t, J=2.0 Hz, 1H), 4.12 (s, 3H), 4.08 (s, 3H). MS (ES+) m/e 593 [M+H]⁺.

### Example 148

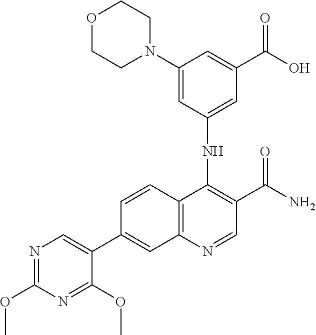 [SMILES: COc1ncc(-c2ccc3c(Nc4cc(C(=O)O)cc(N5CCOCC5)c4)c(C(N)=O)cnc3c2)c(OC)n1]

### 3-({3-(aminocarbonyl)-7-[2,4-bis(methyloxy)-5-pyrimidinyl]-4-quinolinyl}amino)-5-(4-morpholinyl)benzoic acid

### a) methyl 3-(4-morpholinyl)-5-nitrobenzoate

Under nitrogen, methyl 3-bromo-5-nitrobenzoate (250 mg, 0.961 mmol), morpholine (0.101 mL, 1.154 mmol), cesium carbonate (470 mg, 1.442 mmol), palladium (II) acetate (21.58 mg, 0.096 mmol), and tri-tertbutylphosphine (1M in hexane, 0.077 mL, 0.077 mmol) were added directly to a 50 mL round-bottom flask. Toluene (5 mL) was then added and the mixture stirred at ambient temperature for 2 hours. Purification by reverse-phase HPLC (10-90% acetonitrile/water w/0.01% TFA) afforded impure methyl 3-(4-morpholinyl)-5-nitrobenzoate (256 mg) as a pale yellow solid. MS (ES+) m/e 267 [M+H]⁺.

### b) 3-amino-5-(4-morpholinyl)benzoic acid

methyl 3-(4-morpholinyl)-5-nitrobenzoate (256 mg, 0.961 mmol) was added to a stirred solution of 6N aqueous sodium hydroxide (0.320 mL, 1.922 mmol) in methanol (3 mL) under nitrogen. Palladium-on-charcoal (102 mg, 0.961 mmol) was then added and the flask purged with hydrogen. The reaction was stirred overnight, filtered, and purified by reverse-phase HPLC (10-80% acetonitrile/water w/0.1% TFA) to the title compound (40 mg, 0.180 mmol, 18.73% yield) as a pale yellow syrup. ¹H NMR (METHANOL-d₄) δ 7.62 (d, J=1.0 Hz, 1H), 7.41 (s, 1H), 7.08 (s, 1H), 3.78-3.97 (m, 4H), 3.21-3.30 (m, 4H).

### c) 3-({3-(aminocarbonyl)-7-[2,4-bis(methyloxy)-5-pyrimidinyl]-4-quinolinyl}amino)-5-(4-morpholinyl)benzoic acid

3-amino-5-(4-morpholinyl)benzoic acid (33.8 mg, 0.152 mmol) was added to a stirred solution of 7-[2,4-bis(methyloxy)-5-pyrimidinyl]-4-chloro-3-quinolinecarboxamide (example 1b, 35 mg, 0.102 mmol) in acetic acid (2 mL) at rt. The reaction was stirred for 24 hours, then the precipitate was filtered, washed with acetic acid, and dried in vacuo. Purification by reverse-phase HPLC (10-50% acetonitrile/water w/0.1% TFA) afforded the title compound (25.5 mg, 0.048 mmol, 47.3% yield) as a yellow solid. ¹H NMR (METHANOL-d₄): 8.96 (s, 1H), 8.56 (s, 1H), 8.18 (d, J=1.5 Hz, 1H), 8.05 (d, J=8.8 Hz, 1H), 7.79 (dd, J=9.0, 1.6 Hz, 1H), 7.59-7.68 (m, 1H), 7.43 (d, J=1.8 Hz, 1H), 7.19 (s, 1H), 4.13 (s, 3H), 4.10 (s, 3H), 3.79-3.88 (m, 4H), 3.21-3.29 (m, 4H).

### Example 149

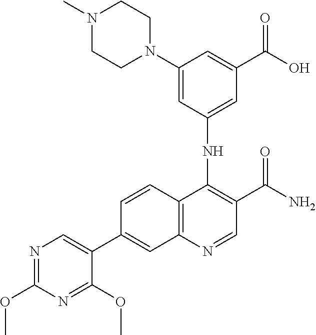 [SMILES: COc1ncc(-c2ccc3c(Nc4cc(C(=O)O)cc(N5CCN(C)CC5)c4)c(C(N)=O)cnc3c2)c(OC)n1]

### 3-({3-(aminocarbonyl)-7-[2,4-bis(methyloxy)-5-pyrimidinyl]-4-quinolinyl}amino)-5-(4-methyl-1-piperazinyl)benzoic acid

### a) methyl 3-(4-methyl-1-piperazinyl)-5-nitrobenzoate

Under nitrogen, methyl 3-bromo-5-nitrobenzoate (200 mg, 0.769 mmol), 1-methylpiperazine (0.078 mL, 0.699 mmol), cesium carbonate (342 mg, 1.048 mmol), palladium (II) acetate (15.69 mg, 0.070 mmol), and tri-tertbutylphosphine (10 wt % in hexane, 11.29 mg, 0.056 mmol) were added directly to a 50 mL round-bottom flask. Toluene (5 mL) was then added and the mixture stirred at 80° C. overnight. Ethyl acetate was then added and the organic phase washed with saturated aqueous sodium bicarbonate, water, and brine. The combined organic fractions were then purified by silica gel chromatography (0-10% dichloromethane/methanol) to give the title compound (73 mg, 0.261 mmol, 37.4% yield) as a yellow solid. MS (ES+) m/e 280 [M+H]⁺.

### b) 3-amino-5-(4-methyl-1-piperazinyl)benzoic acid

Crude methyl 3-(4-methyl-1-piperazinyl)-5-nitrobenzoate (73 mg, 0.261 mmol) was added to a stirred solution of 6N aqueous sodium hydroxide (0.087 mL, 0.523 mmol) in methanol (3 mL) under nitrogen. Palladium-on-charcoal (2.78 mg, 0.026 mmol) was then added and the flask purged with hydrogen. The reaction was stirred overnight, filtered, and purified by reverse-phase HPLC (10-80% acetonitrile/water w/0.1% TFA) to give the title compound (52 mg, 0.221 mmol, 85% yield). ¹H NMR (METHANOL-d₄): 7.53 (s, 1H), 7.43 (s, 1H), 7.11 (s, 1H), 3.54-3.73 (m, 4H), 3.12-3.30 (m, 4H), 2.98 (s, 3H).

### c) 3-({3-(aminocarbonyl)-7-[2,4-bis(methyloxy)-5-pyrimidinyl]-4-quinolinyl}amino)-5-(4-methyl-1-piperazinyl)benzoic acid

3-amino-5-(4-methyl-1-piperazinyl)benzoic acid (171 mg, 0.725 mmol) was added to a stirred solution of 7-[2,4-bis(methyloxy)-5-pyrimidinyl]-4-chloro-3-quinolinecarboxamide (example 1b, 125 mg, 0.363 mmol) in acetic acid (2 mL) at rt. The reaction was stirred for 24 hours, then the precipitate was filtered, washed with acetic acid, and dried in vacuo. Purification by reverse-phase HPLC (10-50% acetonitrile/water w/0.1% TFA) afforded the title compound (52 mg, 0.079 mmol, 21.81% yield) as a yellow solid. ¹H NMR (METHANOL-d₄): 8.95 (s, 1H), 8.57 (s, 1H), 8.22 (d, J=1.5 Hz, 1H), 8.12 (d, J=9.1 Hz, 1H), 7.80 (dd, J=9.0, 1.6 Hz, 1H), 7.61-7.69 (m, 1H), 7.51 (d, J=1.5 Hz, 1H), 7.27 (t, J=2.0 Hz, 1H), 4.15 (s, 3H), 4.12 (s, 3H), 3.97 (d, J=12.6 Hz, 2H), 3.63 (d, J=11.4 Hz, 2H), 3.15-3.30 (m, 4H), 2.98 (s, 3H).

### Example 150

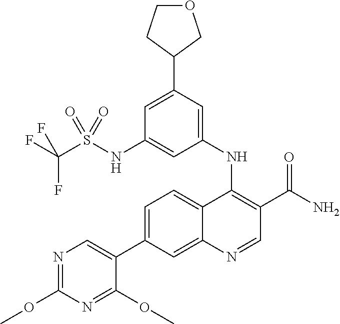 [SMILES: COc1ncc(-c2ccc3c(Nc4cc(NS(=O)(=O)C(F)(F)F)cc(C5CCOC5)c4)c(C(N)=O)cnc3c2)c(OC)n1]

### 7-[2,4-bis(methyloxy)-5-pyrimidinyl]-4-[(3-(tetrahydro-3-furanyl)-5-{[(trifluoromethyl)sulfonyl]amino}phenyl)amino]-3-quinolinecarboxamide

To a suspension of 1,1,1-trifluoro-N-[3-(3-furanyl)-5-nitrophenyl]methanesulfonamide (example 79b, 150 mg, 0.446 mmol) in acetic acid (10 mL) was added palladium-on-carbon (47.5 mg, 0.446 mmol). The reaction was hydrogenated on Parr shaker at 40 psi hydrogen for 3 h. The catalyst was removed by filtration, and 7-[2,4-bis(methyloxy)-5-pyrimidinyl]-4-chloro-3-quinolinecarboxamide (example 1b, 138 mg, 0.401 mmol) was added to the filtrate. The reaction mixture was kept stirring overnight and was purified by preparative HPLC (YMC 75×30 mm column, 0.1% TFA in water and 0.1% TFA in acetonitrile) to afford the title compound (50 mg, 0.059 mmol, 13.24% yield) as a yellow solid. ¹H NMR (400 MHz, MeOD) δ ppm 8.97 (s, 1H), 8.48 (s, 1H), 8.12 (d, J=1.5 Hz, 1H), 7.90 (d, J=9.1 Hz, 1H), 7.67 (dd, J=9.0, 1.6 Hz, 1H), 7.07-7.11 (m, 1H), 7.02 (s, 1H), 7.02 (s, 1H), 4.10 (s, 3H), 4.07 (s, 3H), 4.03-4.06 (m, 1H), 3.98 (td, J=8.2, 4.8 Hz, 1H), 3.85 (q, J=7.6 Hz, 1H), 3.69 (dd, J=8.6, 6.6 Hz, 1H), 3.43 (quin, J=7.4 Hz, 1H), 2.38 (m, 1H), 1.96 (m, 1H). MS (ES+) m/e 619 [M+H]⁺.

### Example 151

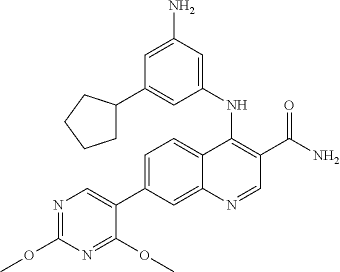 [SMILES: COc1ncc(-c2ccc3c(Nc4cc(N)cc(C5CCCC5)c4)c(C(N)=O)cnc3c2)c(OC)n1]

### 4-[(3-amino-5-cyclopentyl phenyl)amino]-7-[2,4-bis(methyloxy)-5-pyrimidinyl]-3-quinolinecarboxamide

### a) 1-(1-cyclopenten-1-yl)-3,5-dinitrobenzene

To a suspension of 1-bromo-3,5-dinitrobenzene (1000 mg, 4.05 mmol), 1-cyclopenten-1-ylboronic acid (906 mg, 8.10 mmol) and potassium carbonate (1119 mg, 8.10 mmol) in 1,4-dioxane (12 mL) and water (4.00 mL) was added tetrakis(triphenylphosphine)palladium(0) (468 mg, 0.405 mmol). The mixture was heated to 120° C. for 20 minutes under microwave irradiation. After cooling, the mixture was diluted with water, and extracted with ethyl acetate. The organic layer was dried over MgSO₄, filtered, concentrated under reduced pressure and purified through flash chromatography (0-100% ethyl acetate in hexanes) to afford the title compound (550 mg, 2.348 mmol, 58.0% yield) as an off pink solid. ¹H NMR (400 MHz, CHLOROFORM-d) ppm 8.87 (t, J=2.0 Hz, 1H), 8.54 (d, J=2.3 Hz, 2H), 6.53-6.62 (m, 1H), 2.77-2.85 (m, 2H), 2.66 (tq, J=7.5, 2.5 Hz, 2H), 2.09-2.19 (m, 2H). MS (ES+) m/e 235 [M+H]⁺.

### b) 4-[(3-amino-5-cyclopentylphenyl)amino]-7-[2,4-bis(methyloxy)-5-pyrimidinyl]-3-quinolinecarboxamide

To a mixture of 1-(1-cyclopenten-1-yl)-3,5-dinitrobenzene (100 mg, 0.427 mmol) and palladium-on-carbon (4.54 mg, 0.043 mmol) was added acetic acid (10 mL). The mixture was hydrogenated under 40 psi hydrogen on a Parr shaker for 3 h. After filtration, 7-[2,4-bis(methyloxy)-5-pyrimidinyl]-4-chloro-3-quinolinecarboxamide (example 1b, 147 mg, 0.427 mmol) was added to the filtrate. The reaction was kept stirring at room temperature overnight. The precipitate was collected by filtration and washed with ether to afford the title compound (35 mg, 0.072 mmol, 16.92% yield) as an off white solid. ¹H NMR (400 MHz, MeOD) δ ppm 9.00 (s, 1H), 8.57 (s, 1H), 8.21 (d, J=1.5 Hz, 1H), 8.11 (d, J=9.1 Hz, 1H), 7.83 (dd, J=9.1, 1.8 Hz, 1H), 7.27 (s, 1H), 7.23 (s, 1H), 7.15-7.20 (m, 1H), 4.14 (s, 3H), 4.10 (s, 3H), 3.02-3.16 (m, 1H), 2.06-2.17 (m, 2H), 1.78-1.88 (m, 2H), 1.68-1.78 (m, 2H), 1.54-1.66 (m, 2H). MS (ES+) m/e 485 [M+H]⁺.

### Example 152

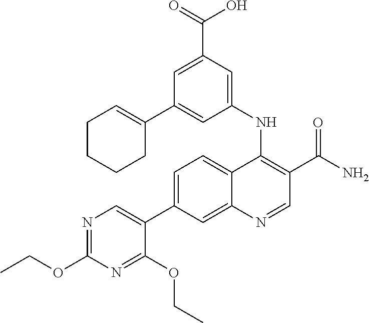 [SMILES: CCOc1ncc(-c2ccc3c(Nc4cc(C(=O)O)cc(C5=CCCCC5)c4)c(C(N)=O)cnc3c2)c(OCC)n1]

### 3-({3-(aminocarbonyl)-7-[2,4-bis(ethyloxy)-5-pyrimidinyl]-4-quinolinyl}amino)-5-(1-cyclohexen-1-yl)benzoic acid, trifluoroacetate salt

### a) 5-bromo-2,4-bis(ethyloxy)pyrimidine

Sodium ethoxide (21% wt in ethanol) (1.819 mL, 4.87 mmol) was added to a slurry of 5-bromo-2,4-dichloropyrimidine (500 mg, 2.194 mmol) in ethanol (10 mL) and the mixture stirred at reflux under nitrogen for 2 h. After cooling down, the mixture was concentrated under vacuum and washed with water. The obtained solid was washed with water and filtered to obtain the title compound (0.267 g, 49%) as a granular white solid. ¹H NMR (400 MHz, DMSO-d₆) δ ppm 8.47 (s, 1H), 4.42 (q, J=7.1 Hz, 2H), 4.31 (q, J=7.1 Hz, 2H), 1.33-1.37 (m, 3H), 1.28-1.33 (m, 3H). MS (ES+) m/e 247/249 [M+H]⁺.

### b) [2,4-bis(ethyloxy)-5-pyrimidinyl]boronic acid

A solution of 5-bromo-2,4-bis(ethyloxy)pyrimidine (217 mg, 0.878 mmol), bis(pinacolato)diboron (268 mg, 1.054 mmol), potassium acetate (259 mg, 2.63 mmol) and tetrakis(triphenylphosphine)palladium(0) (45.7 mg, 0.040 mmol) in 1,4-dioxane (10 ml) was stirred, while heating overnight to 115° C. using an oil bath. Upon cooling, the reaction mixture was used directly in the next experiment. MS (ES+) m/e 213 [M+H]⁺.

### c) 3-({3-(aminocarbonyl)-7-[2,4-bis(ethyloxy)-5-pyrimidinyl]-4-quinolinyl}amino)-5-(1-cyclohexen-1-yl)benzoic acid

A mixture of 7-bromo-4-chloro-3-quinolinecarboxamide (example 1a, 160 mg, 0.560 mmol) and 3-amino-5-(1-cyclohexen-1-yl)benzoic acid (example 54a, 86 mg, 0.339 mmol) in acetic acid (10 mL) was stirred at room temperature overnight. The resulting solid was filtered, washed with water and purified using Reverse Phase HPLC (0.1% TFA in acetonitrile and water) to obtain the intermediate bromo amide as a yellow solid, MS (ES+) m/e 466/468 [M+H]⁺. This solid was added to the reaction mixture from example 152b. The mixture was stirred, while heating to 115° C. overnight. The mixture was allowed to cool to room temperature and filtered. The resulting filtrate was diluted with brine and extracted with ethyl acetate. The extracts were dried over magnesium sulfate, filtered and concentrated. The residue was purified using Reverse Phase HPLC (0.1% TFA in acetonitrile and water) to obtain the title compound (22 mg, 0.033 mmol, 5.88% yield) as a yellow solid. ¹H NMR (400 MHz, DMSO-d₆) δ ppm 13.11 (br. s., 1H), 8.97 (s, 1H), 8.63 (s, 1H), 8.23 (s, 2H), 8.17 (d, J=8.3 Hz, 1H), 7.81-7.90 (m, 1H), 7.77-7.82 (m, 1H), 7.74 (br. s., 1H), 7.56-7.66 (m, 1H), 7.49 (br. s., 1H), 6.25 (br. s., 1H), 4.48 (q, J=7.1 Hz, 2H), 4.41 (q, J=7.1 Hz, 2H), 2.29-2.41 (m, 2H), 2.17 (dd, J=5.6, 4.0 Hz, 2H), 1.67-1.79 (m, 2H), 1.53-1.65 (m, 2H), 1.36 (td, J=7.1, 3.3 Hz, 6H). MS (ES+) m/e 554 [M+H]⁺.

### Example 153

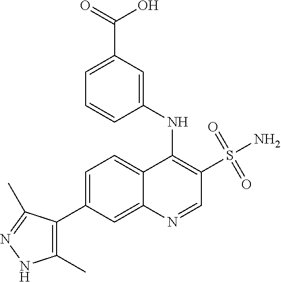 [SMILES: Cc1n[nH]c(C)c1-c1ccc2c(Nc3cccc(C(=O)O)c3)c(S(N)(=O)=O)cnc2c1]

### 3-{[3-(aminosulfonyl)-7-(3,5-dimethyl-1H-pyrazol-4-yl)-4-quinolinyl]amino}benzoic acid

A mixture of ethyl 3-{[3-(aminosulfonyl)-7-bromo-4-quinolinyl]amino}benzoate (example 52c, 0.130 g, 0.289 mmol), 3,5-dimethylpyrazole-4-boronic acid pinacol ester (0.128 g, 0.577 mmol), potassium carbonate (0.120 g, 0.866 mmol), bis(tri-t-butylphosphine)palladium(0) (0.010 g, 0.020 mmol), 1,4-dioxane (1.5 mL) and water (0.43 mL) was stirred at 150° C. in a microwave reactor for 0.5 h, then cooled. The supernatant liquor was chromatographed (silica gel, 2-10% methanol/dichloromethane) to give the intermediate ester (0.074 g). The ester was dissolved in methanol (7 mL) and 1M aqueous sodium hydroxide (2.00 mL, 2.00 mmol) added. The mixture was stirred at room temperature for 18 h. The methanol was removed under reduced pressure and water (10 mL) added. The solution was filtered, then acidified to pH 3 with 1M aqueous hydrochloric acid. The precipitate was filtered off, washed with water and dried to give the title compound (0.055 g, 44%) as a yellow solid. ¹H NMR (400 MHz, DMSO-d₆) ppm 2.27 (s, 6H) 7.17 (dd, J=7.96, 1.89 Hz, 1H) 7.40 (t, J=7.83 Hz, 1H) 7.44 (dd, J=8.84, 1.77 Hz, 1H) 7.50 (s, 1H) 7.61 (d, J=7.83 Hz, 1H) 7.64 (d, J=8.84 Hz, 1H) 7.85 (s, 2H) 7.89 (d, J=1.52 Hz, 1H) 8.40 (s, 1H) 9.11 (s, 1H) 12.75 (br. s., 2H).

### Example 154

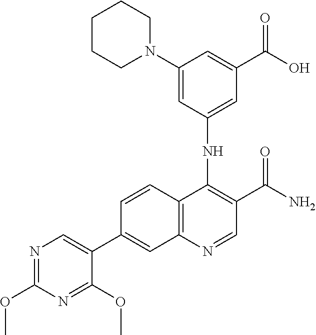 [SMILES: COc1ncc(-c2ccc3c(Nc4cc(C(=O)O)cc(N5CCCCC5)c4)c(C(N)=O)cnc3c2)c(OC)n1]

### 3-({3-(aminocarbonyl)-7-[2,4-bis(methyloxy)-5-pyrimidinyl]-4-quinolinyl}amino-5-(1-piperidinyl)benzoic acid

### a) 3-amino-5-(1-piperidinyl)benzoic acid

Under nitrogen, methyl 3-bromo-5-nitrobenzoate (250 mg, 0.961 mmol), piperidine (0.114 mL, 1.154 mmol), cesium carbonate (470 mg, 1.442 mmol), palladium (II) acetate (21.58 mg, 0.096 mmol), and tri-tertbutylphosphine (1M in hexane, 0.077 mL, 0.077 mmol) were added directly to a 50 mL round-bottom flask. Toluene (7 mL) was then added and the mixture stirred overnight at 80° C. The mixture was diluted with ethyl acetate and washed with saturated aqueous sodium bicarbonate, water, and brine. The combined organic fractions were concentrated under reduced pressure. Purification by reverse-phase HPLC (10-90% acetonitrile/water w/0.1% TFA) afforded the intermediate nitro compound. In a 50 mL round-bottom flask under nitrogen, the intermediate nitro compound, palladium-on-charcoal (102 mg, 0.961 mmol) and methanol (3 mL) were added and the flask purged with hydrogen. The reaction was stirred at room temperature overnight. The catalyst was filtered and 6N aqueous sodium hydroxide (0.080 mL, 0.481 mmol) was added and the mixture stirred at 50° C. overnight. The solvent was removed under reduced pressure to give the title compound (49.7 mg, 0.226 mmol, 23.47% yield). MS (ES+) m/e 221 [M+H]⁺.

### b) 3-({3-(aminocarbonyl)-7-[2,4-bis(methyloxy)-5-pyrimidinyl]-4-quinolinyl}amino)-5-(1-piperidinyl)benzoic acid

3-amino-5-(1-piperidinyl)benzoic acid (47.9 mg, 0.218 mmol) was added to a stirred solution of 7-[2,4-bis(methyloxy)-5-pyrimidinyl]-4-chloro-3-quinolinecarboxamide (example 1b, 50 mg, 0.145 mmol) in acetic acid (2 mL) at rt. The reaction was stirred for 24 hours, then the precipitate was filtered, washed with acetic acid, and dried in vacuo. Purification by reverse-phase HPLC (10-50% acetonitrile/water w/0.1% TFA) afforded the title compound (21 mg, 0.040 mmol, 27.4% yield) as a yellow solid. ¹H NMR (METHANOL-d₄): 8.97 (s, 1H), 8.59 (s, 1H), 8.14-8.28 (m, 2H), 7.94 (d, J=1.3 Hz, 1H), 7.89 (dd, J=9.1, 1.8 Hz, 1H), 7.76 (s, 1H), 7.55 (t, J=2.1 Hz, 1H), 4.15 (s, 3H), 4.11 (s, 3H), 3.46-3.54 (m, 4H), 1.84-1.93 (m, 4H), 1.65-1.78 (m, 2H).

### Example 155

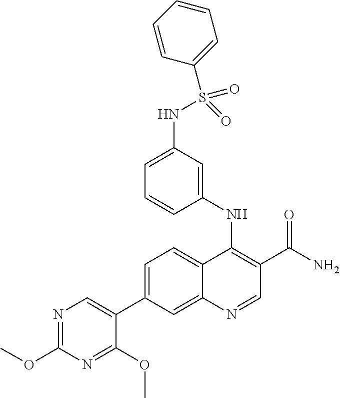 [SMILES: COc1ncc(-c2ccc3c(Nc4cccc(NS(=O)(=O)c5ccccc5)c4)c(C(N)=O)cnc3c2)c(OC)n1]

### 7-[2,4-bis(methyloxy)-5-pyrimidinyl]-4-({3-[(phenylsulfonyl)amino]phenyl}amino)-3-quinolinecarboxamide

### a) N-(3-aminophenyl)benzenesulfonamide

A solution of 1,1-dimethylethyl (3-aminophenyl)carbamate (200 mg, 0.960 mmol) and pyridine (388 μl, 4.80 mmol) in acetonitrile (10 mL) was kept at −30° C. while adding benzenesulfonylchloride (0.123 mL, 0.960 mmol) dropwise. The solution was stirred until it reached room temperature (ca. 3 h), then diluted with brine and extracted with ethyl acetate. The residue (light purple, MS (ES+) m/e 349 [M+H]⁺) was dissolved into dichloromethane and treated with trifluoroacetic acid (370 μl, 4.80 mmol). The reaction was stirred at room temperature overnight, then diluted with water and extracted using dichloromethane. The organic portion contained some desired product but most of it resided in the aqueous layer. Therefore this layer was treated with solid sodium bicarbonate and extracted with dichloromethane. The combined organic portions were dried over magnesium sulfate and filtered to obtain the title compound (313 mg, 1.261 mmol, 131% yield) as a thick purple gum, which was used directly in the next step. ¹H NMR (400 MHz, DMSO-d₆) ppm 10.06 (s, 1H), 8.59-8.78 (m, 1H), 7.99 (tt, J=7.7, 1.7 Hz, 1H), 7.73-7.80 (m, 2H), 7.50-7.63 (m, 3H), 6.88 (t, J=8.0 Hz, 1H), 6.49 (t, J=2.1 Hz, 1H), 6.36 (ddd, J=8.0, 2.0, 0.9 Hz, 1H), 6.31 (ddd, J=8.0, 2.1, 0.8 Hz, 1H). MS (ES+) m/e 249 [M+H]⁺.

### b) 7-[2,4-bis(methyloxy)-5-pyrimidinyl]-4-({3-[(phenylsulfonyl)amino]phenyl}amino)-3-quinolinecarboxamide

A mixture of N-(3-aminophenyl)benzenesulfonamide (313 mg, 1.261 mmol) and 7-[2,4-bis(methyloxy)-5-pyrimidinyl]-4-chloro-3-quinolinecarboxamide (example 1b, 333 mg, 0.966 mmol) in acetic acid (10 mL) was stirred at room temperature for 2 h. The resulting solid was filtered, then washed with ethyl ether and purified using Reverse Phase Gilson (0.1% TFA in acetonitrile and water) to obtain the title compound (0.107 g, 15%) as a bright yellow solid. MS (ES+) m/e 557 [M+H]⁺.

### Example 156

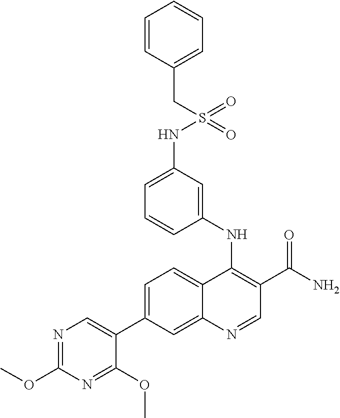 [SMILES: COc1ncc(-c2ccc3c(Nc4cccc(NS(=O)(=O)Cc5ccccc5)c4)c(C(N)=O)cnc3c2)c(OC)n1]

### 7-[2,4-bis(methyloxy)-5-pyrimidinyl]-4-[3-{[(phenylmethyl)sulfonyl]amino}phenyl)amino]-3-quinolinecarboxamide

### a) N-(3-aminophenyl)-1-phenylmethanesulfonamide

A solution of 1,1-dimethylethyl (3-aminophenyl)carbamate (200 mg, 0.960 mmol) and pyridine (388 μl, 4.80 mmol) in acetonitrile (10 mL) was kept at −30° C. while adding benzylsulfonylchloride (183 mg, 0.960 mmol) dropwise. The solution was stirred until it reached room temperature, then diluted with brine and extracted with ethyl acetate. The residue after evaporation of solvent (light orange, sticky oil) was dissolved into dichloromethane and treated with trifluoroacetic acid (370 μl, 4.80 mmol). The reaction was stirred at room temperature overnight, then diluted with aqueous sodium bicarbonate and extracted with dichloromethane. The solvent was removed under reduced pressure to give the title compound (0.107 g, 43%) as a yellow solid. ¹H NMR (400 MHz, DMSO-d₆) δ ppm 9.53 (s, 1H), 7.31-7.43 (m, 4H), 7.22-7.30 (m, 2H), 6.95 (t, J=8.0 Hz, 1H), 6.51 (t, J=2.0 Hz, 1H), 6.37 (dd, J=7.8, 1.3 Hz, 1H), 6.29 (dd, J=7.8, 1.3 Hz, 1H), 5.85-5.95 (m, 1H), 4.38 (s, 2H). MS (ES+) m/e 263 [M+H]⁺.

### b) 7-[2,4-bis(methyloxy)-5-pyrimidinyl]-4-[(3-{[(phenylmethyl)sulfonyl]amion}phenyl)amino]-3-quinolinecarboxamide

A mixture of N-(3-aminophenyl)-1-phenylmethanesulfonamide (107 mg, 0.408 mmol) and 7-[2,4-bis(methyloxy)-5-pyrimidinyl]-4-chloro-3-quinolinecarboxamide (example 1b, 141 mg, 0.408 mmol) in acetic acid (10 mL) was stirred at room temperature overnight. The resulting solid was filtered, then washed with ethyl ether and purified using Reverse Phase HPLC (0.1% TFA in acetonitrile and water) to obtain the title compound (0.035 g, 15%) as a yellow solid. ¹H NMR (400 MHz, DMSO-d₆) δ ppm 11.93 (br. s., 1H), 10.05 (s, 1H), 9.05 (s, 1H), 8.57 (s, 1H), 8.37 (br. s., 1H), 8.21 (d, J=1.8 Hz, 1H), 7.98 (d, J=8.8 Hz, 1H), 7.92 (br. s., 1H), 7.79 (d, J=8.1 Hz, 1H), 7.34-7.40 (m, 1H), 7.28-7.33 (m, 3H), 7.20-7.26 (m, 2H), 7.04-7.13 (m, 2H), 6.99 (d, 1H), 4.47 (s, 2H), 3.97 (s, 6H). MS (ES+) m/e 571 [M+H]⁺.

### Example 157

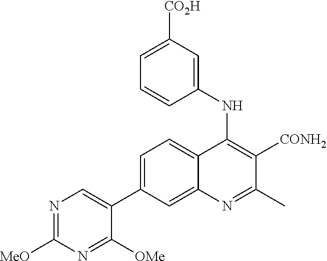 [SMILES: COc1ncc(-c2ccc3c(Nc4cccc(C(=O)O)c4)c(C(N)=O)c(C)nc3c2)c(OC)n1]

### 3-({3-(aminocarbonyl)-7-[2,4-bis(methyloxy)-5-pyrimidinyl]-2-methyl-4-quinolinyl}amino)benzoic acid

### a) diethyl {1-[(3-bromophenyl)amino]ethylidene}propanedioate

A solution of 3-bromoaniline (6.40 g, 37.2 mmol) and diethyl acetylmalonate (8.35 g, 41.3 mmol) in ethanol (30 mL) was stirred under reflux for 24 h. After cooling, the solvent was removed under reduced pressure. The residue was chromatographed (silica gel, 10-90% ethyl acetate/hexane) to give the title compound (5.09 g, 46% pure, 18%) contaminated with diethyl malonate. ¹H NMR (400 MHz, DMSO-d₆) δ ppm 1.16-1.24 (m, 6H) 2.05 (s, 3H) 4.07-4.17 (m, 4H) 7.24-7.29 (m, 1H) 7.35 (t, J=7.96 Hz, 1H) 7.45 (d, J=8.08 Hz, 1H) 7.52 (t, J=1.89 Hz, 1H) 10.88 (s, 1H). The material was used in the next step without further purification.

### b) ethyl 7-bromo-4-hydroxy-2-methyl-3-quinolinecarboxylate

Diethyl {1-[(3-bromophenyl)amino]ethylidene}propanedioate (5.09 g, 46% pure, 6.57 mmol) was added in portions to boiling diphenyl ether (30 mL) with stirring. After the addition, the solution was stirred under reflux for 0.5 h, then cooled and diluted with ether (30 mL). The precipitate was filtered off, washed with ether and dried to give the title compound (1.57 g, 90% pure, 69%) as a brown solid. ¹H NMR (400 MHz, DMSO-d₆) δ ppm 1.27 (t, J=7.07 Hz, 3H) 2.39 (s, 3H) 4.24 (q, J=7.07 Hz, 2H) 7.50 (dd, J=8.59, 1.77 Hz, 1H) 7.71 (d, J=2.02 Hz, 1H) 7.97 (d, J=8.59 Hz, 1H) 11.91 (s, 1H).

### c) 7-bromo-4-hydroxy-2-methyl-3-quinolinecarboxylic acid

A mixture of ethyl 7-bromo-4-hydroxy-2-methyl-3-quinolinecarboxylate (1.57 g, 5.06 mmol), 1M aqueous sodium hydroxide (25.3 mL, 25.3 mmol) and ethanol (70 mL) was stirred under reflux for 60 h, then cooled. The mixture was concentrated under reduced pressure to remove most of the ethanol, then diluted with water (80 mL) and acidified to pH 3 with 6M aqueous hydrochloric acid. The mixture was extracted with ethyl acetate. The extracts were washed with 0.5M aq potassium carbonate and the aqueous extracts filtered, then acidified to pH 3 with 6M aqueous hydrochloric acid. The solid was filtered off, washed with water and dried to give the title compound (1.16 g, 81%) as a brown solid. ¹H NMR (400 MHz, DMSO-d₆) δ ppm 2.89 (s, 3H) 7.72 (dd, J=8.59, 1.77 Hz, 1H) 7.96 (d, J=1.77 Hz, 1H) 8.16 (d, J=8.84 Hz, 1H) 13.20 (br. s., 1H) 16.18 (br. s., 1H).

### d) 7-bromo-4-chloro-2-methyl-3-quinolinecarboxamide

A mixture of 7-bromo-4-hydroxy-2-methyl-3-quinolinecarboxylic acid (1.15 g, 4.08 mmol) and phosphorus oxychloride (11 mL) was stirred under reflux for 3 h, then cooled. The solvent was removed under reduced pressure and the residue azeotroped twice with toluene. The flask was cooled in cold water and 0.5M ammonia in dioxane (82 mL) added rapidly with stirring. The mixture was stirred 1 h at room temperature, then the solvent removed under reduced pressure. The residue was slurried in water (120 mL). The solid was filtered off, washed with water and dried to give the title compound (1.05 g, 53% pure, 46%) as a solid, contaminated with the 3-carboxylic acid as the major impurity. NMR of the desired amide: ¹H NMR (400 MHz, DMSO-d₆) δ ppm 2.66 (s, 3H) 7.91 (dd, J=9.09, 2.02 Hz, 1H) 8.02 (br. s., 1H) 8.13 (d, J=4.80 Hz, 1H) 8.21 (br. s., 1H) 8.26 (d, J=1.77 Hz, 1H).

### e) ethyl 3-{[3-(aminocarbonyl)-7-bromo-2-methyl-4-quinolinyl]amino}benzoate

A mixture of 7-bromo-4-chloro-2-methyl-3-quinolinecarboxamide (0.850 g, 2.84 mmol), ethyl 3-aminobenzoate (0.703 g, 4.26 mmol) and acetic acid (18 mL) was stirred in a heating block set at 50° C. for 4 h, then cooled. The solvent was removed under reduced pressure. The residue was slurried in 1M aqueous hydrochloric acid (50 mL) and the mixture washed with ethyl acetate. The washings were back extracted with 1M aqueous hydrochloric acid. The aqueous phase was adjusted to pH 9 with 0.5M aqueous potassium carbonate and extracted with ethyl acetate. The extracts were dried (MgSO₄) and evaporated under reduced pressure. The residue was chromatographed (silica gel, 30-100% ethyl acetate/hexane, then 10% methanol/dichloromethane) to give the title compound (0.259 g, 21%) as a brown powder. ¹H NMR (400 MHz, DMSO-d₆) δ ppm 1.28 (t, J=7.07 Hz, 3H) 2.64 (s, 3H) 4.27 (q, J=7.07 Hz, 2H) 6.98 (dd, J=8.21, 1.64 Hz, 1H) 7.25 (t, J=7.96 Hz, 1H) 7.41 (d, J=7.83 Hz, 1H) 7.47 (t, J=1.89 Hz, 1H) 7.57 (d, J=1.26 Hz, 1H) 7.59 (dd, J=8.84, 2.02 Hz, 1H) 7.77 (d, J=9.09 Hz, 1H) 7.85 (s, 1H) 8.13 (d, J=2.02 Hz, 1H) 8.54 (s, 1H).

### f) 3-({3-(aminocarbonyl)-7-[2,4-bis(methyloxy)-5-pyrimidinyl]-2-methyl-4-quinolinyl}amino)benzoic acid

A mixture of ethyl 3-{[3-(aminocarbonyl)-7-bromo-2-methyl-4-quinolinyl]amino}benzoate (0.130 g, 0.304 mmol), [2,4-bis(methyloxy)-5-pyrimidinyl]boronic acid (0.112 g, 0.607 mmol), potassium carbonate (0.168 g, 1.21 mmol), tetrakis(triphenylphosphine)palladium(0) (0.018 g, 0.015 mmol), 1,4-dioxane (2 mL) and water (0.61 mL) was stirred at 150° C. in a microwave reactor for 0.5 h, then cooled. The supernatant liquor was chromatographed (silica gel, 2-10% methanol/dichloromethane) to give the intermediate ester. 1M aqueous sodium hydroxide (2.36 mL, 2.36 mmol) was added to a stirred solution of the intermediate ester in methanol (12 mL) and the mixture stirred at room temperature for 18 h. The methanol was removed under reduced pressure and water (5 mL) added. The pH was adjusted to 3 with 6M aqueous hydrochloric acid. The mixture was stirred 0.5 h and the precipitate filtered off, washed with water and dried to give the title compound (0.097 g, 70%) as a yellow solid. ¹H NMR (400 MHz, DMSO-d₆) δ ppm 2.66 (s, 3H) 3.98 (s, 3H) 4.00 (s, 3H) 7.04 (br. s., 1H) 7.27 (br. s., 1H) 7.44 (br. s., 2H) 7.57 (br. s., 1H) 7.68 (br. s., 1H) 7.86 (br. s., 2H) 8.14 (d, J=1.26 Hz, 1H) 8.39 (br. s., 1H) 8.59 (s, 1H).

### Example 158

### Capsule Composition

An oral dosage form for administering the present invention is produced by filing a standard two piece hard gelatin capsule with the ingredients in the proportions shown in Table I, below.

TABLE I

| INGREDIENTS | AMOUNTS |
|---|---|
| 3-({3-(aminocarbonyl)-7-[2,4-bis(methyloxy)-5- | 7 mg |
| pyrimidinyl]-4-quinolinyl}amino)benzoic acid | |
| (Compound of Example 6) | |
| Lactose | 53 mg |
| Talc | 16 mg |
| Magnesium Stearate | 4 mg |

### Example 159

### Injectable Parenteral Composition

An injectable form for administering the present invention is produced by stirring 1.7% by weight of 5-{[3-(aminocarbonyl)-7-(3,5-dimethyl-1H-pyrazol-4-yl)-4-quinolinyl]amino}-1,3-benzenedicarboxylic acid (Compound of Example 5) in 10% by volume propylene glycol in water.

### Example 160

### Tablet Composition

The sucrose, calcium sulfate dihydrate and a lactate dehydrogenase A inhibitor as shown in Table II below, are mixed and granulated in the proportions shown with a 10% gelatin solution. The wet granules are screened, dried, mixed with the starch, talc and stearic acid, screened and compressed into a tablet.

TABLE II

| | |
|---|---|
| INGREDIENTS | AMOUNTS |

| | | |
|---|---|---|
| 5-({3-(aminocarbonyl)-7-[2,4-bis(methyloxy)-5- | 12 | mg |
| pyrimidinyl]-4-quinolinyl}amino)-1,3- | | |
| benzenedicarboxylic acid (Compound of Example 4) | | |
| calcium sulfate dihydrate | 30 | mg |
| sucrose | 4 | mg |
| starch | 2 | mg |
| talc | 1 | mg |
| stearic acid | 0.5 | mg |

### Biological Activity

### Enzymatic Assay

Assay buffer (50 mM potassium phosphate, pH 7.0, 50 mM NaCl, 0.25 mM CHAPS, 0.0025% BSA) is added to one column of a standard assay plate stamped with DMSO solutions of test compounds. A solution of LDH-A and NAD+ (final concentrations: 300 pM LDH-A and 150 uM NAD+) in assay buffer is added to remaining plate wells containing DMSO solutions of test compounds and DMSO only control wells and the mixtures incubated for 10 min at room temperature. A solution of lactate, resazurin, diaphorase (final concentrations: 2 mM lactate, 6.25 uM resazurin, 0.4 U/mL diaphorase) in assay buffer is added to all wells and the mixtures incubated for 30 min at room temperature. Stop solution (2 uL, components: assay buffer and 25 mM oxamate) is added to each well and the fluoresecence intensity increase (FLINT) measured at 590 nm on a ViewLux®imager.

### Cell-Based Assay

A 384 well plate is stamped with DMSO solutions of test compounds and DMSO only controls. Frozen U2OS cells are thawed in assay medium (DMEM/F12 (HAM's) 1:1 with L-glutamine with 15 mM Hepes/1% FBS without phenol red). The cell suspension is centrifuged for 3 min at 1200 rpm. The supernatant is removed and the cell pellet re-suspended in 5-10 mL assay medium (volume may vary). 1.5 mL is removed from the re-suspended cell pellet and counted on Cedex counter. The remaining cell pellet is re-suspended to 0.75×10⁶ cells/mL in appropriate volume of assay medium. 0.75×10⁶ cells/mL cell suspension is gently stirred to keep in suspension. 20 uL of 0.75×10⁶ cells/mL cell suspension is plated to pre-stamped compound plate except column 18 which is left blank. 20 uL assay media is added to column 18. Plates are centrifuged at 500 rpm for 20-30 sec, placed into incubator and incubated overnight (18-24 hr) at 37° C. with 5% carbon dioxide.

5 uL cell medium is transferred from cell compound plate to Greiner low volume black 384 plate using a Cybio CyBi®-Well 384-Channel Pipettor. The plate is centrifuged for 30 sec at 500 rpm. 5 uL of 2× enzyme mix (components: 1 mM thiamine pyrophosphate, 2 U/mL horseradish peroxidase in Tris pH 7.5, 0.08 U/mL pyruvate oxidase in HEPES pH 7.5, 0.2 mM Amplex UltraRed made up in assay buffer (50 mM sodium phosphate pH 7.5 and 5 mM magnesium chloride) containing 0.01% BSA) is added into the low volume black 384 plate containing 5 uL cell medium. The plate is centrifuge for 30 sec at 500 rpm (protected from light), then incubated in the dark at room temperature for 60 min. The plate is read on ViewLux® imager for fluorescence intensity at excitation/emission wavelengths of 525/598, respectively.

Compounds of the invention are tested for activity against lactate dehydrogenase A in the above assay.

The compounds of Examples 1 to 157 were tested generally according to either the above Enzymatic Assay or the above Cell-based Assay and in at least one set of experimental runs exhibited an average pIC₅₀ value ≧5.0.

The compound of Example 60 was tested generally according to the above lactate dehydrogenase A enzyme assay and in at least one set of experimental runs exhibited an average lactate dehydrogenase A pIC₅₀ value of 7.2 against lactate dehydrogenase A.

While the preferred embodiments of the invention are illustrated by the above, it is to be understood that the invention is not limited to the precise instructions herein disclosed and that the right to all modifications coming within the scope of the following claims is reserved.

1. A compound according to Formula I: 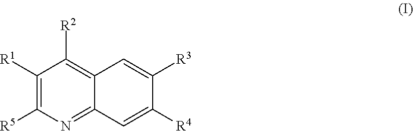
wherein:
R¹ is selected from: —COOH, —CONH₂, —SO₂NH₂, —CONHCH₂R¹⁰, —NHCONH₂, —CONCH₂thiazole and tetrazole,
where R¹⁰ is selected from:
—COOH,
phenyl,
phenyl substituted with from one to three substituents independently selected from: —OH, —COOH, —NH₂, —OCH₃, —SO₂NH₂, —NHCOCH₃ and —CF₃,
cyclopentyl, and
cyclopentyl substituted with from one to three substituents independently selected from: —OH, —COOH, —NH₂, —OCH₃, —SO₂NH₂, —NHCOCH₃ and —CF₃;
R² is selected from: —NHphenyl and —NHpyridinyl,
where
the pyridinyl is substituted with from one to three substituents independently selected from: —COOH, C₁-C₆alkyl —F, —Cl, —Br, and —I,
the phenyl is substituted with from one to three substituents independently selected from:
C₁-C₆alkyl,
—F,
—Cl,
—Br,
—I,
—OH,
—COOH,
phenyl,
phenyl substituted with from one to three substituents independently selected from: C₁-C₆alkyl, —OCH₃, —CF₃, —F, —Cl, —Br and —I,
heteroaryl,
heteroaryl substituted with from one to three substituents independently selected from: C₁-C₆alkyl, —OCH₃, —CF₃, —F, —Cl, —Br and —I,
cycloalkyl,
cycloalkyl substituted with from one to three substituents independently selected from: C₁-C₆alkyl, —OCH₃, —CF₃, —F, —Cl, —Br and —I,
heterocycloalkyl,
heterocylcoalkyl substituted with from one to three substituents independently selected from: C₁-C₆alkyl, —OCH₃, —CF₃, —F, —Cl, —Br and —I,
—B(OH)₂,
—NHS(O)₂CF₃,
—NHS(O)₂phenyl,
—NHS(O)₂CH₂-phenyl,
—S(O)₂OH,
—CH₂COOH,
—CO₂CH₃,
—CO₂CH₂CH₃,
—NH₂,
—OCH₃,
—CO₂ethyl,
—NO₂,
—NH₂,
—NHCONH₂,
—NHCOCF₃,
—C≡N,
—CH₃,
—CONH₂, and
—CF₃;
R³ is selected from:
hydrogen,
—OCH₃,
—F,
—Cl,
—Br,
—I,
pyrimidinyl,
pyrimidinyl substituted with from one to three substituents independently selected from: —OCH₃ and —OH, and —CH₃;
R⁴ is selected from: —F, —Cl, —Br, —I, —CH═CH₂, aryl, substituted aryl, heteroaryl, substituted heteroaryl, heterocycloalkyl, and substituted heterocycloalkyl; and
R⁵ is selected from: H, C₁-C₄alkyl, and —CF₃;
or a salt thereof including a pharmaceutically acceptable salt thereof;
provided the compound is not 3-{[3-(aminocarbonyl)-7-(3,5-dimethyl-4-isoxazolyl)-6-(methyloxy)-4-quinolinyl]amino}benzoic acid.

2. A compound of Formula (I), as described in claim 1, wherein:
R³ is selected from:
hydrogen, and
—OCH₃,
or a salt thereof including a pharmaceutically acceptable salt thereof.

3. A compound of Formula (I), as described in claim 1, wherein:
R⁴ is selected from: fluoro, —CH═CH₂, aryl, substituted aryl, heteroaryl, substituted heteroaryl, heterocycloalkyl, and substituted heterocycloalkyl,
where,
aryl is selected from: phenyl, naphthalene, tetrahydronaphthalene and biphenyl,
heteroaryl is selected form: pyridine, pyrazole, pyrimidine, isoxazole, oxazole, indiazole, indole, thienyl, furan, pyrazin, pyridazine, imidazole, and thiazole,
heterocycloalkyl is selected from: piperazine and pyrrolidine;
or a salt thereof including a pharmaceutically acceptable salt thereof.

4. A compound of claim 1 selected from:
5-({3-(aminocarbonyl)-7-[2,4-bis(methyloxy)-5-pyrimidinyl]-4-quinolinyl}amino)-3-biphenylcarboxylic acid;
5-({3-(aminocarbonyl)-7-[2,6-bis(methyloxy)-3-pyridinyl]-4-quinolinyl}amino)-1,3-benzenedicarboxylic acid;
3-({3-(aminosulfonyl)-7-[2,4-bis(methyloxy)-5-pyrimidinyl]-4-quinolinyl}amino)-5-cyclopentylbenzoic acid;
5-({3-(aminocarbonyl)-7-[2,4-bis(methyloxy)-5-pyrimidinyl]-4-quinolinyl}amino)-1,3-benzenedicarboxylic acid;
5-{[3-(aminocarbonyl)-7-(3,5-dimethyl-1H-pyrazol-4-yl)-4-quinolinyl]amino}-1,3-benzenedicarboxylic acid;
3-({3-(aminocarbonyl)-7-[2,4-bis(methyloxy)-5-pyrimidinyl]-4-quinolinyl}amino)benzoic acid;
5-{[3-(aminocarbonyl)-7-(3,5-dimethyl-4-isoxazolyl)-4-quinolinyl]amino}-1,3-benzenedicarboxylic acid;
3-({3-(aminocarbonyl)-7-[2,6-bis(methyloxy)-3-pyridinyl]-4-quinolinyl}amino)benzoic acid;
3-{[3-(aminocarbonyl)-7-(3,5-dimethyl-1H-pyrazol-4-yl)-6-(methyloxy)-4-quinolinyl]amino}benzoic acid;
3-{[3-(aminocarbonyl)-7-(3,5-dimethyl-1H-pyrazol-4-yl)-4-quinolinyl]amino}benzoic acid;
5-{[3-({[(3-carboxyphenyl)methyl]amino}carbonyl)-7-(3,5-dimethyl-1H-pyrazol-4-yl)-4-quinolinyl]amino}-1,3-benzenedicarboxylic acid;
3-{[3-(aminocarbonyl)-6-(methyloxy)-7-(4-pyridinyl)-4-quinolinyl]amino}benzoic acid;
3-[({[4-[(3-carboxyphenyl)amino]-7-(3,5-dimethyl-4-isoxazolyl)-3-quinolinyl]carbonyl}amino)methyl]benzoic acid;
3-{[3-(aminocarbonyl)-7-(1,3,5-trimethyl-1H-pyrazol-4-yl)-4-quinolinyl]amino}benzoic acid;
3-({7-(3,5-dimethyl-4-isoxazolyl)-3-[({[3-(trifluoromethyl)phenyl]methyl}amino)carbonyl]-4-quinolinyl}amino)benzoic acid;
3-{[3-(aminocarbonyl)-7-(3-methyl-1H-pyrazol-4-yl)-4-quinolinyl]amino}benzoic acid;
3-{[3-(aminocarbonyl)-7-(4-hydroxyphenyl)-4-quinolinyl]amino}benzoic acid;
3-{[3-(aminocarbonyl)-7-(3,5-dimethyl-4-isoxazolyl)-4-quinolinyl]amino}-5-(methyloxy)benzoic acid;
3-{[3-[({[3,5-bis(methyloxy)phenyl]methyl}amino)carbonyl]-7-(3,5-dimethyl-4-isoxazolyl)-4-quinolinyl]amino}benzoic acid;
3-{[3-(aminocarbonyl)-7-(3,5-dimethyl-1H-pyrazol-4-yl)-4-quinolinyl]amino}-5-[(methyloxy)carbonyl]benzoic acid;
3-{[3-(aminocarbonyl)-7-(3,5-dimethyl-4-isoxazolyl)-4-quinolinyl]amino}benzoic acid;
3-{[3-(aminocarbonyl)-7-(4-pyridinyl)-4-quinolinyl]amino}benzoic acid;
3-({3-(aminocarbonyl)-7-[4-(methyloxy)phenyl]-4-quinolinyl}amino)benzoic acid;
3-({3-(aminocarbonyl)-7-[2-(methyloxy)phenyl]-4-quinolinyl}amino)benzoic acid;
3-{[3-(aminocarbonyl)-7-(2-methyl-3-pyridinyl)-4-quinolinyl]amino}benzoic acid;
3-({3-(aminocarbonyl)-7-[2-chloro-6-(methyloxy)-3-pyridinyl]-4-quinolinyl}amino)benzoic acid;
3-{[3-(aminocarbonyl)-7-(2,5-difluorophenyl)-4-quinolinyl]amino}benzoic acid;
3-{[3-(aminocarbonyl)-7-(3,5-dimethyl-4-isoxazolyl)-4-quinolinyl]amino}-5-nitrobenzoic acid;
3-{[3-(aminocarbonyl)-7-(4-methyl-3-pyridinyl)-4-quinolinyl]amino}benzoic acid;
3-({3-(aminocarbonyl)-7-[2-(methyloxy)-3-pyridinyl]-4-quinolinyl}amino)benzoic acid;
3-{[3-(aminocarbonyl)-7-phenyl-4-quinolinyl]amino}benzoic acid;
3-{[3-(aminocarbonyl)-7-(1,3-oxazol-2-yl)-4-quinolinyl]amino}benzoic acid;
3-{[3-(aminocarbonyl)-7-(2-pyridinyl)-4-quinolinyl]amino}benzoic acid;
3-amino-5-{[3-(aminocarbonyl)-7-(3,5-dimethyl-4-isoxazolyl)-4-quinolinyl]amino}benzoic acid;
3-({3-(aminocarbonyl)-7-[3-(methyloxy)phenyl]-4-quinolinyl}amino)benzoic acid;
5-{[3-(aminocarbonyl)-7-(3,5-dimethyl-4-isoxazolyl)-4-quinolinyl]amino}-2-hydroxybenzoic acid;
5-{[3-(aminocarbonyl)-7-(3,5-dimethyl-4-isoxazolyl)-4-quinolinyl]amino}-2-methylbenzoic acid;
3-({3-(aminocarbonyl)-7-[2-(methyloxy)-4-pyridinyl]-4-quinolinyl}amino)benzoic acid;
3-({3-(aminocarbonyl)-7-[2,4-bis(methyloxy)phenyl]-4-quinolinyl}amino)benzoic acid;
3-{[3-(aminocarbonyl)-7-(1-methyl-1H-pyrazol-4-yl)-4-quinolinyl]amino}benzoic acid;
3-({7-(3,5-dimethyl-4-isoxazolyl)-3-[({[4-(methyloxy)phenyl]methyl}amino)carbonyl]-4-quinolinyl}amino)benzoic acid;
3-{[3-(aminocarbonyl)-7-(3,5-dimethyl-4-isoxazolyl)-4-quinolinyl]amino}-4-(methyloxy)benzoic acid;
3-{[3-(aminocarbonyl)-6-(methyloxy)-7-phenyl-4-quinolinyl]amino}benzoic acid;
3-{[3-(aminocarbonyl)-7-(2-methyl-4-pyridinyl)-4-quinolinyl]amino}benzoic acid;
3-{[3-(aminocarbonyl)-7-(3-hydroxyphenyl)-4-quinolinyl]amino}benzoic acid;
3-({3-(aminocarbonyl)-7-[2-(1-piperazinyl)-4-pyridinyl]-4-quinolinyl}amino)benzoic acid;
3-{[3-(aminocarbonyl)-7-(1-methyl-1H-indazol-7-yl)-4-quinolinyl]amino}benzoic acid;
3-{[3-(aminocarbonyl)-7-(5-pyrimidinyl)-4-quinolinyl]amino}benzoic acid;
3-({3-(aminocarbonyl)-7-[6-(aminocarbonyl)-3-pyridinyl]-4-quinolinyl}amino)benzoic acid;
3-({3-(aminocarbonyl)-7-[2,4-bis(methyloxy)-5-pyrimidinyl]-4-quinolinyl}amino)-5-[2,3-bis(methyloxy)-4-pyridinyl]benzoic acid;
3-({3-(aminocarbonyl)-7-[2,4-bis(methyloxy)-5-pyrimidinyl]-4-quinolinyl}amino)-5-(2-furanyl)benzoic acid;
3-({3-(aminosulfonyl)-7-[2,4-bis(methyloxy)-5-pyrimidinyl]-4-quinolinyl}amino)benzoic acid;
3-({3-(aminocarbonyl)-7-[2,4-bis(methyloxy)-5-pyrimidinyl]-4-quinolinyl}amino)-5-cyclopentylbenzoic acid;
3-({3-(aminocarbonyl)-7-[2,4-bis(methyloxy)-5-pyrimidinyl]-4-quinolinyl}amino)-5-cyclohexylbenzoic acid;
3-({3-(aminocarbonyl)-7-[2,4-bis(methyloxy)-5-pyrimidinyl]-4-quinolinyl}amino)-5-[2-methyl-6-(methyloxy)-4-pyridinyl]benzoic acid;
3-({3-(aminocarbonyl)-7-[2,4-bis(methyloxy)-5-pyrimidinyl]-4-quinolinyl}amino)-5-(2-methylpropyl)benzoic acid;
5-({3-(aminocarbonyl)-7-[2,4-bis(methyloxy)-5-pyrimidinyl]-4-quinolinyl}amino)-3′,4′-bis(methyloxy)-3-biphenylcarboxylic acid;
3-({3-(aminocarbonyl)-7-[2,4-bis(methyloxy)-5-pyrimidinyl]-4-quinolinyl}amino)-5-(1,2-dimethyl-1-propen-1-yl)benzoic acid;
3-({3-(aminocarbonyl)-7-[2,4-bis(methyloxy)-5-pyrimidinyl]-4-quinolinyl}amino)-5-(1-cyclohexen-1-yl)benzoic acid;
3-({3-(aminocarbonyl)-7-[2,4-bis(methyloxy)-5-pyrimidinyl]-4-quinolinyl}amino)-5-(1,2-dimethylpropyl)benzoic acid;
3-({3-(aminocarbonyl)-7-[2,4-bis(methyloxy)-5-pyrimidinyl]-4-quinolinyl}amino)-5-(3-furanyl)benzoic acid;
3-({3-(aminocarbonyl)-7-[2,4-bis(methyloxy)-5-pyrimidinyl]-4-quinolinyl}amino)-5-(1-cyclopenten-1-yl)benzoic acid;
3-({3-(aminocarbonyl)-7-[2,4-bis(methyloxy)-5-pyrimidinyl]-4-quinolinyl}amino)-5-(2-methyl-1-propen-1-yl)benzoic acid;
3-({3-(aminocarbonyl)-7-[2,4-bis(methyloxy)-5-pyrimidinyl]-4-quinolinyl}amino)-5-ethylbenzoic acid;
3-({3-(aminocarbonyl)-7-[2,4-bis(methyloxy)-5-pyrimidinyl]-4-quinolinyl}amino)-5-(1-methylethyl)benzoic acid;
3-({3-(aminocarbonyl)-7-[2,4-bis(methyloxy)-5-pyrimidinyl]-4-quinolinyl}amino)-5-(3,6-dihydro-2H-pyran-4-yl)benzoic acid;
3-({3-(aminocarbonyl)-7-[2,4-bis(methyloxy)-5-pyrimidinyl]-4-quinolinyl}amino)-5-[2-(methyloxy)-6-(trifluoromethyl)-4-pyridinyl]benzoic acid;
3-({3-(aminocarbonyl)-7-[2,4-bis(methyloxy)-5-pyrimidinyl]-4-quinolinyl}amino)-5-(tetrahydro-2H-pyran-4-yl)benzoic acid;
3-({3-(aminocarbonyl)-7-[2,4-bis(methyloxy)-5-pyrimidinyl]-4-quinolinyl}amino)-5-(4-pyridinyl)benzoic acid;
5-({3-(aminocarbonyl)-7-[2,4-bis(methyloxy)-5-pyrimidinyl]-4-quinolinyl}amino)-4′-(1,1-dimethylethyl)-3-biphenylcarboxylic acid;
5-({3-(aminocarbonyl)-7-[2,4-bis(methyloxy)-5-pyrimidinyl]-4-quinolinyl}amino)-4′-(trifluoromethyl)-3-biphenylcarboxylic acid;
3-({3-(aminocarbonyl)-7-[2,4-bis(methyloxy)-5-pyrimidinyl]-4-quinolinyl}amino)-5-(1-methylethenyl)benzoic acid;
3-({3-(aminocarbonyl)-7-[2,4,6-tris(methyloxy)-5-pyrimidinyl]-4-quinolinyl}amino)benzoic acid;
7-[2,4-bis(methyloxy)-5-pyrimidinyl]-4-[(3-(2-furanyl)-5-{[(trifluoromethyl)sulfonyl]amino}phenyl)amino]-3-quinolinecarboxamide;
methyl 3-({3-(aminocarbonyl)-7-[2,4-bis(methyloxy)-5-pyrimidinyl]-4-quinolinyl}amino)-5-{[(trifluoromethyl)sulfonyl]amino}benzoate;
7-[2,4-bis(methyloxy)-5-pyrimidinyl]-4-[(5-{[(trifluoromethyl)sulfonyl]amino}-3-biphenylyl)amino]-3-quinolinecarboxamide;
3-({3-(aminocarbonyl)-7-[2,4-bis(ethyloxy)-5-pyrimidinyl]-4-quinolinyl}amino)benzoic acid;
7-[2,4-bis(methyloxy)-5-pyrimidinyl]-4-[(3-{[(trifluoromethyl)sulfonyl]amino}phenyl)amino]-3-quinolinecarboxamide;
7-[2,4-bis(methyloxy)-5-pyrimidinyl]-4-[(3-(3-furanyl)-5-{[(trifluoromethyl)sulfonyl]amino}phenyl)amino]-3-quinolinecarboxamide;
5-({3-(aminocarbonyl)-7-[2,4-bis(methyloxy)-5-pyrimidinyl]-4-quinolinyl}amino)-4′-chloro-3-biphenylcarboxylic acid;
5-({3-(aminocarbonyl)-7-[2,4-bis(methyloxy)-5-pyrimidinyl]-4-quinolinyl}amino)-2-(3-furanyl)benzoic acid;
3-{[3′-(aminocarbonyl)-2-(methyloxy)-3,7′-biquinolin-4′-yl]amino}benzoic acid;
5-({3-(aminocarbonyl)-7-[2,4-bis(methyloxy)-5-pyrimidinyl]-4-quinolinyl}amino)-2-(2-furanyl)benzoic acid;
3-[(3-(aminocarbonyl)-7-{2,4-bis[(phenylmethyl)oxy]-5-pyrimidinyl}-4-quinolinyl)amino]benzoic acid;
4-({3-(aminocarbonyl)-7-[2,4-bis(methyloxy)-5-pyrimidinyl]-4-quinolinyl}amino)-2-biphenylcarboxylic acid;
5-{[3-(aminocarbonyl)-7-(3,5-dimethyl-4-isoxazolyl)-4-quinolinyl]amino}-3-biphenylcarboxylic acid;
3-{[3-(aminocarbonyl)-7-(3,5-dimethyl-4-isoxazolyl)-4-quinolinyl]amino}-5-(2-furanyl)benzoic acid;
3-{[3-(aminocarbonyl)-7-(2,4-dioxo-1,2,3,4-tetrahydro-5-pyrimidinyl)-4-quinolinyl]amino}benzoic acid;
3-{[3-(aminocarbonyl)-7-(2,3-dihydroxy-4-pyridinyl)-4-quinolinyl]amino}benzoic acid;
4-{3-(aminocarbonyl)-4-[(3-carboxyphenyl)amino]-7-quinolinyl}-2-pyridinecarboxylic acid;
ethyl 5-({3-(aminocarbonyl)-7-[2,4-bis(methyloxy)-5-pyrimidinyl]-4-quinolinyl}amino)-2′-fluoro-6′-(methyloxy)-3-biphenylcarboxylate;
3-({3-(aminocarbonyl)-7-[4-(methyloxy)-3-pyridinyl]-4-quinolinyl}amino)benzoic acid;
3-{[3-(aminocarbonyl)-7-(1H-pyrrolo[2,3-b]pyridin-5-yl)-4-quinolinyl]amino}benzoic acid;
3-{[3-(aminocarbonyl)-7-(3,5-dimethyl-4-isoxazolyl)-4-quinolinyl]amino}-5-[(methyloxy)carbonyl]benzoic acid;
ethyl 3-({3-(aminocarbonyl)-7-[2,4-bis(methyloxy)-5-pyrimidinyl]-4-quinolinyl}amino)benzoate;
3-{[3′-(aminocarbonyl)-3,7′-biquinolin-4′-yl]amino}benzoic acid;
3-({3-(aminocarbonyl)-7-[5-(hydroxymethyl)-1,3-dimethyl-1H-pyrazol-4-yl]-4-quinolinyl}amino)benzoic acid;
3-({3-(aminocarbonyl)-7-[5-(ethyloxy)-3-methyl-1H-pyrazol-4-yl]-4-quinolinyl}amino)benzoic acid;
3-[(3-(aminocarbonyl)-7-{2,4-bis[(1-methylethyl)oxy]-5-pyrimidinyl}-4-quinolinyl)amino]benzoic acid;
3-{[3-(aminocarbonyl)-7-(1H-indol-3-yl)-4-quinolinyl]amino}benzoic acid;
3-{[3-(aminocarbonyl)-7-(3,5-dimethyl-4-isoxazolyl)-4-quinolinyl]amino}-5-(dihydroxyboranyl)benzoic acid;
3-({3-(aminocarbonyl)-7-[2,3-bis(methyloxy)-4-pyridinyl]-4-quinolinyl}amino)benzoic acid;
4-{[3-(aminocarbonyl)-7-(3,5-dimethyl-4-isoxazolyl)-4-quinolinyl]amino}-1,2-benzenedicarboxylic acid;
3-{[3-(aminocarbonyl)-7-(3,5-dimethyl-4-isoxazolyl)-4-quinolinyl]amino}-5-chlorobenzoic acid;
3-{[3-(aminocarbonyl)-7-(3,5-dimethyl-4-isoxazolyl)-4-quinolinyl]amino}-5-(3-furanyl)benzoic acid;
3-{[3-(aminocarbonyl)-7-(3,5-dimethyl-4-isoxazolyl)-4-quinolinyl]amino}-5-iodobenzoic acid;
3-({3-(aminocarbonyl)-7-[2-methyl-6-(methyloxy)-4-pyridinyl]-4-quinolinyl}amino)benzoic acid;
3-({3-(aminocarbonyl)-7-[5-(methyloxy)-3-pyridinyl]-4-quinolinyl}amino)benzoic acid;
3-{[3-(aminocarbonyl)-7-(6-cyano-3-pyridinyl)-4-quinolinyl]amino}benzoic acid;
3-{[3-(aminocarbonyl)-7-(3,5-dimethyl-4-isoxazolyl)-4-quinolinyl]amino}-5-(trifluoromethyl)benzoic acid;
3-{[3-(aminocarbonyl)-7-(3,5-dimethyl-4-isoxazolyl)-4-quinolinyl]amino}-5-hydroxybenzoic acid;
3-{[3-(aminocarbonyl)-7-(2-chloro-6-methyl-4-pyridinyl)-4-quinolinyl]amino}benzoic acid;
3-{[3-(aminocarbonyl)-7-(4-oxo-1,4-dihydro-6-quinazolinyl)-4-quinolinyl]amino}benzoic acid;
3-{[3-(aminocarbonyl)-7-(2-oxo-1,2-dihydro-5-pyrimidinyl)-4-quinolinyl]amino}benzoic acid;
3-{[3-(aminocarbonyl)-7-(3,5-dimethyl-4-isoxazolyl)-4-quinolinyl]amino}-5-bromobenzoic acid;
3-{[3-[({[4-(acetylamino)phenyl]methyl}amino)carbonyl]-7-(3,5-dimethyl-4-isoxazolyl)-4-quinolinyl]amino}benzoic acid;
ethyl 3-({3-[(aminocarbonyl)amino]-7-[2,4-bis(methyloxy)-5-pyrimidinyl]-4-quinolinyl}amino)benzoate;
4-({3-[(aminocarbonyl)amino]phenyl}amino)-7-[2,4-bis(methyloxy)-5-pyrimidinyl]-3-quinolinecarboxamide;
methyl 3-({3-(aminocarbonyl)-7-[2,4-bis(methyloxy)-5-pyrimidinyl]-4-quinolinyl}amino)-5-cyanobenzoate;
methyl 5-({3-(aminocarbonyl)-7-[2,4-bis(methyloxy)-5-pyrimidinyl]-4-quinolinyl}amino)-2-(2-furanyl)benzoate;
methyl 4-({3-(aminocarbonyl)-7-[2,4-bis(methyloxy)-5-pyrimidinyl]-4-quinolinyl}amino)-2-biphenylcarboxylate;
3-({3-(aminocarbonyl)-7-[2,6-bis(methyloxy)-4-pyrimidinyl]-4-quinolinyl}amino)benzoic acid;
3-({3-(aminocarbonyl)-7-[2-(methyloxy)-5-pyrimidinyl]-4-quinolinyl}amino)benzoic acid;
3-{[3-(aminocarbonyl)-7-(2-chloro-1-methyl-1H-imidazol-5-yl)-4-quinolinyl]amino}benzoic acid;
3-{[3-(aminocarbonyl)-7-(2-amino-5-pyrimidinyl)-4-quinolinyl]amino}benzoic acid;
7-[2,4-bis(methyloxy)-5-pyrimidinyl]-4-({3-[(trifluoroacetyl)amino]phenyl}amino)-3-quinolinecarboxamide;
3-({3-(aminocarbonyl)-7-[2-(aminocarbonyl)-4-pyridinyl]-4-quinolinyl}amino)benzoic acid;
3-[(7-(3,5-dimethyl-4-isoxazolyl)-3-{[(2-hydroxycyclopentyl)amino]carbonyl}-4-quinolinyl)amino]benzoic acid;
7-[2,4-bis(methyloxy)-5-pyrimidinyl]-4-[(3-bromo-5-nitrophenyl)amino]-3-quinolinecarboxamide;
ethyl 3-({3-(aminocarbonyl)-7-[2-methyl-6-(methyloxy)-4-pyridinyl]-4-quinolinyl}amino)benzoate;
7-(3,5-dimethyl-4-isoxazolyl)-4-[(3-{[(trifluoromethyl)sulfonyl]amino}phenyl)amino]-3-quinolinecarboxamide;
3-{[3-(aminocarbonyl)-7-(4-amino-2-chloro-5-pyrimidinyl)-4-quinolinyl]amino}benzoic acid;
3-({3-(aminocarbonyl)-7-[2-(methyloxy)-6-(trifluoromethyl)-4-pyridinyl]-4-quinolinyl}amino)benzoic acid;
3-({3-(aminocarbonyl)-6-[2,4-bis(methyloxy)-5-pyrimidinyl]-4-quinolinyl}amino)benzoic acid;
5-{[3-(aminocarbonyl)-7-(3,5-dimethyl-4-isoxazolyl)-4-quinolinyl]amino}-2-chlorobenzoic acid;
3-{[3-(aminocarbonyl)-7-(2-cyano-5-pyrimidinyl)-4-quinolinyl]amino}benzoic acid;
3-{[3-[({[4-(aminosulfonyl)phenyl]methyl}amino)carbonyl]-7-(3,5-dimethyl-4-isoxazolyl)-4-quinolinyl]amino}benzoic acid;
3-{[3-(aminocarbonyl)-7-(3,5-dimethyl-4-isoxazolyl)-4-quinolinyl]amino}benzenesulfonic acid;
3-({3-(aminocarbonyl)-7-[4-amino-2-(methyloxy)-5-pyrimidinyl]-4-quinolinyl}amino)benzoic acid;
methyl 5-({3-(aminocarbonyl)-7-[2,4-bis(methyloxy)-5-pyrimidinyl]-4-quinolinyl}amino)-3-biphenylcarboxylate;
3-{[3-(aminocarbonyl)-7-(1,2-dimethyl-1H-imidazol-5-yl)-4-quinolinyl]amino}benzoic acid;
5-{[3-(aminocarbonyl)-7-(3,5-dimethyl-4-isoxazolyl)-4-quinolinyl]amino}-3-pyridinecarboxylic acid;
3-({3-(aminocarbonyl)-7-[4-(dimethylamino)-2-(methyloxy)-5-pyrimidinyl]-4-quinolinyl}amino)benzoic acid;
ethyl 3-[(3-(aminocarbonyl)-7-{2,4-bis[(phenylmethyl)oxy]-5-pyrimidinyl}-4-quinolinyl)amino]benzoate;
3-({2-amino-3-(aminocarbonyl)-7-[2,4-bis(methyloxy)-5-pyrimidinyl]-4-quinolinyl}amino)benzoic acid;
7-[2,4-bis(methyloxy)-5-pyrimidinyl]-4-[(3-(methyloxy)-5-{[(trifluoromethyl)sulfonyl]amino}phenyl)amino]-3-quinolinecarboxamide;
3-({3-(aminocarbonyl)-7-[2,4-bis(methyloxy)-5-pyrimidinyl]-4-quinolinyl}amino)-5-{[(trifluoromethyl)sulfonyl]amino}benzoic acid;
3-({3-(aminocarbonyl)-7-[2,4-bis(methyloxy)-5-pyrimidinyl]-4-quinolinyl}amino)-5-(4-morpholinyl)benzoic acid;
3-({3-(aminocarbonyl)-7-[2,4-bis(methyloxy)-5-pyrimidinyl]-4-quinolinyl}amino)-5-(4-methyl-1-piperazinyl)benzoic acid;
7-[2,4-bis(methyloxy)-5-pyrimidinyl]-4-[(3-(tetrahydro-3-furanyl)-5-{[(trifluoromethyl)sulfonyl]amino}phenyl)amino]-3-quinolinecarboxamide;
4-[(3-amino-5-cyclopentylphenyl)amino]-7-[2,4-bis(methyloxy)-5-pyrimidinyl]-3-quinolinecarboxamide;
3-({3-(aminocarbonyl)-7-[2,4-bis(ethyloxy)-5-pyrimidinyl]-4-quinolinyl}amino)-5-(1-cyclohexen-1-yl)benzoic acid;
3-{[3-(aminosulfonyl)-7-(3,5-dimethyl-1H-pyrazol-4-yl)-4-quinolinyl]amino}benzoic acid;
3-({3-(aminocarbonyl)-7-[2,4-bis(methyloxy)-5-pyrimidinyl]-4-quinolinyl}amino)-5-(1-piperidinyl)benzoic acid;
7-[2,4-bis(methyloxy)-5-pyrimidinyl]-4-({3-[(phenylsulfonyl)amino]phenyl}amino)-3-quinolinecarboxamide;
7-[2,4-bis(methyloxy)-5-pyrimidinyl]-4-[(3-{[(phenylmethyl)sulfonyl]amino}phenyl)amino]-3-quinolinecarboxamide; and
3-({3-(aminocarbonyl)-7-[2,4-bis(methyloxy)-5-pyrimidinyl]-2-methyl-4-quinolinyl}amino)benzoic acid;
or a salt thereof including a pharmaceutically acceptable salt thereof.

5. A compound of claim 1 selected from:
5-({3-(aminocarbonyl)-7-[2,4-bis(methyloxy)-5-pyrimidinyl]-4-quinolinyl}amino)-3-biphenylcarboxylic acid;
5-({3-(aminocarbonyl)-7-[2,6-bis(methyloxy)-3-pyridinyl]-4-quinolinyl}amino)-1,3-benzenedicarboxylic acid;
3-({3-(aminosulfonyl)-7-[2,4-bis(methyloxy)-5-pyrimidinyl]-4-quinolinyl}amino)-5-cyclopentylbenzoic acid;
3-({3-(aminosulfonyl)-7-[2,4-bis(methyloxy)-5-pyrimidinyl]-4-quinolinyl}amino)benzoic acid; and
3-({3-(aminocarbonyl)-7-[2,4-bis(methyloxy)-5-pyrimidinyl]-4-quinolinyl}amino)-5-cyclopentylbenzoic acid;
or a salt thereof including a pharmaceutically acceptable salt thereof.

6. A pharmaceutical composition comprising a compound according to claim 1 or a pharmaceutically acceptable salt thereof and a pharmaceutically acceptable excipient.

7. A method of treating or lessening the severity of cancer in a mammal in need thereof, which comprises administering to such mammal a therapeutically effective amount of a compound of Formula I, as described in claim 1 or a pharmaceutically acceptable salt thereof.

8. The method of claim 7 wherein the mammal is a human.

9. A method of treating or lessening the severity of cancer in a mammal in need thereof, which comprises administering to such mammal a therapeutically effective amount of a compound of claim 4 or a pharmaceutically acceptable salt thereof.

10. The method of claim 9 wherein the mammal is a human.

11. The method according to claim 7 wherein said cancer is selected from: brain (gliomas), glioblastomas, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

12. The method according to claim 9 wherein said cancer is selected from brain (gliomas), glioblastomas, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

13. (canceled)

14. The method of inhibiting lactate dehydrogenase A activity in a mammal in need thereof, which comprises administering to such mammal a therapeutically effective amount of a compound of Formula I, as described in claim 1 or a pharmaceutically acceptable salt thereof.

15. The method of claim 14 wherein the mammal is a human.

16. A method of treating cancer in a human in need thereof, which comprises:
administering to such human a therapeutically effective amount of
a) a compound of Formula (I), as described in claim 1 or a pharmaceutically acceptable salt thereof; and
b) at least one anti-neoplastic agent.

17. The method of claim 16, wherein the at least one anti-neoplastic agent is selected from the group consisting essentially of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors; non-receptor tyrosine kinase angiogenesis inhibitors; immunotherapeutic agents; proapoptotic agents; and cell cycle signaling inhibitors.

18-36. (canceled)

37. The method according to claim 7 wherein said cancer is selected from: brain (gliomas), glioblastomas, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid,
Lymphoblastic T cell leukemia, Chronic myelogenous leukemia, Chronic lymphocytic leukemia, Hairy-cell leukemia, acute lymphoblastic leukemia, acute myelogenous leukemia, Chronic neutrophilic leukemia, Acute lymphoblastic T cell leukemia, Plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia,
malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

38. A process for preparing a pharmaceutical composition containing a pharmaceutically acceptable excipient and an effective amount of a compound of Formula (I) as described in claim 1 or a pharmaceutically acceptable salt thereof, which process comprises bringing the compound of Formula (I) or a pharmaceutically acceptable salt thereof into association with a pharmaceutically acceptable excipient.
